SEQUENCE LISTING (1107 sequences)

SEQ ID NO: 1 - DNA, 24 bp, synthetic construct
  misc_feature 1..24: note = DNA encoding guide RNA
  source 1..24: mol_type = other DNA
ttcctcatgggtccaggatgcgtt

SEQ ID NO: 2 - DNA, 23 bp, synthetic construct
  misc_feature 1..23: note = DNA encoding guide RNA
  source 1..23: mol_type = other DNA
tacatctatatattcctcatggg

SEQ ID NO: 3 - DNA, 5094 bp, synthetic construct
  misc_feature 1..5094: note = DNA encoding nickase
  source 1..5094: mol_type = other DNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctgaccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatggtgaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcattatcccccaccagatccacctgggagagctgcacgccattctgcggcggcagggcgatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggctgagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggcggccacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgaaagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagggcaacagcgataagctgatcgccagaaagaaggactgggaccctaagaagtacggcggcttcaacagccccaccgtggcctattctgtgctggtggtggccaaagtggaaaagggcaagtccaagaaactgaagagtgtgaaagagctgctggggatcaccatcatggaaagaagcagcttcgagaagaatcccatcggctttctggaagccaagggctacaaagaagtgaaaaaggacctgatcatcaagctgcctaagtactccctgttcgagctggaaaacggccggaagagaatgctggcctctgccagcgtgctgcataagggaaacgaactggccctgccctccaaatatgtgaacttcctgtacctggccagccactatgagaagctgaagggctccagcgaggataataaacagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagccagcgccgccttcaagtactttgacaccaccatcggccggaagctgtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 4 - protein, 1695 aa, synthetic construct
  REGION 1..1695: note = Cas9 nickase
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLTIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIGFLEAKGYKEVKKDLIIKLPKYSLFELENGRKRMLASASVLHKGNELALPSKYVNFLYLASHYEKLKGSSEDNKQKQLFVEQHKHYLDEIIEQISEFSKRVILADANLDKVLSAYNKHRDKPIREQAENIIHLFTLTNLGASAAFKYFDTTIGRKLYTSTKEVLDATLIHQSITGLYETRIDLSQLGGDSGGSKRTADGSEFEPKKKRKVPVIITITIEFKPADQPRLCLLVASHLLFAPPPCLPPWKVPLPLSFPNKMRKLHRIVVGVILFWGVGWGRTARGRIGKTIAGMLGMRWALW

SEQ ID NO: 5 - DNA, 5094 bp, synthetic construct
  misc_feature 1..5094: note = DNA encoding nickase
  source 1..5094: mol_type = other DNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgtgagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccagagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaagagtacagatctaccaaggaggtgctagacgcgacactgattcaccaatccatcacgggattatatgaaactcggatagatttgtcacagcttgggggtgactctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 6 - protein, 1695 aa, synthetic construct
  REGION 1..1695: note = Cas9 nickase
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKEYRSTKEVLDATLIHQSITGLYETRIDLSQLGGDSGGSKRTADGSEFEPKKKRKVPVIITITIEFKPADQPRLCLLVASHLLFAPPPCLPPWKVPLPLSFPNKMRKLHRIVVGVILFWGVGWGRTARGRIGKTIAGMLGMRWALW

SEQ ID NO: 7 - DNA, 5161 bp, synthetic construct
  misc_feature 1..5161: note = DNA encoding nickase
  source 1..5161: mol_type = other DNA
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcgggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtccctcgggctgacccccaactttaaatctaacttcgacctggccgaagataccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagctctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaatcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgagaaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagatcagaagaaagctattgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaagcagctcaagaggcgccgatatacaggatgggggcggctgtcaagaaaactgatcaatgggatccgagacaagcagagtggaaagacaatcctggattttcttaagtccgatggatttgccaaccggaacttcattcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaaccacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaacagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggtttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggctaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgtgctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacactaccatagacagaaagcggtacacctctacaaaggaggtcctggacgccacactgattcatcagtcaattacggggctctatgaaacaagaatcgacctctctcagctcggtggagacccggcctgtacgagacacggatcgacctgtctcagctgggaggtgactctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 8 - protein, 1662 aa, synthetic construct
  REGION 1..1662: note = Cas9 nickase
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGTRSTPLGSLSAQTASAGPSLRTSTRCRAKNSKFWAIPIATARRTSLAPSCSTPGRRPKPRGSKEQHGADIPAERIGSATCRRSLVMRWLRWMTLSSIGWRSPFWWRRIKSTSATQSLAISWTRWRTMKSTQPYIIGRSLTVLIRLTCGSISRWRISNFGDTSSSRGTTQTTAMSTNSLSNWFRLTISFSKRTRSTHPELTPKQSALGCPNPGGSKTSSHSSLGRRRTACLVILSPCPSGPPTLNLTSTWPKIPSFNAKTPTMMISTICWPRSATSTQTFFWRQRTCQTPFCVIFCETRRSPKLRALVSSSMMSTTKTLCRPLSDSNCLRSTRKFSSISLKMATPDTLTAEQARRNFTNLLSPSWKKWTAPRSCWSLTEKICCANSALSTMESSPTRFTWANCTLSSGGKRISTPFKITGKRLRKSSHFGYPTMAPSPGEIPDSRGLANQKRPSLPGTSRKSWIRGPLPSPSSKGLTLIKICLTKRCFLNTLCCTSTSQFITSSPRSNTSQKGESQHSCLEIRRKLLWTSSSRRTGKLPNSSKKTISKRLNVSTLLKSAEWRIASTHPWERITISKSLKTRTSWTMRRTRTFLRTLSSPLRCLKIGRLKNAKLTLISSTTKSSSSRGADIQDGGGCQENSMGSETSRVERQSWIFLSPMDLPTGTSFSSMMTLSPLRRTSRKHKFLARGTVFTSTSLILQVAQLSKREYCRPLRSWMNSSKWEGISPRISLSRWPERTKPPRRDRRTVGKGRGLKRVKNWGPKSLRNTQLKTPSFRMRSSTCTTCRTAGTCTWIRNWTSIGSPTTTWIISCPSLFSKMILLIIKCQDPIKTEGRVITSPQKKLSRKKIIGGSCTPNSHNGSSIILRLNEVACLSWIKPVSSKGSLLRHARSPSTWPKFSIHATPSTMKMTNFERKLLLSLSWSQISERTFSFIRERSTITTMRMMPTMQWALHLSKNIPSLNLNLFTETIKCTMLGKSQSLSRKARPPLSTSFTAILIFSRPRLHWPMERFGSDHLSKQTEKQEKSCGTRVGISRQSGRSCPCRRTSLKRPKYRPEASPRKVSSRKGTATSSHAKKIGTPRNTADSILLQSLTVYWLWLKWRKGSLKNSKASRNCWASQSWSDQASKKTPSTFSRRKDIKRSKKTSSLSFPSTLSLSLKTAGNECSLVRACCRKVTSWHCPLNTLISCIWPATMKSSKGLPKIMSRSSCSWNNTNTTLMRSSSKANSPKESSPTLTSIRCFLLTISTGISPSGSRQKTLSTCLLPTWARLQPSSTSTLPTESGTPLQRRSWTPHFISQLRGSMKQESTSLSSVEPACTRHGSTCLSWEVTLAAQKEPPTAANSSPRRRGKSNRSSSPSPLSLNPLISLDCAFLPAICCLPLPRAFLDPGRCHSHCPFLIKGNCIALSEVSFYSGGWGGAGQQGGGLGRQQACWGCGGLY

SEQ ID NO: 9 - DNA, 22 bp, synthetic construct
  misc_feature 1..22: note = Structural abnormalities observed in CRISPR/Cas9-edited Jurkat T cells
  misc_feature 20
  source 1..22: mol_type = other DNA
ttacatctatatattcctcngg

SEQ ID NO: 10 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattgctttgg

SEQ ID NO: 11 - DNA, 22 bp, synthetic construct
  misc_feature 1..22: note = Structural abnormalities observed in CRISPR/Cas9-edited Jurkat T cells
  source 1..22: mol_type = other DNA
cttcagctatatcttcctctgg

SEQ ID NO: 12 - DNA, 22 bp, synthetic construct
  misc_feature 1..22: note = Structural abnormalities observed in CRISPR/Cas9-edited Jurkat T cells
  source 1..22: mol_type = other DNA
ttacatctatagatcactttgg

SEQ ID NO: 13 - DNA, 22 bp, synthetic construct
  misc_feature 1..22: note = Structural abnormalities observed in CRISPR/Cas9-edited Jurkat T cells
  source 1..22: mol_type = other DNA
ttacctccaaatgttcctctgg

SEQ ID NO: 14 - DNA, 22 bp, synthetic construct
  misc_feature 1..22: note = Structural abnormalities observed in CRISPR/Cas9-edited Jurkat T cells
  source 1..22: mol_type = other DNA
ttatatcaatataatcatctgg

SEQ ID NO: 15 - DNA, 22 bp, synthetic construct
  misc_feature 1..22: note = Structural abnormalities observed in CRISPR/Cas9-edited Jurkat T cells
  source 1..22: mol_type = other DNA
ttacctccaaatgttcctctgg

SEQ ID NO: 16 - DNA, 22 bp, synthetic construct
  misc_feature 1..22: note = Structural abnormalities observed in CRISPR/Cas9-edited Jurkat T cells
  source 1..22: mol_type = other DNA
ttacatgtatatatacttttgg

SEQ ID NO: 17 - DNA, 22 bp, synthetic construct
  misc_feature 1..22: note = Structural abnormalities observed in CRISPR/Cas9-edited Jurkat T cells
  source 1..22: mol_type = other DNA
ttttttctatctattcctcagg

SEQ ID NO: 18 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 19 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 20 - DNA, 22 bp, synthetic construct
  misc_feature 1..22: note = Structural abnormalities observed in CRISPR/Cas9-edited Jurkat T cells
  source 1..22: mol_type = other DNA
taacaaccatctattcctctgg

SEQ ID NO: 21 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 22 - DNA, 22 bp, synthetic construct
  misc_feature 1..22: note = Structural abnormalities observed in CRISPR/Cas9-edited Jurkat T cells
  source 1..22: mol_type = other DNA
ttacatgtatacatttcccggg

SEQ ID NO: 23 - DNA, 22 bp, synthetic construct
  misc_feature 1..22: note = Structural abnormalities observed in CRISPR/Cas9-edited Jurkat T cells
  source 1..22: mol_type = other DNA
tttaatttatacattcctcagg

SEQ ID NO: 24 - DNA, 22 bp, synthetic construct
  misc_feature 1..22: note = Structural abnormalities observed in CRISPR/Cas9-edited Jurkat T cells
  source 1..22: mol_type = other DNA
ttacaactattaattcctaggg

SEQ ID NO: 25 - DNA, 22 bp, synthetic construct
  misc_feature 1..22: note = Structural abnormalities observed in CRISPR/Cas9-edited Jurkat T cells
  source 1..22: mol_type = other DNA
tgacatataattattcctctgg

SEQ ID NO: 26 - DNA, 22 bp, synthetic construct
  misc_feature 1..22: note = Structural abnormalities observed in CRISPR/Cas9-edited Jurkat T cells
  source 1..22: mol_type = other DNA
ttatatctatagattcttttgg

SEQ ID NO: 27 - DNA, 22 bp, synthetic construct
  misc_feature 1..22: note = Structural abnormalities observed in CRISPR/Cas9-edited Jurkat T cells
  source 1..22: mol_type = other DNA
tttcctctatttcttcctcagg

SEQ ID NO: 28 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctctgg

SEQ ID NO: 29 - DNA, 22 bp, synthetic construct
  misc_feature 1..22: note = Structural abnormalities observed in CRISPR/Cas9-edited Jurkat T cells
  source 1..22: mol_type = other DNA
tgtcatctatatattcctaggg

SEQ ID NO: 30 - DNA, 22 bp, synthetic construct
  misc_feature 1..22: note = Structural abnormalities observed in CRISPR/Cas9-edited Jurkat T cells
  source 1..22: mol_type = other DNA
ttacatctataaattaccttgg

SEQ ID NO: 31 - DNA, 22 bp, synthetic construct
  misc_feature 1..22: note = Structural abnormalities observed in CRISPR/Cas9-edited Jurkat T cells
  source 1..22: mol_type = other DNA
ttacatctataaattaccttgg

SEQ ID NO: 32 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 33 - DNA, 22 bp, synthetic construct
  misc_feature 1..22: note = Structural abnormalities observed in CRISPR/Cas9-edited Jurkat T cells
  source 1..22: mol_type = other DNA
ttacatatatttattgctatgg

SEQ ID NO: 34 - DNA, 22 bp, synthetic construct
  misc_feature 1..22: note = Structural abnormalities observed in CRISPR/Cas9-edited Jurkat T cells
  source 1..22: mol_type = other DNA
ttacatctatatactctccagg

SEQ ID NO: 35 - DNA, 22 bp, synthetic construct
  misc_feature 1..22: note = Structural abnormalities observed in CRISPR/Cas9-edited Jurkat T cells
  source 1..22: mol_type = other DNA
ttatctctagatattccactgg

SEQ ID NO: 36 - DNA, 22 bp, synthetic construct
  misc_feature 1..22: note = Structural abnormalities observed in CRISPR/Cas9-edited Jurkat T cells
  source 1..22: mol_type = other DNA
atacatctatactttcttcagg

SEQ ID NO: 37 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 38 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 39 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattgctctgg

SEQ ID NO: 40 - DNA, 22 bp, synthetic construct
  misc_feature 1..22: note = Structural abnormalities observed in CRISPR/Cas9-edited Jurkat T cells
  source 1..22: mol_type = other DNA
ctacatctatgcattcttctgg

SEQ ID NO: 41 - DNA, 22 bp, synthetic construct
  misc_feature 1..22: note = Structural abnormalities observed in CRISPR/Cas9-edited Jurkat T cells
  source 1..22: mol_type = other DNA
ttgcttctatatattccaaggg

SEQ ID NO: 42 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 43 - DNA, 22 bp, synthetic construct
  misc_feature 1..22: note = Structural abnormalities observed in CRISPR/Cas9-edited Jurkat T cells
  source 1..22: mol_type = other DNA
ttatatctataaattgctatgg

SEQ ID NO: 44 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 45 - DNA, 22 bp, synthetic construct
  misc_feature 1..22: note = Structural abnormalities observed in CRISPR/Cas9-edited Jurkat T cells
  source 1..22: mol_type = other DNA
tttcttatatagattcctctgg

SEQ ID NO: 46 - DNA, 22 bp, synthetic construct
  misc_feature 1..22: note = Structural abnormalities observed in CRISPR/Cas9-edited Jurkat T cells
  source 1..22: mol_type = other DNA
ttccagatatatattcatctgg

SEQ ID NO: 47 - DNA, 22 bp, synthetic construct
  misc_feature 1..22: note = Structural abnormalities observed in CRISPR/Cas9-edited Jurkat T cells
  source 1..22: mol_type = other DNA
taaaatttatatattccttggg

SEQ ID NO: 48 - DNA, 22 bp, synthetic construct
  misc_feature 1..22: note = Structural abnormalities observed in CRISPR/Cas9-edited Jurkat T cells
  source 1..22: mol_type = other DNA
gtcaatctatatattcttctgg

SEQ ID NO: 49 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattgctttgg

SEQ ID NO: 50 - DNA, 22 bp, synthetic construct
  misc_feature 1..22: note = Structural abnormalities observed in CRISPR/Cas9-edited Jurkat T cells
  source 1..22: mol_type = other DNA
caacatctctatattcctgtgg

SEQ ID NO: 51 - DNA, 22 bp, synthetic construct
  misc_feature 1..22: note = Structural abnormalities observed in CRISPR/Cas9-edited Jurkat T cells
  source 1..22: mol_type = other DNA
ttacttctttttcttcctcagg

SEQ ID NO: 52 - DNA, 22 bp, synthetic construct
  misc_feature 1..22: note = Structural abnormalities observed in CRISPR/Cas9-edited Jurkat T cells
  source 1..22: mol_type = other DNA
ttgcttctttattttcctctgg

SEQ ID NO: 53 - DNA, 22 bp, synthetic construct
  misc_feature 1..22: note = Structural abnormalities observed in CRISPR/Cas9-edited Jurkat T cells
  source 1..22: mol_type = other DNA
ttacatctgcatcctcctctgg

SEQ ID NO: 54 - DNA, 22 bp, synthetic construct
  misc_feature 1..22: note = Structural abnormalities observed in CRISPR/Cas9-edited Jurkat T cells
  source 1..22: mol_type = other DNA
ttccatctctcttttcctctgg

SEQ ID NO: 55 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 56 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 57 - DNA, 22 bp, synthetic construct
  misc_feature 1..22: note = Structural abnormalities observed in CRISPR/Cas9-edited Jurkat T cells
  source 1..22: mol_type = other DNA
ttatgtatatatattccttagg

SEQ ID NO: 58 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 59 - DNA, 22 bp, synthetic construct
  misc_feature 1..22: note = Structural abnormalities observed in CRISPR/Cas9-edited Jurkat T cells
  source 1..22: mol_type = other DNA
ttgtatctataaattcctttgg

SEQ ID NO: 60 - DNA, 22 bp, synthetic construct
  misc_feature 1..22: note = Structural abnormalities observed in CRISPR/Cas9-edited Jurkat T cells
  source 1..22: mol_type = other DNA
tttcatctttatttttctctgg

SEQ ID NO: 61 - DNA, 22 bp, synthetic construct
  misc_feature 1..22: note = Structural abnormalities observed in CRISPR/Cas9-edited Jurkat T cells
  source 1..22: mol_type = other DNA
ttacttgtatatattcctgggg

SEQ ID NO: 62 - DNA, 22 bp, synthetic construct
  misc_feature 1..22: note = Structural abnormalities observed in CRISPR/Cas9-edited Jurkat T cells
  source 1..22: mol_type = other DNA
ctacatctgtacattccccagg

SEQ ID NO: 63 - DNA, 22 bp, synthetic construct
  misc_feature 1..22: note = Structural abnormalities observed in CRISPR/Cas9-edited Jurkat T cells
  source 1..22: mol_type = other DNA
tttcatctctataatcctcagg

SEQ ID NO: 64 - DNA, 22 bp, synthetic construct
  misc_feature 1..22: note = Structural abnormalities observed in CRISPR/Cas9-edited Jurkat T cells
  source 1..22: mol_type = other DNA
ttacctccaaatgttcctctgg

SEQ ID NO: 65 - DNA, 22 bp, synthetic construct
  misc_feature 1..22: note = Structural abnormalities observed in CRISPR/Cas9-edited Jurkat T cells
  source 1..22: mol_type = other DNA
ttacctccaaatgttcctctgg

SEQ ID NO: 66 - DNA, 22 bp, synthetic construct
  misc_feature 1..22: note = Structural abnormalities observed in CRISPR/Cas9-edited Jurkat T cells
  source 1..22: mol_type = other DNA
ttacctccaaatgttcctctgg

SEQ ID NO: 67 - DNA, 22 bp, synthetic construct
  misc_feature 1..22: note = Structural abnormalities observed in CRISPR/Cas9-edited Jurkat T cells
  source 1..22: mol_type = other DNA
atagatctacatatttctcagg

SEQ ID NO: 68 - DNA, 22 bp, synthetic construct
  misc_feature 1..22: note = Structural abnormalities observed in CRISPR/Cas9-edited Jurkat T cells
  source 1..22: mol_type = other DNA
ttacctccaaatgttcctctgg

SEQ ID NO: 69 - DNA, 22 bp, synthetic construct
  misc_feature 1..22: note = Structural abnormalities observed in CRISPR/Cas9-edited Jurkat T cells
  source 1..22: mol_type = other DNA
ttacctccaaatgttcctctgg

SEQ ID NO: 70 - DNA, 22 bp, synthetic construct
  misc_feature 1..22: note = Structural abnormalities observed in CRISPR/Cas9-edited Jurkat T cells
  source 1..22: mol_type = other DNA
atactttaatatattcctctgg

SEQ ID NO: 71 - DNA, 22 bp, synthetic construct
  misc_feature 1..22: note = Structural abnormalities observed in CRISPR/Cas9-edited Jurkat T cells
  source 1..22: mol_type = other DNA
ttatatttatatatttttctgg

SEQ ID NO: 72 - DNA, 22 bp, synthetic construct
  misc_feature 1..22: note = Structural abnormalities observed in CRISPR/Cas9-edited Jurkat T cells
  source 1..22: mol_type = other DNA
ttaaatctatgaattcctgggg

SEQ ID NO: 73 - DNA, 22 bp, synthetic construct
  misc_feature 1..22: note = Structural abnormalities observed in CRISPR/Cas9-edited Jurkat T cells
  source 1..22: mol_type = other DNA
ttagatctgtagattcctttgg

SEQ ID NO: 74 - DNA, 22 bp, synthetic construct
  misc_feature 1..22: note = Structural abnormalities observed in CRISPR/Cas9-edited Jurkat T cells
  source 1..22: mol_type = other DNA
atatatatatatattcctatgg

SEQ ID NO: 75 - DNA, 22 bp, synthetic construct
  misc_feature 1..22: note = Structural abnormalities observed in CRISPR/Cas9-edited Jurkat T cells
  source 1..22: mol_type = other DNA
atatatatatatattcatcagg

SEQ ID NO: 76 - DNA, 22 bp, synthetic construct
  misc_feature 1..22: note = Structural abnormalities observed in CRISPR/Cas9-edited Jurkat T cells
  source 1..22: mol_type = other DNA
ttagatctataaattactttgg

SEQ ID NO: 77 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 78 - DNA, 22 bp, synthetic construct
  misc_feature 1..22: note = Structural abnormalities observed in CRISPR/Cas9-edited Jurkat T cells
  source 1..22: mol_type = other DNA
tgtcatctatatcttcttctgg

SEQ ID NO: 79 - DNA, 22 bp, synthetic construct
  misc_feature 1..22: note = Structural abnormalities observed in CRISPR/Cas9-edited Jurkat T cells
  source 1..22: mol_type = other DNA
ttacagctctataatactctgg

SEQ ID NO: 80 - DNA, 22 bp, synthetic construct
  misc_feature 1..22: note = Structural abnormalities observed in CRISPR/Cas9-edited Jurkat T cells
  source 1..22: mol_type = other DNA
ttacatttatttatgcttcagg

SEQ ID NO: 81 - DNA, 22 bp, synthetic construct
  misc_feature 1..22: note = Structural abnormalities observed in CRISPR/Cas9-edited Jurkat T cells
  source 1..22: mol_type = other DNA
ttataactaaatattactctgg

SEQ ID NO: 82 - DNA, 22 bp, synthetic construct
  misc_feature 1..22: note = Structural abnormalities observed in CRISPR/Cas9-edited Jurkat T cells
  source 1..22: mol_type = other DNA
ttacatgtatatattaatatgg

SEQ ID NO: 83 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattgctttgg

SEQ ID NO: 84 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattacttagg

SEQ ID NO: 85 - DNA, 22 bp, synthetic construct
  misc_feature 1..22: note = Structural abnormalities observed in CRISPR/Cas9-edited Jurkat T cells
  source 1..22: mol_type = other DNA
tttcatctttggattcctcagg

SEQ ID NO: 86 - DNA, 22 bp, synthetic construct
  misc_feature 1..22: note = Structural abnormalities observed in CRISPR/Cas9-edited Jurkat T cells
  source 1..22: mol_type = other DNA
tttcatttatatatccatcagg

SEQ ID NO: 87 - DNA, 22 bp, synthetic construct
  misc_feature 1..22: note = Structural abnormalities observed in CRISPR/Cas9-edited Jurkat T cells
  source 1..22: mol_type = other DNA
tttcagctatgtcttcctcggg

SEQ ID NO: 88 - DNA, 22 bp, synthetic construct
  misc_feature 1..22: note = Structural abnormalities observed in CRISPR/Cas9-edited Jurkat T cells
  source 1..22: mol_type = other DNA
ttaaatctatatattgctttgg

SEQ ID NO: 89 - DNA, 22 bp, synthetic construct
  misc_feature 1..22: note = Structural abnormalities observed in CRISPR/Cas9-edited Jurkat T cells
  source 1..22: mol_type = other DNA
ataaatctatatattgctttgg

SEQ ID NO: 90 - DNA, 22 bp, synthetic construct
  misc_feature 1..22: note = Structural abnormalities observed in CRISPR/Cas9-edited Jurkat T cells
  source 1..22: mol_type = other DNA
tttcatctatgtatccatcagg

SEQ ID NO: 91 - DNA, 22 bp, synthetic construct
  misc_feature 1..22: note = Structural abnormalities observed in CRISPR/Cas9-edited Jurkat T cells
  source 1..22: mol_type = other DNA
ttacatctgtatattcaactgg

SEQ ID NO: 92 - DNA, 22 bp, synthetic construct
  misc_feature 1..22: note = Structural abnormalities observed in CRISPR/Cas9-edited Jurkat T cells
  source 1..22: mol_type = other DNA
ttgaatctatagattgctctgg

SEQ ID NO: 93 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattgctttgg

SEQ ID NO: 94 - DNA, 22 bp, synthetic construct
  misc_feature 1..22: note = Structural abnormalities observed in CRISPR/Cas9-edited Jurkat T cells
  source 1..22: mol_type = other DNA
ttactttgatatattccacagg

SEQ ID NO: 95 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 96 - DNA, 22 bp, synthetic construct
  misc_feature 1..22: note = Structural abnormalities observed in CRISPR/Cas9-edited Jurkat T cells
  source 1..22: mol_type = other DNA
taacatatatacatacctcagg

SEQ ID NO: 97 - DNA, 22 bp, synthetic construct
  misc_feature 1..22: note = Structural abnormalities observed in CRISPR/Cas9-edited Jurkat T cells
  source 1..22: mol_type = other DNA
ttccaactatatggtcctctgg

SEQ ID NO: 98 - DNA, 22 bp, synthetic construct
  misc_feature 1..22: note = Structural abnormalities observed in CRISPR/Cas9-edited Jurkat T cells
  source 1..22: mol_type = other DNA
ttacttctatatcttcttatgg

SEQ ID NO: 99 - DNA, 22 bp, synthetic construct
  misc_feature 1..22: note = Structural abnormalities observed in CRISPR/Cas9-edited Jurkat T cells
  source 1..22: mol_type = other DNA
ttacacatatacatttctcagg

SEQ ID NO: 100 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 101 - DNA, 22 bp, synthetic construct
  misc_feature 1..22: note = Structural abnormalities observed in CRISPR/Cas9-edited Jurkat T cells
  source 1..22: mol_type = other DNA
ttatatctattttttcctatgg

SEQ ID NO: 102 - DNA, 22 bp, synthetic construct
  misc_feature 1..22: note = Structural abnormalities observed in CRISPR/Cas9-edited Jurkat T cells
  source 1..22: mol_type = other DNA
ctacaactttttattcctctgg

SEQ ID NO: 103 - DNA, 22 bp, synthetic construct
  misc_feature 1..22: note = Structural abnormalities observed in CRISPR/Cas9-edited Jurkat T cells
  source 1..22: mol_type = other DNA
ttacatctgtagattgctttgg

SEQ ID NO: 104 - DNA, 22 bp, synthetic construct
  misc_feature 1..22: note = Structural abnormalities observed in CRISPR/Cas9-edited Jurkat T cells
  source 1..22: mol_type = other DNA
tcacatttatataatgctctgg

SEQ ID NO: 105 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 106 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactctgg

SEQ ID NO: 107 - DNA, 22 bp, synthetic construct
  misc_feature 1..22: note = Structural abnormalities observed in CRISPR/Cas9-edited Jurkat T cells
  source 1..22: mol_type = other DNA
ttacatctttatattattgagg

SEQ ID NO: 108 - DNA, 22 bp, synthetic construct
  misc_feature 1..22: note = Structural abnormalities observed in CRISPR/Cas9-edited Jurkat T cells
  source 1..22: mol_type = other DNA
ttacctttatattttcatcagg

SEQ ID NO: 109 - DNA, 22 bp, synthetic construct
  misc_feature 1..22: note = Structural abnormalities observed in CRISPR/Cas9-edited Jurkat T cells
  source 1..22: mol_type = other DNA
ttacctttatattttcatcagg

SEQ ID NO: 110 - DNA, 22 bp, synthetic construct
  misc_feature 1..22: note = Structural abnormalities observed in CRISPR/Cas9-edited Jurkat T cells
  source 1..22: mol_type = other DNA
ttacctttatattttcatcagg

SEQ ID NO: 111 - DNA, 22 bp, synthetic construct
  misc_feature 1..22: note = Structural abnormalities observed in CRISPR/Cas9-edited Jurkat T cells
  source 1..22: mol_type = other DNA
ttacctttatattttcatcagg

SEQ ID NO: 112 - DNA, 22 bp, synthetic construct
  misc_feature 1..22: note = Structural abnormalities observed in CRISPR/Cas9-edited Jurkat T cells
  source 1..22: mol_type = other DNA
tcacatctatagcttgctcagg

SEQ ID NO: 113 - DNA, 22 bp, synthetic construct
  misc_feature 1..22: note = Structural abnormalities observed in CRISPR/Cas9-edited Jurkat T cells
  source 1..22: mol_type = other DNA
gaacatctatatattcatttgg

SEQ ID NO: 114 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 115 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattgctttgg

SEQ ID NO: 116 - DNA, 22 bp, synthetic construct
  misc_feature 1..22: note = Structural abnormalities observed in CRISPR/Cas9-edited Jurkat T cells
  source 1..22: mol_type = other DNA
taatatatatatattcccctgg

SEQ ID NO: 117 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattgctttgg

SEQ ID NO: 118 - DNA, 22 bp, synthetic construct
  misc_feature 1..22: note = Structural abnormalities observed in CRISPR/Cas9-edited Jurkat T cells
  source 1..22: mol_type = other DNA
ttacatctatgacttcatctgg

SEQ ID NO: 119 - DNA, 22 bp, synthetic construct
  misc_feature 1..22: note = Structural abnormalities observed in CRISPR/Cas9-edited Jurkat T cells
  source 1..22: mol_type = other DNA
ttaaatctttatattgctttgg

SEQ ID NO: 120 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 121 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 122 - DNA, 22 bp, synthetic construct
  misc_feature 1..22: note = Structural abnormalities observed in CRISPR/Cas9-edited Jurkat T cells
  source 1..22: mol_type = other DNA
ttacatctaaatattcaaaagg

SEQ ID NO: 123 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 124 - DNA, 22 bp, synthetic construct
  misc_feature 1..22: note = Structural abnormalities observed in CRISPR/Cas9-edited Jurkat T cells
  source 1..22: mol_type = other DNA
taacttctacgtattcctcagg

SEQ ID NO: 125 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattcctttgg

SEQ ID NO: 126 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 127 - DNA, 22 bp, synthetic construct
  misc_feature 1..22: note = Structural abnormalities observed in CRISPR/Cas9-edited Jurkat T cells
  source 1..22: mol_type = other DNA
tttcatctctatattcctaggg

SEQ ID NO: 128 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 129 - DNA, 22 bp, synthetic construct
  misc_feature 1..22: note = Structural abnormalities observed in CRISPR/Cas9-edited Jurkat T cells
  source 1..22: mol_type = other DNA
ttacatttatatatacttacgg

SEQ ID NO: 130 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 131 - DNA, 22 bp, synthetic construct
  misc_feature 1..22: note = Structural abnormalities observed in CRISPR/Cas9-edited Jurkat T cells
  source 1..22: mol_type = other DNA
ttgaatctatagattgctctgg

SEQ ID NO: 132 - DNA, 22 bp, synthetic construct
  misc_feature 1..22: note = Structural abnormalities observed in CRISPR/Cas9-edited Jurkat T cells
  source 1..22: mol_type = other DNA
atacatcaaaattttcctctgg

SEQ ID NO: 133 - DNA, 22 bp, synthetic construct
  misc_feature 1..22: note = Structural abnormalities observed in CRISPR/Cas9-edited Jurkat T cells
  source 1..22: mol_type = other DNA
ttacatctataatctccttggg

SEQ ID NO: 134 - DNA, 22 bp, synthetic construct
  misc_feature 1..22: note = Structural abnormalities observed in CRISPR/Cas9-edited Jurkat T cells
  source 1..22: mol_type = other DNA
atacatgtatatattcataagg

SEQ ID NO: 135 - DNA, 22 bp, synthetic construct
  misc_feature 1..22: note = Structural abnormalities observed in CRISPR/Cas9-edited Jurkat T cells
  source 1..22: mol_type = other DNA
ttacaactaaatgttcttcagg

SEQ ID NO: 136 - DNA, 22 bp, synthetic construct
  misc_feature 1..22: note = Structural abnormalities observed in CRISPR/Cas9-edited Jurkat T cells
  source 1..22: mol_type = other DNA
ttatatgtctatattcatctgg

SEQ ID NO: 137 - DNA, 22 bp, synthetic construct
  misc_feature 1..22: note = Structural abnormalities observed in CRISPR/Cas9-edited Jurkat T cells
  source 1..22: mol_type = other DNA
ttatatatatatatttctgggg

SEQ ID NO: 138 - DNA, 22 bp, synthetic construct
  misc_feature 1..22: note = Structural abnormalities observed in CRISPR/Cas9-edited Jurkat T cells
  source 1..22: mol_type = other DNA
tgacatctatatagtactgagg

SEQ ID NO: 139 - DNA, 22 bp, synthetic construct
  misc_feature 1..22: note = Structural abnormalities observed in CRISPR/Cas9-edited Jurkat T cells
  source 1..22: mol_type = other DNA
ttacatccacttatttctcagg

SEQ ID NO: 140 - DNA, 22 bp, synthetic construct
  misc_feature 1..22: note = Structural abnormalities observed in CRISPR/Cas9-edited Jurkat T cells
  source 1..22: mol_type = other DNA
gtacatctctatatttcccagg

SEQ ID NO: 141 - DNA, 22 bp, synthetic construct
  misc_feature 1..22: note = Structural abnormalities observed in CRISPR/Cas9-edited Jurkat T cells
  source 1..22: mol_type = other DNA
atatatatatatatttctctgg

SEQ ID NO: 142 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 143 - DNA, 22 bp, synthetic construct
  misc_feature 1..22: note = Structural abnormalities observed in CRISPR/Cas9-edited Jurkat T cells
  source 1..22: mol_type = other DNA
atacatctttaaattccactgg

SEQ ID NO: 144 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 145 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 146 - DNA, 22 bp, synthetic construct
  misc_feature 1..22: note = Structural abnormalities observed in CRISPR/Cas9-edited Jurkat T cells
  source 1..22: mol_type = other DNA
ttacaactttattttccttagg

SEQ ID NO: 147 - DNA, 22 bp, synthetic construct
  misc_feature 1..22: note = Structural abnormalities observed in CRISPR/Cas9-edited Jurkat T cells
  source 1..22: mol_type = other DNA
tttcttctaattattcctctgg

SEQ ID NO: 148 - DNA, 22 bp, synthetic construct
  misc_feature 1..22: note = Structural abnormalities observed in CRISPR/Cas9-edited Jurkat T cells
  source 1..22: mol_type = other DNA
ttacctctatattttcttctgg

SEQ ID NO: 149 - DNA, 22 bp, synthetic construct
  misc_feature 1..22: note = Structural abnormalities observed in CRISPR/Cas9-edited Jurkat T cells
  source 1..22: mol_type = other DNA
ttacatcaatataatccaatgg

SEQ ID NO: 150 - DNA, 22 bp, synthetic construct
  misc_feature 1..22: note = Structural abnormalities observed in CRISPR/Cas9-edited Jurkat T cells
  source 1..22: mol_type = other DNA
ttaaatctatatattgttctgg

SEQ ID NO: 151 - DNA, 22 bp, synthetic construct
  misc_feature 1..22: note = Structural abnormalities observed in CRISPR/Cas9-edited Jurkat T cells
  source 1..22: mol_type = other DNA
ttaaatctacatattactctgg

SEQ ID NO: 152 - DNA, 22 bp, synthetic construct
  misc_feature 1..22: note = Structural abnormalities observed in CRISPR/Cas9-edited Jurkat T cells
  source 1..22: mol_type = other DNA
ttccatgtatacattgctctgg

SEQ ID NO: 153 - DNA, 22 bp, synthetic construct
  misc_feature 1..22: note = Structural abnormalities observed in CRISPR/Cas9-edited Jurkat T cells
  source 1..22: mol_type = other DNA
ttatatctatatgctcatcagg

SEQ ID NO: 154 - DNA, 22 bp, synthetic construct
  misc_feature 1..22: note = Structural abnormalities observed in CRISPR/Cas9-edited Jurkat T cells
  source 1..22: mol_type = other DNA
ttacatttataaattacccagg

SEQ ID NO: 155 - DNA, 22 bp, synthetic construct
  misc_feature 1..22: note = Structural abnormalities observed in CRISPR/Cas9-edited Jurkat T cells
  source 1..22: mol_type = other DNA
atatatctttatattccccagg

SEQ ID NO: 156 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtagattcctttgg

SEQ ID NO: 157 - DNA, 22 bp, synthetic construct
  misc_feature 1..22: note = Structural abnormalities observed in CRISPR/Cas9-edited Jurkat T cells
  source 1..22: mol_type = other DNA
ttagaactacattttcctctgg

SEQ ID NO: 158 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 159 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 160 - DNA, 22 bp, synthetic construct
  misc_feature 1..22: note = Structural abnormalities observed in CRISPR/Cas9-edited Jurkat T cells
  source 1..22: mol_type = other DNA
ttatatccagatatacctctgg

SEQ ID NO: 161 - DNA, 22 bp, synthetic construct
  misc_feature 1..22: note = Structural abnormalities observed in CRISPR/Cas9-edited Jurkat T cells
  source 1..22: mol_type = other DNA
ttgcatccttaaattcctctgg

SEQ ID NO: 162 - DNA, 22 bp, synthetic construct
  misc_feature 1..22: note = Structural abnormalities observed in CRISPR/Cas9-edited Jurkat T cells
  source 1..22: mol_type = other DNA
atttatctataaattcctcagg

SEQ ID NO: 163 - DNA, 22 bp, synthetic construct
  misc_feature 1..22: note = Structural abnormalities observed in CRISPR/Cas9-edited Jurkat T cells
  source 1..22: mol_type = other DNA
ttacatttttaaattactctgg

SEQ ID NO: 164 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 165 - DNA, 22 bp, synthetic construct
  misc_feature 1..22: note = Structural abnormalities observed in CRISPR/Cas9-edited Jurkat T cells
  source 1..22: mol_type = other DNA
taacatctatgaatccctcagg

SEQ ID NO: 166 - DNA, 22 bp, synthetic construct
  misc_feature 1..22: note = Structural abnormalities observed in CRISPR/Cas9-edited Jurkat T cells
  source 1..22: mol_type = other DNA
ttaaatttatatctgcctcagg

SEQ ID NO: 167 - DNA, 22 bp, synthetic construct
  misc_feature 1..22: note = Structural abnormalities observed in CRISPR/Cas9-edited Jurkat T cells
  source 1..22: mol_type = other DNA
ttacatctccattttcctgtgg

SEQ ID NO: 168 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattacttagg

SEQ ID NO: 169 - DNA, 22 bp, synthetic construct
  misc_feature 1..22: note = Structural abnormalities observed in CRISPR/Cas9-edited Jurkat T cells
  source 1..22: mol_type = other DNA
ttacatttctatcttcctttgg

SEQ ID NO: 170 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 171 - DNA, 22 bp, synthetic construct
  misc_feature 1..22: note = Structural abnormalities observed in CRISPR/Cas9-edited Jurkat T cells
  source 1..22: mol_type = other DNA
ttacatctataaattgctttgg

SEQ ID NO: 172 - DNA, 22 bp, synthetic construct
  misc_feature 1..22: note = Structural abnormalities observed in CRISPR/Cas9-edited Jurkat T cells
  source 1..22: mol_type = other DNA
tgacctatatatatttctcagg

SEQ ID NO: 173 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 174 - DNA, 22 bp, synthetic construct
  misc_feature 1..22: note = Structural abnormalities observed in CRISPR/Cas9-edited Jurkat T cells
  source 1..22: mol_type = other DNA
gtacatctttatatttctctgg

SEQ ID NO: 175 - DNA, 22 bp, synthetic construct
  misc_feature 1..22: note = Structural abnormalities observed in CRISPR/Cas9-edited Jurkat T cells
  source 1..22: mol_type = other DNA
tttacactatatattcctctgg

SEQ ID NO: 176 - DNA, 22 bp, synthetic construct
  misc_feature 1..22: note = Structural abnormalities observed in CRISPR/Cas9-edited Jurkat T cells
  source 1..22: mol_type = other DNA
tttcaactatttattcttcggg

SEQ ID NO: 177 - DNA, 22 bp, synthetic construct
  misc_feature 1..22: note = Structural abnormalities observed in CRISPR/Cas9-edited Jurkat T cells
  source 1..22: mol_type = other DNA
ttgcctctttatattcctcagg

SEQ ID NO: 178 - DNA, 22 bp, synthetic construct
  misc_feature 1..22: note = Structural abnormalities observed in CRISPR/Cas9-edited Jurkat T cells
  source 1..22: mol_type = other DNA
taacatttatatatttctgtgg

SEQ ID NO: 179 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattgctctgg

SEQ ID NO: 180 - DNA, 22 bp, synthetic construct
  misc_feature 1..22: note = Structural abnormalities observed in CRISPR/Cas9-edited Jurkat T cells
  source 1..22: mol_type = other DNA
ttaaatctatataacactcagg

SEQ ID NO: 181 - DNA, 22 bp, synthetic construct
  misc_feature 1..22: note = Structural abnormalities observed in CRISPR/Cas9-edited Jurkat T cells
  source 1..22: mol_type = other DNA
ttaaatgtatatattcatcagg

SEQ ID NO: 182 - DNA, 22 bp, synthetic construct
  misc_feature 1..22: note = Structural abnormalities observed in CRISPR/Cas9-edited Jurkat T cells
  source 1..22: mol_type = other DNA
ttagatgtatatattcttgggg

SEQ ID NO: 183 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 184 - DNA, 22 bp, synthetic construct
  misc_feature 1..22: note = Structural abnormalities observed in CRISPR/Cas9-edited Jurkat T cells
  source 1..22: mol_type = other DNA
gtacatctgtcttttcctcagg

SEQ ID NO: 185 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtatattactttgg

SEQ ID NO: 186 - DNA, 22 bp, synthetic construct
  misc_feature 1..22: note = Structural abnormalities observed in CRISPR/Cas9-edited Jurkat T cells
  source 1..22: mol_type = other DNA
ttacatctattgcttcccctgg

SEQ ID NO: 187 - DNA, 22 bp, synthetic construct
  misc_feature 1..22: note = Structural abnormalities observed in CRISPR/Cas9-edited Jurkat T cells
  source 1..22: mol_type = other DNA
ttatatatatatattgttctgg

SEQ ID NO: 188 - DNA, 22 bp, synthetic construct
  misc_feature 1..22: note = Structural abnormalities observed in CRISPR/Cas9-edited Jurkat T cells
  source 1..22: mol_type = other DNA
ttacatctacagattccagagg

SEQ ID NO: 189 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctctgg

SEQ ID NO: 190 - DNA, 22 bp, synthetic construct
  misc_feature 1..22: note = Structural abnormalities observed in CRISPR/Cas9-edited Jurkat T cells
  source 1..22: mol_type = other DNA
tttcttctagatattactctgg

SEQ ID NO: 191 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattcctttgg

SEQ ID NO: 192 - DNA, 22 bp, synthetic construct
  misc_feature 1..22: note = Structural abnormalities observed in CRISPR/Cas9-edited Jurkat T cells
  source 1..22: mol_type = other DNA
ttacaaatatttattcctaggg

SEQ ID NO: 193 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 194 - DNA, 22 bp, synthetic construct
  misc_feature 1..22: note = Structural abnormalities observed in CRISPR/Cas9-edited Jurkat T cells
  source 1..22: mol_type = other DNA
ttacatatttaaattccccagg

SEQ ID NO: 195 - DNA, 22 bp, synthetic construct
  misc_feature 1..22: note = Structural abnormalities observed in CRISPR/Cas9-edited Jurkat T cells
  source 1..22: mol_type = other DNA
tttcctctattttttcctcagg

SEQ ID NO: 196 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 197 - DNA, 22 bp, synthetic construct
  misc_feature 1..22: note = Structural abnormalities observed in CRISPR/Cas9-edited Jurkat T cells
  source 1..22: mol_type = other DNA
tgacatcaacatattactcagg

SEQ ID NO: 198 - DNA, 22 bp, synthetic construct
  misc_feature 1..22: note = Structural abnormalities observed in CRISPR/Cas9-edited Jurkat T cells
  source 1..22: mol_type = other DNA
ttacttctttatactcttcagg

SEQ ID NO: 199 - DNA, 22 bp, synthetic construct
  misc_feature 1..22: note = Structural abnormalities observed in CRISPR/Cas9-edited Jurkat T cells
  source 1..22: mol_type = other DNA
ttacatctagagattgttctgg

SEQ ID NO: 200 - DNA, 22 bp, synthetic construct
  misc_feature 1..22: note = Structural abnormalities observed in CRISPR/Cas9-edited Jurkat T cells
  source 1..22: mol_type = other DNA
atacatctctaaattgctcagg

SEQ ID NO: 201 - DNA, 22 bp, synthetic construct
  misc_feature 1..22: note = Structural abnormalities observed in CRISPR/Cas9-edited Jurkat T cells
  source 1..22: mol_type = other DNA
tttcatctatattcttctcagg

SEQ ID NO: 202 - DNA, 22 bp, synthetic construct
  misc_feature 1..22: note = Structural abnormalities observed in CRISPR/Cas9-edited Jurkat T cells
  source 1..22: mol_type = other DNA
taaaatctatatattactgtgg

SEQ ID NO: 203 - DNA, 22 bp, synthetic construct
  misc_feature 1..22: note = Structural abnormalities observed in CRISPR/Cas9-edited Jurkat T cells
  source 1..22: mol_type = other DNA
ttacttctatatctcactcagg

SEQ ID NO: 204 - DNA, 22 bp, synthetic construct
  misc_feature 1..22: note = Structural abnormalities observed in CRISPR/Cas9-edited Jurkat T cells
  source 1..22: mol_type = other DNA
ctacatttatctattcatctgg

SEQ ID NO: 205 - DNA, 22 bp, synthetic construct
  misc_feature 1..22: note = Structural abnormalities observed in CRISPR/Cas9-edited Jurkat T cells
  source 1..22: mol_type = other DNA
taacatttatttatacctcagg

SEQ ID NO: 206 - DNA, 22 bp, synthetic construct
  misc_feature 1..22: note = Structural abnormalities observed in CRISPR/Cas9-edited Jurkat T cells
  source 1..22: mol_type = other DNA
ttacataaacatattcatctgg

SEQ ID NO: 207 - DNA, 22 bp, synthetic construct
  misc_feature 1..22: note = Structural abnormalities observed in CRISPR/Cas9-edited Jurkat T cells
  source 1..22: mol_type = other DNA
ttagaactatataagcctctgg

SEQ ID NO: 208 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtagattcctttgg

SEQ ID NO: 209 - DNA, 22 bp, synthetic construct
  misc_feature 1..22: note = Structural abnormalities observed in CRISPR/Cas9-edited Jurkat T cells
  source 1..22: mol_type = other DNA
ttaaacctatatattgctttgg

SEQ ID NO: 210 - DNA, 22 bp, synthetic construct
  misc_feature 1..22: note = Structural abnormalities observed in CRISPR/Cas9-edited Jurkat T cells
  source 1..22: mol_type = other DNA
tttcatctctaaattccccggg

SEQ ID NO: 211 - DNA, 22 bp, synthetic construct
  misc_feature 1..22: note = Structural abnormalities observed in CRISPR/Cas9-edited Jurkat T cells
  source 1..22: mol_type = other DNA
tttcatctatgtatccctaggg

SEQ ID NO: 212 - DNA, 22 bp, synthetic construct
  misc_feature 1..22: note = Structural abnormalities observed in CRISPR/Cas9-edited Jurkat T cells
  source 1..22: mol_type = other DNA
ttaaaagtatatgttcctcagg

SEQ ID NO: 213 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 214 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 215 - DNA, 22 bp, synthetic construct
  misc_feature 1..22: note = Structural abnormalities observed in CRISPR/Cas9-edited Jurkat T cells
  source 1..22: mol_type = other DNA
ggacaaatatatattcctctgg

SEQ ID NO: 216 - DNA, 22 bp, synthetic construct
  misc_feature 1..22: note = Structural abnormalities observed in CRISPR/Cas9-edited Jurkat T cells
  source 1..22: mol_type = other DNA
ttatatctataaatcactctgg

SEQ ID NO: 217 - DNA, 22 bp, synthetic construct
  misc_feature 1..22: note = Structural abnormalities observed in CRISPR/Cas9-edited Jurkat T cells
  source 1..22: mol_type = other DNA
ttgaatctatacattgctctgg

SEQ ID NO: 218 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattgctctgg

SEQ ID NO: 219 - DNA, 22 bp, synthetic construct
  misc_feature 1..22: note = Structural abnormalities observed in CRISPR/Cas9-edited Jurkat T cells
  source 1..22: mol_type = other DNA
ttccaactctaaattcctcagg

SEQ ID NO: 220 - DNA, 22 bp, synthetic construct
  misc_feature 1..22: note = Structural abnormalities observed in CRISPR/Cas9-edited Jurkat T cells
  source 1..22: mol_type = other DNA
ttgcatctatatattgctttgg

SEQ ID NO: 221 - DNA, 22 bp, synthetic construct
  misc_feature 1..22: note = Structural abnormalities observed in CRISPR/Cas9-edited Jurkat T cells
  source 1..22: mol_type = other DNA
tttcatctatatgttcatttgg

SEQ ID NO: 222 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatgttcatcagg

SEQ ID NO: 223 - DNA, 22 bp, synthetic construct
  misc_feature 1..22: note = Structural abnormalities observed in CRISPR/Cas9-edited Jurkat T cells
  source 1..22: mol_type = other DNA
atatatctagatatttctcagg

SEQ ID NO: 224 - DNA, 22 bp, synthetic construct
  misc_feature 1..22: note = Structural abnormalities observed in CRISPR/Cas9-edited Jurkat T cells
  source 1..22: mol_type = other DNA
atatatatatatagtcctcagg

SEQ ID NO: 225 - DNA, 22 bp, synthetic construct
  misc_feature 1..22: note = Structural abnormalities observed in CRISPR/Cas9-edited Jurkat T cells
  source 1..22: mol_type = other DNA
atacatttatatatttatctgg

SEQ ID NO: 226 - DNA, 22 bp, synthetic construct
  misc_feature 1..22: note = Structural abnormalities observed in CRISPR/Cas9-edited Jurkat T cells
  source 1..22: mol_type = other DNA
ttaaatccatctactcctcagg

SEQ ID NO: 227 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 228 - DNA, 22 bp, synthetic construct
  misc_feature 1..22: note = Structural abnormalities observed in CRISPR/Cas9-edited Jurkat T cells
  source 1..22: mol_type = other DNA
ttacatcaaagttttcctctgg

SEQ ID NO: 229 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 230 - DNA, 22 bp, synthetic construct
  misc_feature 1..22: note = Structural abnormalities observed in CRISPR/Cas9-edited Jurkat T cells
  source 1..22: mol_type = other DNA
ttacttatataaatttctctgg

SEQ ID NO: 231 - DNA, 22 bp, synthetic construct
  misc_feature 1..22: note = Structural abnormalities observed in CRISPR/Cas9-edited Jurkat T cells
  source 1..22: mol_type = other DNA
ttacttatataaatttctctgg

SEQ ID NO: 232 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 233 - DNA, 22 bp, synthetic construct
  misc_feature 1..22: note = Structural abnormalities observed in CRISPR/Cas9-edited Jurkat T cells
  source 1..22: mol_type = other DNA
ttacatctttatattattgagg

SEQ ID NO: 234 - DNA, 22 bp, synthetic construct
  misc_feature 1..22: note = Structural abnormalities observed in CRISPR/Cas9-edited Jurkat T cells
  source 1..22: mol_type = other DNA
ttacatctttatattattgagg

SEQ ID NO: 235 - DNA, 22 bp, synthetic construct
  misc_feature 1..22: note = Structural abnormalities observed in CRISPR/Cas9-edited Jurkat T cells
  source 1..22: mol_type = other DNA
ttacttctctataatccactgg

SEQ ID NO: 236 - DNA, 22 bp, synthetic construct
  misc_feature 1..22: note = Structural abnormalities observed in CRISPR/Cas9-edited Jurkat T cells
  source 1..22: mol_type = other DNA
ttaaagctatatattactttgg

SEQ ID NO: 237 - DNA, 22 bp, synthetic construct
  misc_feature 1..22: note = Structural abnormalities observed in CRISPR/Cas9-edited Jurkat T cells
  source 1..22: mol_type = other DNA
ttacatctgtaaattgctttgg

SEQ ID NO: 238 - DNA, 22 bp, synthetic construct
  misc_feature 1..22: note = Structural abnormalities observed in CRISPR/Cas9-edited Jurkat T cells
  source 1..22: mol_type = other DNA
atacatgtagatattcatctgg

SEQ ID NO: 239 - DNA, 22 bp, synthetic construct
  misc_feature 1..22: note = Structural abnormalities observed in CRISPR/Cas9-edited Jurkat T cells
  source 1..22: mol_type = other DNA
ttacttatataaatttctctgg

SEQ ID NO: 240 - DNA, 22 bp, synthetic construct
  misc_feature 1..22: note = Structural abnormalities observed in CRISPR/Cas9-edited Jurkat T cells
  source 1..22: mol_type = other DNA
ttacatctgtagattgctttgg

SEQ ID NO: 241 - DNA, 22 bp, synthetic construct
  misc_feature 1..22: note = Structural abnormalities observed in CRISPR/Cas9-edited Jurkat T cells
  source 1..22: mol_type = other DNA
ttacttatataaatttctctgg

SEQ ID NO: 242 - DNA, 22 bp, synthetic construct
  misc_feature 1..22: note = Structural abnormalities observed in CRISPR/Cas9-edited Jurkat T cells
  source 1..22: mol_type = other DNA
ttacttatataaatttctctgg

SEQ ID NO: 243 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 244 - DNA, 22 bp, synthetic construct
  misc_feature 1..22: note = Structural abnormalities observed in CRISPR/Cas9-edited Jurkat T cells
  source 1..22: mol_type = other DNA
ttacttatataaatttctctgg

SEQ ID NO: 245 - DNA, 22 bp, synthetic construct
  misc_feature 1..22: note = Structural abnormalities observed in CRISPR/Cas9-edited Jurkat T cells
  source 1..22: mol_type = other DNA
ttaaatatacattttcctcagg

SEQ ID NO: 246 - DNA, 22 bp, synthetic construct
  misc_feature 1..22: note = Structural abnormalities observed in CRISPR/Cas9-edited Jurkat T cells
  source 1..22: mol_type = other DNA
ttacttatataaatttctctgg

SEQ ID NO: 247 - DNA, 22 bp, synthetic construct
  misc_feature 1..22: note = Structural abnormalities observed in CRISPR/Cas9-edited Jurkat T cells
  source 1..22: mol_type = other DNA
ttacttatataaatttctctgg

SEQ ID NO: 248 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 249 - DNA, 22 bp, synthetic construct
  misc_feature 1..22: note = Structural abnormalities observed in CRISPR/Cas9-edited Jurkat T cells
  source 1..22: mol_type = other DNA
atatatatatatattccacagg

SEQ ID NO: 250 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtatattcctatgg

SEQ ID NO: 251 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattgctctgg

SEQ ID NO: 252 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 253 - DNA, 22 bp, synthetic construct
  misc_feature 1..22: note = Structural abnormalities observed in CRISPR/Cas9-edited Jurkat T cells
  source 1..22: mol_type = other DNA
tcacatctttagattcctaagg

SEQ ID NO: 254 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattgctttgg

SEQ ID NO: 255 - DNA, 22 bp, synthetic construct
  misc_feature 1..22: note = Structural abnormalities observed in CRISPR/Cas9-edited Jurkat T cells
  source 1..22: mol_type = other DNA
tcacatctatatatttctgtgg

SEQ ID NO: 256 - DNA, 22 bp, synthetic construct
  misc_feature 1..22: note = Structural abnormalities observed in CRISPR/Cas9-edited Jurkat T cells
  source 1..22: mol_type = other DNA
ttacataattattttcctctgg

SEQ ID NO: 257 - DNA, 22 bp, synthetic construct
  misc_feature 1..22: note = Structural abnormalities observed in CRISPR/Cas9-edited Jurkat T cells
  source 1..22: mol_type = other DNA
ttacatccagataatcatctgg

SEQ ID NO: 258 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtatattgctttgg

SEQ ID NO: 259 - DNA, 22 bp, synthetic construct
  misc_feature 1..22: note = Structural abnormalities observed in CRISPR/Cas9-edited Jurkat T cells
  source 1..22: mol_type = other DNA
ttccatctccatactcctctgg

SEQ ID NO: 260 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 261 - DNA, 22 bp, synthetic construct
  misc_feature 1..22: note = Structural abnormalities observed in CRISPR/Cas9-edited Jurkat T cells
  source 1..22: mol_type = other DNA
ttaaatttagatattcctaagg

SEQ ID NO: 262 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 263 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 264 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 265 - DNA, 22 bp, synthetic construct
  misc_feature 1..22: note = Structural abnormalities observed in CRISPR/Cas9-edited Jurkat T cells
  source 1..22: mol_type = other DNA
ttatatttatatcttactcagg

SEQ ID NO: 266 - DNA, 22 bp, synthetic construct
  misc_feature 1..22: note = Structural abnormalities observed in CRISPR/Cas9-edited Jurkat T cells
  source 1..22: mol_type = other DNA
ttacttctttattttactcagg

SEQ ID NO: 267 - DNA, 22 bp, synthetic construct
  misc_feature 1..22: note = Structural abnormalities observed in CRISPR/Cas9-edited Jurkat T cells
  source 1..22: mol_type = other DNA
ttagatctatatattgtccagg

SEQ ID NO: 268 - DNA, 22 bp, synthetic construct
  misc_feature 1..22: note = Structural abnormalities observed in CRISPR/Cas9-edited Jurkat T cells
  source 1..22: mol_type = other DNA
ttacatgtatatattgttttgg

SEQ ID NO: 269 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 270 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 271 - DNA, 22 bp, synthetic construct
  misc_feature 1..22: note = Structural abnormalities observed in CRISPR/Cas9-edited Jurkat T cells
  source 1..22: mol_type = other DNA
ttacatccatgacttcctcagg

SEQ ID NO: 272 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 273 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 274 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 275 - DNA, 22 bp, synthetic construct
  misc_feature 1..22: note = Structural abnormalities observed in CRISPR/Cas9-edited Jurkat T cells
  source 1..22: mol_type = other DNA
ttacaactaaatatttctgtgg

SEQ ID NO: 276 - DNA, 22 bp, synthetic construct
  misc_feature 1..22: note = Structural abnormalities observed in CRISPR/Cas9-edited Jurkat T cells
  source 1..22: mol_type = other DNA
ttacatctataaaatcaacagg

SEQ ID NO: 277 - DNA, 22 bp, synthetic construct
  misc_feature 1..22: note = Structural abnormalities observed in CRISPR/Cas9-edited Jurkat T cells
  source 1..22: mol_type = other DNA
ttgcatctctatgttcatcagg

SEQ ID NO: 278 - DNA, 22 bp, synthetic construct
  misc_feature 1..22: note = Structural abnormalities observed in CRISPR/Cas9-edited Jurkat T cells
  source 1..22: mol_type = other DNA
ttacatcaatatatcactaagg

SEQ ID NO: 279 - DNA, 22 bp, synthetic construct
  misc_feature 1..22: note = Structural abnormalities observed in CRISPR/Cas9-edited Jurkat T cells
  source 1..22: mol_type = other DNA
tttcatgtttatgttcctcagg

SEQ ID NO: 280 - DNA, 22 bp, synthetic construct
  misc_feature 1..22: note = Structural abnormalities observed in CRISPR/Cas9-edited Jurkat T cells
  source 1..22: mol_type = other DNA
ttaaacctatagattgctctgg

SEQ ID NO: 281 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 282 - DNA, 22 bp, synthetic construct
  misc_feature 1..22: note = Structural abnormalities observed in CRISPR/Cas9-edited Jurkat T cells
  source 1..22: mol_type = other DNA
ttacatttattttttcctatgg

SEQ ID NO: 283 - DNA, 22 bp, synthetic construct
  misc_feature 1..22: note = Structural abnormalities observed in CRISPR/Cas9-edited Jurkat T cells
  source 1..22: mol_type = other DNA
ttacataaatacattcttctgg

SEQ ID NO: 284 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtagattgctctgg

SEQ ID NO: 285 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 286 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 287 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 288 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 289 - DNA, 22 bp, synthetic construct
  misc_feature 1..22: note = Structural abnormalities observed in CRISPR/Cas9-edited Jurkat T cells
  source 1..22: mol_type = other DNA
atatctctatagattcctctgg

SEQ ID NO: 290 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 291 - DNA, 22 bp, synthetic construct
  misc_feature 1..22: note = Structural abnormalities observed in CRISPR/Cas9-edited Jurkat T cells
  source 1..22: mol_type = other DNA
ttatatttatattttactcagg

SEQ ID NO: 292 - DNA, 22 bp, synthetic construct
  misc_feature 1..22: note = Structural abnormalities observed in CRISPR/Cas9-edited Jurkat T cells
  source 1..22: mol_type = other DNA
ttaaatccataaattcctctgg

SEQ ID NO: 293 - DNA, 22 bp, synthetic construct
  misc_feature 1..22: note = Structural abnormalities observed in CRISPR/Cas9-edited Jurkat T cells
  source 1..22: mol_type = other DNA
ttaaatatatatatacctgtgg

SEQ ID NO: 294 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 295 - DNA, 22 bp, synthetic construct
  misc_feature 1..22: note = Structural abnormalities observed in CRISPR/Cas9-edited Jurkat T cells
  source 1..22: mol_type = other DNA
ttaaatatatatatgcctcagg

SEQ ID NO: 296 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 297 - DNA, 22 bp, synthetic construct
  misc_feature 1..22: note = Structural abnormalities observed in CRISPR/Cas9-edited Jurkat T cells
  source 1..22: mol_type = other DNA
ttaaatctataggttgctctgg

SEQ ID NO: 298 - DNA, 22 bp, synthetic construct
  misc_feature 1..22: note = Structural abnormalities observed in CRISPR/Cas9-edited Jurkat T cells
  source 1..22: mol_type = other DNA
ttacaactaaatagtccttagg

SEQ ID NO: 299 - DNA, 22 bp, synthetic construct
  misc_feature 1..22: note = Structural abnormalities observed in CRISPR/Cas9-edited Jurkat T cells
  source 1..22: mol_type = other DNA
tcacatttatataatcctttgg

SEQ ID NO: 300 - DNA, 22 bp, synthetic construct
  misc_feature 1..22: note = Structural abnormalities observed in CRISPR/Cas9-edited Jurkat T cells
  source 1..22: mol_type = other DNA
ttgcatctggatattactctgg

SEQ ID NO: 301 - DNA, 22 bp, synthetic construct
  misc_feature 1..22: note = Structural abnormalities observed in CRISPR/Cas9-edited Jurkat T cells
  source 1..22: mol_type = other DNA
atatatctatatttccctctgg

SEQ ID NO: 302 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattgctctgg

SEQ ID NO: 303 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 304 - DNA, 22 bp, synthetic construct
  misc_feature 1..22: note = Structural abnormalities observed in CRISPR/Cas9-edited Jurkat T cells
  source 1..22: mol_type = other DNA
ttacatgtttttattccttagg

SEQ ID NO: 305 - DNA, 22 bp, synthetic construct
  misc_feature 1..22: note = Structural abnormalities observed in CRISPR/Cas9-edited Jurkat T cells
  source 1..22: mol_type = other DNA
ttacatcaatttaattctcagg

SEQ ID NO: 306 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 307 - DNA, 22 bp, synthetic construct
  misc_feature 1..22: note = Structural abnormalities observed in CRISPR/Cas9-edited Jurkat T cells
  source 1..22: mol_type = other DNA
cttcatctataccttcctcagg

SEQ ID NO: 308 - DNA, 22 bp, synthetic construct
  misc_feature 1..22: note = Structural abnormalities observed in CRISPR/Cas9-edited Jurkat T cells
  source 1..22: mol_type = other DNA
ttatatctataaattactttgg

SEQ ID NO: 309 - DNA, 22 bp, synthetic construct
  misc_feature 1..22: note = Structural abnormalities observed in CRISPR/Cas9-edited Jurkat T cells
  source 1..22: mol_type = other DNA
ttgtatgtatgtattcctcagg

SEQ ID NO: 310 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattgctctgg

SEQ ID NO: 311 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 312 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 313 - DNA, 22 bp, synthetic construct
  misc_feature 1..22: note = Structural abnormalities observed in CRISPR/Cas9-edited Jurkat T cells
  source 1..22: mol_type = other DNA
ttccaactacatatttctctgg

SEQ ID NO: 314 - DNA, 22 bp, synthetic construct
  misc_feature 1..22: note = Structural abnormalities observed in CRISPR/Cas9-edited Jurkat T cells
  source 1..22: mol_type = other DNA
tcaaatctataaattgctctgg

SEQ ID NO: 315 - DNA, 22 bp, synthetic construct
  misc_feature 1..22: note = Structural abnormalities observed in CRISPR/Cas9-edited Jurkat T cells
  source 1..22: mol_type = other DNA
atacatttataaattgctctgg

SEQ ID NO: 316 - DNA, 22 bp, synthetic construct
  misc_feature 1..22: note = Structural abnormalities observed in CRISPR/Cas9-edited Jurkat T cells
  source 1..22: mol_type = other DNA
ttgcatctatacattgctttgg

SEQ ID NO: 317 - DNA, 22 bp, synthetic construct
  misc_feature 1..22: note = Structural abnormalities observed in CRISPR/Cas9-edited Jurkat T cells
  source 1..22: mol_type = other DNA
taaaatttatatattcctgtgg

SEQ ID NO: 318 - DNA, 22 bp, synthetic construct
  misc_feature 1..22: note = Structural abnormalities observed in CRISPR/Cas9-edited Jurkat T cells
  source 1..22: mol_type = other DNA
tcacataaaaatattcctcagg

SEQ ID NO: 319 - DNA, 22 bp, synthetic construct
  misc_feature 1..22: note = Structural abnormalities observed in CRISPR/Cas9-edited Jurkat T cells
  source 1..22: mol_type = other DNA
atatatatatatattccacagg

SEQ ID NO: 320 - DNA, 22 bp, synthetic construct
  misc_feature 1..22: note = Structural abnormalities observed in CRISPR/Cas9-edited Jurkat T cells
  source 1..22: mol_type = other DNA
gtatatatatatattcatcagg

SEQ ID NO: 321 - DNA, 22 bp, synthetic construct
  misc_feature 1..22: note = Structural abnormalities observed in CRISPR/Cas9-edited Jurkat T cells
  source 1..22: mol_type = other DNA
atacattaatatattcctgtgg

SEQ ID NO: 322 - DNA, 22 bp, synthetic construct
  misc_feature 1..22: note = Structural abnormalities observed in CRISPR/Cas9-edited Jurkat T cells
  source 1..22: mol_type = other DNA
ttatatctgtagattcctttgg

SEQ ID NO: 323 - DNA, 22 bp, synthetic construct
  misc_feature 1..22: note = Structural abnormalities observed in CRISPR/Cas9-edited Jurkat T cells
  source 1..22: mol_type = other DNA
ccacatcaatatatccctctgg

SEQ ID NO: 324 - DNA, 22 bp, synthetic construct
  misc_feature 1..22: note = Structural abnormalities observed in CRISPR/Cas9-edited Jurkat T cells
  source 1..22: mol_type = other DNA
ttaaatcttttttttcctctgg

SEQ ID NO: 325 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 326 - DNA, 22 bp, synthetic construct
  misc_feature 1..22: note = Structural abnormalities observed in CRISPR/Cas9-edited Jurkat T cells
  source 1..22: mol_type = other DNA
ttatatcgataaattcttcagg

SEQ ID NO: 327 - DNA, 22 bp, synthetic construct
  misc_feature 1..22: note = Structural abnormalities observed in CRISPR/Cas9-edited Jurkat T cells
  source 1..22: mol_type = other DNA
ttacacctatactttcccctgg

SEQ ID NO: 328 - DNA, 22 bp, synthetic construct
  misc_feature 1..22: note = Structural abnormalities observed in CRISPR/Cas9-edited Jurkat T cells
  source 1..22: mol_type = other DNA
ttgcatctatatctcccactgg

SEQ ID NO: 329 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 330 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattactttgg

SEQ ID NO: 331 - DNA, 22 bp, synthetic construct
  misc_feature 1..22: note = Structural abnormalities observed in CRISPR/Cas9-edited Jurkat T cells
  source 1..22: mol_type = other DNA
ttatatctacagattcctcagg

SEQ ID NO: 332 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 333 - DNA, 22 bp, synthetic construct
  misc_feature 1..22: note = Structural abnormalities observed in CRISPR/Cas9-edited Jurkat T cells
  source 1..22: mol_type = other DNA
attcatctatatattcatgggg

SEQ ID NO: 334 - DNA, 22 bp, synthetic construct
  misc_feature 1..22: note = Structural abnormalities observed in CRISPR/Cas9-edited Jurkat T cells
  source 1..22: mol_type = other DNA
gtacttttatatgttcctctgg

SEQ ID NO: 335 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 336 - DNA, 22 bp, synthetic construct
  misc_feature 1..22: note = Structural abnormalities observed in CRISPR/Cas9-edited Jurkat T cells
  source 1..22: mol_type = other DNA
tttcatctatacattcttttgg

SEQ ID NO: 337 - DNA, 22 bp, synthetic construct
  misc_feature 1..22: note = Structural abnormalities observed in CRISPR/Cas9-edited Jurkat T cells
  source 1..22: mol_type = other DNA
taatatgtatatattgctctgg

SEQ ID NO: 338 - DNA, 22 bp, synthetic construct
  misc_feature 1..22: note = Structural abnormalities observed in CRISPR/Cas9-edited Jurkat T cells
  source 1..22: mol_type = other DNA
ttatatctagatattcttatgg

SEQ ID NO: 339 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 340 - DNA, 22 bp, synthetic construct
  misc_feature 1..22: note = Structural abnormalities observed in CRISPR/Cas9-edited Jurkat T cells
  source 1..22: mol_type = other DNA
atacatatatatattcttaggg

SEQ ID NO: 341 - DNA, 22 bp, synthetic construct
  misc_feature 1..22: note = Structural abnormalities observed in CRISPR/Cas9-edited Jurkat T cells
  source 1..22: mol_type = other DNA
ttacatgtctatattccaaggg

SEQ ID NO: 342 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 343 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 344 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 345 - DNA, 22 bp, synthetic construct
  misc_feature 1..22: note = Structural abnormalities observed in CRISPR/Cas9-edited Jurkat T cells
  source 1..22: mol_type = other DNA
ttacatgtagatatagctctgg

SEQ ID NO: 346 - DNA, 22 bp, synthetic construct
  misc_feature 1..22: note = Structural abnormalities observed in CRISPR/Cas9-edited Jurkat T cells
  source 1..22: mol_type = other DNA
atatatatatatattcttcagg

SEQ ID NO: 347 - DNA, 22 bp, synthetic construct
  misc_feature 1..22: note = Structural abnormalities observed in CRISPR/Cas9-edited Jurkat T cells
  source 1..22: mol_type = other DNA
ttaaacctatatatttctttgg

SEQ ID NO: 348 - DNA, 22 bp, synthetic construct
  misc_feature 1..22: note = Structural abnormalities observed in CRISPR/Cas9-edited Jurkat T cells
  source 1..22: mol_type = other DNA
ttacatggatacattcttcagg

SEQ ID NO: 349 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 350 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 351 - DNA, 22 bp, synthetic construct
  misc_feature 1..22: note = Structural abnormalities observed in CRISPR/Cas9-edited Jurkat T cells
  source 1..22: mol_type = other DNA
ttaaaaccatatcttcctctgg

SEQ ID NO: 352 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 353 - DNA, 22 bp, synthetic construct
  misc_feature 1..22: note = Structural abnormalities observed in CRISPR/Cas9-edited Jurkat T cells
  source 1..22: mol_type = other DNA
ttaaataaatatattcctttgg

SEQ ID NO: 354 - DNA, 22 bp, synthetic construct
  misc_feature 1..22: note = Structural abnormalities observed in CRISPR/Cas9-edited Jurkat T cells
  source 1..22: mol_type = other DNA
atatatatatatattcctctgg

SEQ ID NO: 355 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaatttctctgg

SEQ ID NO: 356 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 357 - DNA, 22 bp, synthetic construct
  misc_feature 1..22: note = Structural abnormalities observed in CRISPR/Cas9-edited Jurkat T cells
  source 1..22: mol_type = other DNA
tttcatgtatatattcatgagg

SEQ ID NO: 358 - DNA, 22 bp, synthetic construct
  misc_feature 1..22: note = Structural abnormalities observed in CRISPR/Cas9-edited Jurkat T cells
  source 1..22: mol_type = other DNA
ttctatctaaaaattcctctgg

SEQ ID NO: 359 - DNA, 22 bp, synthetic construct
  misc_feature 1..22: note = Structural abnormalities observed in CRISPR/Cas9-edited Jurkat T cells
  source 1..22: mol_type = other DNA
ataaatctgtatattcctttgg

SEQ ID NO: 360 - DNA, 22 bp, synthetic construct
  misc_feature 1..22: note = Structural abnormalities observed in CRISPR/Cas9-edited Jurkat T cells
  source 1..22: mol_type = other DNA
ttaaatctagatgttcctgagg

SEQ ID NO: 361 - DNA, 22 bp, synthetic construct
  misc_feature 1..22: note = Structural abnormalities observed in CRISPR/Cas9-edited Jurkat T cells
  source 1..22: mol_type = other DNA
atatatatatatattccacagg

SEQ ID NO: 362 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 363 - DNA, 22 bp, synthetic construct
  misc_feature 1..22: note = Structural abnormalities observed in CRISPR/Cas9-edited Jurkat T cells
  source 1..22: mol_type = other DNA
ttaaatccaaatatgcctctgg

SEQ ID NO: 364 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 365 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 366 - DNA, 22 bp, synthetic construct
  misc_feature 1..22: note = Structural abnormalities observed in CRISPR/Cas9-edited Jurkat T cells
  source 1..22: mol_type = other DNA
ttaaatctctacattcctttgg

SEQ ID NO: 367 - DNA, 22 bp, synthetic construct
  misc_feature 1..22: note = Structural abnormalities observed in CRISPR/Cas9-edited Jurkat T cells
  source 1..22: mol_type = other DNA
ttccaccaatgtattcctctgg

SEQ ID NO: 368 - DNA, 22 bp, synthetic construct
  misc_feature 1..22: note = Structural abnormalities observed in CRISPR/Cas9-edited Jurkat T cells
  source 1..22: mol_type = other DNA
ttacctccatatatccccctgg

SEQ ID NO: 369 - DNA, 22 bp, synthetic construct
  misc_feature 1..22: note = Structural abnormalities observed in CRISPR/Cas9-edited Jurkat T cells
  source 1..22: mol_type = other DNA
tttcatatatatgttcatcagg

SEQ ID NO: 370 - DNA, 22 bp, synthetic construct
  misc_feature 1..22: note = Structural abnormalities observed in CRISPR/Cas9-edited Jurkat T cells
  source 1..22: mol_type = other DNA
ttatatttataaattcctcagg

SEQ ID NO: 371 - DNA, 22 bp, synthetic construct
  misc_feature 1..22: note = Structural abnormalities observed in CRISPR/Cas9-edited Jurkat T cells
  source 1..22: mol_type = other DNA
ttacatctataaattaccttgg

SEQ ID NO: 372 - DNA, 22 bp, synthetic construct
  misc_feature 1..22: note = Structural abnormalities observed in CRISPR/Cas9-edited Jurkat T cells
  source 1..22: mol_type = other DNA
atatatctctatatttctctgg

SEQ ID NO: 373 - DNA, 22 bp, synthetic construct
  misc_feature 1..22: note = Structural abnormalities observed in CRISPR/Cas9-edited Jurkat T cells
  source 1..22: mol_type = other DNA
ttaaggctatatgttcctctgg

SEQ ID NO: 374 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 375 - DNA, 22 bp, synthetic construct
  misc_feature 1..22: note = Structural abnormalities observed in CRISPR/Cas9-edited Jurkat T cells
  source 1..22: mol_type = other DNA
taacatctacatatttctgtgg

SEQ ID NO: 376 - DNA, 22 bp, synthetic construct
  misc_feature 1..22: note = Structural abnormalities observed in CRISPR/Cas9-edited Jurkat T cells
  source 1..22: mol_type = other DNA
ttatccctatatattccactgg

SEQ ID NO: 377 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattacttcgg

SEQ ID NO: 378 - DNA, 22 bp, synthetic construct
  misc_feature 1..22: note = Structural abnormalities observed in CRISPR/Cas9-edited Jurkat T cells
  source 1..22: mol_type = other DNA
tcacatctatctcttgctcagg

SEQ ID NO: 379 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 380 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 381 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 382 - DNA, 22 bp, synthetic construct
  misc_feature 1..22: note = Structural abnormalities observed in CRISPR/Cas9-edited Jurkat T cells
  source 1..22: mol_type = other DNA
ttacaaataaatattcttctgg

SEQ ID NO: 383 - DNA, 22 bp, synthetic construct
  misc_feature 1..22: note = Structural abnormalities observed in CRISPR/Cas9-edited Jurkat T cells
  source 1..22: mol_type = other DNA
ttgcttccatatatacctcagg

SEQ ID NO: 384 - DNA, 22 bp, synthetic construct
  misc_feature 1..22: note = Structural abnormalities observed in CRISPR/Cas9-edited Jurkat T cells
  source 1..22: mol_type = other DNA
tagcatttatatattcctctgg

SEQ ID NO: 385 - DNA, 22 bp, synthetic construct
  misc_feature 1..22: note = Structural abnormalities observed in CRISPR/Cas9-edited Jurkat T cells
  source 1..22: mol_type = other DNA
tttcatatatttattcatctgg

SEQ ID NO: 386 - DNA, 22 bp, synthetic construct
  misc_feature 1..22: note = Structural abnormalities observed in CRISPR/Cas9-edited Jurkat T cells
  source 1..22: mol_type = other DNA
ttacatctgtatagtgctatgg

SEQ ID NO: 387 - DNA, 22 bp, synthetic construct
  misc_feature 1..22: note = Structural abnormalities observed in CRISPR/Cas9-edited Jurkat T cells
  source 1..22: mol_type = other DNA
ttaaaaatatattttcctctgg

SEQ ID NO: 388 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 389 - DNA, 22 bp, synthetic construct
  misc_feature 1..22: note = Structural abnormalities observed in CRISPR/Cas9-edited Jurkat T cells
  source 1..22: mol_type = other DNA
atatatatatatatttctctgg

SEQ ID NO: 390 - DNA, 22 bp, synthetic construct
  misc_feature 1..22: note = Structural abnormalities observed in CRISPR/Cas9-edited Jurkat T cells
  source 1..22: mol_type = other DNA
ttatgtttatatgttcctcagg

SEQ ID NO: 391 - DNA, 22 bp, synthetic construct
  misc_feature 1..22: note = Structural abnormalities observed in CRISPR/Cas9-edited Jurkat T cells
  source 1..22: mol_type = other DNA
ttacatctatatgtacccaagg

SEQ ID NO: 392 - DNA, 22 bp, synthetic construct
  misc_feature 1..22: note = Structural abnormalities observed in CRISPR/Cas9-edited Jurkat T cells
  source 1..22: mol_type = other DNA
ttacatagatatattcatttgg

SEQ ID NO: 393 - DNA, 22 bp, synthetic construct
  misc_feature 1..22: note = Structural abnormalities observed in CRISPR/Cas9-edited Jurkat T cells
  source 1..22: mol_type = other DNA
ttatatctatggattactctgg

SEQ ID NO: 394 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtatattgctttgg

SEQ ID NO: 395 - DNA, 22 bp, synthetic construct
  misc_feature 1..22: note = Structural abnormalities observed in CRISPR/Cas9-edited Jurkat T cells
  source 1..22: mol_type = other DNA
atatatatatatattcctacgg

SEQ ID NO: 396 - DNA, 22 bp, synthetic construct
  misc_feature 1..22: note = Structural abnormalities observed in CRISPR/Cas9-edited Jurkat T cells
  source 1..22: mol_type = other DNA
ttacttttatagattccttagg

SEQ ID NO: 397 - DNA, 22 bp, synthetic construct
  misc_feature 1..22: note = Structural abnormalities observed in CRISPR/Cas9-edited Jurkat T cells
  source 1..22: mol_type = other DNA
ttgtatctgtatgttcctctgg

SEQ ID NO: 398 - DNA, 22 bp, synthetic construct
  misc_feature 1..22: note = Structural abnormalities observed in CRISPR/Cas9-edited Jurkat T cells
  source 1..22: mol_type = other DNA
tttcatttatatattcccaggg

SEQ ID NO: 399 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 400 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 401 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 402 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 403 - DNA, 22 bp, synthetic construct
  misc_feature 1..22: note = Structural abnormalities observed in CRISPR/Cas9-edited Jurkat T cells
  source 1..22: mol_type = other DNA
ttagatctatatatttctttgg

SEQ ID NO: 404 - DNA, 22 bp, synthetic construct
  misc_feature 1..22: note = Structural abnormalities observed in CRISPR/Cas9-edited Jurkat T cells
  source 1..22: mol_type = other DNA
ttatatatatatattgctaagg

SEQ ID NO: 405 - DNA, 22 bp, synthetic construct
  misc_feature 1..22: note = Structural abnormalities observed in CRISPR/Cas9-edited Jurkat T cells
  source 1..22: mol_type = other DNA
ttacatctacagattgctttgg

SEQ ID NO: 406 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 407 - DNA, 22 bp, synthetic construct
  misc_feature 1..22: note = Structural abnormalities observed in CRISPR/Cas9-edited Jurkat T cells
  source 1..22: mol_type = other DNA
attcatctttatattcctaagg

SEQ ID NO: 408 - DNA, 22 bp, synthetic construct
  misc_feature 1..22: note = Structural abnormalities observed in CRISPR/Cas9-edited Jurkat T cells
  source 1..22: mol_type = other DNA
ttacatcaatttaattctcagg

SEQ ID NO: 409 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 410 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtatattgctttgg

SEQ ID NO: 411 - DNA, 22 bp, synthetic construct
  misc_feature 1..22: note = Structural abnormalities observed in CRISPR/Cas9-edited Jurkat T cells
  source 1..22: mol_type = other DNA
atgcatctacatattccccagg

SEQ ID NO: 412 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtatattgctttgg

SEQ ID NO: 413 - DNA, 22 bp, synthetic construct
  misc_feature 1..22: note = Structural abnormalities observed in CRISPR/Cas9-edited Jurkat T cells
  source 1..22: mol_type = other DNA
ttacatccattttttcctttgg

SEQ ID NO: 414 - DNA, 22 bp, synthetic construct
  misc_feature 1..22: note = Structural abnormalities observed in CRISPR/Cas9-edited Jurkat T cells
  source 1..22: mol_type = other DNA
ttatttctatatcttcctcagg

SEQ ID NO: 415 - DNA, 22 bp, synthetic construct
  misc_feature 1..22: note = Structural abnormalities observed in CRISPR/Cas9-edited Jurkat T cells
  source 1..22: mol_type = other DNA
taacatttgtatgttcctcagg

SEQ ID NO: 416 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 417 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 418 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 419 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctctgg

SEQ ID NO: 420 - DNA, 22 bp, synthetic construct
  misc_feature 1..22: note = Structural abnormalities observed in CRISPR/Cas9-edited Jurkat T cells
  source 1..22: mol_type = other DNA
ttacttctatatatacatgagg

SEQ ID NO: 421 - DNA, 22 bp, synthetic construct
  misc_feature 1..22: note = Structural abnormalities observed in CRISPR/Cas9-edited Jurkat T cells
  source 1..22: mol_type = other DNA
ttaattttatatattcatcagg

SEQ ID NO: 422 - DNA, 22 bp, synthetic construct
  misc_feature 1..22: note = Structural abnormalities observed in CRISPR/Cas9-edited Jurkat T cells
  source 1..22: mol_type = other DNA
ttgaatctatagatccctctgg

SEQ ID NO: 423 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 424 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 425 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtagattcttctgg

SEQ ID NO: 426 - DNA, 22 bp, synthetic construct
  misc_feature 1..22: note = Structural abnormalities observed in CRISPR/Cas9-edited Jurkat T cells
  source 1..22: mol_type = other DNA
ttacatctttatattattgagg

SEQ ID NO: 427 - DNA, 22 bp, synthetic construct
  misc_feature 1..22: note = Structural abnormalities observed in CRISPR/Cas9-edited Jurkat T cells
  source 1..22: mol_type = other DNA
tggcatctataccttcctccgg

SEQ ID NO: 428 - DNA, 22 bp, synthetic construct
  misc_feature 1..22: note = Structural abnormalities observed in CRISPR/Cas9-edited Jurkat T cells
  source 1..22: mol_type = other DNA
ttgaatcaatatattcttcagg

SEQ ID NO: 429 - DNA, 22 bp, synthetic construct
  misc_feature 1..22: note = Structural abnormalities observed in CRISPR/Cas9-edited Jurkat T cells
  source 1..22: mol_type = other DNA
atatttctatatattcctcggg

SEQ ID NO: 430 - DNA, 22 bp, synthetic construct
  misc_feature 1..22: note = Structural abnormalities observed in CRISPR/Cas9-edited Jurkat T cells
  source 1..22: mol_type = other DNA
ttaaatctatacattgctttgg

SEQ ID NO: 431 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 432 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 433 - DNA, 22 bp, synthetic construct
  misc_feature 1..22: note = Structural abnormalities observed in CRISPR/Cas9-edited Jurkat T cells
  source 1..22: mol_type = other DNA
taaaatctatagattgctctgg

SEQ ID NO: 434 - DNA, 22 bp, synthetic construct
  misc_feature 1..22: note = Structural abnormalities observed in CRISPR/Cas9-edited Jurkat T cells
  source 1..22: mol_type = other DNA
taacatatatatttttctctgg

SEQ ID NO: 435 - DNA, 22 bp, synthetic construct
  misc_feature 1..22: note = Structural abnormalities observed in CRISPR/Cas9-edited Jurkat T cells
  source 1..22: mol_type = other DNA
ttagatgtataaattgctctgg

SEQ ID NO: 436 - DNA, 22 bp, synthetic construct
  misc_feature 1..22: note = Structural abnormalities observed in CRISPR/Cas9-edited Jurkat T cells
  source 1..22: mol_type = other DNA
ttaaatctatacattgctttgg

SEQ ID NO: 437 - DNA, 22 bp, synthetic construct
  misc_feature 1..22: note = Structural abnormalities observed in CRISPR/Cas9-edited Jurkat T cells
  source 1..22: mol_type = other DNA
atatatatatatatttctcagg

SEQ ID NO: 438 - DNA, 22 bp, synthetic construct
  misc_feature 1..22: note = Structural abnormalities observed in CRISPR/Cas9-edited Jurkat T cells
  source 1..22: mol_type = other DNA
ttacatctgtagattgctttgg

SEQ ID NO: 439 - DNA, 22 bp, synthetic construct
  misc_feature 1..22: note = Structural abnormalities observed in CRISPR/Cas9-edited Jurkat T cells
  source 1..22: mol_type = other DNA
ttacatctatatattaatgtgg

SEQ ID NO: 440 - DNA, 22 bp, synthetic construct
  misc_feature 1..22: note = Structural abnormalities observed in CRISPR/Cas9-edited Jurkat T cells
  source 1..22: mol_type = other DNA
ttaaatctatacattgctttgg

SEQ ID NO: 441 - DNA, 22 bp, synthetic construct
  misc_feature 1..22: note = Structural abnormalities observed in CRISPR/Cas9-edited Jurkat T cells
  source 1..22: mol_type = other DNA
tcacatatatataattctctgg

SEQ ID NO: 442 - DNA, 22 bp, synthetic construct
  misc_feature 1..22: note = Structural abnormalities observed in CRISPR/Cas9-edited Jurkat T cells
  source 1..22: mol_type = other DNA
tttcctctatattttcatctgg

SEQ ID NO: 443 - DNA, 22 bp, synthetic construct
  misc_feature 1..22: note = Structural abnormalities observed in CRISPR/Cas9-edited Jurkat T cells
  source 1..22: mol_type = other DNA
atatatatatatattcttctgg

SEQ ID NO: 444 - DNA, 22 bp, synthetic construct
  misc_feature 1..22: note = Structural abnormalities observed in CRISPR/Cas9-edited Jurkat T cells
  source 1..22: mol_type = other DNA
ttacatctgtagattgctttgg

SEQ ID NO: 445 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 446 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 447 - DNA, 22 bp, synthetic construct
  misc_feature 1..22: note = Structural abnormalities observed in CRISPR/Cas9-edited Jurkat T cells
  source 1..22: mol_type = other DNA
ttaaatctttaaattactctgg

SEQ ID NO: 448 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 449 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 450 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 451 - DNA, 22 bp, synthetic construct
  misc_feature 1..22: note = Structural abnormalities observed in CRISPR/Cas9-edited Jurkat T cells
  source 1..22: mol_type = other DNA
tcacataaatttattcctctgg

SEQ ID NO: 452 - DNA, 22 bp, synthetic construct
  misc_feature 1..22: note = Structural abnormalities observed in CRISPR/Cas9-edited Jurkat T cells
  source 1..22: mol_type = other DNA
gtacatctctagtttcctcagg

SEQ ID NO: 453 - DNA, 22 bp, synthetic construct
  misc_feature 1..22: note = Structural abnormalities observed in CRISPR/Cas9-edited Jurkat T cells
  source 1..22: mol_type = other DNA
ttacctctataaaatgctctgg

SEQ ID NO: 454 - DNA, 22 bp, synthetic construct
  misc_feature 1..22: note = Structural abnormalities observed in CRISPR/Cas9-edited Jurkat T cells
  source 1..22: mol_type = other DNA
atatatatatatattcatcagg

SEQ ID NO: 455 - DNA, 22 bp, synthetic construct
  misc_feature 1..22: note = Structural abnormalities observed in CRISPR/Cas9-edited Jurkat T cells
  source 1..22: mol_type = other DNA
tttcagctgtttattcctcagg

SEQ ID NO: 456 - DNA, 22 bp, synthetic construct
  misc_feature 1..22: note = Structural abnormalities observed in CRISPR/Cas9-edited Jurkat T cells
  source 1..22: mol_type = other DNA
ttacatccacacatgcctctgg

SEQ ID NO: 457 - DNA, 22 bp, synthetic construct
  misc_feature 1..22: note = Structural abnormalities observed in CRISPR/Cas9-edited Jurkat T cells
  source 1..22: mol_type = other DNA
ttgcatctgtacattcctttgg

SEQ ID NO: 458 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattgctttgg

SEQ ID NO: 459 - DNA, 22 bp, synthetic construct
  misc_feature 1..22: note = Structural abnormalities observed in CRISPR/Cas9-edited Jurkat T cells
  source 1..22: mol_type = other DNA
ttatttgtatatattcctgggg

SEQ ID NO: 460 - DNA, 22 bp, synthetic construct
  misc_feature 1..22: note = Structural abnormalities observed in CRISPR/Cas9-edited Jurkat T cells
  source 1..22: mol_type = other DNA
ttacatgcatattttgctccgg

SEQ ID NO: 461 - DNA, 22 bp, synthetic construct
  misc_feature 1..22: note = Structural abnormalities observed in CRISPR/Cas9-edited Jurkat T cells
  source 1..22: mol_type = other DNA
tttcatcaaaatattgctctgg

SEQ ID NO: 462 - DNA, 22 bp, synthetic construct
  misc_feature 1..22: note = Structural abnormalities observed in CRISPR/Cas9-edited Jurkat T cells
  source 1..22: mol_type = other DNA
ttacatgttgatattcctcagg

SEQ ID NO: 463 - DNA, 22 bp, synthetic construct
  misc_feature 1..22: note = Structural abnormalities observed in CRISPR/Cas9-edited Jurkat T cells
  source 1..22: mol_type = other DNA
atatatatatatattcctaagg

SEQ ID NO: 464 - DNA, 22 bp, synthetic construct
  misc_feature 1..22: note = Structural abnormalities observed in CRISPR/Cas9-edited Jurkat T cells
  source 1..22: mol_type = other DNA
ttatatatatatttttctctgg

SEQ ID NO: 465 - DNA, 22 bp, synthetic construct
  misc_feature 1..22: note = Structural abnormalities observed in CRISPR/Cas9-edited Jurkat T cells
  source 1..22: mol_type = other DNA
atacctttatatatccctctgg

SEQ ID NO: 466 - DNA, 22 bp, synthetic construct
  misc_feature 1..22: note = Structural abnormalities observed in CRISPR/Cas9-edited Jurkat T cells
  source 1..22: mol_type = other DNA
ttacatgtatatattaatttgg

SEQ ID NO: 467 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 468 - DNA, 22 bp, synthetic construct
  misc_feature 1..22: note = Structural abnormalities observed in CRISPR/Cas9-edited Jurkat T cells
  source 1..22: mol_type = other DNA
atatatatatatattcctaagg

SEQ ID NO: 469 - DNA, 22 bp, synthetic construct
  misc_feature 1..22: note = Structural abnormalities observed in CRISPR/Cas9-edited Jurkat T cells
  source 1..22: mol_type = other DNA
ttacagaaatatattccccagg

SEQ ID NO: 470 - DNA, 22 bp, synthetic construct
  misc_feature 1..22: note = Structural abnormalities observed in CRISPR/Cas9-edited Jurkat T cells
  source 1..22: mol_type = other DNA
gtatatatatatatttctcagg

SEQ ID NO: 471 - DNA, 22 bp, synthetic construct
  misc_feature 1..22: note = Structural abnormalities observed in CRISPR/Cas9-edited Jurkat T cells
  source 1..22: mol_type = other DNA
tcacctctatctattccactgg

SEQ ID NO: 472 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattgctctgg

SEQ ID NO: 473 - DNA, 22 bp, synthetic construct
  misc_feature 1..22: note = Structural abnormalities observed in CRISPR/Cas9-edited Jurkat T cells
  source 1..22: mol_type = other DNA
ttacctctataaattcctttgg

SEQ ID NO: 474 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 475 - DNA, 22 bp, synthetic construct
  misc_feature 1..22: note = Structural abnormalities observed in CRISPR/Cas9-edited Jurkat T cells
  source 1..22: mol_type = other DNA
ttaaatctatatatatcactgg

SEQ ID NO: 476 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 477 - DNA, 22 bp, synthetic construct
  misc_feature 1..22: note = Structural abnormalities observed in CRISPR/Cas9-edited Jurkat T cells
  source 1..22: mol_type = other DNA
ttacatgtatttatttctgggg

SEQ ID NO: 478 - DNA, 22 bp, synthetic construct
  misc_feature 1..22: note = Structural abnormalities observed in CRISPR/Cas9-edited Jurkat T cells
  source 1..22: mol_type = other DNA
ttgcatctatatattcctttgg

SEQ ID NO: 479 - DNA, 22 bp, synthetic construct
  misc_feature 1..22: note = Structural abnormalities observed in CRISPR/Cas9-edited Jurkat T cells
  source 1..22: mol_type = other DNA
ttacatctctaagttactcagg

SEQ ID NO: 480 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattgctttgg

SEQ ID NO: 481 - DNA, 22 bp, synthetic construct
  misc_feature 1..22: note = Structural abnormalities observed in CRISPR/Cas9-edited Jurkat T cells
  source 1..22: mol_type = other DNA
ttctatctatttattcatcagg

SEQ ID NO: 482 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 483 - DNA, 22 bp, synthetic construct
  misc_feature 1..22: note = Structural abnormalities observed in CRISPR/Cas9-edited Jurkat T cells
  source 1..22: mol_type = other DNA
ctccatctatatgctcctctgg

SEQ ID NO: 484 - DNA, 22 bp, synthetic construct
  misc_feature 1..22: note = Structural abnormalities observed in CRISPR/Cas9-edited Jurkat T cells
  source 1..22: mol_type = other DNA
ctccagctatattttcctcagg

SEQ ID NO: 485 - DNA, 22 bp, synthetic construct
  misc_feature 1..22: note = Structural abnormalities observed in CRISPR/Cas9-edited Jurkat T cells
  source 1..22: mol_type = other DNA
ttacatttatatatgcctttgg

SEQ ID NO: 486 - DNA, 22 bp, synthetic construct
  misc_feature 1..22: note = Structural abnormalities observed in CRISPR/Cas9-edited Jurkat T cells
  source 1..22: mol_type = other DNA
ctacatccataatttcctcagg

SEQ ID NO: 487 - DNA, 22 bp, synthetic construct
  misc_feature 1..22: note = Structural abnormalities observed in CRISPR/Cas9-edited Jurkat T cells
  source 1..22: mol_type = other DNA
tttcatctttatagtactcagg

SEQ ID NO: 488 - DNA, 22 bp, synthetic construct
  misc_feature 1..22: note = Structural abnormalities observed in CRISPR/Cas9-edited Jurkat T cells
  source 1..22: mol_type = other DNA
tttcatttgtatattcttcagg

SEQ ID NO: 489 - DNA, 22 bp, synthetic construct
  misc_feature 1..22: note = Structural abnormalities observed in CRISPR/Cas9-edited Jurkat T cells
  source 1..22: mol_type = other DNA
ttatgtctatctgttcctctgg

SEQ ID NO: 490 - DNA, 22 bp, synthetic construct
  misc_feature 1..22: note = Structural abnormalities observed in CRISPR/Cas9-edited Jurkat T cells
  source 1..22: mol_type = other DNA
tgatatatatatcttcctccgg

SEQ ID NO: 491 - DNA, 22 bp, synthetic construct
  misc_feature 1..22: note = Structural abnormalities observed in CRISPR/Cas9-edited Jurkat T cells
  source 1..22: mol_type = other DNA
ttacatctttatattattgagg

SEQ ID NO: 492 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtatattgctttgg

SEQ ID NO: 493 - DNA, 22 bp, synthetic construct
  misc_feature 1..22: note = Structural abnormalities observed in CRISPR/Cas9-edited Jurkat T cells
  source 1..22: mol_type = other DNA
ttacccccatatatgcctctgg

SEQ ID NO: 494 - DNA, 22 bp, synthetic construct
  misc_feature 1..22: note = Structural abnormalities observed in CRISPR/Cas9-edited Jurkat T cells
  source 1..22: mol_type = other DNA
ttagttctataaattcccctgg

SEQ ID NO: 495 - DNA, 22 bp, synthetic construct
  misc_feature 1..22: note = Structural abnormalities observed in CRISPR/Cas9-edited Jurkat T cells
  source 1..22: mol_type = other DNA
ttagatctatttataccttggg

SEQ ID NO: 496 - DNA, 22 bp, synthetic construct
  misc_feature 1..22: note = Structural abnormalities observed in CRISPR/Cas9-edited Jurkat T cells
  source 1..22: mol_type = other DNA
tgccatctatatatttttctgg

SEQ ID NO: 497 - DNA, 22 bp, synthetic construct
  misc_feature 1..22: note = Structural abnormalities observed in CRISPR/Cas9-edited Jurkat T cells
  source 1..22: mol_type = other DNA
ttacacctaaatattaatctgg

SEQ ID NO: 498 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 499 - DNA, 22 bp, synthetic construct
  misc_feature 1..22: note = Structural abnormalities observed in CRISPR/Cas9-edited Jurkat T cells
  source 1..22: mol_type = other DNA
ttatatatattttttcctctgg

SEQ ID NO: 500 - DNA, 22 bp, synthetic construct
  misc_feature 1..22: note = Structural abnormalities observed in CRISPR/Cas9-edited Jurkat T cells
  source 1..22: mol_type = other DNA
tttcctctataaattactcggg

SEQ ID NO: 501 - DNA, 22 bp, synthetic construct
  misc_feature 1..22: note = Structural abnormalities observed in CRISPR/Cas9-edited Jurkat T cells
  source 1..22: mol_type = other DNA
taacctctgtttattcctcggg

SEQ ID NO: 502 - DNA, 22 bp, synthetic construct
  misc_feature 1..22: note = Structural abnormalities observed in CRISPR/Cas9-edited Jurkat T cells
  source 1..22: mol_type = other DNA
ttacctctatatttccctcagg

SEQ ID NO: 503 - DNA, 22 bp, synthetic construct
  misc_feature 1..22: note = Structural abnormalities observed in CRISPR/Cas9-edited Jurkat T cells
  source 1..22: mol_type = other DNA
atagatctacatatttctcagg

SEQ ID NO: 504 - DNA, 22 bp, synthetic construct
  misc_feature 1..22: note = Structural abnormalities observed in CRISPR/Cas9-edited Jurkat T cells
  source 1..22: mol_type = other DNA
tttcatttatatatccctaggg

SEQ ID NO: 505 - DNA, 22 bp, synthetic construct
  misc_feature 1..22: note = Structural abnormalities observed in CRISPR/Cas9-edited Jurkat T cells
  source 1..22: mol_type = other DNA
ttaaagctgtatattgctctgg

SEQ ID NO: 506 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 507 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 508 - DNA, 22 bp, synthetic construct
  misc_feature 1..22: note = Structural abnormalities observed in CRISPR/Cas9-edited Jurkat T cells
  source 1..22: mol_type = other DNA
tgacatctacaaactcctctgg

SEQ ID NO: 509 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 510 - DNA, 22 bp, synthetic construct
  misc_feature 1..22: note = Structural abnormalities observed in CRISPR/Cas9-edited Jurkat T cells
  source 1..22: mol_type = other DNA
taacagctatagtttcctctgg

SEQ ID NO: 511 - DNA, 22 bp, synthetic construct
  misc_feature 1..22: note = Structural abnormalities observed in CRISPR/Cas9-edited Jurkat T cells
  source 1..22: mol_type = other DNA
tcccaattatatattcctccgg

SEQ ID NO: 512 - DNA, 22 bp, synthetic construct
  misc_feature 1..22: note = Structural abnormalities observed in CRISPR/Cas9-edited Jurkat T cells
  source 1..22: mol_type = other DNA
ttttatctatagcttcctctgg

SEQ ID NO: 513 - DNA, 22 bp, synthetic construct
  misc_feature 1..22: note = Structural abnormalities observed in CRISPR/Cas9-edited Jurkat T cells
  source 1..22: mol_type = other DNA
atacatctataatttcctgggg

SEQ ID NO: 514 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 515 - DNA, 22 bp, synthetic construct
  misc_feature 1..22: note = Structural abnormalities observed in CRISPR/Cas9-edited Jurkat T cells
  source 1..22: mol_type = other DNA
ttatatttatatatttctgtgg

SEQ ID NO: 516 - DNA, 22 bp, synthetic construct
  misc_feature 1..22: note = Structural abnormalities observed in CRISPR/Cas9-edited Jurkat T cells
  source 1..22: mol_type = other DNA
ttacatctatagatcactttgg

SEQ ID NO: 517 - DNA, 22 bp, synthetic construct
  misc_feature 1..22: note = Structural abnormalities observed in CRISPR/Cas9-edited Jurkat T cells
  source 1..22: mol_type = other DNA
ttccttctatctattcctgtgg

SEQ ID NO: 518 - DNA, 22 bp, synthetic construct
  misc_feature 1..22: note = Structural abnormalities observed in CRISPR/Cas9-edited Jurkat T cells
  source 1..22: mol_type = other DNA
taacatctatattgttctctgg

SEQ ID NO: 519 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 520 - DNA, 22 bp, synthetic construct
  misc_feature 1..22: note = Structural abnormalities observed in CRISPR/Cas9-edited Jurkat T cells
  source 1..22: mol_type = other DNA
ttttatccatatatccctcagg

SEQ ID NO: 521 - DNA, 22 bp, synthetic construct
  misc_feature 1..22: note = Structural abnormalities observed in CRISPR/Cas9-edited Jurkat T cells
  source 1..22: mol_type = other DNA
tgaaatctatagattactctgg

SEQ ID NO: 522 - DNA, 22 bp, synthetic construct
  misc_feature 1..22: note = Structural abnormalities observed in CRISPR/Cas9-edited Jurkat T cells
  source 1..22: mol_type = other DNA
taccatttatattttcctcggg

SEQ ID NO: 523 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattacttggg

SEQ ID NO: 524 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 525 - DNA, 22 bp, synthetic construct
  misc_feature 1..22: note = Structural abnormalities observed in CRISPR/Cas9-edited Jurkat T cells
  source 1..22: mol_type = other DNA
cttaatctatatattcctgtgg

SEQ ID NO: 526 - DNA, 22 bp, synthetic construct
  misc_feature 1..22: note = Structural abnormalities observed in CRISPR/Cas9-edited Jurkat T cells
  source 1..22: mol_type = other DNA
ttacatcaatatattaatgagg

SEQ ID NO: 527 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactgtgg

SEQ ID NO: 528 - DNA, 22 bp, synthetic construct
  misc_feature 1..22: note = Structural abnormalities observed in CRISPR/Cas9-edited Jurkat T cells
  source 1..22: mol_type = other DNA
tgccatctatatatttctttgg

SEQ ID NO: 529 - DNA, 22 bp, synthetic construct
  misc_feature 1..22: note = Structural abnormalities observed in CRISPR/Cas9-edited Jurkat T cells
  source 1..22: mol_type = other DNA
ttccacctatataatactctgg

SEQ ID NO: 530 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 531 - DNA, 22 bp, synthetic construct
  misc_feature 1..22: note = Structural abnormalities observed in CRISPR/Cas9-edited Jurkat T cells
  source 1..22: mol_type = other DNA
ttacttctctataatccactgg

SEQ ID NO: 532 - DNA, 22 bp, synthetic construct
  misc_feature 1..22: note = Structural abnormalities observed in CRISPR/Cas9-edited Jurkat T cells
  source 1..22: mol_type = other DNA
tttcatctatagattgctttgg

SEQ ID NO: 533 - DNA, 22 bp, synthetic construct
  misc_feature 1..22: note = Structural abnormalities observed in CRISPR/Cas9-edited Jurkat T cells
  source 1..22: mol_type = other DNA
ttacttctctataatccactgg

SEQ ID NO: 534 - DNA, 22 bp, synthetic construct
  misc_feature 1..22: note = Structural abnormalities observed in CRISPR/Cas9-edited Jurkat T cells
  source 1..22: mol_type = other DNA
ttacttctctataatccactgg

SEQ ID NO: 535 - DNA, 22 bp, synthetic construct
  misc_feature 1..22: note = Structural abnormalities observed in CRISPR/Cas9-edited Jurkat T cells
  source 1..22: mol_type = other DNA
ttacttctctataatccactgg

SEQ ID NO: 536 - DNA, 22 bp, synthetic construct
  misc_feature 1..22: note = Structural abnormalities observed in CRISPR/Cas9-edited Jurkat T cells
  source 1..22: mol_type = other DNA
taacatctctatattactaggg

SEQ ID NO: 537 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 538 - DNA, 22 bp, synthetic construct
  misc_feature 1..22: note = Structural abnormalities observed in CRISPR/Cas9-edited Jurkat T cells
  source 1..22: mol_type = other DNA
ctacatctaaatatacatcagg

SEQ ID NO: 539 - DNA, 22 bp, synthetic construct
  misc_feature 1..22: note = Structural abnormalities observed in CRISPR/Cas9-edited Jurkat T cells
  source 1..22: mol_type = other DNA
tcacatctttaacttcctcagg

SEQ ID NO: 540 - DNA, 22 bp, synthetic construct
  misc_feature 1..22: note = Structural abnormalities observed in CRISPR/Cas9-edited Jurkat T cells
  source 1..22: mol_type = other DNA
ttataaatatatattcctatgg

SEQ ID NO: 541 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 542 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattacttagg

SEQ ID NO: 543 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattacttagg

SEQ ID NO: 544 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 545 - DNA, 22 bp, synthetic construct
  misc_feature 1..22: note = Structural abnormalities observed in CRISPR/Cas9-edited Jurkat T cells
  source 1..22: mol_type = other DNA
ttgaatctatagatttctctgg

SEQ ID NO: 546 - DNA, 22 bp, synthetic construct
  misc_feature 1..22: note = Structural abnormalities observed in CRISPR/Cas9-edited Jurkat T cells
  source 1..22: mol_type = other DNA
ttaaatatatatattcaactgg

SEQ ID NO: 547 - DNA, 22 bp, synthetic construct
  misc_feature 1..22: note = Structural abnormalities observed in CRISPR/Cas9-edited Jurkat T cells
  source 1..22: mol_type = other DNA
ttagctctatacatttctcagg

SEQ ID NO: 548 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 549 - DNA, 22 bp, synthetic construct
  misc_feature 1..22: note = Structural abnormalities observed in CRISPR/Cas9-edited Jurkat T cells
  source 1..22: mol_type = other DNA
ttacatatatattttccattgg

SEQ ID NO: 550 - DNA, 22 bp, synthetic construct
  misc_feature 1..22: note = Structural abnormalities observed in CRISPR/Cas9-edited Jurkat T cells
  source 1..22: mol_type = other DNA
ttacatttgtatattgctttgg

SEQ ID NO: 551 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 552 - DNA, 22 bp, synthetic construct
  misc_feature 1..22: note = Structural abnormalities observed in CRISPR/Cas9-edited Jurkat T cells
  source 1..22: mol_type = other DNA
ttacctctatatagtattctgg

SEQ ID NO: 553 - DNA, 22 bp, synthetic construct
  misc_feature 1..22: note = Structural abnormalities observed in CRISPR/Cas9-edited Jurkat T cells
  source 1..22: mol_type = other DNA
ttccatctttatcttccacagg

SEQ ID NO: 554 - DNA, 22 bp, synthetic construct
  misc_feature 1..22: note = Structural abnormalities observed in CRISPR/Cas9-edited Jurkat T cells
  source 1..22: mol_type = other DNA
atatatctatatacacctcagg

SEQ ID NO: 555 - DNA, 22 bp, synthetic construct
  misc_feature 1..22: note = Structural abnormalities observed in CRISPR/Cas9-edited Jurkat T cells
  source 1..22: mol_type = other DNA
ttgcagctttacattcctctgg

SEQ ID NO: 556 - DNA, 22 bp, synthetic construct
  misc_feature 1..22: note = Structural abnormalities observed in CRISPR/Cas9-edited Jurkat T cells
  source 1..22: mol_type = other DNA
ccacatctgtatcttcctcagg

SEQ ID NO: 557 - DNA, 22 bp, synthetic construct
  misc_feature 1..22: note = Structural abnormalities observed in CRISPR/Cas9-edited Jurkat T cells
  source 1..22: mol_type = other DNA
ttaagtctatatattgctttgg

SEQ ID NO: 558 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 559 - DNA, 22 bp, synthetic construct
  misc_feature 1..22: note = Structural abnormalities observed in CRISPR/Cas9-edited Jurkat T cells
  source 1..22: mol_type = other DNA
ttaaatctatatattgctttgg

SEQ ID NO: 560 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 561 - DNA, 22 bp, synthetic construct
  misc_feature 1..22: note = Structural abnormalities observed in CRISPR/Cas9-edited Jurkat T cells
  source 1..22: mol_type = other DNA
tatcatctatatatgcttccgg

SEQ ID NO: 562 - DNA, 22 bp, synthetic construct
  misc_feature 1..22: note = Structural abnormalities observed in CRISPR/Cas9-edited Jurkat T cells
  source 1..22: mol_type = other DNA
atacatatatatattcaacagg

SEQ ID NO: 563 - DNA, 22 bp, synthetic construct
  misc_feature 1..22: note = Structural abnormalities observed in CRISPR/Cas9-edited Jurkat T cells
  source 1..22: mol_type = other DNA
atacatatatatattcaacagg

SEQ ID NO: 564 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 565 - DNA, 22 bp, synthetic construct
  misc_feature 1..22: note = Structural abnormalities observed in CRISPR/Cas9-edited Jurkat T cells
  source 1..22: mol_type = other DNA
ttacatttaaatctttctcagg

SEQ ID NO: 566 - DNA, 22 bp, synthetic construct
  misc_feature 1..22: note = Structural abnormalities observed in CRISPR/Cas9-edited Jurkat T cells
  source 1..22: mol_type = other DNA
ttagttgtatatcttcctcagg

SEQ ID NO: 567 - DNA, 22 bp, synthetic construct
  misc_feature 1..22: note = Structural abnormalities observed in CRISPR/Cas9-edited Jurkat T cells
  source 1..22: mol_type = other DNA
ttaaatctatacattactctgg

SEQ ID NO: 568 - DNA, 22 bp, synthetic construct
  misc_feature 1..22: note = Structural abnormalities observed in CRISPR/Cas9-edited Jurkat T cells
  source 1..22: mol_type = other DNA
ctaaatctaaatagtcctctgg

SEQ ID NO: 569 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtacattgctctgg

SEQ ID NO: 570 - DNA, 22 bp, synthetic construct
  misc_feature 1..22: note = Structural abnormalities observed in CRISPR/Cas9-edited Jurkat T cells
  source 1..22: mol_type = other DNA
atccatctctaaattcctctgg

SEQ ID NO: 571 - DNA, 22 bp, synthetic construct
  misc_feature 1..22: note = Structural abnormalities observed in CRISPR/Cas9-edited Jurkat T cells
  source 1..22: mol_type = other DNA
ttatatatatatataactcggg

SEQ ID NO: 572 - DNA, 22 bp, synthetic construct
  misc_feature 1..22: note = Structural abnormalities observed in CRISPR/Cas9-edited Jurkat T cells
  source 1..22: mol_type = other DNA
ttacatctgtagattgctttgg

SEQ ID NO: 573 - DNA, 22 bp, synthetic construct
  misc_feature 1..22: note = Structural abnormalities observed in CRISPR/Cas9-edited Jurkat T cells
  source 1..22: mol_type = other DNA
ttgcatctaaatatttctgtgg

SEQ ID NO: 574 - DNA, 22 bp, synthetic construct
  misc_feature 1..22: note = Structural abnormalities observed in CRISPR/Cas9-edited Jurkat T cells
  source 1..22: mol_type = other DNA
ttgcatctatatattcatcagg

SEQ ID NO: 575 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 576 - DNA, 22 bp, synthetic construct
  misc_feature 1..22: note = Structural abnormalities observed in CRISPR/Cas9-edited Jurkat T cells
  source 1..22: mol_type = other DNA
ttacatttatatatttattggg

SEQ ID NO: 577 - DNA, 22 bp, synthetic construct
  misc_feature 1..22: note = Structural abnormalities observed in CRISPR/Cas9-edited Jurkat T cells
  source 1..22: mol_type = other DNA
ttccatttttatatccctctgg

SEQ ID NO: 578 - DNA, 22 bp, synthetic construct
  misc_feature 1..22: note = Structural abnormalities observed in CRISPR/Cas9-edited Jurkat T cells
  source 1..22: mol_type = other DNA
gtacctctacatattcatctgg

SEQ ID NO: 579 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 580 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtatattgctttgg

SEQ ID NO: 581 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtaaattcctttgg

SEQ ID NO: 582 - DNA, 22 bp, synthetic construct
  misc_feature 1..22: note = Structural abnormalities observed in CRISPR/Cas9-edited Jurkat T cells
  source 1..22: mol_type = other DNA
ttgcatttatattttcccctgg

SEQ ID NO: 583 - DNA, 22 bp, synthetic construct
  misc_feature 1..22: note = Structural abnormalities observed in CRISPR/Cas9-edited Jurkat T cells
  source 1..22: mol_type = other DNA
tgacatcaatatctgcctctgg

SEQ ID NO: 584 - DNA, 22 bp, synthetic construct
  misc_feature 1..22: note = Structural abnormalities observed in CRISPR/Cas9-edited Jurkat T cells
  source 1..22: mol_type = other DNA
ttacatgtaaatatccctttgg

SEQ ID NO: 585 - DNA, 22 bp, synthetic construct
  misc_feature 1..22: note = Structural abnormalities observed in CRISPR/Cas9-edited Jurkat T cells
  source 1..22: mol_type = other DNA
atacatcttcatattcatctgg

SEQ ID NO: 586 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtatatttctttgg

SEQ ID NO: 587 - DNA, 22 bp, synthetic construct
  misc_feature 1..22: note = Structural abnormalities observed in CRISPR/Cas9-edited Jurkat T cells
  source 1..22: mol_type = other DNA
ttaaatctatctttttctcagg

SEQ ID NO: 588 - DNA, 22 bp, synthetic construct
  misc_feature 1..22: note = Structural abnormalities observed in CRISPR/Cas9-edited Jurkat T cells
  source 1..22: mol_type = other DNA
ctacatttttatattcctctgg

SEQ ID NO: 589 - DNA, 22 bp, synthetic construct
  misc_feature 1..22: note = Structural abnormalities observed in CRISPR/Cas9-edited Jurkat T cells
  source 1..22: mol_type = other DNA
ttccattgatgtattcctctgg

SEQ ID NO: 590 - DNA, 22 bp, synthetic construct
  misc_feature 1..22: note = Structural abnormalities observed in CRISPR/Cas9-edited Jurkat T cells
  source 1..22: mol_type = other DNA
atatatatatatattcctaagg

SEQ ID NO: 591 - DNA, 22 bp, synthetic construct
  misc_feature 1..22: note = Structural abnormalities observed in CRISPR/Cas9-edited Jurkat T cells
  source 1..22: mol_type = other DNA
ctacatctatagattgctttgg

SEQ ID NO: 592 - DNA, 22 bp, synthetic construct
  misc_feature 1..22: note = Structural abnormalities observed in CRISPR/Cas9-edited Jurkat T cells
  source 1..22: mol_type = other DNA
ttacagccatataatcccctgg

SEQ ID NO: 593 - DNA, 22 bp, synthetic construct
  misc_feature 1..22: note = Structural abnormalities observed in CRISPR/Cas9-edited Jurkat T cells
  source 1..22: mol_type = other DNA
ctacatctttatatgtctctgg

SEQ ID NO: 594 - DNA, 22 bp, synthetic construct
  misc_feature 1..22: note = Structural abnormalities observed in CRISPR/Cas9-edited Jurkat T cells
  source 1..22: mol_type = other DNA
ttaaagctatatattggtcagg

SEQ ID NO: 595 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 596 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 597 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 598 - DNA, 22 bp, synthetic construct
  misc_feature 1..22: note = Structural abnormalities observed in CRISPR/Cas9-edited Jurkat T cells
  source 1..22: mol_type = other DNA
ttacatctgtaaattgctttgg

SEQ ID NO: 599 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 600 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 601 - DNA, 22 bp, synthetic construct
  misc_feature 1..22: note = Structural abnormalities observed in CRISPR/Cas9-edited Jurkat T cells
  source 1..22: mol_type = other DNA
ttgcatcagtatattcctgagg

SEQ ID NO: 602 - DNA, 22 bp, synthetic construct
  misc_feature 1..22: note = Structural abnormalities observed in CRISPR/Cas9-edited Jurkat T cells
  source 1..22: mol_type = other DNA
tcacatctatgtatttctttgg

SEQ ID NO: 603 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactatgg

SEQ ID NO: 604 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattgctttgg

SEQ ID NO: 605 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattgctctgg

SEQ ID NO: 606 - DNA, 22 bp, synthetic construct
  misc_feature 1..22: note = Structural abnormalities observed in CRISPR/Cas9-edited Jurkat T cells
  source 1..22: mol_type = other DNA
ttaaatctatacattgctttgg

SEQ ID NO: 607 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 608 - DNA, 22 bp, synthetic construct
  misc_feature 1..22: note = Structural abnormalities observed in CRISPR/Cas9-edited Jurkat T cells
  source 1..22: mol_type = other DNA
ttatatctataaattactttgg

SEQ ID NO: 609 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 610 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 611 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 612 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 613 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 614 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 615 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 616 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 617 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 618 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtagattgctctgg

SEQ ID NO: 619 - DNA, 22 bp, synthetic construct
  misc_feature 1..22: note = Structural abnormalities observed in CRISPR/Cas9-edited Jurkat T cells
  source 1..22: mol_type = other DNA
ttacttctctttatttctctgg

SEQ ID NO: 620 - DNA, 22 bp, synthetic construct
  misc_feature 1..22: note = Structural abnormalities observed in CRISPR/Cas9-edited Jurkat T cells
  source 1..22: mol_type = other DNA
ttgaatctatagattccttagg

SEQ ID NO: 621 - DNA, 22 bp, synthetic construct
  misc_feature 1..22: note = Structural abnormalities observed in CRISPR/Cas9-edited Jurkat T cells
  source 1..22: mol_type = other DNA
ttacatctgtaaattgctttgg

SEQ ID NO: 622 - DNA, 22 bp, synthetic construct
  misc_feature 1..22: note = Structural abnormalities observed in CRISPR/Cas9-edited Jurkat T cells
  source 1..22: mol_type = other DNA
atatatatatatattcctctgg

SEQ ID NO: 623 - DNA, 22 bp, synthetic construct
  misc_feature 1..22: note = Structural abnormalities observed in CRISPR/Cas9-edited Jurkat T cells
  source 1..22: mol_type = other DNA
ttaaacctatatattgctttgg

SEQ ID NO: 624 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 625 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 626 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 627 - DNA, 22 bp, synthetic construct
  misc_feature 1..22: note = Structural abnormalities observed in CRISPR/Cas9-edited Jurkat T cells
  source 1..22: mol_type = other DNA
tgacagctatttatttctcagg

SEQ ID NO: 628 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 629 - DNA, 22 bp, synthetic construct
  misc_feature 1..22: note = Structural abnormalities observed in CRISPR/Cas9-edited Jurkat T cells
  source 1..22: mol_type = other DNA
atacacttttatattcctctgg

SEQ ID NO: 630 - DNA, 22 bp, synthetic construct
  misc_feature 1..22: note = Structural abnormalities observed in CRISPR/Cas9-edited Jurkat T cells
  source 1..22: mol_type = other DNA
ttgcatctgtatattacttggg

SEQ ID NO: 631 - DNA, 22 bp, synthetic construct
  misc_feature 1..22: note = Structural abnormalities observed in CRISPR/Cas9-edited Jurkat T cells
  source 1..22: mol_type = other DNA
ttctatgtgtatattcctctgg

SEQ ID NO: 632 - DNA, 22 bp, synthetic construct
  misc_feature 1..22: note = Structural abnormalities observed in CRISPR/Cas9-edited Jurkat T cells
  source 1..22: mol_type = other DNA
ttacatctgattattcttctgg

SEQ ID NO: 633 - DNA, 22 bp, synthetic construct
  misc_feature 1..22: note = Structural abnormalities observed in CRISPR/Cas9-edited Jurkat T cells
  source 1..22: mol_type = other DNA
ttaactctatatattcctgggg

SEQ ID NO: 634 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatatttcttagg

SEQ ID NO: 635 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 636 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 637 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 638 - DNA, 22 bp, synthetic construct
  misc_feature 1..22: note = Structural abnormalities observed in CRISPR/Cas9-edited Jurkat T cells
  source 1..22: mol_type = other DNA
caaaatctatatattcttctgg

SEQ ID NO: 639 - DNA, 22 bp, synthetic construct
  misc_feature 1..22: note = Structural abnormalities observed in CRISPR/Cas9-edited Jurkat T cells
  source 1..22: mol_type = other DNA
ttacatttatactatcctcagg

SEQ ID NO: 640 - DNA, 22 bp, synthetic construct
  misc_feature 1..22: note = Structural abnormalities observed in CRISPR/Cas9-edited Jurkat T cells
  source 1..22: mol_type = other DNA
ttacaactatataatattctgg

SEQ ID NO: 641 - DNA, 22 bp, synthetic construct
  misc_feature 1..22: note = Structural abnormalities observed in CRISPR/Cas9-edited Jurkat T cells
  source 1..22: mol_type = other DNA
taagatctataaatacctccgg

SEQ ID NO: 642 - DNA, 22 bp, synthetic construct
  misc_feature 1..22: note = Structural abnormalities observed in CRISPR/Cas9-edited Jurkat T cells
  source 1..22: mol_type = other DNA
ttacatatgtatatacctgtgg

SEQ ID NO: 643 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtagattgctctgg

SEQ ID NO: 644 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaatttctttgg

SEQ ID NO: 645 - DNA, 22 bp, synthetic construct
  misc_feature 1..22: note = Structural abnormalities observed in CRISPR/Cas9-edited Jurkat T cells
  source 1..22: mol_type = other DNA
ttgcatctatagattgctttgg

SEQ ID NO: 646 - DNA, 22 bp, synthetic construct
  misc_feature 1..22: note = Structural abnormalities observed in CRISPR/Cas9-edited Jurkat T cells
  source 1..22: mol_type = other DNA
tttcatctagatatatctcagg

SEQ ID NO: 647 - DNA, 22 bp, synthetic construct
  misc_feature 1..22: note = Structural abnormalities observed in CRISPR/Cas9-edited Jurkat T cells
  source 1..22: mol_type = other DNA
atatatctatgtatccctcagg

SEQ ID NO: 648 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 649 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 650 - DNA, 22 bp, synthetic construct
  misc_feature 1..22: note = Structural abnormalities observed in CRISPR/Cas9-edited Jurkat T cells
  source 1..22: mol_type = other DNA
ttgaatctatagattgctctgg

SEQ ID NO: 651 - DNA, 22 bp, synthetic construct
  misc_feature 1..22: note = Structural abnormalities observed in CRISPR/Cas9-edited Jurkat T cells
  source 1..22: mol_type = other DNA
ttacaacaatatattgttctgg

SEQ ID NO: 652 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 653 - DNA, 22 bp, synthetic construct
  misc_feature 1..22: note = Structural abnormalities observed in CRISPR/Cas9-edited Jurkat T cells
  source 1..22: mol_type = other DNA
ttatatatatatattcatatgg

SEQ ID NO: 654 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 655 - DNA, 22 bp, synthetic construct
  misc_feature 1..22: note = Structural abnormalities observed in CRISPR/Cas9-edited Jurkat T cells
  source 1..22: mol_type = other DNA
ttacaactaaatatttttcagg

SEQ ID NO: 656 - DNA, 22 bp, synthetic construct
  misc_feature 1..22: note = Structural abnormalities observed in CRISPR/Cas9-edited Jurkat T cells
  source 1..22: mol_type = other DNA
ttacatatatgtatacatcagg

SEQ ID NO: 657 - DNA, 22 bp, synthetic construct
  misc_feature 1..22: note = Structural abnormalities observed in CRISPR/Cas9-edited Jurkat T cells
  source 1..22: mol_type = other DNA
ttatatgaatatattcctctgg

SEQ ID NO: 658 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 659 - DNA, 22 bp, synthetic construct
  misc_feature 1..22: note = Structural abnormalities observed in CRISPR/Cas9-edited Jurkat T cells
  source 1..22: mol_type = other DNA
tcacatctctgcattcctctgg

SEQ ID NO: 660 - DNA, 22 bp, synthetic construct
  misc_feature 1..22: note = Structural abnormalities observed in CRISPR/Cas9-edited Jurkat T cells
  source 1..22: mol_type = other DNA
ttacagctaaattttcctctgg

SEQ ID NO: 661 - DNA, 22 bp, synthetic construct
  misc_feature 1..22: note = Structural abnormalities observed in CRISPR/Cas9-edited Jurkat T cells
  source 1..22: mol_type = other DNA
ttaaatgtctttattcctcagg

SEQ ID NO: 662 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 663 - DNA, 22 bp, synthetic construct
  misc_feature 1..22: note = Structural abnormalities observed in CRISPR/Cas9-edited Jurkat T cells
  source 1..22: mol_type = other DNA
ttacacttatacattactctgg

SEQ ID NO: 664 - DNA, 22 bp, synthetic construct
  misc_feature 1..22: note = Structural abnormalities observed in CRISPR/Cas9-edited Jurkat T cells
  source 1..22: mol_type = other DNA
ttacatctgtctggtcctcagg

SEQ ID NO: 665 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 666 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 667 - DNA, 22 bp, synthetic construct
  misc_feature 1..22: note = Structural abnormalities observed in CRISPR/Cas9-edited Jurkat T cells
  source 1..22: mol_type = other DNA
ttacttctcgatattcccctgg

SEQ ID NO: 668 - DNA, 22 bp, synthetic construct
  misc_feature 1..22: note = Structural abnormalities observed in CRISPR/Cas9-edited Jurkat T cells
  source 1..22: mol_type = other DNA
ttaaatttatatatttctttgg

SEQ ID NO: 669 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 670 - DNA, 22 bp, synthetic construct
  misc_feature 1..22: note = Structural abnormalities observed in CRISPR/Cas9-edited Jurkat T cells
  source 1..22: mol_type = other DNA
ttgaatctgtatattcctttgg

SEQ ID NO: 671 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 672 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 673 - DNA, 22 bp, synthetic construct
  misc_feature 1..22: note = Structural abnormalities observed in CRISPR/Cas9-edited Jurkat T cells
  source 1..22: mol_type = other DNA
ttacacctgtagatttctctgg

SEQ ID NO: 674 - DNA, 22 bp, synthetic construct
  misc_feature 1..22: note = Structural abnormalities observed in CRISPR/Cas9-edited Jurkat T cells
  source 1..22: mol_type = other DNA
tttaatctgtatattcctttgg

SEQ ID NO: 675 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 676 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 677 - DNA, 22 bp, synthetic construct
  misc_feature 1..22: note = Structural abnormalities observed in CRISPR/Cas9-edited Jurkat T cells
  source 1..22: mol_type = other DNA
caaaatctatatatttctcggg

SEQ ID NO: 678 - DNA, 22 bp, synthetic construct
  misc_feature 1..22: note = Structural abnormalities observed in CRISPR/Cas9-edited Jurkat T cells
  source 1..22: mol_type = other DNA
ttatgtctatatagtcctaagg

SEQ ID NO: 679 - DNA, 22 bp, synthetic construct
  misc_feature 1..22: note = Structural abnormalities observed in CRISPR/Cas9-edited Jurkat T cells
  source 1..22: mol_type = other DNA
ttaactcaacatattcctcagg

SEQ ID NO: 680 - DNA, 22 bp, synthetic construct
  misc_feature 1..22: note = Structural abnormalities observed in CRISPR/Cas9-edited Jurkat T cells
  source 1..22: mol_type = other DNA
ttccttctatatattgctctgg

SEQ ID NO: 681 - DNA, 22 bp, synthetic construct
  misc_feature 1..22: note = Structural abnormalities observed in CRISPR/Cas9-edited Jurkat T cells
  source 1..22: mol_type = other DNA
ttacaactgtatgtttctcagg

SEQ ID NO: 682 - DNA, 22 bp, synthetic construct
  misc_feature 1..22: note = Structural abnormalities observed in CRISPR/Cas9-edited Jurkat T cells
  source 1..22: mol_type = other DNA
ttacatctttacatttcactgg

SEQ ID NO: 683 - DNA, 22 bp, synthetic construct
  misc_feature 1..22: note = Structural abnormalities observed in CRISPR/Cas9-edited Jurkat T cells
  source 1..22: mol_type = other DNA
ttaaaaatttatattcctctgg

SEQ ID NO: 684 - DNA, 22 bp, synthetic construct
  misc_feature 1..22: note = Structural abnormalities observed in CRISPR/Cas9-edited Jurkat T cells
  source 1..22: mol_type = other DNA
ttgaatcgatattttcctcagg

SEQ ID NO: 685 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 686 - DNA, 22 bp, synthetic construct
  misc_feature 1..22: note = Structural abnormalities observed in CRISPR/Cas9-edited Jurkat T cells
  source 1..22: mol_type = other DNA
tttgatatataaattcctctgg

SEQ ID NO: 687 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 688 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 689 - DNA, 22 bp, synthetic construct
  misc_feature 1..22: note = Structural abnormalities observed in CRISPR/Cas9-edited Jurkat T cells
  source 1..22: mol_type = other DNA
ctacatatacatattcctatgg

SEQ ID NO: 690 - DNA, 22 bp, synthetic construct
  misc_feature 1..22: note = Structural abnormalities observed in CRISPR/Cas9-edited Jurkat T cells
  source 1..22: mol_type = other DNA
ttgcatctgtatatccctttgg

SEQ ID NO: 691 - DNA, 22 bp, synthetic construct
  misc_feature 1..22: note = Structural abnormalities observed in CRISPR/Cas9-edited Jurkat T cells
  source 1..22: mol_type = other DNA
ttacatctatatacttccaagg

SEQ ID NO: 692 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 693 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaatttctctgg

SEQ ID NO: 694 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 695 - DNA, 22 bp, synthetic construct
  misc_feature 1..22: note = Structural abnormalities observed in CRISPR/Cas9-edited Jurkat T cells
  source 1..22: mol_type = other DNA
ctacacctataaattcctttgg

SEQ ID NO: 696 - DNA, 22 bp, synthetic construct
  misc_feature 1..22: note = Structural abnormalities observed in CRISPR/Cas9-edited Jurkat T cells
  source 1..22: mol_type = other DNA
atatatatatatattccacagg

SEQ ID NO: 697 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 698 - DNA, 22 bp, synthetic construct
  misc_feature 1..22: note = Structural abnormalities observed in CRISPR/Cas9-edited Jurkat T cells
  source 1..22: mol_type = other DNA
atacatatatatatttctaagg

SEQ ID NO: 699 - DNA, 22 bp, synthetic construct
  misc_feature 1..22: note = Structural abnormalities observed in CRISPR/Cas9-edited Jurkat T cells
  source 1..22: mol_type = other DNA
atacatctatatattgccctgg

SEQ ID NO: 700 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattgctttgg

SEQ ID NO: 701 - DNA, 22 bp, synthetic construct
  misc_feature 1..22: note = Structural abnormalities observed in CRISPR/Cas9-edited Jurkat T cells
  source 1..22: mol_type = other DNA
ttcaatctatttagtcctctgg

SEQ ID NO: 702 - DNA, 22 bp, synthetic construct
  misc_feature 1..22: note = Structural abnormalities observed in CRISPR/Cas9-edited Jurkat T cells
  source 1..22: mol_type = other DNA
ttacatcaatttaattctcagg

SEQ ID NO: 703 - DNA, 22 bp, synthetic construct
  misc_feature 1..22: note = Structural abnormalities observed in CRISPR/Cas9-edited Jurkat T cells
  source 1..22: mol_type = other DNA
tcacatataaatattcatctgg

SEQ ID NO: 704 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 705 - DNA, 22 bp, synthetic construct
  misc_feature 1..22: note = Structural abnormalities observed in CRISPR/Cas9-edited Jurkat T cells
  source 1..22: mol_type = other DNA
attcatctctttattcctccgg

SEQ ID NO: 706 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 707 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattgctttgg

SEQ ID NO: 708 - DNA, 22 bp, synthetic construct
  misc_feature 1..22: note = Structural abnormalities observed in CRISPR/Cas9-edited Jurkat T cells
  source 1..22: mol_type = other DNA
gtacatctatgtatacctaagg

SEQ ID NO: 709 - DNA, 22 bp, synthetic construct
  misc_feature 1..22: note = Structural abnormalities observed in CRISPR/Cas9-edited Jurkat T cells
  source 1..22: mol_type = other DNA
ttccatctatttatctctctgg

SEQ ID NO: 710 - DNA, 22 bp, synthetic construct
  misc_feature 1..22: note = Structural abnormalities observed in CRISPR/Cas9-edited Jurkat T cells
  source 1..22: mol_type = other DNA
ctgcatccatatatacctcagg

SEQ ID NO: 711 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 712 - DNA, 22 bp, synthetic construct
  misc_feature 1..22: note = Structural abnormalities observed in CRISPR/Cas9-edited Jurkat T cells
  source 1..22: mol_type = other DNA
ttactactatagattcctttgg

SEQ ID NO: 713 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattgctctgg

SEQ ID NO: 714 - DNA, 22 bp, synthetic construct
  misc_feature 1..22: note = Structural abnormalities observed in CRISPR/Cas9-edited Jurkat T cells
  source 1..22: mol_type = other DNA
gaacttctatttattcctcagg

SEQ ID NO: 715 - DNA, 22 bp, synthetic construct
  misc_feature 1..22: note = Structural abnormalities observed in CRISPR/Cas9-edited Jurkat T cells
  source 1..22: mol_type = other DNA
ttgtatctacatattcctctgg

SEQ ID NO: 716 - DNA, 22 bp, synthetic construct
  misc_feature 1..22: note = Structural abnormalities observed in CRISPR/Cas9-edited Jurkat T cells
  source 1..22: mol_type = other DNA
tcacatctattcatgcctctgg

SEQ ID NO: 717 - DNA, 22 bp, synthetic construct
  misc_feature 1..22: note = Structural abnormalities observed in CRISPR/Cas9-edited Jurkat T cells
  source 1..22: mol_type = other DNA
ttacatctgtaaattgctttgg

SEQ ID NO: 718 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 719 - DNA, 22 bp, synthetic construct
  misc_feature 1..22: note = Structural abnormalities observed in CRISPR/Cas9-edited Jurkat T cells
  source 1..22: mol_type = other DNA
ttacatcaatatctttatctgg

SEQ ID NO: 720 - DNA, 22 bp, synthetic construct
  misc_feature 1..22: note = Structural abnormalities observed in CRISPR/Cas9-edited Jurkat T cells
  source 1..22: mol_type = other DNA
attcatctataaattccactgg

SEQ ID NO: 721 - DNA, 22 bp, synthetic construct
  misc_feature 1..22: note = Structural abnormalities observed in CRISPR/Cas9-edited Jurkat T cells
  source 1..22: mol_type = other DNA
ttaaatctatatattgttttgg

SEQ ID NO: 722 - DNA, 22 bp, synthetic construct
  misc_feature 1..22: note = Structural abnormalities observed in CRISPR/Cas9-edited Jurkat T cells
  source 1..22: mol_type = other DNA
atacatatatatattattctgg

SEQ ID NO: 723 - DNA, 22 bp, synthetic construct
  misc_feature 1..22: note = Structural abnormalities observed in CRISPR/Cas9-edited Jurkat T cells
  source 1..22: mol_type = other DNA
ttacatttacatattactatgg

SEQ ID NO: 724 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 725 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 726 - DNA, 22 bp, synthetic construct
  misc_feature 1..22: note = Structural abnormalities observed in CRISPR/Cas9-edited Jurkat T cells
  source 1..22: mol_type = other DNA
ttccatctatctatttcactgg

SEQ ID NO: 727 - DNA, 22 bp, synthetic construct
  misc_feature 1..22: note = Structural abnormalities observed in CRISPR/Cas9-edited Jurkat T cells
  source 1..22: mol_type = other DNA
atacatctatgtatcccactgg

SEQ ID NO: 728 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 729 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 730 - DNA, 22 bp, synthetic construct
  misc_feature 1..22: note = Structural abnormalities observed in CRISPR/Cas9-edited Jurkat T cells
  source 1..22: mol_type = other DNA
ttacatgtattaattcctgagg

SEQ ID NO: 731 - DNA, 22 bp, synthetic construct
  misc_feature 1..22: note = Structural abnormalities observed in CRISPR/Cas9-edited Jurkat T cells
  source 1..22: mol_type = other DNA
atacctctttatattcgtcagg

SEQ ID NO: 732 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 733 - DNA, 22 bp, synthetic construct
  misc_feature 1..22: note = Structural abnormalities observed in CRISPR/Cas9-edited Jurkat T cells
  source 1..22: mol_type = other DNA
gtacctagatatattcctctgg

SEQ ID NO: 734 - DNA, 22 bp, synthetic construct
  misc_feature 1..22: note = Structural abnormalities observed in CRISPR/Cas9-edited Jurkat T cells
  source 1..22: mol_type = other DNA
ttaaatatatccattcctctgg

SEQ ID NO: 735 - DNA, 22 bp, synthetic construct
  misc_feature 1..22: note = Structural abnormalities observed in CRISPR/Cas9-edited Jurkat T cells
  source 1..22: mol_type = other DNA
ttaattctacatattcctaagg

SEQ ID NO: 736 - DNA, 22 bp, synthetic construct
  misc_feature 1..22: note = Structural abnormalities observed in CRISPR/Cas9-edited Jurkat T cells
  source 1..22: mol_type = other DNA
tgacatttttagattcctcagg

SEQ ID NO: 737 - DNA, 22 bp, synthetic construct
  misc_feature 1..22: note = Structural abnormalities observed in CRISPR/Cas9-edited Jurkat T cells
  source 1..22: mol_type = other DNA
ttacaagtttatatttctctgg

SEQ ID NO: 738 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 739 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactctgg

SEQ ID NO: 740 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 741 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 742 - DNA, 22 bp, synthetic construct
  misc_feature 1..22: note = Structural abnormalities observed in CRISPR/Cas9-edited Jurkat T cells
  source 1..22: mol_type = other DNA
gtatatatatatattccttagg

SEQ ID NO: 743 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 744 - DNA, 22 bp, synthetic construct
  misc_feature 1..22: note = Structural abnormalities observed in CRISPR/Cas9-edited Jurkat T cells
  source 1..22: mol_type = other DNA
tttcccctatgtattcctctgg

SEQ ID NO: 745 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 746 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 747 - DNA, 22 bp, synthetic construct
  misc_feature 1..22: note = Structural abnormalities observed in CRISPR/Cas9-edited Jurkat T cells
  source 1..22: mol_type = other DNA
tatgatctttatattcctctgg

SEQ ID NO: 748 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctctgg

SEQ ID NO: 749 - DNA, 22 bp, synthetic construct
  misc_feature 1..22: note = Structural abnormalities observed in CRISPR/Cas9-edited Jurkat T cells
  source 1..22: mol_type = other DNA
gtacatctgtttattccttagg

SEQ ID NO: 750 - DNA, 22 bp, synthetic construct
  misc_feature 1..22: note = Structural abnormalities observed in CRISPR/Cas9-edited Jurkat T cells
  source 1..22: mol_type = other DNA
ttacgtatatatatacatctgg

SEQ ID NO: 751 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattacttcgg

SEQ ID NO: 752 - DNA, 22 bp, synthetic construct
  misc_feature 1..22: note = Structural abnormalities observed in CRISPR/Cas9-edited Jurkat T cells
  source 1..22: mol_type = other DNA
tgatttctatatattcctgagg

SEQ ID NO: 753 - DNA, 22 bp, synthetic construct
  misc_feature 1..22: note = Structural abnormalities observed in CRISPR/Cas9-edited Jurkat T cells
  source 1..22: mol_type = other DNA
atatatatatatattcctctgg

SEQ ID NO: 754 - DNA, 22 bp, synthetic construct
  misc_feature 1..22: note = Structural abnormalities observed in CRISPR/Cas9-edited Jurkat T cells
  source 1..22: mol_type = other DNA
agacaactatattttcctcagg

SEQ ID NO: 755 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtagattactctgg

SEQ ID NO: 756 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 757 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 758 - DNA, 22 bp, synthetic construct
  misc_feature 1..22: note = Structural abnormalities observed in CRISPR/Cas9-edited Jurkat T cells
  source 1..22: mol_type = other DNA
ttacctctatatatttctgagg

SEQ ID NO: 759 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattacttggg

SEQ ID NO: 760 - DNA, 22 bp, synthetic construct
  misc_feature 1..22: note = Structural abnormalities observed in CRISPR/Cas9-edited Jurkat T cells
  source 1..22: mol_type = other DNA
gtgcatccatatattcctttgg

SEQ ID NO: 761 - DNA, 22 bp, synthetic construct
  misc_feature 1..22: note = Structural abnormalities observed in CRISPR/Cas9-edited Jurkat T cells
  source 1..22: mol_type = other DNA
ttggatctataaattactctgg

SEQ ID NO: 762 - DNA, 22 bp, synthetic construct
  misc_feature 1..22: note = Structural abnormalities observed in CRISPR/Cas9-edited Jurkat T cells
  source 1..22: mol_type = other DNA
ttaaatctatatattactttgg

SEQ ID NO: 763 - DNA, 22 bp, synthetic construct
  misc_feature 1..22: note = Structural abnormalities observed in CRISPR/Cas9-edited Jurkat T cells
  source 1..22: mol_type = other DNA
ttacagctatatatccctttgg

SEQ ID NO: 764 - DNA, 22 bp, synthetic construct
  misc_feature 1..22: note = Structural abnormalities observed in CRISPR/Cas9-edited Jurkat T cells
  source 1..22: mol_type = other DNA
ttaaatatctagattcctcagg

SEQ ID NO: 765 - DNA, 22 bp, synthetic construct
  misc_feature 1..22: note = Structural abnormalities observed in CRISPR/Cas9-edited Jurkat T cells
  source 1..22: mol_type = other DNA
tgaaatctatatattgctttgg

SEQ ID NO: 766 - DNA, 22 bp, synthetic construct
  misc_feature 1..22: note = Structural abnormalities observed in CRISPR/Cas9-edited Jurkat T cells
  source 1..22: mol_type = other DNA
ttacatctacatagtcatttgg

SEQ ID NO: 767 - DNA, 22 bp, synthetic construct
  misc_feature 1..22: note = Structural abnormalities observed in CRISPR/Cas9-edited Jurkat T cells
  source 1..22: mol_type = other DNA
ttaaatctatatattaatttgg

SEQ ID NO: 768 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtaaattactctgg

SEQ ID NO: 769 - DNA, 22 bp, synthetic construct
  misc_feature 1..22: note = Structural abnormalities observed in CRISPR/Cas9-edited Jurkat T cells
  source 1..22: mol_type = other DNA
ttacatctgtaaattgctttgg

SEQ ID NO: 770 - DNA, 22 bp, synthetic construct
  misc_feature 1..22: note = Structural abnormalities observed in CRISPR/Cas9-edited Jurkat T cells
  source 1..22: mol_type = other DNA
ttacttctgtatattactgtgg

SEQ ID NO: 771 - DNA, 22 bp, synthetic construct
  misc_feature 1..22: note = Structural abnormalities observed in CRISPR/Cas9-edited Jurkat T cells
  source 1..22: mol_type = other DNA
ttacatctctaaattgctttgg

SEQ ID NO: 772 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 773 - DNA, 22 bp, synthetic construct
  misc_feature 1..22: note = Structural abnormalities observed in CRISPR/Cas9-edited Jurkat T cells
  source 1..22: mol_type = other DNA
ttacatacatatatgcctgtgg

SEQ ID NO: 774 - DNA, 22 bp, synthetic construct
  misc_feature 1..22: note = Structural abnormalities observed in CRISPR/Cas9-edited Jurkat T cells
  source 1..22: mol_type = other DNA
ttacgtctatgtatacctatgg

SEQ ID NO: 775 - DNA, 22 bp, synthetic construct
  misc_feature 1..22: note = Structural abnormalities observed in CRISPR/Cas9-edited Jurkat T cells
  source 1..22: mol_type = other DNA
ttacacctatagattccctggg

SEQ ID NO: 776 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 777 - DNA, 22 bp, synthetic construct
  misc_feature 1..22: note = Structural abnormalities observed in CRISPR/Cas9-edited Jurkat T cells
  source 1..22: mol_type = other DNA
atacatgtacatatgcctctgg

SEQ ID NO: 778 - DNA, 22 bp, synthetic construct
  misc_feature 1..22: note = Structural abnormalities observed in CRISPR/Cas9-edited Jurkat T cells
  source 1..22: mol_type = other DNA
atatatatatatatgcctcggg

SEQ ID NO: 779 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 780 - DNA, 22 bp, synthetic construct
  misc_feature 1..22: note = Structural abnormalities observed in CRISPR/Cas9-edited Jurkat T cells
  source 1..22: mol_type = other DNA
ttaaatctataagttcctttgg

SEQ ID NO: 781 - DNA, 22 bp, synthetic construct
  misc_feature 1..22: note = Structural abnormalities observed in CRISPR/Cas9-edited Jurkat T cells
  source 1..22: mol_type = other DNA
atatatatatatattcctctgg

SEQ ID NO: 782 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 783 - DNA, 22 bp, synthetic construct
  misc_feature 1..22: note = Structural abnormalities observed in CRISPR/Cas9-edited Jurkat T cells
  source 1..22: mol_type = other DNA
ttacatctgtaaattgctttgg

SEQ ID NO: 784 - DNA, 22 bp, synthetic construct
  misc_feature 1..22: note = Structural abnormalities observed in CRISPR/Cas9-edited Jurkat T cells
  source 1..22: mol_type = other DNA
ttacatctatatattattgagg

SEQ ID NO: 785 - DNA, 22 bp, synthetic construct
  misc_feature 1..22: note = Structural abnormalities observed in CRISPR/Cas9-edited Jurkat T cells
  source 1..22: mol_type = other DNA
atccatctctaaattcctctgg

SEQ ID NO: 786 - DNA, 22 bp, synthetic construct
  misc_feature 1..22: note = Structural abnormalities observed in CRISPR/Cas9-edited Jurkat T cells
  source 1..22: mol_type = other DNA
ttccatctataaattgctttgg

SEQ ID NO: 787 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 788 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 789 - DNA, 22 bp, synthetic construct
  misc_feature 1..22: note = Structural abnormalities observed in CRISPR/Cas9-edited Jurkat T cells
  source 1..22: mol_type = other DNA
taacatcattacattcctcagg

SEQ ID NO: 790 - DNA, 22 bp, synthetic construct
  misc_feature 1..22: note = Structural abnormalities observed in CRISPR/Cas9-edited Jurkat T cells
  source 1..22: mol_type = other DNA
atatatatatatattccactgg

SEQ ID NO: 791 - DNA, 22 bp, synthetic construct
  misc_feature 1..22: note = Structural abnormalities observed in CRISPR/Cas9-edited Jurkat T cells
  source 1..22: mol_type = other DNA
atatatatatatattccactgg

SEQ ID NO: 792 - DNA, 22 bp, synthetic construct
  misc_feature 1..22: note = Structural abnormalities observed in CRISPR/Cas9-edited Jurkat T cells
  source 1..22: mol_type = other DNA
ttacatctgcacattcttctgg

SEQ ID NO: 793 - DNA, 22 bp, synthetic construct
  misc_feature 1..22: note = Structural abnormalities observed in CRISPR/Cas9-edited Jurkat T cells
  source 1..22: mol_type = other DNA
ttaagtctatagactcctcagg

SEQ ID NO: 794 - DNA, 22 bp, synthetic construct
  misc_feature 1..22: note = Structural abnormalities observed in CRISPR/Cas9-edited Jurkat T cells
  source 1..22: mol_type = other DNA
ttacatgtatatatttatgggg

SEQ ID NO: 795 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaatttctttgg

SEQ ID NO: 796 - DNA, 22 bp, synthetic construct
  misc_feature 1..22: note = Structural abnormalities observed in CRISPR/Cas9-edited Jurkat T cells
  source 1..22: mol_type = other DNA
ccatatcaatatattcctctgg

SEQ ID NO: 797 - DNA, 22 bp, synthetic construct
  misc_feature 1..22: note = Structural abnormalities observed in CRISPR/Cas9-edited Jurkat T cells
  source 1..22: mol_type = other DNA
ttacttctatttgttcctatgg

SEQ ID NO: 798 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 799 - DNA, 22 bp, synthetic construct
  misc_feature 1..22: note = Structural abnormalities observed in CRISPR/Cas9-edited Jurkat T cells
  source 1..22: mol_type = other DNA
ttaaatctatatattgctttgg

SEQ ID NO: 800 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtatattgctttgg

SEQ ID NO: 801 - DNA, 22 bp, synthetic construct
  misc_feature 1..22: note = Structural abnormalities observed in CRISPR/Cas9-edited Jurkat T cells
  source 1..22: mol_type = other DNA
catcatctatatattccttggg

SEQ ID NO: 802 - DNA, 22 bp, synthetic construct
  misc_feature 1..22: note = Structural abnormalities observed in CRISPR/Cas9-edited Jurkat T cells
  source 1..22: mol_type = other DNA
gtatatatatatattccccagg

SEQ ID NO: 803 - DNA, 22 bp, synthetic construct
  misc_feature 1..22: note = Structural abnormalities observed in CRISPR/Cas9-edited Jurkat T cells
  source 1..22: mol_type = other DNA
ttacatatatatatacttgggg

SEQ ID NO: 804 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactatgg

SEQ ID NO: 805 - DNA, 22 bp, synthetic construct
  misc_feature 1..22: note = Structural abnormalities observed in CRISPR/Cas9-edited Jurkat T cells
  source 1..22: mol_type = other DNA
ttatatatatatatttttctgg

SEQ ID NO: 806 - DNA, 22 bp, synthetic construct
  misc_feature 1..22: note = Structural abnormalities observed in CRISPR/Cas9-edited Jurkat T cells
  source 1..22: mol_type = other DNA
ttacatcaataaattcatttgg

SEQ ID NO: 807 - DNA, 22 bp, synthetic construct
  misc_feature 1..22: note = Structural abnormalities observed in CRISPR/Cas9-edited Jurkat T cells
  source 1..22: mol_type = other DNA
tttcatatgtatattcctttgg

SEQ ID NO: 808 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 809 - DNA, 22 bp, synthetic construct
  misc_feature 1..22: note = Structural abnormalities observed in CRISPR/Cas9-edited Jurkat T cells
  source 1..22: mol_type = other DNA
tttcttatatatattcctgggg

SEQ ID NO: 810 - DNA, 22 bp, synthetic construct
  misc_feature 1..22: note = Structural abnormalities observed in CRISPR/Cas9-edited Jurkat T cells
  source 1..22: mol_type = other DNA
ttatatatatataatcctatgg

SEQ ID NO: 811 - DNA, 22 bp, synthetic construct
  misc_feature 1..22: note = Structural abnormalities observed in CRISPR/Cas9-edited Jurkat T cells
  source 1..22: mol_type = other DNA
ttacatctataaattgctttgg

SEQ ID NO: 812 - DNA, 22 bp, synthetic construct
  misc_feature 1..22: note = Structural abnormalities observed in CRISPR/Cas9-edited Jurkat T cells
  source 1..22: mol_type = other DNA
ttacatctgtatatttcctggg

SEQ ID NO: 813 - DNA, 22 bp, synthetic construct
  misc_feature 1..22: note = Structural abnormalities observed in CRISPR/Cas9-edited Jurkat T cells
  source 1..22: mol_type = other DNA
tttcatctctgtatttctcagg

SEQ ID NO: 814 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 815 - DNA, 22 bp, synthetic construct
  misc_feature 1..22: note = Structural abnormalities observed in CRISPR/Cas9-edited Jurkat T cells
  source 1..22: mol_type = other DNA
tttctcctatatgttcctctgg

SEQ ID NO: 816 - DNA, 22 bp, synthetic construct
  misc_feature 1..22: note = Structural abnormalities observed in CRISPR/Cas9-edited Jurkat T cells
  source 1..22: mol_type = other DNA
ttacatctttatacttctttgg

SEQ ID NO: 817 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 818 - DNA, 22 bp, synthetic construct
  misc_feature 1..22: note = Structural abnormalities observed in CRISPR/Cas9-edited Jurkat T cells
  source 1..22: mol_type = other DNA
ttacatttatatattattaagg

SEQ ID NO: 819 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattgctctgg

SEQ ID NO: 820 - DNA, 22 bp, synthetic construct
  misc_feature 1..22: note = Structural abnormalities observed in CRISPR/Cas9-edited Jurkat T cells
  source 1..22: mol_type = other DNA
tttcatttaaatagtcctctgg

SEQ ID NO: 821 - DNA, 22 bp, synthetic construct
  misc_feature 1..22: note = Structural abnormalities observed in CRISPR/Cas9-edited Jurkat T cells
  source 1..22: mol_type = other DNA
ttacatctatttatactgcagg

SEQ ID NO: 822 - DNA, 22 bp, synthetic construct
  misc_feature 1..22: note = Structural abnormalities observed in CRISPR/Cas9-edited Jurkat T cells
  source 1..22: mol_type = other DNA
ctacatcaatatattcatttgg

SEQ ID NO: 823 - DNA, 22 bp, synthetic construct
  misc_feature 1..22: note = Structural abnormalities observed in CRISPR/Cas9-edited Jurkat T cells
  source 1..22: mol_type = other DNA
ttttatgtatatattcatcagg

SEQ ID NO: 824 - DNA, 22 bp, synthetic construct
  misc_feature 1..22: note = Structural abnormalities observed in CRISPR/Cas9-edited Jurkat T cells
  source 1..22: mol_type = other DNA
ttacatctataacttactttgg

SEQ ID NO: 825 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 826 - DNA, 22 bp, synthetic construct
  misc_feature 1..22: note = Structural abnormalities observed in CRISPR/Cas9-edited Jurkat T cells
  source 1..22: mol_type = other DNA
ttaaatctaaatattactatgg

SEQ ID NO: 827 - DNA, 22 bp, synthetic construct
  misc_feature 1..22: note = Structural abnormalities observed in CRISPR/Cas9-edited Jurkat T cells
  source 1..22: mol_type = other DNA
ttacaactatagataccttagg

SEQ ID NO: 828 - DNA, 22 bp, synthetic construct
  misc_feature 1..22: note = Structural abnormalities observed in CRISPR/Cas9-edited Jurkat T cells
  source 1..22: mol_type = other DNA
ttgaatctgtatattcctttgg

SEQ ID NO: 829 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaatttctttgg

SEQ ID NO: 830 - DNA, 22 bp, synthetic construct
  misc_feature 1..22: note = Structural abnormalities observed in CRISPR/Cas9-edited Jurkat T cells
  source 1..22: mol_type = other DNA
atacatatatatatttctttgg

SEQ ID NO: 831 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 832 - DNA, 22 bp, synthetic construct
  misc_feature 1..22: note = Structural abnormalities observed in CRISPR/Cas9-edited Jurkat T cells
  source 1..22: mol_type = other DNA
ttgcatccatagattcctttgg

SEQ ID NO: 833 - DNA, 22 bp, synthetic construct
  misc_feature 1..22: note = Structural abnormalities observed in CRISPR/Cas9-edited Jurkat T cells
  source 1..22: mol_type = other DNA
ttggatctatatattgctgggg

SEQ ID NO: 834 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 835 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 836 - DNA, 22 bp, synthetic construct
  misc_feature 1..22: note = Structural abnormalities observed in CRISPR/Cas9-edited Jurkat T cells
  source 1..22: mol_type = other DNA
tggcatctatataatcctgagg

SEQ ID NO: 837 - DNA, 22 bp, synthetic construct
  misc_feature 1..22: note = Structural abnormalities observed in CRISPR/Cas9-edited Jurkat T cells
  source 1..22: mol_type = other DNA
tcacatctataatttcccccgg

SEQ ID NO: 838 - DNA, 22 bp, synthetic construct
  misc_feature 1..22: note = Structural abnormalities observed in CRISPR/Cas9-edited Jurkat T cells
  source 1..22: mol_type = other DNA
ttacatgtataagttcttcagg

SEQ ID NO: 839 - DNA, 22 bp, synthetic construct
  misc_feature 1..22: note = Structural abnormalities observed in CRISPR/Cas9-edited Jurkat T cells
  source 1..22: mol_type = other DNA
tcacattcataaattcctcagg

SEQ ID NO: 840 - DNA, 22 bp, synthetic construct
  misc_feature 1..22: note = Structural abnormalities observed in CRISPR/Cas9-edited Jurkat T cells
  source 1..22: mol_type = other DNA
ttagatcaatttattcctaggg

SEQ ID NO: 841 - DNA, 22 bp, synthetic construct
  misc_feature 1..22: note = Structural abnormalities observed in CRISPR/Cas9-edited Jurkat T cells
  source 1..22: mol_type = other DNA
atgcatctatatattccttggg

SEQ ID NO: 842 - DNA, 22 bp, synthetic construct
  misc_feature 1..22: note = Structural abnormalities observed in CRISPR/Cas9-edited Jurkat T cells
  source 1..22: mol_type = other DNA
attcatctttatattccccagg

SEQ ID NO: 843 - DNA, 22 bp, synthetic construct
  misc_feature 1..22: note = Structural abnormalities observed in CRISPR/Cas9-edited Jurkat T cells
  source 1..22: mol_type = other DNA
tggcatatttatattcctcagg

SEQ ID NO: 844 - DNA, 22 bp, synthetic construct
  misc_feature 1..22: note = Structural abnormalities observed in CRISPR/Cas9-edited Jurkat T cells
  source 1..22: mol_type = other DNA
ttatatctataaattactttgg

SEQ ID NO: 845 - DNA, 22 bp, synthetic construct
  misc_feature 1..22: note = Structural abnormalities observed in CRISPR/Cas9-edited Jurkat T cells
  source 1..22: mol_type = other DNA
ataaatttatatatccctctgg

SEQ ID NO: 846 - DNA, 22 bp, synthetic construct
  misc_feature 1..22: note = Structural abnormalities observed in CRISPR/Cas9-edited Jurkat T cells
  source 1..22: mol_type = other DNA
ttaaatgtatatgttactcggg

SEQ ID NO: 847 - DNA, 22 bp, synthetic construct
  misc_feature 1..22: note = Structural abnormalities observed in CRISPR/Cas9-edited Jurkat T cells
  source 1..22: mol_type = other DNA
ttacatgaatatattgcacagg

SEQ ID NO: 848 - DNA, 22 bp, synthetic construct
  misc_feature 1..22: note = Structural abnormalities observed in CRISPR/Cas9-edited Jurkat T cells
  source 1..22: mol_type = other DNA
ttacatctacatgattctcagg

SEQ ID NO: 849 - DNA, 22 bp, synthetic construct
  misc_feature 1..22: note = Structural abnormalities observed in CRISPR/Cas9-edited Jurkat T cells
  source 1..22: mol_type = other DNA
gtacatttaaatattccccagg

SEQ ID NO: 850 - DNA, 22 bp, synthetic construct
  misc_feature 1..22: note = Structural abnormalities observed in CRISPR/Cas9-edited Jurkat T cells
  source 1..22: mol_type = other DNA
ttacatttatttagtcctgtgg

SEQ ID NO: 851 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 852 - DNA, 22 bp, synthetic construct
  misc_feature 1..22: note = Structural abnormalities observed in CRISPR/Cas9-edited Jurkat T cells
  source 1..22: mol_type = other DNA
ttaaatctatacattactttgg

SEQ ID NO: 853 - DNA, 22 bp, synthetic construct
  misc_feature 1..22: note = Structural abnormalities observed in CRISPR/Cas9-edited Jurkat T cells
  source 1..22: mol_type = other DNA
tttcatccaggtattcctcagg

SEQ ID NO: 854 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtatattgctttgg

SEQ ID NO: 855 - DNA, 22 bp, synthetic construct
  misc_feature 1..22: note = Structural abnormalities observed in CRISPR/Cas9-edited Jurkat T cells
  source 1..22: mol_type = other DNA
tttcatatctatattcatcggg

SEQ ID NO: 856 - DNA, 22 bp, synthetic construct
  misc_feature 1..22: note = Structural abnormalities observed in CRISPR/Cas9-edited Jurkat T cells
  source 1..22: mol_type = other DNA
tcacatctgtatattctactgg

SEQ ID NO: 857 - DNA, 22 bp, synthetic construct
  misc_feature 1..22: note = Structural abnormalities observed in CRISPR/Cas9-edited Jurkat T cells
  source 1..22: mol_type = other DNA
atatatttatgtattcctcagg

SEQ ID NO: 858 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattcattagg

SEQ ID NO: 859 - DNA, 22 bp, synthetic construct
  misc_feature 1..22: note = Structural abnormalities observed in CRISPR/Cas9-edited Jurkat T cells
  source 1..22: mol_type = other DNA
ttacatctttatattattgagg

SEQ ID NO: 860 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 861 - DNA, 22 bp, synthetic construct
  misc_feature 1..22: note = Structural abnormalities observed in CRISPR/Cas9-edited Jurkat T cells
  source 1..22: mol_type = other DNA
ttacagctattcattccacagg

SEQ ID NO: 862 - DNA, 22 bp, synthetic construct
  misc_feature 1..22: note = Structural abnormalities observed in CRISPR/Cas9-edited Jurkat T cells
  source 1..22: mol_type = other DNA
ttacatctagaaaatcctaagg

SEQ ID NO: 863 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 864 - DNA, 22 bp, synthetic construct
  misc_feature 1..22: note = Structural abnormalities observed in CRISPR/Cas9-edited Jurkat T cells
  source 1..22: mol_type = other DNA
ttccatctttactttcctcggg

SEQ ID NO: 865 - DNA, 22 bp, synthetic construct
  misc_feature 1..22: note = Structural abnormalities observed in CRISPR/Cas9-edited Jurkat T cells
  source 1..22: mol_type = other DNA
ttacatctatatgttgttacgg

SEQ ID NO: 866 - DNA, 22 bp, synthetic construct
  misc_feature 1..22: note = Structural abnormalities observed in CRISPR/Cas9-edited Jurkat T cells
  source 1..22: mol_type = other DNA
tcaaatgtatatatttctctgg

SEQ ID NO: 867 - DNA, 22 bp, synthetic construct
  misc_feature 1..22: note = Structural abnormalities observed in CRISPR/Cas9-edited Jurkat T cells
  source 1..22: mol_type = other DNA
ttaaatctatatatttcttggg

SEQ ID NO: 868 - DNA, 22 bp, synthetic construct
  misc_feature 1..22: note = Structural abnormalities observed in CRISPR/Cas9-edited Jurkat T cells
  source 1..22: mol_type = other DNA
atgcatatatattttcctcagg

SEQ ID NO: 869 - DNA, 22 bp, synthetic construct
  misc_feature 1..22: note = Structural abnormalities observed in CRISPR/Cas9-edited Jurkat T cells
  source 1..22: mol_type = other DNA
ttacatatatagattcatttgg

SEQ ID NO: 870 - DNA, 22 bp, synthetic construct
  misc_feature 1..22: note = Structural abnormalities observed in CRISPR/Cas9-edited Jurkat T cells
  source 1..22: mol_type = other DNA
ttaaatctatgtattgctttgg

SEQ ID NO: 871 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 872 - DNA, 22 bp, synthetic construct
  misc_feature 1..22: note = Structural abnormalities observed in CRISPR/Cas9-edited Jurkat T cells
  source 1..22: mol_type = other DNA
tgatatctatatcttcctgtgg

SEQ ID NO: 873 - DNA, 22 bp, synthetic construct
  misc_feature 1..22: note = Structural abnormalities observed in CRISPR/Cas9-edited Jurkat T cells
  source 1..22: mol_type = other DNA
ttagatctatctattcaacagg

SEQ ID NO: 874 - DNA, 22 bp, synthetic construct
  misc_feature 1..22: note = Structural abnormalities observed in CRISPR/Cas9-edited Jurkat T cells
  source 1..22: mol_type = other DNA
ttacatataaatatttctcagg

SEQ ID NO: 875 - DNA, 22 bp, synthetic construct
  misc_feature 1..22: note = Structural abnormalities observed in CRISPR/Cas9-edited Jurkat T cells
  source 1..22: mol_type = other DNA
ttaactctatttattcctaagg

SEQ ID NO: 876 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 877 - DNA, 22 bp, synthetic construct
  misc_feature 1..22: note = Structural abnormalities observed in CRISPR/Cas9-edited Jurkat T cells
  source 1..22: mol_type = other DNA
ttacctctggatattccactgg

SEQ ID NO: 878 - DNA, 22 bp, synthetic construct
  misc_feature 1..22: note = Structural abnormalities observed in CRISPR/Cas9-edited Jurkat T cells
  source 1..22: mol_type = other DNA
ttatatctgtaaatacctctgg

SEQ ID NO: 879 - DNA, 22 bp, synthetic construct
  misc_feature 1..22: note = Structural abnormalities observed in CRISPR/Cas9-edited Jurkat T cells
  source 1..22: mol_type = other DNA
ttacattaatatattcatgtgg

SEQ ID NO: 880 - DNA, 22 bp, synthetic construct
  misc_feature 1..22: note = Structural abnormalities observed in CRISPR/Cas9-edited Jurkat T cells
  source 1..22: mol_type = other DNA
ttaaagatatatattcctgagg

SEQ ID NO: 881 - DNA, 22 bp, synthetic construct
  misc_feature 1..22: note = Structural abnormalities observed in CRISPR/Cas9-edited Jurkat T cells
  source 1..22: mol_type = other DNA
ttacttgtgtagattcctcggg

SEQ ID NO: 882 - DNA, 22 bp, synthetic construct
  misc_feature 1..22: note = Structural abnormalities observed in CRISPR/Cas9-edited Jurkat T cells
  source 1..22: mol_type = other DNA
ttagatcaatataatcatctgg

SEQ ID NO: 883 - DNA, 22 bp, synthetic construct
  misc_feature 1..22: note = Structural abnormalities observed in CRISPR/Cas9-edited Jurkat T cells
  source 1..22: mol_type = other DNA
ttatatctataaattactttgg

SEQ ID NO: 884 - DNA, 22 bp, synthetic construct
  misc_feature 1..22: note = Structural abnormalities observed in CRISPR/Cas9-edited Jurkat T cells
  source 1..22: mol_type = other DNA
ttgtatctatatttttctcagg

SEQ ID NO: 885 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 886 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 887 - DNA, 22 bp, synthetic construct
  misc_feature 1..22: note = Structural abnormalities observed in CRISPR/Cas9-edited Jurkat T cells
  source 1..22: mol_type = other DNA
ttacatctgtaaattgctttgg

SEQ ID NO: 888 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattgctttgg

SEQ ID NO: 889 - DNA, 22 bp, synthetic construct
  misc_feature 1..22: note = Structural abnormalities observed in CRISPR/Cas9-edited Jurkat T cells
  source 1..22: mol_type = other DNA
ttccatctatgtgttcctctgg

SEQ ID NO: 890 - DNA, 22 bp, synthetic construct
  misc_feature 1..22: note = Structural abnormalities observed in CRISPR/Cas9-edited Jurkat T cells
  source 1..22: mol_type = other DNA
atatatatatatattcctctgg

SEQ ID NO: 891 - DNA, 22 bp, synthetic construct
  misc_feature 1..22: note = Structural abnormalities observed in CRISPR/Cas9-edited Jurkat T cells
  source 1..22: mol_type = other DNA
ttaaatgtataaattcctctgg

SEQ ID NO: 892 - DNA, 22 bp, synthetic construct
  misc_feature 1..22: note = Structural abnormalities observed in CRISPR/Cas9-edited Jurkat T cells
  source 1..22: mol_type = other DNA
atacatctttatattcctatgg

SEQ ID NO: 893 - DNA, 22 bp, synthetic construct
  misc_feature 1..22: note = Structural abnormalities observed in CRISPR/Cas9-edited Jurkat T cells
  source 1..22: mol_type = other DNA
taacatctatataatcctgagg

SEQ ID NO: 894 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtatattgctctgg

SEQ ID NO: 895 - DNA, 22 bp, synthetic construct
  misc_feature 1..22: note = Structural abnormalities observed in CRISPR/Cas9-edited Jurkat T cells
  source 1..22: mol_type = other DNA
ttacaaatatatattccactgg

SEQ ID NO: 896 - DNA, 22 bp, synthetic construct
  misc_feature 1..22: note = Structural abnormalities observed in CRISPR/Cas9-edited Jurkat T cells
  source 1..22: mol_type = other DNA
ttgcctctttatattcctcagg

SEQ ID NO: 897 - DNA, 22 bp, synthetic construct
  misc_feature 1..22: note = Structural abnormalities observed in CRISPR/Cas9-edited Jurkat T cells
  source 1..22: mol_type = other DNA
ttaaatctgcatattcctctgg

SEQ ID NO: 898 - DNA, 22 bp, synthetic construct
  misc_feature 1..22: note = Structural abnormalities observed in CRISPR/Cas9-edited Jurkat T cells
  source 1..22: mol_type = other DNA
ttatatctatatataactctgg

SEQ ID NO: 899 - DNA, 22 bp, synthetic construct
  misc_feature 1..22: note = Structural abnormalities observed in CRISPR/Cas9-edited Jurkat T cells
  source 1..22: mol_type = other DNA
ttacatctctataatcctcagg

SEQ ID NO: 900 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattcctttgg

SEQ ID NO: 901 - DNA, 22 bp, synthetic construct
  misc_feature 1..22: note = Structural abnormalities observed in CRISPR/Cas9-edited Jurkat T cells
  source 1..22: mol_type = other DNA
ttacatctatagattgctttgg

SEQ ID NO: 902 - DNA, 22 bp, synthetic construct
  misc_feature 1..22: note = Structural abnormalities observed in CRISPR/Cas9-edited Jurkat T cells
  source 1..22: mol_type = other DNA
atgcatatatatattcctcagg

SEQ ID NO: 903 - DNA, 22 bp, synthetic construct
  misc_feature 1..22: note = Structural abnormalities observed in CRISPR/Cas9-edited Jurkat T cells
  source 1..22: mol_type = other DNA
ttacatctgtattttccaaagg

SEQ ID NO: 904 - DNA, 22 bp, synthetic construct
  misc_feature 1..22: note = Structural abnormalities observed in CRISPR/Cas9-edited Jurkat T cells
  source 1..22: mol_type = other DNA
ttatatgtatatattactatgg

SEQ ID NO: 905 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 906 - DNA, 22 bp, synthetic construct
  misc_feature 1..22: note = Structural abnormalities observed in CRISPR/Cas9-edited Jurkat T cells
  source 1..22: mol_type = other DNA
ttagttctataacttcctctgg

SEQ ID NO: 907 - DNA, 22 bp, synthetic construct
  misc_feature 1..22: note = Structural abnormalities observed in CRISPR/Cas9-edited Jurkat T cells
  source 1..22: mol_type = other DNA
ttacaaatctttattcctctgg

SEQ ID NO: 908 - DNA, 22 bp, synthetic construct
  misc_feature 1..22: note = Structural abnormalities observed in CRISPR/Cas9-edited Jurkat T cells
  source 1..22: mol_type = other DNA
ttacatataaatattcttctgg

SEQ ID NO: 909 - DNA, 22 bp, synthetic construct
  misc_feature 1..22: note = Structural abnormalities observed in CRISPR/Cas9-edited Jurkat T cells
  source 1..22: mol_type = other DNA
tcagatccaaatattcctcagg

SEQ ID NO: 910 - DNA, 22 bp, synthetic construct
  misc_feature 1..22: note = Structural abnormalities observed in CRISPR/Cas9-edited Jurkat T cells
  source 1..22: mol_type = other DNA
atacatatatatattattcagg

SEQ ID NO: 911 - DNA, 22 bp, synthetic construct
  misc_feature 1..22: note = Structural abnormalities observed in CRISPR/Cas9-edited Jurkat T cells
  source 1..22: mol_type = other DNA
ctacttctgtctattcctctgg

SEQ ID NO: 912 - DNA, 22 bp, synthetic construct
  misc_feature 1..22: note = Structural abnormalities observed in CRISPR/Cas9-edited Jurkat T cells
  source 1..22: mol_type = other DNA
ttgcatttatatatgcctttgg

SEQ ID NO: 913 - DNA, 22 bp, synthetic construct
  misc_feature 1..22: note = Structural abnormalities observed in CRISPR/Cas9-edited Jurkat T cells
  source 1..22: mol_type = other DNA
ttacatatatacaatccacagg

SEQ ID NO: 914 - DNA, 22 bp, synthetic construct
  misc_feature 1..22: note = Structural abnormalities observed in CRISPR/Cas9-edited Jurkat T cells
  source 1..22: mol_type = other DNA
tttcatatatatattcatgagg

SEQ ID NO: 915 - DNA, 22 bp, synthetic construct
  misc_feature 1..22: note = Structural abnormalities observed in CRISPR/Cas9-edited Jurkat T cells
  source 1..22: mol_type = other DNA
ctaactctatattttcctcagg

SEQ ID NO: 916 - DNA, 22 bp, synthetic construct
  misc_feature 1..22: note = Structural abnormalities observed in CRISPR/Cas9-edited Jurkat T cells
  source 1..22: mol_type = other DNA
tttcatctgtattttcttctgg

SEQ ID NO: 917 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 918 - DNA, 22 bp, synthetic construct
  misc_feature 1..22: note = Structural abnormalities observed in CRISPR/Cas9-edited Jurkat T cells
  source 1..22: mol_type = other DNA
taaaatctatattttactctgg

SEQ ID NO: 919 - DNA, 22 bp, synthetic construct
  misc_feature 1..22: note = Structural abnormalities observed in CRISPR/Cas9-edited Jurkat T cells
  source 1..22: mol_type = other DNA
taagatctgtatatttctctgg

SEQ ID NO: 920 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 921 - DNA, 22 bp, synthetic construct
  misc_feature 1..22: note = Structural abnormalities observed in CRISPR/Cas9-edited Jurkat T cells
  source 1..22: mol_type = other DNA
ggacatctatatacttctcagg

SEQ ID NO: 922 - DNA, 22 bp, synthetic construct
  misc_feature 1..22: note = Structural abnormalities observed in CRISPR/Cas9-edited Jurkat T cells
  source 1..22: mol_type = other DNA
gtacttctatatatttcacagg

SEQ ID NO: 923 - DNA, 22 bp, synthetic construct
  misc_feature 1..22: note = Structural abnormalities observed in CRISPR/Cas9-edited Jurkat T cells
  source 1..22: mol_type = other DNA
tgaaatttatttattcctctgg

SEQ ID NO: 924 - DNA, 22 bp, synthetic construct
  misc_feature 1..22: note = Structural abnormalities observed in CRISPR/Cas9-edited Jurkat T cells
  source 1..22: mol_type = other DNA
ttaaatctatatattactttgg

SEQ ID NO: 925 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 926 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 927 - DNA, 22 bp, synthetic construct
  misc_feature 1..22: note = Structural abnormalities observed in CRISPR/Cas9-edited Jurkat T cells
  source 1..22: mol_type = other DNA
ttacatatatacactactcagg

SEQ ID NO: 928 - DNA, 22 bp, synthetic construct
  misc_feature 1..22: note = Structural abnormalities observed in CRISPR/Cas9-edited Jurkat T cells
  source 1..22: mol_type = other DNA
ttacatgtgcattttcctcagg

SEQ ID NO: 929 - DNA, 22 bp, synthetic construct
  misc_feature 1..22: note = Structural abnormalities observed in CRISPR/Cas9-edited Jurkat T cells
  source 1..22: mol_type = other DNA
ttccatttaaatatttctcagg

SEQ ID NO: 930 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 931 - DNA, 22 bp, synthetic construct
  misc_feature 1..22: note = Structural abnormalities observed in CRISPR/Cas9-edited Jurkat T cells
  source 1..22: mol_type = other DNA
tcacaactatctattccactgg

SEQ ID NO: 932 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 933 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 934 - DNA, 22 bp, synthetic construct
  misc_feature 1..22: note = Structural abnormalities observed in CRISPR/Cas9-edited Jurkat T cells
  source 1..22: mol_type = other DNA
ttacagctttaaattcgtctgg

SEQ ID NO: 935 - DNA, 22 bp, synthetic construct
  misc_feature 1..22: note = Structural abnormalities observed in CRISPR/Cas9-edited Jurkat T cells
  source 1..22: mol_type = other DNA
ttacatcttgaaagtcctctgg

SEQ ID NO: 936 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 937 - DNA, 22 bp, synthetic construct
  misc_feature 1..22: note = Structural abnormalities observed in CRISPR/Cas9-edited Jurkat T cells
  source 1..22: mol_type = other DNA
ttagatctatagattaatctgg

SEQ ID NO: 938 - DNA, 22 bp, synthetic construct
  misc_feature 1..22: note = Structural abnormalities observed in CRISPR/Cas9-edited Jurkat T cells
  source 1..22: mol_type = other DNA
ttatatctataaattgcttcgg

SEQ ID NO: 939 - DNA, 22 bp, synthetic construct
  misc_feature 1..22: note = Structural abnormalities observed in CRISPR/Cas9-edited Jurkat T cells
  source 1..22: mol_type = other DNA
tgatttctatatattcctaggg

SEQ ID NO: 940 - DNA, 22 bp, synthetic construct
  misc_feature 1..22: note = Structural abnormalities observed in CRISPR/Cas9-edited Jurkat T cells
  source 1..22: mol_type = other DNA
ttacatctaaataatcttaagg

SEQ ID NO: 941 - DNA, 22 bp, synthetic construct
  misc_feature 1..22: note = Structural abnormalities observed in CRISPR/Cas9-edited Jurkat T cells
  source 1..22: mol_type = other DNA
ttaaatcaatatatttcccagg

SEQ ID NO: 942 - DNA, 22 bp, synthetic construct
  misc_feature 1..22: note = Structural abnormalities observed in CRISPR/Cas9-edited Jurkat T cells
  source 1..22: mol_type = other DNA
tcacctatttatattcctcagg

SEQ ID NO: 943 - DNA, 22 bp, synthetic construct
  misc_feature 1..22: note = Structural abnormalities observed in CRISPR/Cas9-edited Jurkat T cells
  source 1..22: mol_type = other DNA
ttatatctatctcttcctaggg

SEQ ID NO: 944 - DNA, 22 bp, synthetic construct
  misc_feature 1..22: note = Structural abnormalities observed in CRISPR/Cas9-edited Jurkat T cells
  source 1..22: mol_type = other DNA
ttagatctgtgaattcctctgg

SEQ ID NO: 945 - DNA, 22 bp, synthetic construct
  misc_feature 1..22: note = Structural abnormalities observed in CRISPR/Cas9-edited Jurkat T cells
  source 1..22: mol_type = other DNA
ttaaatgttaatattcctcagg

SEQ ID NO: 946 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 947 - DNA, 22 bp, synthetic construct
  misc_feature 1..22: note = Structural abnormalities observed in CRISPR/Cas9-edited Jurkat T cells
  source 1..22: mol_type = other DNA
ttgaatctatacattcctttgg

SEQ ID NO: 948 - DNA, 22 bp, synthetic construct
  misc_feature 1..22: note = Structural abnormalities observed in CRISPR/Cas9-edited Jurkat T cells
  source 1..22: mol_type = other DNA
ctacatctacagattcttcagg

SEQ ID NO: 949 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 950 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 951 - DNA, 22 bp, synthetic construct
  misc_feature 1..22: note = Structural abnormalities observed in CRISPR/Cas9-edited Jurkat T cells
  source 1..22: mol_type = other DNA
taacgtgtatattttcctcagg

SEQ ID NO: 952 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 953 - DNA, 22 bp, synthetic construct
  misc_feature 1..22: note = Structural abnormalities observed in CRISPR/Cas9-edited Jurkat T cells
  source 1..22: mol_type = other DNA
ttatatctgtatatttctatgg

SEQ ID NO: 954 - DNA, 22 bp, synthetic construct
  misc_feature 1..22: note = Structural abnormalities observed in CRISPR/Cas9-edited Jurkat T cells
  source 1..22: mol_type = other DNA
ttacctggaaatattcctctgg

SEQ ID NO: 955 - DNA, 22 bp, synthetic construct
  misc_feature 1..22: note = Structural abnormalities observed in CRISPR/Cas9-edited Jurkat T cells
  source 1..22: mol_type = other DNA
ttccatctataaattactttgg

SEQ ID NO: 956 - DNA, 22 bp, synthetic construct
  misc_feature 1..22: note = Structural abnormalities observed in CRISPR/Cas9-edited Jurkat T cells
  source 1..22: mol_type = other DNA
ttatatatatgtatgcctcagg

SEQ ID NO: 957 - DNA, 22 bp, synthetic construct
  misc_feature 1..22: note = Structural abnormalities observed in CRISPR/Cas9-edited Jurkat T cells
  source 1..22: mol_type = other DNA
atatatatatatattcctttgg

SEQ ID NO: 958 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 959 - DNA, 22 bp, synthetic construct
  misc_feature 1..22: note = Structural abnormalities observed in CRISPR/Cas9-edited Jurkat T cells
  source 1..22: mol_type = other DNA
ttataactttatattgctcagg

SEQ ID NO: 960 - DNA, 22 bp, synthetic construct
  misc_feature 1..22: note = Structural abnormalities observed in CRISPR/Cas9-edited Jurkat T cells
  source 1..22: mol_type = other DNA
ctatatgtacatattcctctgg

SEQ ID NO: 961 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 962 - DNA, 22 bp, synthetic construct
  misc_feature 1..22: note = Structural abnormalities observed in CRISPR/Cas9-edited Jurkat T cells
  source 1..22: mol_type = other DNA
ttacatctagatcttcttccgg

SEQ ID NO: 963 - DNA, 22 bp, synthetic construct
  misc_feature 1..22: note = Structural abnormalities observed in CRISPR/Cas9-edited Jurkat T cells
  source 1..22: mol_type = other DNA
ttagttctttatcttcctcagg

SEQ ID NO: 964 - DNA, 22 bp, synthetic construct
  misc_feature 1..22: note = Structural abnormalities observed in CRISPR/Cas9-edited Jurkat T cells
  source 1..22: mol_type = other DNA
gtacatttatatagtcctgtgg

SEQ ID NO: 965 - DNA, 22 bp, synthetic construct
  misc_feature 1..22: note = Structural abnormalities observed in CRISPR/Cas9-edited Jurkat T cells
  source 1..22: mol_type = other DNA
ttacatctgtagattgctttgg

SEQ ID NO: 966 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtagattcctttgg

SEQ ID NO: 967 - DNA, 22 bp, synthetic construct
  misc_feature 1..22: note = Structural abnormalities observed in CRISPR/Cas9-edited Jurkat T cells
  source 1..22: mol_type = other DNA
ttacatttataaattactttgg

SEQ ID NO: 968 - DNA, 22 bp, synthetic construct
  misc_feature 1..22: note = Structural abnormalities observed in CRISPR/Cas9-edited Jurkat T cells
  source 1..22: mol_type = other DNA
ttacatctgtaaattgctttgg

SEQ ID NO: 969 - DNA, 22 bp, synthetic construct
  misc_feature 1..22: note = Structural abnormalities observed in CRISPR/Cas9-edited Jurkat T cells
  source 1..22: mol_type = other DNA
ttacatgtttatattcagcagg

SEQ ID NO: 970 - DNA, 22 bp, synthetic construct
  misc_feature 1..22: note = Structural abnormalities observed in CRISPR/Cas9-edited Jurkat T cells
  source 1..22: mol_type = other DNA
ttacatctgtatcattctctgg

SEQ ID NO: 971 - DNA, 22 bp, synthetic construct
  misc_feature 1..22: note = Structural abnormalities observed in CRISPR/Cas9-edited Jurkat T cells
  source 1..22: mol_type = other DNA
ttacatctatatgatattcagg

SEQ ID NO: 972 - DNA, 22 bp, synthetic construct
  misc_feature 1..22: note = Structural abnormalities observed in CRISPR/Cas9-edited Jurkat T cells
  source 1..22: mol_type = other DNA
ttacatttatataatactttgg

SEQ ID NO: 973 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 974 - DNA, 22 bp, synthetic construct
  misc_feature 1..22: note = Structural abnormalities observed in CRISPR/Cas9-edited Jurkat T cells
  source 1..22: mol_type = other DNA
taaaattaatatattcctctgg

SEQ ID NO: 975 - DNA, 22 bp, synthetic construct
  misc_feature 1..22: note = Structural abnormalities observed in CRISPR/Cas9-edited Jurkat T cells
  source 1..22: mol_type = other DNA
ttacatctttccattccactgg

SEQ ID NO: 976 - DNA, 22 bp, synthetic construct
  misc_feature 1..22: note = Structural abnormalities observed in CRISPR/Cas9-edited Jurkat T cells
  source 1..22: mol_type = other DNA
ttaaatccttatagtcctctgg

SEQ ID NO: 977 - DNA, 22 bp, synthetic construct
  misc_feature 1..22: note = Structural abnormalities observed in CRISPR/Cas9-edited Jurkat T cells
  source 1..22: mol_type = other DNA
ttacatttatacattaatctgg

SEQ ID NO: 978 - DNA, 22 bp, synthetic construct
  misc_feature 1..22: note = Structural abnormalities observed in CRISPR/Cas9-edited Jurkat T cells
  source 1..22: mol_type = other DNA
ttagatctataaattcatttgg

SEQ ID NO: 979 - DNA, 22 bp, synthetic construct
  misc_feature 1..22: note = Structural abnormalities observed in CRISPR/Cas9-edited Jurkat T cells
  source 1..22: mol_type = other DNA
ttataactgtatattccttggg

SEQ ID NO: 980 - DNA, 22 bp, synthetic construct
  misc_feature 1..22: note = Structural abnormalities observed in CRISPR/Cas9-edited Jurkat T cells
  source 1..22: mol_type = other DNA
atacatatatatattgctcagg

SEQ ID NO: 981 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 982 - DNA, 22 bp, synthetic construct
  misc_feature 1..22: note = Structural abnormalities observed in CRISPR/Cas9-edited Jurkat T cells
  source 1..22: mol_type = other DNA
ttacatctttaagttcctcagg

SEQ ID NO: 983 - DNA, 22 bp, synthetic construct
  misc_feature 1..22: note = Structural abnormalities observed in CRISPR/Cas9-edited Jurkat T cells
  source 1..22: mol_type = other DNA
ttaaatctatatactgctcagg

SEQ ID NO: 984 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 985 - DNA, 22 bp, synthetic construct
  misc_feature 1..22: note = Structural abnormalities observed in CRISPR/Cas9-edited Jurkat T cells
  source 1..22: mol_type = other DNA
ttccatctttaaattgctcagg

SEQ ID NO: 986 - DNA, 22 bp, synthetic construct
  misc_feature 1..22: note = Structural abnormalities observed in CRISPR/Cas9-edited Jurkat T cells
  source 1..22: mol_type = other DNA
gtacagctatatttccctctgg

SEQ ID NO: 987 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 988 - DNA, 22 bp, synthetic construct
  misc_feature 1..22: note = Structural abnormalities observed in CRISPR/Cas9-edited Jurkat T cells
  source 1..22: mol_type = other DNA
ttatattaatatagtcctcagg

SEQ ID NO: 989 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattacttggg

SEQ ID NO: 990 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtatattgctttgg

SEQ ID NO: 991 - DNA, 22 bp, synthetic construct
  misc_feature 1..22: note = Structural abnormalities observed in CRISPR/Cas9-edited Jurkat T cells
  source 1..22: mol_type = other DNA
ttacatttacacaatcctctgg

SEQ ID NO: 992 - DNA, 22 bp, synthetic construct
  misc_feature 1..22: note = Structural abnormalities observed in CRISPR/Cas9-edited Jurkat T cells
  source 1..22: mol_type = other DNA
ttacctttgtattttcctctgg

SEQ ID NO: 993 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 994 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattactttgg

SEQ ID NO: 995 - DNA, 22 bp, synthetic construct
  misc_feature 1..22: note = Structural abnormalities observed in CRISPR/Cas9-edited Jurkat T cells
  source 1..22: mol_type = other DNA
tcacagctctacattcctctgg

SEQ ID NO: 996 - DNA, 22 bp, synthetic construct
  misc_feature 1..22: note = Structural abnormalities observed in CRISPR/Cas9-edited Jurkat T cells
  source 1..22: mol_type = other DNA
atacatatatatatttatcagg

SEQ ID NO: 997 - DNA, 22 bp, synthetic construct
  misc_feature 1..22: note = Structural abnormalities observed in CRISPR/Cas9-edited Jurkat T cells
  source 1..22: mol_type = other DNA
ttgcatctatagattgttcagg

SEQ ID NO: 998 - DNA, 22 bp, synthetic construct
  misc_feature 1..22: note = Structural abnormalities observed in CRISPR/Cas9-edited Jurkat T cells
  source 1..22: mol_type = other DNA
cttcatttatatattcctaagg

SEQ ID NO: 999 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 1000 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 1001 - DNA, 22 bp, synthetic construct
  misc_feature 1..22: note = Structural abnormalities observed in CRISPR/Cas9-edited Jurkat T cells
  source 1..22: mol_type = other DNA
atatatatatattttcctctgg

SEQ ID NO: 1002 - DNA, 22 bp, synthetic construct
  misc_feature 1..22: note = Structural abnormalities observed in CRISPR/Cas9-edited Jurkat T cells
  source 1..22: mol_type = other DNA
ttgcatctatagattgctttgg

SEQ ID NO: 1003 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 1004 - DNA, 22 bp, synthetic construct
  misc_feature 1..22: note = Structural abnormalities observed in CRISPR/Cas9-edited Jurkat T cells
  source 1..22: mol_type = other DNA
ctacatacatatatttctctgg

SEQ ID NO: 1005 - DNA, 22 bp, synthetic construct
  misc_feature 1..22: note = Structural abnormalities observed in CRISPR/Cas9-edited Jurkat T cells
  source 1..22: mol_type = other DNA
tttcctccatatattccacagg

SEQ ID NO: 1006 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 1007 - DNA, 22 bp, synthetic construct
  misc_feature 1..22: note = Structural abnormalities observed in CRISPR/Cas9-edited Jurkat T cells
  source 1..22: mol_type = other DNA
ttacatctatatattagtttgg

SEQ ID NO: 1008 - DNA, 22 bp, synthetic construct
  misc_feature 1..22: note = Structural abnormalities observed in CRISPR/Cas9-edited Jurkat T cells
  source 1..22: mol_type = other DNA
ttccctgtgtatattcctctgg

SEQ ID NO: 1009 - DNA, 22 bp, synthetic construct
  misc_feature 1..22: note = Structural abnormalities observed in CRISPR/Cas9-edited Jurkat T cells
  source 1..22: mol_type = other DNA
tttcttctgtattttcctctgg

SEQ ID NO: 1010 - DNA, 22 bp, synthetic construct
  misc_feature 1..22: note = Structural abnormalities observed in CRISPR/Cas9-edited Jurkat T cells
  source 1..22: mol_type = other DNA
ttgaatctatagattcctttgg

SEQ ID NO: 1011 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 1012 - DNA, 22 bp, synthetic construct
  misc_feature 1..22: note = Structural abnormalities observed in CRISPR/Cas9-edited Jurkat T cells
  source 1..22: mol_type = other DNA
ttaaatttataaattcctttgg

SEQ ID NO: 1013 - DNA, 22 bp, synthetic construct
  misc_feature 1..22: note = Structural abnormalities observed in CRISPR/Cas9-edited Jurkat T cells
  source 1..22: mol_type = other DNA
ataaatctatatatttctgtgg

SEQ ID NO: 1014 - DNA, 22 bp, synthetic construct
  misc_feature 1..22: note = Structural abnormalities observed in CRISPR/Cas9-edited Jurkat T cells
  source 1..22: mol_type = other DNA
ttacatttacacattccttggg

SEQ ID NO: 1015 - DNA, 22 bp, synthetic construct
  misc_feature 1..22: note = Structural abnormalities observed in CRISPR/Cas9-edited Jurkat T cells
  source 1..22: mol_type = other DNA
ttagatccatatatacttcagg

SEQ ID NO: 1016 - DNA, 22 bp, synthetic construct
  misc_feature 1..22: note = Structural abnormalities observed in CRISPR/Cas9-edited Jurkat T cells
  source 1..22: mol_type = other DNA
ttaaatctatacattgctttgg

SEQ ID NO: 1017 - DNA, 22 bp, synthetic construct
  misc_feature 1..22: note = Structural abnormalities observed in CRISPR/Cas9-edited Jurkat T cells
  source 1..22: mol_type = other DNA
ttagatctgcatattccttggg

SEQ ID NO: 1018 - DNA, 22 bp, synthetic construct
  misc_feature 1..22: note = Structural abnormalities observed in CRISPR/Cas9-edited Jurkat T cells
  source 1..22: mol_type = other DNA
ttgaatctatagattgctctgg

SEQ ID NO: 1019 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 1020 - DNA, 22 bp, synthetic construct
  misc_feature 1..22: note = Structural abnormalities observed in CRISPR/Cas9-edited Jurkat T cells
  source 1..22: mol_type = other DNA
ttacaactataatttccacagg

SEQ ID NO: 1021 - DNA, 22 bp, synthetic construct
  misc_feature 1..22: note = Structural abnormalities observed in CRISPR/Cas9-edited Jurkat T cells
  source 1..22: mol_type = other DNA
ttactactatctaatcctcagg

SEQ ID NO: 1022 - DNA, 22 bp, synthetic construct
  misc_feature 1..22: note = Structural abnormalities observed in CRISPR/Cas9-edited Jurkat T cells
  source 1..22: mol_type = other DNA
ttacttctaaatcttcctgggg

SEQ ID NO: 1023 - DNA, 22 bp, synthetic construct
  misc_feature 1..22: note = Structural abnormalities observed in CRISPR/Cas9-edited Jurkat T cells
  source 1..22: mol_type = other DNA
ttacatctgtagattgctttgg

SEQ ID NO: 1024 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 1025 - DNA, 22 bp, synthetic construct
  misc_feature 1..22: note = Structural abnormalities observed in CRISPR/Cas9-edited Jurkat T cells
  source 1..22: mol_type = other DNA
ttgcatctataaattactttgg

SEQ ID NO: 1026 - DNA, 22 bp, synthetic construct
  misc_feature 1..22: note = Structural abnormalities observed in CRISPR/Cas9-edited Jurkat T cells
  source 1..22: mol_type = other DNA
tttcatctatgttttcttctgg

SEQ ID NO: 1027 - DNA, 22 bp, synthetic construct
  misc_feature 1..22: note = Structural abnormalities observed in CRISPR/Cas9-edited Jurkat T cells
  source 1..22: mol_type = other DNA
taacacctagatattcatctgg

SEQ ID NO: 1028 - DNA, 22 bp, synthetic construct
  misc_feature 1..22: note = Structural abnormalities observed in CRISPR/Cas9-edited Jurkat T cells
  source 1..22: mol_type = other DNA
ttatatttatttattccactgg

SEQ ID NO: 1029 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattgctttgg

SEQ ID NO: 1030 - DNA, 22 bp, synthetic construct
  misc_feature 1..22: note = Structural abnormalities observed in CRISPR/Cas9-edited Jurkat T cells
  source 1..22: mol_type = other DNA
ttacattgatatattgcttagg

SEQ ID NO: 1031 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 1032 - DNA, 22 bp, synthetic construct
  misc_feature 1..22: note = Structural abnormalities observed in CRISPR/Cas9-edited Jurkat T cells
  source 1..22: mol_type = other DNA
ttaaatctatacattacttagg

SEQ ID NO: 1033 - DNA, 22 bp, synthetic construct
  misc_feature 1..22: note = Structural abnormalities observed in CRISPR/Cas9-edited Jurkat T cells
  source 1..22: mol_type = other DNA
gtaaacctatattttcctctgg

SEQ ID NO: 1034 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattacttggg

SEQ ID NO: 1035 - DNA, 22 bp, synthetic construct
  misc_feature 1..22: note = Structural abnormalities observed in CRISPR/Cas9-edited Jurkat T cells
  source 1..22: mol_type = other DNA
ttgcatctgtatattgctttgg

SEQ ID NO: 1036 - DNA, 22 bp, synthetic construct
  misc_feature 1..22: note = Structural abnormalities observed in CRISPR/Cas9-edited Jurkat T cells
  source 1..22: mol_type = other DNA
tttcatccattaattcctctgg

SEQ ID NO: 1037 - DNA, 22 bp, synthetic construct
  misc_feature 1..22: note = Structural abnormalities observed in CRISPR/Cas9-edited Jurkat T cells
  source 1..22: mol_type = other DNA
tttcatgtctatattcatcagg

SEQ ID NO: 1038 - DNA, 22 bp, synthetic construct
  misc_feature 1..22: note = Structural abnormalities observed in CRISPR/Cas9-edited Jurkat T cells
  source 1..22: mol_type = other DNA
ttgcatctatatcttgcccagg

SEQ ID NO: 1039 - DNA, 22 bp, synthetic construct
  misc_feature 1..22: note = Structural abnormalities observed in CRISPR/Cas9-edited Jurkat T cells
  source 1..22: mol_type = other DNA
ttacatgtagaaattcttctgg

SEQ ID NO: 1040 - DNA, 22 bp, synthetic construct
  misc_feature 1..22: note = Structural abnormalities observed in CRISPR/Cas9-edited Jurkat T cells
  source 1..22: mol_type = other DNA
ttccttctttatattcttcagg

SEQ ID NO: 1041 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 1042 - DNA, 22 bp, synthetic construct
  misc_feature 1..22: note = Structural abnormalities observed in CRISPR/Cas9-edited Jurkat T cells
  source 1..22: mol_type = other DNA
ttacatatatatgttcattggg

SEQ ID NO: 1043 - DNA, 22 bp, synthetic construct
  misc_feature 1..22: note = Structural abnormalities observed in CRISPR/Cas9-edited Jurkat T cells
  source 1..22: mol_type = other DNA
aaaaatctatatattccttagg

SEQ ID NO: 1044 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaatttctttgg

SEQ ID NO: 1045 - DNA, 22 bp, synthetic construct
  misc_feature 1..22: note = Structural abnormalities observed in CRISPR/Cas9-edited Jurkat T cells
  source 1..22: mol_type = other DNA
tttcatccatagatccctcagg

SEQ ID NO: 1046 - DNA, 22 bp, synthetic construct
  misc_feature 1..22: note = Structural abnormalities observed in CRISPR/Cas9-edited Jurkat T cells
  source 1..22: mol_type = other DNA
ttacatttatattttcacctgg

SEQ ID NO: 1047 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattgctttgg

SEQ ID NO: 1048 - DNA, 22 bp, synthetic construct
  misc_feature 1..22: note = Structural abnormalities observed in CRISPR/Cas9-edited Jurkat T cells
  source 1..22: mol_type = other DNA
atacatctatttagtactctgg

SEQ ID NO: 1049 - DNA, 22 bp, synthetic construct
  misc_feature 1..22: note = Structural abnormalities observed in CRISPR/Cas9-edited Jurkat T cells
  source 1..22: mol_type = other DNA
tgacatctatatcttattcagg

SEQ ID NO: 1050 - DNA, 22 bp, synthetic construct
  misc_feature 1..22: note = Structural abnormalities observed in CRISPR/Cas9-edited Jurkat T cells
  source 1..22: mol_type = other DNA
gtaaatctatatatttatctgg

SEQ ID NO: 1051 - DNA, 22 bp, synthetic construct
  misc_feature 1..22: note = Structural abnormalities observed in CRISPR/Cas9-edited Jurkat T cells
  source 1..22: mol_type = other DNA
ttccatctctaaattcctaggg

SEQ ID NO: 1052 - DNA, 22 bp, synthetic construct
  misc_feature 1..22: note = Structural abnormalities observed in CRISPR/Cas9-edited Jurkat T cells
  source 1..22: mol_type = other DNA
ttgcatctgtatattgctttgg

SEQ ID NO: 1053 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 1054 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattcctttgg

SEQ ID NO: 1055 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 1056 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 1057 - DNA, 22 bp, synthetic construct
  misc_feature 1..22: note = Structural abnormalities observed in CRISPR/Cas9-edited Jurkat T cells
  source 1..22: mol_type = other DNA
atatatctatatataccgcagg

SEQ ID NO: 1058 - DNA, 22 bp, synthetic construct
  misc_feature 1..22: note = Structural abnormalities observed in CRISPR/Cas9-edited Jurkat T cells
  source 1..22: mol_type = other DNA
ttgaatctatatattactttgg

SEQ ID NO: 1059 - DNA, 22 bp, synthetic construct
  misc_feature 1..22: note = Structural abnormalities observed in CRISPR/Cas9-edited Jurkat T cells
  source 1..22: mol_type = other DNA
tgacatatatatatatctcagg

SEQ ID NO: 1060 - DNA, 22 bp, synthetic construct
  misc_feature 1..22: note = Structural abnormalities observed in CRISPR/Cas9-edited Jurkat T cells
  source 1..22: mol_type = other DNA
tgacatatatatatatctcagg

SEQ ID NO: 1061 - DNA, 22 bp, synthetic construct
  misc_feature 1..22: note = Structural abnormalities observed in CRISPR/Cas9-edited Jurkat T cells
  source 1..22: mol_type = other DNA
tgacatatatatatatctcagg

SEQ ID NO: 1062 - DNA, 22 bp, synthetic construct
  misc_feature 1..22: note = Structural abnormalities observed in CRISPR/Cas9-edited Jurkat T cells
  source 1..22: mol_type = other DNA
atacatatatatattcatatgg

SEQ ID NO: 1063 - DNA, 22 bp, synthetic construct
  misc_feature 1..22: note = Structural abnormalities observed in CRISPR/Cas9-edited Jurkat T cells
  source 1..22: mol_type = other DNA
ataaatatatatatttctctgg

SEQ ID NO: 1064 - DNA, 22 bp, synthetic construct
  misc_feature 1..22: note = Structural abnormalities observed in CRISPR/Cas9-edited Jurkat T cells
  source 1..22: mol_type = other DNA
ttacatttatatatgtcactgg

SEQ ID NO: 1065 - DNA, 22 bp, synthetic construct
  misc_feature 1..22: note = Structural abnormalities observed in CRISPR/Cas9-edited Jurkat T cells
  source 1..22: mol_type = other DNA
ttacatcagcatactcctctgg

SEQ ID NO: 1066 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 1067 - DNA, 22 bp, synthetic construct
  misc_feature 1..22: note = Structural abnormalities observed in CRISPR/Cas9-edited Jurkat T cells
  source 1..22: mol_type = other DNA
atacatctagattttccgccgg

SEQ ID NO: 1068 - DNA, 22 bp, synthetic construct
  misc_feature 1..22: note = Structural abnormalities observed in CRISPR/Cas9-edited Jurkat T cells
  source 1..22: mol_type = other DNA
tgacatctacatattctcctgg

SEQ ID NO: 1069 - DNA, 22 bp, synthetic construct
  misc_feature 1..22: note = Structural abnormalities observed in CRISPR/Cas9-edited Jurkat T cells
  source 1..22: mol_type = other DNA
ttaaatctttttattcctttgg

SEQ ID NO: 1070 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 1071 - DNA, 22 bp, synthetic construct
  misc_feature 1..22: note = Structural abnormalities observed in CRISPR/Cas9-edited Jurkat T cells
  source 1..22: mol_type = other DNA
ttaagtcgacatattcctctgg

SEQ ID NO: 1072 - DNA, 22 bp, synthetic construct
  misc_feature 1..22: note = Structural abnormalities observed in CRISPR/Cas9-edited Jurkat T cells
  source 1..22: mol_type = other DNA
atatatctatatatgcctatgg

SEQ ID NO: 1073 - DNA, 22 bp, synthetic construct
  misc_feature 1..22: note = Structural abnormalities observed in CRISPR/Cas9-edited Jurkat T cells
  source 1..22: mol_type = other DNA
ttaaatctatagattgctttgg

SEQ ID NO: 1074 - DNA, 22 bp, synthetic construct
  misc_feature 1..22: note = Structural abnormalities observed in CRISPR/Cas9-edited Jurkat T cells
  source 1..22: mol_type = other DNA
atatatgtatatattccccagg

SEQ ID NO: 1075 - DNA, 22 bp, synthetic construct
  misc_feature 1..22: note = Structural abnormalities observed in CRISPR/Cas9-edited Jurkat T cells
  source 1..22: mol_type = other DNA
ttgcttctatatatttctaagg

SEQ ID NO: 1076 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattgctttgg

SEQ ID NO: 1077 - DNA, 22 bp, synthetic construct
  misc_feature 1..22: note = Structural abnormalities observed in CRISPR/Cas9-edited Jurkat T cells
  source 1..22: mol_type = other DNA
ttaattctatgtatttctctgg

SEQ ID NO: 1078 - DNA, 22 bp, synthetic construct
  misc_feature 1..22: note = Structural abnormalities observed in CRISPR/Cas9-edited Jurkat T cells
  source 1..22: mol_type = other DNA
ttacatctatttagtgctgtgg

SEQ ID NO: 1079 - DNA, 22 bp, synthetic construct
  misc_feature 1..22: note = Structural abnormalities observed in CRISPR/Cas9-edited Jurkat T cells
  source 1..22: mol_type = other DNA
atacatatatatattccttagg

SEQ ID NO: 1080 - DNA, 22 bp, synthetic construct
  misc_feature 1..22: note = Structural abnormalities observed in CRISPR/Cas9-edited Jurkat T cells
  source 1..22: mol_type = other DNA
taacaactatatactcatctgg

SEQ ID NO: 1081 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 1082 - DNA, 22 bp, synthetic construct
  misc_feature 1..22: note = Structural abnormalities observed in CRISPR/Cas9-edited Jurkat T cells
  source 1..22: mol_type = other DNA
tcacatctgtccattcctcagg

SEQ ID NO: 1083 - DNA, 22 bp, synthetic construct
  misc_feature 1..22: note = Structural abnormalities observed in CRISPR/Cas9-edited Jurkat T cells
  source 1..22: mol_type = other DNA
taagatctatatattggtcagg

SEQ ID NO: 1084 - DNA, 22 bp, synthetic construct
  misc_feature 1..22: note = Structural abnormalities observed in CRISPR/Cas9-edited Jurkat T cells
  source 1..22: mol_type = other DNA
ttaaatctataaattactttgg

SEQ ID NO: 1085 - DNA, 22 bp, synthetic construct
  misc_feature 1..22: note = Structural abnormalities observed in CRISPR/Cas9-edited Jurkat T cells
  source 1..22: mol_type = other DNA
ttacatctttatattctcaggg

SEQ ID NO: 1086 - DNA, 22 bp, synthetic construct
  misc_feature 1..22: note = Structural abnormalities observed in CRISPR/Cas9-edited Jurkat T cells
  source 1..22: mol_type = other DNA
taaaatctatatattcctgtgg

SEQ ID NO: 1087 - DNA, 22 bp, synthetic construct
  misc_feature 1..22: note = Structural abnormalities observed in CRISPR/Cas9-edited Jurkat T cells
  source 1..22: mol_type = other DNA
ttacatgtctggattcctcagg

SEQ ID NO: 1088 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattactctgg

SEQ ID NO: 1089 - DNA, 22 bp, synthetic construct
  misc_feature 1..22: note = Structural abnormalities observed in CRISPR/Cas9-edited Jurkat T cells
  source 1..22: mol_type = other DNA
ctacatgtatacatgcctctgg

SEQ ID NO: 1090 - DNA, 22 bp, synthetic construct
  misc_feature 1..22: note = Structural abnormalities observed in CRISPR/Cas9-edited Jurkat T cells
  source 1..22: mol_type = other DNA
ttagatctatgttatcctcagg

SEQ ID NO: 1091 - DNA, 22 bp, synthetic construct
  misc_feature 1..22: note = Structural abnormalities observed in CRISPR/Cas9-edited Jurkat T cells
  source 1..22: mol_type = other DNA
ttctctctatatatttctctgg

SEQ ID NO: 1092 - DNA, 22 bp, synthetic construct
  misc_feature 1..22: note = Structural abnormalities observed in CRISPR/Cas9-edited Jurkat T cells
  source 1..22: mol_type = other DNA
ttgaatctataaattgctctgg

SEQ ID NO: 1093 - DNA, 22 bp, synthetic construct
  misc_feature 1..22: note = Structural abnormalities observed in CRISPR/Cas9-edited Jurkat T cells
  source 1..22: mol_type = other DNA
ttaaatctgtatattgctttgg

SEQ ID NO: 1094 - DNA, 22 bp, synthetic construct
  misc_feature 1..22: note = Structural abnormalities observed in CRISPR/Cas9-edited Jurkat T cells
  source 1..22: mol_type = other DNA
taccaactttatattcctcagg

SEQ ID NO: 1095 - DNA, 20 bp, synthetic construct
  misc_feature 1..20: note = DNA encoding guide RNA
  source 1..20: mol_type = other DNA
cgaggaatatataggtgtaa

SEQ ID NO: 1096 - DNA, 126 bp, synthetic construct
  misc_feature 1..126: note = Donor sequence for homologous recombination
  source 1..126: mol_type = other DNA
acccaaagggttcaggaagcacgtacttcgataatgaacttgcacggtagattctttgtccttgtatatatctgtcccattacatctatatattcctcatgggtccaggatgcgttttcccaggtc

SEQ ID NO: 1097 - DNA, 20 bp, synthetic construct
  misc_feature 1..20: note = PCR primer
  source 1..20: mol_type = other DNA
cttggtgcagatcaaagagc

SEQ ID NO: 1098 - DNA, 19 bp, synthetic construct
  misc_feature 1..19: note = PCR primer
  source 1..19: mol_type = other DNA
ctggtgatgggcttgccac

SEQ ID NO: 1099 - DNA, 126 bp, synthetic construct
  misc_feature 1..126: note = Donor sequence for homologous recombination
  source 1..126: mol_type = other DNA
tgcaataccagcatcacatgggtagagggaacggtgggaacactgctctcagacattacaagactggacctgggaaaacgcatcctggatccacgaggaatatatagatgtaatgggacagatata

SEQ ID NO: 1100 - DNA, 20 bp, synthetic construct
  misc_feature 1..20: note = PCR primer
  source 1..20: mol_type = other DNA
cttggtgcagatcaaagagc

SEQ ID NO: 1101 - DNA, 19 bp, synthetic construct
  misc_feature 1..19: note = PCR primer
  source 1..19: mol_type = other DNA
ctggtgatgggcttgccac

SEQ ID NO: 1102 - DNA, 44 bp, synthetic construct
  misc_feature 1..44: note = PCR primer
  misc_difference 21..24: note = n is a, c, g, or t
  source 1..44: mol_type = other DNA
acacgacgctcttccgatctnnnngaggacagagtgtttgtgaa

SEQ ID NO: 1103 - DNA, 55 bp, synthetic construct
  misc_feature 1..55: note = PCR primer
  source 1..55: mol_type = other DNA
gtgactggagttcagacgtgtgctcttccgatctctctagccagaaagttctcac

SEQ ID NO: 1104 - DNA, 126 bp, synthetic construct; identical to SEQ ID NO: 1096
  misc_feature 1..126: note = Donor sequence for homologous recombination
  source 1..126: mol_type = other DNA

SEQ ID NO: 1105 - DNA, 20 bp, synthetic construct
  misc_feature 1..20: note = PCR primer
  source 1..20: mol_type = other DNA
cttggtgcagatcaaagagc

SEQ ID NO: 1106 - DNA, 19 bp, synthetic construct
  misc_feature 1..19: note = PCR primer
  source 1..19: mol_type = other DNA
ctggtgatgggcttgccac

SEQ ID NO: 1107 - DNA, 3273 bp, synthetic construct
  misc_feature 1..3273: note = Lentiviral vector
  source 1..3273: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggtcgaggatatcggatctagatcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctaggatcaaggtcaggaacagagaaacaggagaatatgggccaaacaggatatctgtggtaagcagttcctgccccgctcagggccaagaacagttggaacaggagaatatgggccaaacaggatatctgtggtaagcagttcctgccccgctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatcgatgaattcgagctcggtacccggggatcccgggtgatcagtcgagctcaagcttcgaattctgcagtcgacggtaccgcgggcccgggatccaccggtcgccaccatggagcacagcaccttcctgtctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccatgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccacctgggcggcaactgggctcggaacaagtgataaagcggccaactcgacgggcccgcggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

(19) United States
(12) Patent Application Publication
(10) Pub. No.: US 2025/0144147 A1
(43) Pub. Date: May 8, 2025

(54) BASE EDITING AND CRISPR/CAS9 GENE EDITING STRATEGIES TO CORRECT CD3 SEVERE COMBINED IMMUNODEFICIENCY IN HEMATOPOIETIC STEM CELLS

(71) Applicant: The Regents of the University of California, Oakland, CA (US)

(72) Inventors: Donald B. KOHN, Tarzana, CA (US); Grace MCAULEY, Los Angeles, CA (US)

(73) Assignee: The Regents of the University of California, Oakland, CA (US)

(21) Appl. No.: 18/832,890

(22) PCT Filed: Jan. 27, 2023
(86) PCT No.: PCT/US2023/011760

§ 371 Date: Jul. 24, 2024

Related U.S. Application Data
(60) Provisional application No. 63/303,812, filed on Jan. 27, 2022.

(51) Int. Cl. A61K 35/28; A61P 37/04; C12N 9/22; C12N 15/11; C12N 15/86; C12N 15/90

(52) U.S. Cl. CPC A61K 35/28; A61P 37/04; C12N 9/22; C12N 15/111; C12N 15/86; C12N 15/907; C12N 2310/20; C12N 2740/15043; C12N 2830/48

(57) ABSTRACT

Provided herein are compositions, systems, and methods to provide two gene editing-based approaches that can be used to correct the CD35 SCID-causing C202T mutation (TGA→CGA). In certain embodiments one approach involves CRISPR/Cas9 homology-directed repair (HDR)-mediated correction with a single-strand oligodeoxynucleotide (ssODN) homologous donor. In certain embodiments another approach comprises Adenine Base Editing (ABE)-correction, to precisely revert the CD35 SCID-causing C202T mutation (TGA→CGA).

### CROSS-REFERENCE TO RELATED APPLICATIONS

This application claims benefit of and priority to U.S. Ser. No. 63/303,812, filed on Jan. 27, 2022, which is incorporated herein by reference in its entirety for all purposes.

### STATEMENT OF GOVERNMENTAL SUPPORT

[Not Applicable]

### INCORPORATION BY REFERENCE OF SEQUENCE LISTING PROVIDED AS ST26 FORMAT XML FILE

[Not Applicable]

### BACKGROUND

CD3δ severe combined immunodeficiency (SCID) is a devastating inborn error of immunity (IEI) caused, in many of the patients, by a homozygous mutation in the CD3D gene (C202T substitution) resulting in a premature nonsense (stop) codon (R68X) and the absence of CD3 δ protein. The CD3 protein complex is a vital component for T-cell signaling and T-cell receptor (TCR) surface expression in the transition from double-negative to single-positive T cells. The absence of the CD3δ chain results in a total arrest of thymocyte development at the double-negative to double-positive stage alongside impaired γ/δ T cells. Patients with CD3 δ SCID present with a complete absence of T cells with present, but non-functional, B cells and NK cells (T−B+NK+ SCID); they are severely susceptible to lethal infections leading to infant mortality if not treated by allogeneic hematopoietic stem cell transplantation (HSCT). However, allogeneic HSCT is often limited by a lack of suitable donors, and to our knowledge, no attempt has been made to permanently correct CD3δ SCID using an ex vivo gene editing strategy for autologous HSCT.

### SUMMARY

Described herein are two gene editing-based approaches that can be used to correct the CD3δ SCID-causing C202T mutation (TGA→CGA). In certain embodiments one approach involves CRISPR/Cas9 homology-directed repair (HDR)-mediated correction with a single-strand oligodeoxynucleotide (ssODN) homologous donor. In certain embodiments another approach comprises Adenine Base Editing (ABE)-correction, to precisely revert the CD3δ SCID-causing C202T mutation (TGA→CGA).

Accordingly, various embodiments provided herein may include, but need not be limited to, one or more of the following:

Embodiment 1: A system for homology-directed repair (HDR)-mediated correction of the C202T mutation that produces CD3δ SCID disease, said system comprising:
a first single-guide RNA (sgRNA) that directs Cas9 cutting upstream of the C2020T mutation;
a second single-guide RNA (sgRNA) that directs Cas9 cutting downstream of the C2020T mutation; and
a single-strand oligodeoxynucleotide (ssODN) homologous donor comprising a nucleotide sequence that corrects the C202T mutation.

Embodiment 2: The system of embodiment 1, wherein said first single-guide RNA comprises a nucleotide sequence that directs Cas9 cutting two base pairs (bp) upstream C202T mutation.

Embodiment 3: The system according to any one of embodiments 1-2, wherein said second single-guide RNA comprises a nucleotide sequence that directs Cas9 cutting five bp downstream of the C202T mutation.

Embodiment 4: The system according to any one of embodiments 1-3, wherein said ssODN is complementary to the nontarget strand with asymmetric homology arms.

Embodiment 5: The system of embodiment 4, wherein said asymmetric homology arms extend 33 bp downstream and 60 bp upstream of the respective sgRNA-guided Cas9 cut site.

Embodiment 6: The system according to any one of embodiments 1-5, wherein said ssODN comprises a silent PAM mutation to prevent continual nuclease activity.

Embodiment 7: The system according to any one of embodiments 1-6, wherein said system comprises a CRISPR protein or a nucleic acid encoding a CRISPR protein.

Embodiment 8: The system of embodiment 7, wherein said system comprises a CRISPR protein.

Embodiment 9: The system of embodiment 7, wherein said system comprises a nucleic acid encoding a CRISPR protein.

Embodiment 10: The system according to any one of embodiments 1-9, wherein said system comprises a CRISPR/cas9 protein or a nucleic acid encoding a CRISPR/cas9 protein.

Embodiment 11: The system of embodiment 10, wherein said system comprises a CRISPR/cas9 protein.

Embodiment 12: The system of embodiment 10, wherein said system comprises a nucleic acid encoding a CRISPR/cas9 protein.

Embodiment 13: The system according to any one of embodiments 1-6, wherein said system is provided as kit comprising one or more containers containing:
said first single-guide RNA (sgRNA);
said second single-guide RNA (sgRNA); and
said single-strand oligodeoxynucleotide (ssODN).

Embodiment 14: The system of embodiment 13, wherein said kit further comprises a container containing a CRISPR protein or a nucleic acid encoding a CRISPR protein.

Embodiment 15: The system of embodiment 14, wherein said kit further comprises a container containing a CRISPR/cas9 protein or a nucleic acid encoding a CRISPR/cas9 protein.

Embodiment 16: A method of correcting a C202T mutation in a mammalian cell using homology-directed repair, said method comprising:
introducing a CRISPR protein, or a nucleic acid comprising a CRISPR protein, and the system according to any one of embodiments 1-6 into said cell; and
culturing said cell to permit homology-directed repair (HDR-mediated correction) of the C202T mutation in said cell to provide a corrected cell.

Embodiment 17: The method of embodiment 16, wherein said method comprises introducing a CRISPR protein into said cell.

Embodiment 18: The method of embodiment 17, wherein said method comprises introducing a CRISPR/cas9 protein into said cell.

Embodiment 19: The method of embodiment 16, wherein said method comprises introducing a nucleic acid that encodes a CRISPR protein into said cell.

Embodiment 20: The method of embodiment 19, wherein said method comprises introducing a nucleic acid that encodes a CRISPR/cas9 protein into said cell.

Embodiment 21: The method according to any one of embodiments 16-20, wherein the cell is a stem/progenitor cell.

Embodiment 22: The method of embodiment 21, wherein said cell is a stem cell derived from bone marrow, and/or from umbilical cord blood, and/or from peripheral blood.

Embodiment 23: The method of embodiment 22, wherein, wherein the cell is a human hematopoietic progenitor cell.

Embodiment 24: The method of embodiment 23, wherein the human hematopoietic progenitor cell is a CD34+ cell.

Embodiment 25: The method according to any one of embodiments 16-24, wherein said cell is from a human subject identified as having CD3δ severe combined immunodeficiency (SCID).

Embodiment 26: The method according to any one of embodiments 16-25, wherein said method further comprises introducing said corrected cell into a subject identified as having CD3δ severe combined immunodeficiency (SCID).

Embodiment 27: The method of embodiment 26, wherein said method restores wildtype levels of CD3δ expression.

Embodiment 28: A method of treating a subject for CD3δ severe combined immunodeficiency (SCID), said method comprising:
providing stem/progenitor cells from said subject;
correcting a C202T mutation in said cells ex vivo using the method according to any one of embodiments 16-20 to produce corrected cells; and
introducing said corrected cells into said subject.

Embodiment 29: The method of embodiment 28, wherein said cell is a stem cell derived from bone marrow, and/or from umbilical cord blood, and/or from peripheral blood.

Embodiment 30: The method of embodiment 29, wherein, wherein the cell is a human hematopoietic progenitor cell.

Embodiment 31: The method of embodiment 30, wherein the human hematopoietic progenitor cell is a CD34+ cell.

Embodiment 32: The method according to any one of embodiments 28-31, wherein subject is a human subject identified as having CD3δ severe combined immunodeficiency (SCID).

Embodiment 33: The method according to any one of embodiments 28-32, wherein said method restores wildtype levels of CD3δ expression and subsequent T-cell development.

Embodiment 34: An adenosine base editor, wherein said base editor is a variant of the wildtype NGG-recognizing Cas9 (D10A) nickase (Cas9n) comprising a combination of amino acid substitutions selected from the group consisting of:
(1) NRTH-ABE8e: A10T, I322V, S409I, E427G, R654L, R753G, R1114G, D1135N, D1180G, G1218S, E1219V, Q1221H, P1249S, E1253K, P1321S, D1332G, and R1335L;
(2) VRER-ABE8e: D1135V, G1218R, R1335E, and T1337R; and
(3) A262T, R324L, S409I, E480K, E543D, M694I, and E1219V.

Embodiment 35: The base editor of embodiment 34, wherein said base editor is a variant of the wildtype NGG-recognizing Cas9 (D10A) nickase (Cas9n) comprising the following amino acid substitutions: A10T, I322V, S409I, E427G, R654L, R753G, R1114G, D1135N, D1180G, G1218S, E1219V, Q1221H, P1249S, E1253K, P1321S, D1332G, and R1335L.

Embodiment 36: The base editor of embodiment 35, wherein said base editor comprises the amino acid sequence of SEQ ID NO:4.

Embodiment 37: The base editor of embodiment 35, wherein said base editor is encoded by the nucleic acid sequence of SEQ ID NO:3.

Embodiment 38: The base editor of embodiment 34, wherein said base editor is a variant of the wildtype NGG-recognizing Cas9 (D10A) nickase (Cas9n) comprising the following amino acid substitutions: D1135V, G1218R, R1335E, and T1337R.

Embodiment 39: The base editor of embodiment 38, wherein said base editor comprises the amino acid sequence of SEQ ID NO:6.

Embodiment 40: The base editor of embodiment 38, wherein said base editor is encoded by the nucleic acid sequence of SEQ ID NO:5.

Embodiment 41: The base editor of embodiment 34, wherein said base editor is a variant of the wildtype NGG-recognizing Cas9 (D10A) nickase (Cas9n) comprising the following amino acid substitutions: A262T, R324L, S409I, E480K, E543D, M694I, and E1219V.

Embodiment 42: The base editor of embodiment 41, wherein said base editor comprises the amino acid sequence of SEQ ID NO:8.

Embodiment 43: The base editor of embodiment 41, wherein said base editor is encoded by the nucleic acid sequence of SEQ ID NO:7.

Embodiment 44: A nucleic acid encoding a base editor according to any one of embodiments 34-43.

Embodiment 45: A system for base-editor-directed repair (BE-mediated correction) of a C202T mutation that produces CD3δ SCID disease, said system comprising:
a base editor according to any one of embodiments 34-44, or a nucleic acid encoding a base editor according to any one of embodiments 34-44; and
a single-guide RNA (sgRNA) that directs said base editor to the location of the nucleic acids encoding the C202T mutation.

Embodiment 46: The system of embodiment 45, wherein said sgRNA comprises the sequence of the G1 (Guide 2T) sgRNA (SEQ ID NO:1).

Embodiment 47: The system of embodiment 45, wherein said sgRNA comprises the sequence of the Guide 5T) sgRNA (SEQ ID NO:2).

Embodiment 48: A method of correcting a C202T mutation in a mammalian cell using Adenine Base Editing (ABE)-correction, said method comprising:
introducing a base editor according to any one of embodiments 34-43, or a nucleic acid encoding a base editor according to any one of embodiments 34-43, and a single-guide RNA (sgRNA) that directs said base editor to the location of the nucleic acids encoding the C202T mutation into said cell; and
culturing said cell to permit base editor (BE) mediated correction of the C202T mutation in said cell to provide a corrected cell.

Embodiment 49: The method of embodiment 48, wherein said method comprises introducing a base editor according to any one of embodiments 34-43 into said cell.

Embodiment 50: The method of embodiment 48, wherein said method comprises introducing a nucleic acid encoding a base editor according to any one of embodiments 34-43 into said cell.

Embodiment 51: The method according to any one of embodiments 48-50, wherein said sgRNA comprises the sequence of the G1 (Guide 2T) sgRNA (SEQ ID NO:1).

Embodiment 52: The method according to any one of embodiments 48-50, wherein said sgRNA comprises the sequence of the Guide 5T sgRNA (SEQ ID NO:2).

Embodiment 53: The method according to any one of embodiments 48-52, wherein the cell is a stem/progenitor cell.

Embodiment 54: The method of embodiment 53, wherein said cell is a stem cell derived from bone marrow, and/or from umbilical cord blood, and/or from peripheral blood.

Embodiment 55: The method of embodiment 54, wherein, wherein the cell is a human hematopoietic progenitor cell.

Embodiment 56: The method of embodiment 55, wherein the human hematopoietic progenitor cell is a CD34+ cell.

Embodiment 57: The method according to any one of embodiments 48-56, wherein said cell is from a human subject identified as having CD3δ severe combined immunodeficiency (SCID).

Embodiment 58: The method according to any one of embodiments 48-57, wherein said method further comprises introducing said corrected cell into a subject identified as having CD3δ severe combined immunodeficiency (SCID).

Embodiment 59: The method of embodiment 58, wherein said method restores wildtype levels of CD3δ expression.

Embodiment 60: A method of treating a subject for CD3δ severe combined immunodeficiency (SCID), said method comprising:
providing stem/progenitor cells from said subject;
correcting a C202T mutation in said cells ex vivo using the method according to any one of embodiments 48-52 to produce corrected cells; and
introducing said corrected cells into said subject.

Embodiment 61: The method of embodiment 60, wherein said cell is a stem cell derived from bone marrow, and/or from umbilical cord blood, and/or from peripheral blood.

Embodiment 62: The method of embodiment 61, wherein, wherein the cell is a human hematopoietic progenitor cell.

Embodiment 63: The method of embodiment 62, wherein the human hematopoietic progenitor cell is a CD34+ cell.

Embodiment 64: The method according to any one of embodiments 60-63, wherein subject is a human subject identified as having CD3δ severe combined immunodeficiency (SCID).

Embodiment 65: The method according to any one of embodiments 60-64, wherein said method restores wildtype levels of CD3δ expression and subsequent T-cell development.

Embodiment 66: A lentivirus for evaluating gene editing correction of the CD3δ SCID-causing C202T mutation, said lentivirus construct comprising the elements illustrated in FIG. 3.

Embodiment 67: The lentivirus of embodiment 66, wherein said lentivirus comprises the sequence of SEQ ID NO:1107.

### Definitions

The terms “subject,” “individual,” and “patient” may be used interchangeably and typically a mammal, in certain embodiments a human or a non-human primate.

### BRIEF DESCRIPTION OF THE DRAWINGS

FIG. 1, panels A-B, illustrates base editing (panel A) and CRISPR/Cas9-mediated editing (panel B) in CD3 KO Jurkat Cells. sgRNA 1 (G1) (panel A) (TTCCTCATGGGTCCAGGATGCGTT, SEQ ID NO:1). sgRNA (panel B): TACATCTATATATTCCTCATGGG, SEQ ID NO:2).

FIG. 2, panels A-B, illustrates restoring CD3 protein complex expression in CD3 KO Jurkat cells.

FIG. 3 schematically illustrates a lentiviral vector to evaluate therapeutic BE reagents in HD 34+ cells.

FIG. 4 illustrates a timeline to evaluate therapeutic BE reagents in HD 34+ cells.

FIG. 5 shows vector copy number (VCN) determination and TIDE analysis of transduced (top panel) and base edited (bottom panel) HD CD34+ cells 6 days post-transduction.

FIG. 6 shows vector copy number (VCN) determination and TIDE analysis of transduced (top panel) and base edited (bottom panel) HD CD34+ cells 14 days post-transduction.

FIG. 7, panels A-G, illustrates that adenine base editing efficiently rescues CD3/TCR expression and signaling in a T cell line disease model. Panel A) ABE catalyzes the transition of adenine (A) to guanine (G). An adenosine deaminase is linked to a Cas9 nickase (Cas9n). The target A is deaminated to an inosine (I) (ACT-to-ICT) through a hydrolysis reaction, while the non-edited strand is nicked by Cas9n, inducing base excision and mismatch repair to permanently correct both strands of DNA. Panel B) Plasmids encoding a CD3D-targeting sgRNA and either ABEmax-NRTH, ABE8e-NRTH, ABE8e-NG, ABE8e-xCas9 (3.7), or ABE8e-VRER were transfected by electroporation into CD3D (C202T) Jurkat T cells. To assess restoration of CD3 by CRISPR/Cas9 HDR-mediated correction, sgRNA and rCas9 protein (RNP) and ssODN donor were co-electroporated into CD3D (C202T) Jurkat T cells. Panels C-D) Editing efficiencies were measured 5 days after electroporation by high-throughput sequencing (HTS) and restoration of CD3 expression was measured by flow cytometry with an anti-CD3 antibody. Panels E-F) To measure CD3/TCR complex activation and signaling, we performed a calcium flux assay and quantified area under the calcium flux curve of treated and untreated CD3D (C202T) Jurkat T cells following stimulation with anti-CD3 and anti-CD28. Panel G) CD3D (C202T) Jurkat T cells treated with RNP+ssODN (CRISPR/Cas9-edited), ABEmax-NRTH and sgRNA, or mock electroporated controls were harvested 24 hours after electroporation for G-banded karyotype analysis. The karyotype image represents one cell edited with Cas9 RNP and ssODN. Below the image, the representative abnormalities observed in this clone is shown as a composite karyotype using the International System for Human Cytogenomic Nomenclature (ISCN). Black arrows indicate clonal structural abnormalities inherent to the pseudo-tetraploid Jurkat T cell line, where, “clonal” is defined as at least two cells with the same chromosomal rearrangement. The red box identifies a clonal deletion of 11q23 distal to the on-target editing site. Panels B, C, E) Data shown as mean±SD of nine replicates from 3 independent experiments. Statistical significance was calculated using non-parametric t-test (****p<0.0001); ns, not significant.

FIG. 8, panels A-J, illustrate the characterization of local bystander and genome-wide off-target editing in CD3D (C202T) Jurkat T cells and CD3δ SCID patient CD34+ HSPCs. Panel A) Schematic representation of the CD3D target with the on-target A at protospacer position 7 (green) along with potential missense bystander edits shown in purple (A18), orange (A15), pink (A0), and blue (A-2) (top amino acid sequence (SEQ ID NO: 3), middle nucleic acid sequence (SEQ ID NO:4), bottom nucleic acid sequence (SEQ ID NO: 5). Potential bystander edits were named by their position in relation to the start of the PAM site (maroon), with position 1 residing most distal to the PAM. Resulting amino acid substitutions of potential bystander edits are shown in red below each respective bystander protospacer position. Panel B) Plasmids encoding the CD3D-targeting sgRNA and either ABEmax-NRTH, ABE8e-NRTH, or ABE8e-NG were delivered by electroporation in CD3D (C202T) Jurkat T cells. Editing efficiencies were measured by HTS at on-target and bystander adenines five days after electroporation. Panels C and D) Proviral maps of lentiviral vectors (LVs) used to characterize the effects of A0 bystander editing. MNDU3 (Myeloproliferative Sarcoma Virus, Negative Control region deleted Long Terminal Repeat promoter) is used to drive expression of the CD3D cDNA (with or without the A0 mutation). Panels E-G) 14 days after transduction, a calcium flux assay was performed to assess restoration of CD3/TCR signaling. LV vector copy number (VCN) was quantified by droplet digital PCR (ddPCR). Panel H) Venn diagram of potential off-target sites assessed by multiplexed-targeted HTS nominated by CIRCLE-seq (blue), Cas-OFFinder (pink), GUIDE-seq (green), and predicted sites for which off-target editing was observed by multiplex-HTS (yellow) in CD36 SCID HSPCs electroporated with ABEmax-NRTH mRNA and CD3D-localizing sgRNA. Panel I) Bar graphs demonstrate the percentage of sequencing reads containing A\hat{ }T-to-G\hat{ }C point mutations within protospacer positions 4-10 at on- and off-target sites in genomic DNA from CD36 SCID HSPCs treated with ABEmax-NRTH mRNA or untreated controls (n=3). Panel J) CIRCLE-seq read counts and alignment to the on-target guide sequence for each validated off-target site. (Top on-target sequence (SEQ ID NO:6)). Panel K) Genomic locations of validated off-target sites. Panels B and G) Data shown as mean±SD of 3 independent experiments. Statistical significance was calculated by non-parametric t-test; ns, not significant.

FIG. 9, panels A-K, shows that engrafted healthy human HSPCs retain high-levels of gene correction in a humanized mouse model. Panel A) Experimental timeline for xenograft studies. Healthy donor (HD) CD34+ HSPCs were pre-stimulated for 24 hours and transduced with lentiviral vector (MNDU3-CD3D c.202C>T cDNA). 24 hours after transduction, HSPCs were electroporated with ABEmax-NRTH mRNA and sgRNA and ˜8×10⁵ treated cells were transplanted into 6-8 weeks-old NBSGW mice (n=10 mice humanized with LV and BE treated HSPCs; see also FIG. 18). Sixteen weeks post-transplant, bone marrow, spleen, and thymus were analyzed by flow cytometry. Panel B) Proviral map of lentiviral disease target for integration in healthy CD34+ HSPCs. Components of the LV are similar to those described in FIG. 8, panels C-D, with the exception of 20 bp codon optimized regions on N- and C-termini (orange boxes) of the CD3D cDNA to allow for specific targeted DNA amplification of the CD3D cDNA (not the endogenous CD3D gene) for base editing analysis. The MNDU3 promoter drives expression of a CD3D cDNA containing the pathogenic CD3D c.202C>T mutation (red line). Sixteen weeks after infusion, engraftment was measured by percentage of human CD45+ cells in recipient mice Panel C) bone marrow, Panel D) spleen, and Panel E) thymus. Abundance of human CD19+ B cells, CD33+ myeloid, CD34+ HSPCs, CD56+NK cells, and CD3+ T cells were measured as percentages of the hCD45+ population in transplant recipient bone marrow (panel F) and spleen (panel G). Panel H) Human CD3−TCRαβ−CD4+ immature SP (CD4 ISP), CD3+TCRαβ+CD4+ (CD4 SP), CD3+TCRαβ+CD8+ single-positive (CD8 SP), CD4-CD8-doublenegative (DN), and CD4+CD8+ double-positive (DP) cells as percentages of the hCD45+ population in recipient mouse thymus. Panel I) CD3D c.202C>T editing efficiency and VCN determined by HTS and ddPCR, respectively, in cells cultured for 14 days after electroporation (pre-transplant) or in whole tissues 16 weeks after transplant. Panel J) HTS of on-target and bystander adenines in the pre-transplant HSPC cell product and bulk tissues post-transplant. Panel K) CD3D c.202C>T editing efficiency in human-derived hematopoietic lineages from mouse bone marrow. Populations were FACS sorted using hCD34+, hCD33+, hCD19+, and hCD56+ antibodies for HSPC, myeloid, B cell, and NK cell collection, respectively. n=2 mice that received untreated cells, n=4 mice that received LV-transduced cells, and n=10 mice that received LV-transduced and edited cells. Data shown as mean±SD; Panel K) one-way ANOVA, non-parametric t-test elsewhere; ns, not significant.

FIG. 10, panels A-H, shows that base-editing of CD3δ SCID CD34+ HSPCs rescues T cell differentiation. Panel A) Workflow of T cell differentiation: HSPCs were isolated from bone marrow of a patient with CD36 SCID and electroporated with ABEmax-NRTH mRNA and sgRNA localizing to the CD3D c.202C>T mutation. Treated cells were aggregated by centrifugation with MS5-hDLL4 stromal cells and installed on a cell culture insert for ATO differentiation. Panel B) HTS editing efficiencies at target and bystander adenines (see FIG. 8, panel A for descriptions of nomenclature) and indels after 5 days of in vitro culture post-electroporation (‘preATO’) and 12-15 weeks after ATO T cell differentiation (‘post-ATO’), UnEd, unedited. A portion of cells were plated in methylcellulose for a CFU assay. Panel C) Clonal editing outcomes determined by HTS of the CD3D target by analysis of individual day 14 CFUs. Exp #1, n=100 CFUs and Exp #2, n=130 CFUs. Mono, monoallelic; Bi, biallelic. Panels D-H) Kinetics of T cell differentiation in ATOs derived from CD34+ HSPC, Panels D and E) Representative flow cytometry profiles of Panel D) CD3+ and TCRαβ+ expression gated on DAPI−CD45+Lin−(CD56−CD14−) TCRγδ−, and CD4 and CD8 expression in panel E) CD3+TCRa0+ cells gated on CD45+Lin−. HD (top), unedited patient (middle), and edited patient (bottom) ATOs (n=6-9 for each time point). Cell counts of total cell output (panel F), CD3+TCRαβ+ (panel g), and SP8 cells (panel H) per ATO (n=6-12 per time point).

FIG. 11, panels A-J, shows that T cell differentiation from CD3δ SCID HSPCs is blocked at the DP stage. Panels A-E) T cell differentiation of HD, unedited patient, and edited patient ATOs, n=6-12 from 4 independent experiments. Panel A) Representative flow cytometry profiles depicting T cell differentiation of DN (green), ISP4 (aqua), and DP-E (blue) populations in cells gated on CD3−TCRa0-cells at weeks 7 and 9. Panel B) Frequency of DN, ISP4, and DP-E cells in CD45+Lin-cells in at week 12. Data shown as mean±SD. Statistical significance was calculated by unpaired nonparametric t-test***p<0.001. Cell counts of DN (panel C), ISP4 (panel D), and DP-E cells (panel E) per ATO. Panels F-J) Cellular Indexing of Transcriptomes and Epitopes by sequencing (CITE-seq) analysis of unedited and edited CD36 SCID patient ATOs and week 8 (n=4). Panel F) Weighted nearest neighbors UMAP (WNN_UMAP) visualizations of annotated populations in unedited (left) and edited (right) patient ATOs. Expression of lineage defining surface proteins (panel G) and RNA (panel H) across clusters. Panel I) Frequency of developing T cell (DN, ISP4, DP-E, DP-L, SP8RO, and SP8RA) and other immune cell (CD34+, NK, innate, pDC, γδ T cell, B cell) subsets in unedited (left) or edited (right) samples. Panel J) WNN_UMAP visualization of no TRA or TRB (grey), TRB only (orange), and both TRA and TRB (purple) expression.

FIG. 12, panels A-F, shows that edited CD3δ SCID ATO-derived T cells express features of maturation without evidence of exhaustion. Panel A) Representative flow cytometry profiles depicting maturation markers (CCR7, CD62L, CD27, CD28, CD45RO, and CD45RA) in cells gated on SP8 cells-CD3+TCRαβ+CD8aα+CD8β+, in week 12 ATOs (n=9, from four independent experiments). Panel B) RNA expression of selected genes (y-axis) across clusters in edited patient ATOs by CITE-seq; Cyt., cytokine. Panel C) Gene Set Enrichment Analysis (GSEA) of differentially expressed genes from GOBP (Gene Ontology Biological Process) and GOCC (Gene Ontology Cellular Compartment) between SP8 T and DN cells from edited ATOs. Dot size represents adjusted p-value (padj; two-sided permutation test). NES, normalized enrichment score; PM, plasma membrane. GSEA plots of representative gene sets alpha beta T cell differentiation (p=0.035) (panel D), and TCR complex (p=1.74E-8) (panel E) in SP8 vs DN T cells from edited ATOs. Panel F) Representative flow cytometry profiles of exhaustion markers in SP8 T cells directly from week 15 HD (n=9) and edited patient ATOs (n=9), and compared to PBMCs (n=3) (PBMC were stimulated with (orange) and without (purple) anti-CD3/28 beads+IL2 for 24 hours (n=9 for ATO groups and n=3 for PBMC controls).

FIG. 13, panels A-K, shows that base editing of CD36 SCID HSPCs generates functional T cells with TCR diversity. Panel A) Calcium flux of cells isolated from HD (green), edited patient (blue), and unedited patient (black) ATOs stimulated with anti-CD3 and anti-CD28. Panel B) Quantified area under the calcium flux curve of HD (green), edited patient (blue), and unedited patient (black) ATO cells. Panels C-F) HD (green) and edited patient (blue) ATOs stimulated with and without anti-CD3/CD28 beads and IL2 for 24 hours (n=6). Panel C) Representative flow cytometry histogram profiling and mean fluorescence intensity (MFI) of IFNγ (panel D), IL-2 (panel E), and TNFα (panel F) production in mature SP8s (zombie-CD45+CD8+CD4-CD45RA+). Production of IFNγ and TNFα with and without stimulation was statistically significant (p<0.01). Production of IL-2 was not statistically significant (p=0.055). Panel G) Activation (upregulation of CD25 and 4-1BB) and panel H) proliferation (CFSE dilution) of isolated HD and edited patient ATO-derived SP8 T cells after culture with anti-CD3/CD28 bead and IL-2 for 5 days. Data is representative of three independent experiments. Panels I-K) Single-cell TCR sequencing by CITE-seq of unedited and edited patient ATOs harvested at week 8, n=2 for each arm. Data are representative of two independent experiments. Panel I) Number of unique TCR clonotypes. Panel J) Frequency of individual TRAV (top) and TRAJ (bottom) usage. Panel K) Heatmap visualization of individual TRAV and TRAJ segments displayed in genomic order from 5′ distal→3′ proximal ends. Statistical significance was calculated by unpaired nonparametric t-test (**p<0.01).

FIG. 14, panels A-F, shows that base-editing efficiently restores CD3 expression without inducing chromothripsis. Panel A) Sanger sequencing traces confirm knockin of the CD3D c.202C>T pathogenic mutation in CD3D alleles with the remaining three alleles containing disruptive indels induced by CRISPR-mediated DSBs. Traces show the sense strand read 5′ to 3′. The black dashed vertical line represents the Cas9 cut site during CRISPR editing, with approximately 150,000 total reads per population. Wildtype Jurkat (SEQ ID NO:7), CdCd(C202T) Jurkat (SEQ ID NO:8). Panel B) Representative flow cytometry plots for CD3 surface expression. Top right: gating strategy to distinguish single cells. Bottom right: live cells. Bottom left: gating strategy to identify cells expressing CD3. SSC-A, side scatter area; FSC-A, forward scatter area; FSC-H, forward scatter height. Panel C) Flow cytometry histogram profiling mean fluorescence intensity (MFI) of CD3 expression in CD3D (C202T) Jurkat T cells (orange), wildtype Jurkat T cells (blue), and ABEmax-NRTH (red), ABE8e-NRTH (purple), ABE8e-NG (green), and RNP+ssODN-treated cells (black). G-banded karyotypes each representing clonal abnormalities observed in cells from mock electroporated (panel D), base-edited with plasmids encoding ABEmax-NRTH and sgRNA (panel E), or edited with Cas9 RNP and ssODN (panel F). Due to the complexity of the cell lines, the clonal abnormalities are described as composite karyotypes (not all indicated abnormalities were identified in all abnormal cells analyzed) using standard cytogenetic nomenclature (ISCN). Diploid, triploid, and tetraploid cells were observed. Black arrows indicate clonal structural abnormalities inherent to the Jurkat T cell line.

FIG. 15, panels A-D, shows that base-editing of OD35 SCID HSPCs reveals minimal local bystander and genome-wide off-target editing. Panel A) Representative flow cytometry plots to measure CD3/TCRab surface expression in CD3D (C202T) Jurkat T cells transduced with either LV expressing WT CD3D cDNA or CD3D cDNA containing the A0 bystander mutation. Top right: gating strategy to distinguish single cells. Bottom right: live cells. Bottom left: gating strategy to identify cells expressing CD3/TCRab. Panel B) Venn diagram of candidate off-target sites predicted by CIRCLE-seq, GUIDE-seq, and Cas-OFFinder. Pie graph depicts the predicted genomic locations of the 57 candidate off-target sites nominated by two or more prediction tools. Panels C-D) Bar graphs demonstrate the percentage of sequencing reads containing A⋅T-to-G⋅C point mutations consistent with adenine base editing and within protospacer positions 4-10 in gDNA from CD36 SCID HSPCs treated with ABEmax-NRTH and sgRNA (peach) or untreated controls (teal). (n=3).

FIG. 16 shows that base-editing of CD36 SCID HSPCs reveals infrequent indel formation. Bar graphs demonstrate percent of sequencing reads containing indels across 200 genome-wide off-target sites. Sites were sequenced with HTS in gDNA from CD36 SCID HSPCs treated with ABEmax-NRTH mRNA (peach) or untreated controls (teal).

FIG. 17, panels A-K, shows that engrafted healthy human HSPCs retain high-levels of gene correction in a humanized mouse model. Pane A) Gating strategy to quantify relative abundances of hematopoietic lineages in recipient mouse bone marrow. Panel B) Gating strategy to determine relative abundances of thymocytes across T cell development in recipient mouse thymi. Sixteen weeks after infusion, engraftment was measured by percentage of human CD45+ cells in recipient mouse bone marrow (panel C), spleen (panel D), and thymus (panel E) for mice receiving untreated cells or ABE8e-NG treated cells. Abundance of human CD19+ B cells, CD33+ myeloid, CD34+ HSPCs, CD56+NK cells, and CD3+ T cells were measured as percentages of the hCD45+ population in transplant recipient bone marrow (panel F) and spleen (panel G). Panel H) Human CD3−TCRαβ−CD4+ immature SP (CD4 ISP), CD3+TCRαβ+CD4+ (CD4 SP), CD3+TCRαβ+CD8+ single-positive (CD8 SP), CD4-CD8-double-negative (DN), and CD4+CD8+ doublepositive (DP) cells as percentages of the hCD45+ population in recipient mouse thymus. Panel I) on-target editing efficiency and VCN determined by HTS and ddPCR, respectively, in cells cultured for 14 days after electroporation (pre-transplant) or in whole tissues 16 weeks after transplant. Panel J) Gating strategy for FACS isolation of CD34+ HSPCs, CD33+ Myeloid, CD19+ B cells, and CD56+NK cells from mouse bone marrow. Panel K) Editing efficiency in human-derived hematopoietic lineages from mouse bone marrow. Populations were FACS sorted using hCD34+, hCD33+, hCD19+, and hCD56+ antibodies for HSPC, myeloid, B cell, and NK cell collection, respectively. n=2 mice that received untreated cells and n=7 mice that received ABE8e-NG edited cells. Data shown as mean±SD; Panel K) one-way ANOVA, non-parametric t-test elsewhere; ns, not significant.

FIG. 18, panels A-C, shows that base-editing can rescue other OD35 SCID-causing mutations in human HSPCs. Panel A) Schematic of the splicing mutation (identified in Ecuador) known to cause CD36 SCID. A homozygous mutation in the splice donor site of intron 2 leads to abnormal splicing and exon 2 skipping.⁴⁴ Panel B) Schematic of the splicing mutation (identified in Japan) known to cause CD36 SCID.⁶⁴ A homozygous mutation in the splice acceptor site of intron 2 leads to abnormal splicing and exon 3 skipping. Panel C) Healthy donor (HD) CD34+ HSPCs were pre-stimulated for 24 hours and transduced with lentiviral vector (containing either the Ecuador or Japan mutation in intron 2). Intron 2 was retained in the LV by positioning the internal expression cassette in reverse orientation. 24 hours after transduction, HSPCs were electroporated with ABE8e-NG, ABE8e-NGG, or BE4max-NG mRNA and sgRNA. Data shown as mean±SD.

FIG. 19, panels A-B, sows that clonogenic potential is retained after base editing. 24 hours following electroporation of CD3S SCID CD34+ HSPCs, a CFU assay was performed to assess hematopoietic potential (panel A) and hematopoietic lineage distribution (panel B) of CD34+ HSPC HD, unedited, and edited patient T cells prior to ATO formation (n=12). Statistics were calculated using a non-parametric t-test; ns, not significant.

FIG. 20, panels A-I, shows that base-editing of OD36 SCID HSPCs rescues T cell differentiation and maturation. Panels A-I)) FACS analysis of HD (green), unedited patient (black), and edited patient (blue) ATOs, n=6-9, from four independent experiments for all data. Panel A) Representative flow cytometry profiles of CD3 and TCRaP co-expression (left), and developing T cell subsets (DN, DP-L, SP8, and SP4) (right) in ATOs at week 15. Panel B) Representative flow cytometry profiles of early CD3 and TCRαβ co-expression at weeks 2, 3, 5, and 7. Frequency of panel C) CD3+TCRαβ+, CD3−TCRαβ−, CD4−CD8−, CD4+CD8−, CD4+CD8+, and CD4−CD8+ cells from CD45+Lin-cells, and panel D) DP-L, CD8a, CD8ap (SP8 T cells), and CD8aa cells from CD45+Lin-cells at week 12. Panel E) Representative flow cytometry profiles of CD8aa and CD8aP cells in cells gated on CD3+TCRaP+CD4− in HD, unedited patient, and edited patient ATOs at weeks 9, 12, and 15. Cell counts of CD3+ (panel F) and TCRαβ+ (panel G) cells per ATO over time. Panel H) Representative flow cytometry profiles of TCRYS expression in cells gated on CD45+Lin− at week 12. Panel I) Cell counts of Y § T cells per ATO over time. Statistical significance was calculated using unpaired nonparametric T tests, **p<0.01; and ***p<0.001.

FIG. 21, panels A-B, illustrates identification of developing T cell and immune cell subsets in unedited and edited CD3δ SCID ATOs by CITE-seq. Surface protein (panel A) and RNA gene expression (panel B) of selected markers across cell subsets in unedited and edited patient ATOs.

FIG. 22, panels A-B, shows that monoallelic and biallelic CD3D correction rescues T cell development. Panel A) WNN_UMAP visualization of unedited (grey), monoallelic (orange), or biallelic (blue) correction of patient ATOs. Panel B) Bar graphs show relative T cell precursor abundances of binned by the presence of unedited, monoallelic, or biallelic correction in single cells. Monoallelic vs. biallelic classification was determined by the presence of RNA strands with or without the CD3D c.202C>T edit. UnEd, unedited; Mono, monoallelic; Bi, biallelic.

FIG. 23, panels A-E, shows that edited OD36 SCID ATO-derived SP8 T and SP4 T cells express features of maturation without evidence of exhaustion. Panel A) Expression of indicated surface proteins (y-axis) across clusters in edited patient ATOs. Panels B and C) Gene Set Enrichment Analysis (GSEA) of differentially expressed genes from GOBP (Gene Ontology Biological Process) and GOCC (Gene Ontology Cellular Compartment) between DP-L and DN (panel B), or SP8 and DP-L cells (panel C). Dot size represents adjusted P-value (Padj; two-sided permutation test). NES, normalized enrichment score; PM, plasma membrane; RNP, ribonucleoprotein; LSU, large ribosomal subunit; SSU, small ribosomal subunit. Panel D) Representative flow cytometry profiles depicting maturation markers (CCR7, CD62L, CD27, CD28, CD45RO, and CD45RA) in cells gated on SP4 cells-CD3+TCRap+CD4+CD8−, in week 12 ATOs (n=9, from four independent experiments). Panel E) Representative flow cytometry profiles of exhaustion markers in SP4 T cells derived from week 15 HD (green) and edited patient (blue) ATOs, and Healthy PBMCs (right) stimulated with (orange) and without (purple) anti-CD3/28 beads+IL2 for 24 hours, n=9 for ATO groups and n=3 for PBMC controls.

FIG. 24, panels A-G, shows that base editing of CD36 SCID HSPCs recapitulates functional T cells with TCR diversity. Panels A-C) Representative flow cytometry profiles of HD (green) and edited patient (blue) ATOs. Panel A) IFNγ, IL-2, and TNFα production in SP8 T cells (CD3+TCRap+CD8a+CD4−CD45RA+) stimulated with or without anti-CD3/CD28 beads and IL2 for 24 hours, n=6. Panel B) CD25 and panel C) 4-1BB expression vs proliferation (CFSE dilution) of MACs isolated ATO-derived SP8 cells after culture without stim, with IL2 alone, anti-CD3/CD28 bead alone, and anti-CD3/CD28 bead+IL-2 for 5 days. Data is representative of three independent experiments. Panels D-H) TCR diversity by CITE-seq of unedited (black) and edited (blue) patient ATOs harvested at week 8, n=2 per arm, two independent experiments. Panel D) TCR diversity measured by CHAO-1 index. Statistical significance was calculated by Hutchinson t-test (*p<5e10-8). Panel E) Chord diagrams depicting interconnection of TCR clonotypes in developing cell subsets in unedited (left) and edited (right) patient ATOs. Area of each segment correlates to the relative abundance of TCR clonotypes in indicated cells subsets. Curved lines indicate TCR clonotypes shared between cell subsets. Panel F) Frequency of individual TRBV (top) and TRBJ (bottom) usage. Panel G) Heatmap visualization of individual TRBV and TRBJ segments displayed in genomic order from 5′ distal→3′ proximal ends.

### DETAILED DESCRIPTION

Here, we describe two gene editing-based approaches to correct CD3δ SCID:
(1) CRISPR/Cas9 homology-directed repair (HDR)-mediated correction with a single-strand oligodeoxynucleotide (ssODN) homologous donor; and
(2) Adenine Base Editing (ABE)-correction, to precisely revert the CD3δ SCID-causing C202T mutation (TGA→CGA) to restore wildtype levels of CD38 expression and subsequent T-cell development.

To investigate the therapeutic efficiency of HDR in CD3δ SCID disease models, we rationally designed two single-guide RNAs (sgRNAs), Guide 2T and Guide 5T, to direct Cas9 cutting two base pairs (bp) upstream and five bp downstream of the C202T mutation, respectively. We designed ssODNs to be complementary to the nontarget strand with asymmetric homology arms (33 bp downstream and 60 bp upstream of the respective sgRNA-guided Cas9 cut site) containing the therapeutic sequence and a silent PAM mutation to prevent continual nuclease activity. Preliminary results show up to 62% precise correction of the CD3D C202T mutation by CRISPR/Cas9 HDR-mediated editing in CD3D C202T K562 cells.

To investigate the therapeutic efficiency of BE in CD3δ SCID disease models, we generated two DNA-targeting sgRNAs to guide multiple ABE8e variants generated in the Kohn Lab. Currently, the highest efficiency ABE, “ABE8e,” is commercially available to recognize the canonical spCas9 NGG protospacer adjacent motif (PAM) sequence. However, BE of the CD3δ SCID-causing C202T mutation is limited to two single-guide RNAs (sgRNAs), both complementary to genomic DNA sequences lacking an appropriate NGG PAM immediately downstream of the protospacer. As such, we have constructed four novel, variant ABE constructs (NG-ABE8e, NRTH-ABE8e, VRER-ABE8e, and xCas9 (3.7)-ABE8e), using Gibson cloning and site-directed mutagenesis procedures, to allow for the use of these two sgRNAs. In relation to the wildtype NGG-recognizing Cas9 (D10A) nickase (Cas9n), our constructs contain the following substitutions:
(1) NRTH-ABE8e: A10T, I322V, S409I, E427G, R654L, R753G, R1114G, D1135N, D1180G, G1218S, E1219V, Q1221H, P1249S, E1253K, P1321S, D1332G, and R1335L (DNA Sequence: SEQ ID NO:3, Protein Sequence: SEQ ID NO:4);
(2) VRER-ABE8e: D1135V, G1218R, R1335E, and T1337R (DNA Sequence: SEQ ID NO:5, Protein Sequence: SEQ ID NO:6); and
(3) A262T, R324L, S409I, E480K, E543D, M694I, and E1219V (DNA Sequence: SEQ ID NO:7, Protein Sequence: SEQ ID NO: 8).

The sequences of the sgRNAs are shown in Table 1.

TABLE 1
Single-guide RNAs (sgRNAs), Guide 2T and Guide
5T, to direct Cas9 cutting two base pairs (bp)
upstream and five bp downstream of the C202T
mutation, respectively.

| Guide | | |
|---|---|---|
| RNA | Sequence | SEQ ID NO: |
| Guide 2T | TTCCTCATGGGTCCAGGATGCGTT | 1 |
| Guide 5T | TACATCTATATATTCCTCATGGG | 2 |

Initial data demonstrate up to 94% TGA to CGA conversion in CD3D C202T K562 cells through treatment with NRTH-ABE8e. These promising data suggest a potentially curative treatment option for gene editing-based autologous HSCT for patients living with CD3δ SCID.

Moreover, as illustrated in Example 2, commercially available primary healthy CD34+ cells were treated with ABE to allow for assessment of stem cell gene modification by xenografting in immune deficient (NSG) mice. We introduced the editing target into HD cells using a lentiviral vector expressing a CD3D cDNA containing the c.C202T mutation. The input CD34+ cells had ˜80% adenine base edits at the target site and the human cells recovered from the xenografted mice 4 months later had similar ˜80% edits in multiple leukocyte lineages, demonstrating the effective gene modification of primary human HSPCs.

Excitingly, by acquiring a bone marrow sample from a CD36 SCID baby we were able to rigorously analyze the molecular and functional impact of applying the ABE technology to a clinically relevant source of HSPC. Using a novel Artificial Thymic Organoid (ATO) system we were able to perform detailed cellular and molecular analysis of T cell development of edited HSPCs. We show that ABE editing in patient HSPCs, fully rescued the development of mature T cells with a diverse TCR repertoire, and that the corrected CD3/TCR complex functioned normally as shown by calcium flux, cytokine production, proliferation, activation and gene expression. We were also able to identify the exact stage of development affected by the CD36 mutation.

### EXAMPLES

The following examples are offered to illustrate, but not to limit the claimed invention.

### Example 1

### Proof of Principle

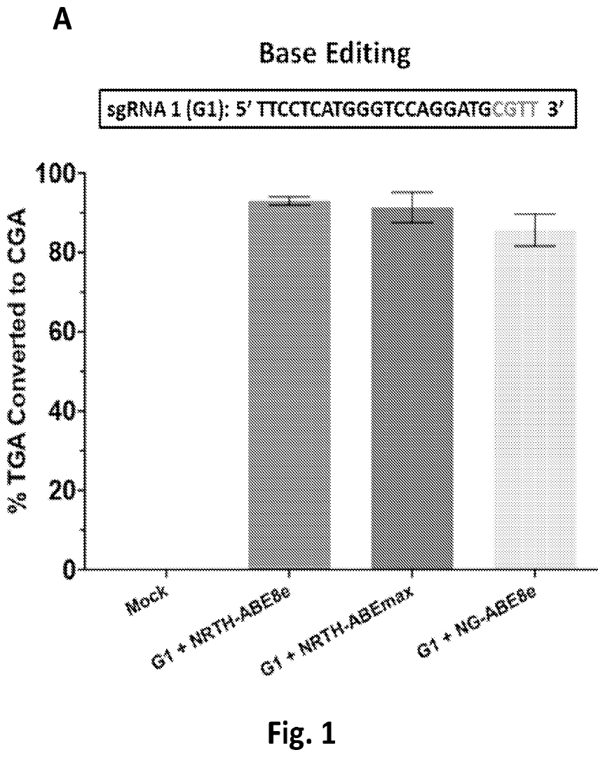

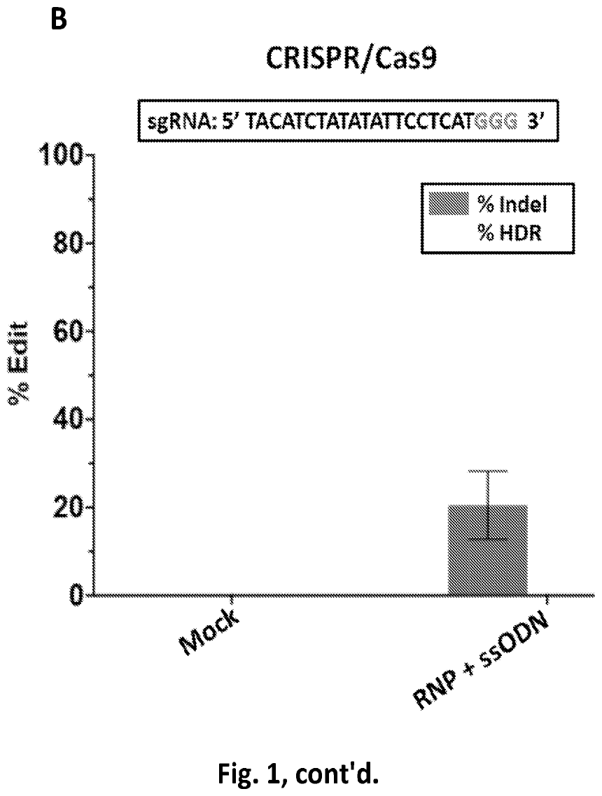

To investigate the therapeutic efficacy of HDR and base editing in the CD3 KO Jurkat T-cell line model, we treated CD3 KO Jurkat T cells with our best performing editing reagents, previously determined in the CD3D C202T K562 cell line. Results show up to 55% precise correction of the CD3D C202T mutation by CRISPR/Cas9 HDR-mediated editing using 1) a rationally designed sgRNA to direct Cas9 nuclease activity two base pairs (bp) upstream of the C202T mutation and 2) an ssODN complementary to the nontarget strand with asymmetric homology arms (33 bp downstream and 60 bp upstream of the respective sgRNA-guided Cas9 cut site) containing the therapeutic sequence and a silent PAM mutation to prevent continual nuclease activity (FIG. 1, panels A-B). Additionally, data demonstrate up to 93%, 94%, and 90% precise correction of the C202T mutation in the CD3 KO Jurkat T-cell line through treatment with NRTH-ABE8e, NRTH-ABEmax, and NG-ABE8e, respectively (FIG. 1, panel A).

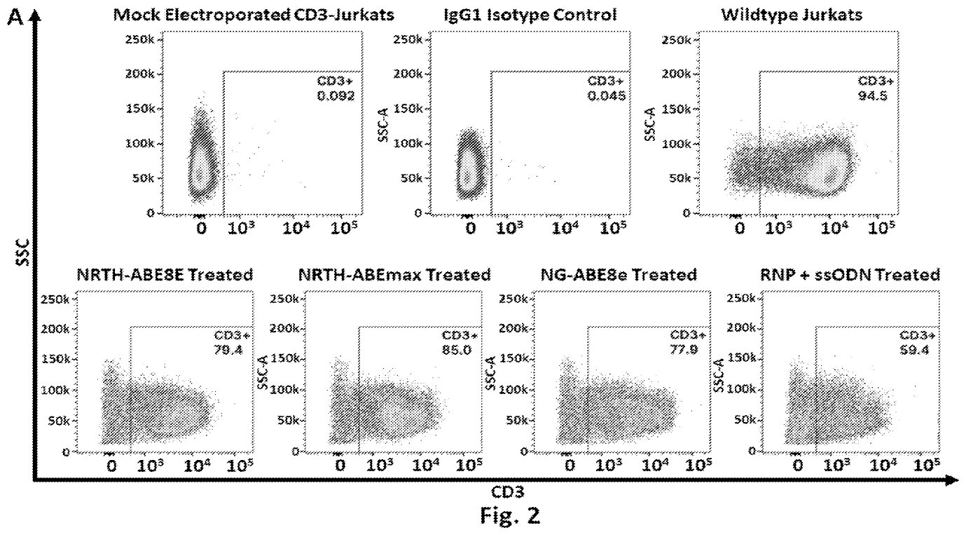

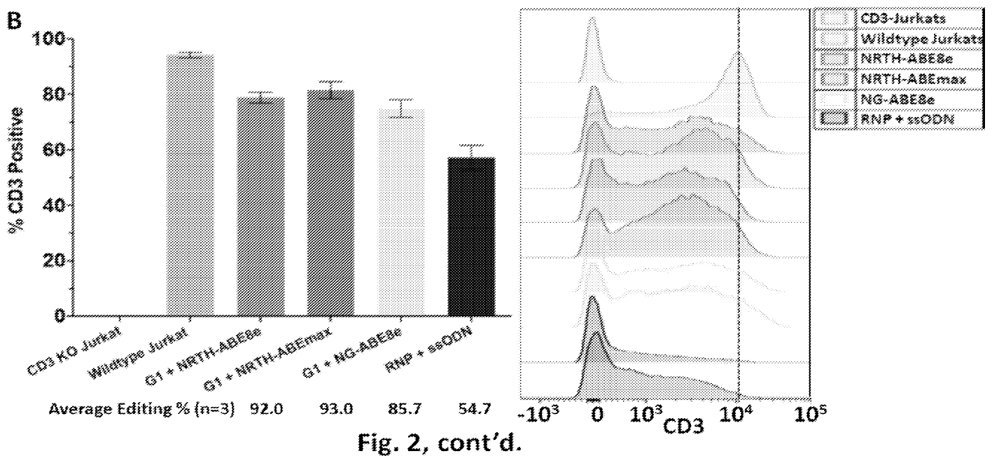

Analysis of edited CD3 KO Jurkat T cells by flow cytometry demonstrated a correlative relationship between restoration of CD3 protein complex expression and editing efficiency. CD3 KO Jurkat T cells treated with NRTH-ABE8E, NRTH-ABEmax, NG-ABE8E, and RNP+ssODN resulted in restored expression of CD3 protein complex in 79.4%, 85%, 77.9%, and 59.4% of manipulated cells (FIG. 2, panels A and B).

Subsequently, we performed a calcium flux assay to assess if the observed restoration of CD3 protein complex was truly functional. During T-cell activation, the engagement of a T cell with an antigen-presenting cell results in rapid cytoskeletal rearrangements and an increase of intracellular calcium concentration. In a resting state, T cells maintain an internal calcium concentration far less than that of the extracellular environment. Therefore, a flux of intracellular calcium concentration is often used as an indicator of T-cell activation in response to a stimulus. Stimulation of CD3 KO Jurkat T cells with anti-CD3 and anti-CD28 antibodies displayed a complete loss of calcium flux when compared to anti-CD3 and anti-CD28 stimulated Wildtype Jurkat T cells. However, CD3 KO Jurkat T cells treated with NRTH-ABE8E, NRTH-ABEmax, NG-ABE8e, or RNP+ssODN, followed by anti-CD3 and anti-CD28 stimulation rescued calcium flux to near-wildtype levels. These data suggest precise editing of the C202T mutation can result in a functional restoration of the CD3/TCR complex.

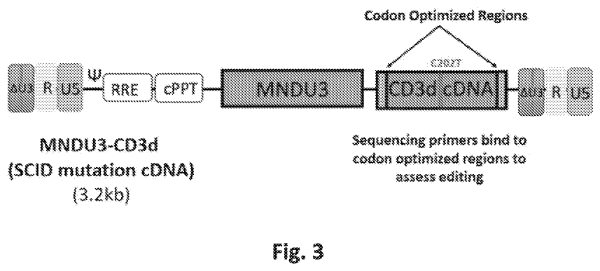

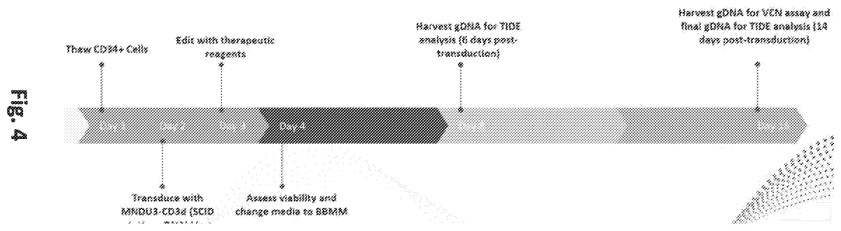

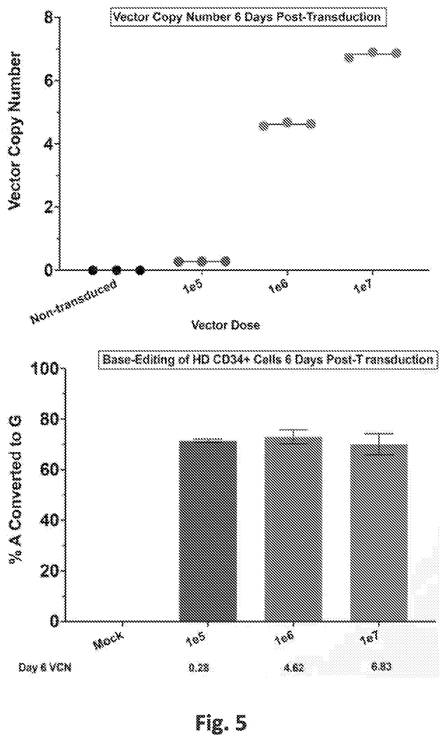

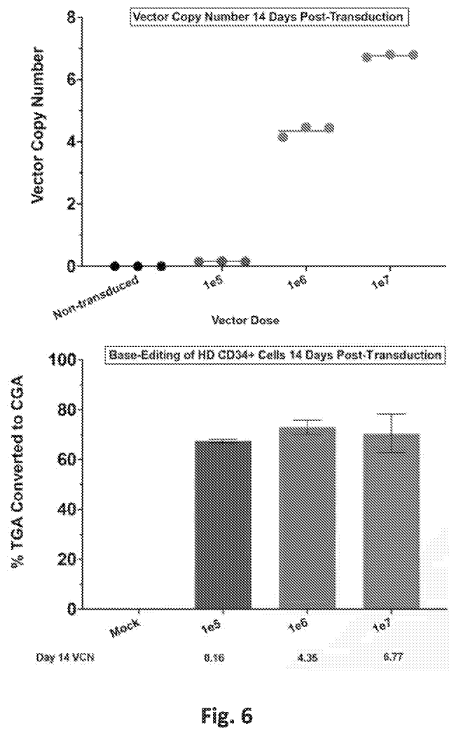

Editing results in the CD3D C202T K562 cell line and the CD3 KO Jurkat T-cell line demonstrated base editing-mediated correction of the CD3δ SCID-causing mutation to be the superior therapeutic approach. Therefore, to investigate the efficacy of our base editing strategy in clinically relevant cells, we utilized a commercially available NG-ABE8E mRNA with our best performing sgRNA (G1) in healthy donor CD34+ HSPCs. sgRNAs used for base editing applications must be complementary to the disease-causing target base. This presents a challenge when testing base editor sgRNAs in clinically relevant cell types if patient HSPCs are not readily or plentifully available. To address this obstacle, we rationally designed an MNDU3-driven CD3D cDNA lentiviral vector containing the CD3δ SCID-causing mutation (C202T). 5′ and 3′ ends of the CD3D cDNA were codon optimized, and forward and reverse primers were designed to bind to these codon-optimized regions, circumventing amplification of the endogenous CD3D gene when assessing editing efficiencies (FIG. 3 and see also SEQ ID NO:1107). CD34+ HSPCs were transduced at three vector doses with the MNDU3-CD3D cDNA lentiviral vector and base editing reagents (NG-ABE8e mRNA and G1) were delivered 24 hours post-transduction by electroporation (FIG. 4). Vector copy number (VCN) and precise base editing of the C202T mutation six days (FIG. 5) and 14 days post-transduction (FIG. 6) displayed stable correction (up to 75%) of the C202T mutation across all VCNs. These data further confirm the efficacy and reproducibility of our base editing-mediated strategy to correct the CD3δ SCID-causing mutation in clinically relevant CD34+ HSPCs, thus enabling advancement to pre-clinical studies in CD3δ SCID patient HSPCs.

Future studies will focus on treating CD3δ SCID patient CD34+ HSPCs with the base editing reagents tested in HD CD34+ HSPCs (NG-ABE8e mRNA and G1) and NRTH-ABEmax mRNA (currently being manufactured by TriLink Biotechnologies) for induced in vitro T-cell differentiation through the artificial thymic organoid (ATO) system. Altogether, these promising results suggest a curative treatment option for gene editing based autologous HSCT for patients living with CD3δ SCID.

### Example 2

### Adenine Base Editing of Hematopoietic Stem Cells Rescues T Cell Development for CD3δ Severe Combined Immune Deficiency

### Abstract for Example 2

CD3δ SCID is a devastating inborn error of immunity caused by mutations in CD3D, encoding the invariant CD3δ chain of the CD3/TCR complex necessary for normal thymopoiesis. We demonstrate an adenine base editing (ABE) strategy to restore CD3δ in autologous hematopoietic stem and progenitor cells (HSPC). Delivery of mRNA encoding a laboratory-evolved ABE and guide RNA into CD3δ SCID patient's HSPCs resulted in 71.2±7.85% (n=3) correction of the pathogenic mutation. Edited HSPCs differentiated in artificial thymic organoids produced mature T cells exhibiting diverse TCR repertoires and TCR-dependent functions. Edited human HSPCs transplanted into immunodeficient mice showed 88% reversion of the CD3D defect in human CD34+ cells isolated from mouse bone marrow after 16 weeks, indicating correction of long-term repopulating HSCs. These findings demonstrate preclinical efficacy of ABE in HSPC for the treatment of CD3δ SCID, providing a foundation for the development of a one-time treatment for CD3δ SCID patients.

### Introduction for Example 2

CD3δ severe combined immune deficiency (SCID) is a life-threatening inborn error of immunity (IEI) caused by biallelic mutations in the autosomal CD3D gene. During normal T cell development, T cell receptor (TCR) assembly begins in the endoplasmic reticulum (ER) as CD3 heterodimers associate with TCR chains for export to the Golgi apparatus, where interactions with the γγ/CD2472 homodimer allow for transport to the cell surface.¹ CD3δ is essential for the productive assembly of TCR complexes; thus, the absence of CD3δ chains results in the intracellular retention of defective TCR ensembles, leading to early arrest of thymopoiesis.¹ A homozygous mutation in CD3D (c.202C>T), predominately found in a Mennonite population, results in a premature stop codon (p.R68X) and the complete absence of CD3δ protein and the CD3/TCR complex. CD3δ SCID patients present with a profound deficiency of circulating, mature αβ and γδ T cells, with present B and NK cells (T−B+NK+ SCID),² often leading to infant mortality.

Allogeneic hematopoietic stem cell transplantation (HSCT) can be curative but may be complicated by limited donor availability, the risk of potentially fatal graft-versus-host disease (GvHD), and treatment-related toxicities.³ In a multi-center study reported in 2011, survival of CD3δ SCID patients undergoing allogeneic HSCT was only 61.5% (n=13) with most patients experiencing acute GvHD and two patients developing chronic GvHD.³

Developing a strategy for autologous HSCT utilizing a patient's own gene-corrected hematopoietic stem and progenitor cells (HSPCs) would abrogate many of the complications associated with allogeneic HSCT. Previous work has explored gene therapy for devastating monogenic IEIs, such as SCID-X1 and adenosine deaminase (ADA)-SCID, through ex vivo lentiviral vector (LV) gene addition or by CRISPR/Cas9 homology-directed repair (HDR) correction of autologous HSPCs).⁴ However, HDR mediated by double-stranded breaks (DSBs) by Cas9 nuclease is cell cycle dependent, is difficult to achieve with high efficiency in long-term HSCs, and carries risks associated with uncontrolled mixtures of indel byproducts, p53 activation, translocations, and loss or rearrangement of large chromosomal segments (chromothripsis).⁵ Although lentiviral (LV) modification of HSCs to restore CD3δ expression could offer a promising clinical strategy, LVs can hypothetically induce oncogenic insertional mutagenesis, and thus, developing a T cell specific LV able to recapitulate the endogenous temporal expression of CD3δ necessary for thymopoiesis may prove difficult.⁶

As an alternative approach, base editing (BE) can correct the pathogenic mutation without requiring donor DNA templates or DSBs and may overcome the limitations of LV gene addition or Cas9 nuclease-mediated HDR. Adenine base editors (ABEs) are comprised of a catalytically impaired Cas9 nickase (Cas9n) fused to a DNA-modifying deaminase enzyme, enabling direct conversion of A⋅T-to-G⋅C base pairs, without introducing DSBs and minimizing indel byproducts.⁷

Here, we describe the development of an ABE approach able to precisely revert the CD3D c.202C>T mutation in 1) a Jurkat T cell line disease model, 2) human CD34+ HSPCs from healthy donors transduced with an LV carrying a CD3D c.202C>T mutation target, and 3) CD34+ HSPCs from a CD3δ SCID patient. We demonstrate highly efficient and specific correction of the CD3D mutation in each cell type, with restoration of CD3δ protein expression and CD3/TCR complex signaling in response to antigenic stimuli. Edited human HSPCs persisted in humanized mouse models, maintaining 88% CD3D c.202C>T correction after sixteen weeks.

We utilized the novel 3D artificial thymic organoid (ATO) system⁸ to determine restoration of CD3 and TCR surface expression in base edited CD3δ SCID HSPCs undergoing in vitro T cell maturation. Previous ATO studies have demonstrated robust and unique recapitulation of thymocyte positive selection with remarkable fidelity to both mouse 9 and human10,11 T cell differentiation in the thymus. ATOs have also been adopted to characterize and diagnose SCIDs that result in T cell lymphopenias like CD3δ SCID. 8 Our results show that edited CD3δ SCID HSPCs produced functional T lymphocytes with diverse TCR repertoire in the ATO. These data suggest an ABE-mediated autologous gene therapy is a promising treatment strategy for CD3δ SCID.

### Results

### Adenine Base Editing Functionally Restores Wildtype Levels of CD3/TCR Expression and Signaling in a Jurkat T Cell Disease Model

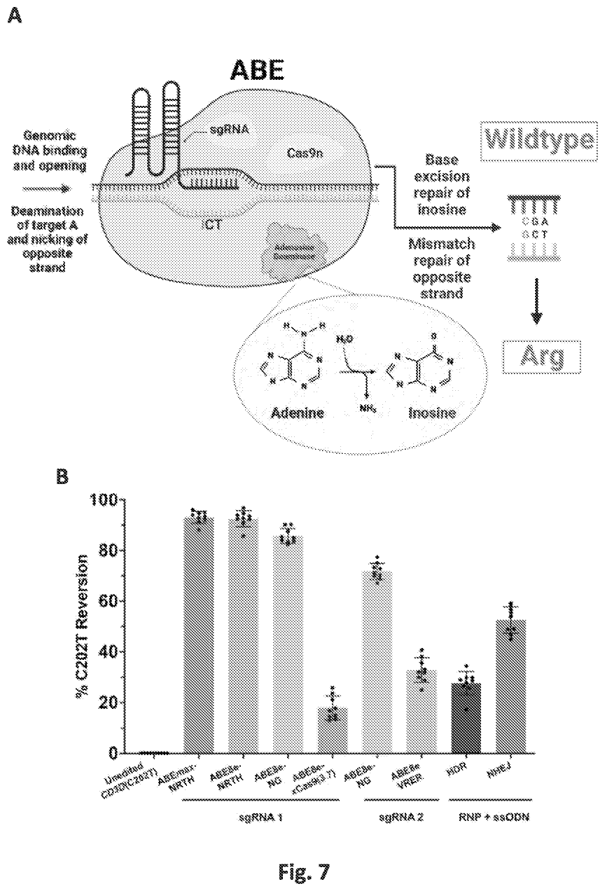

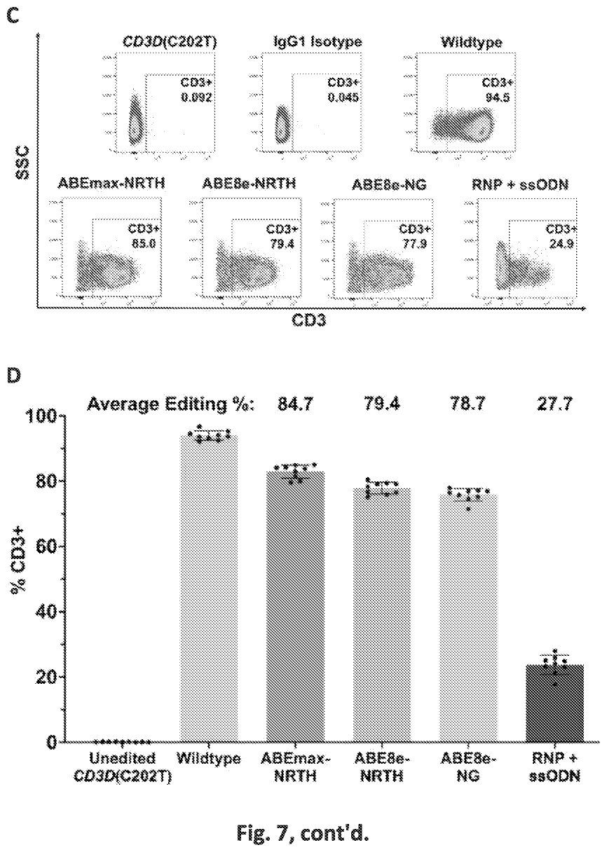

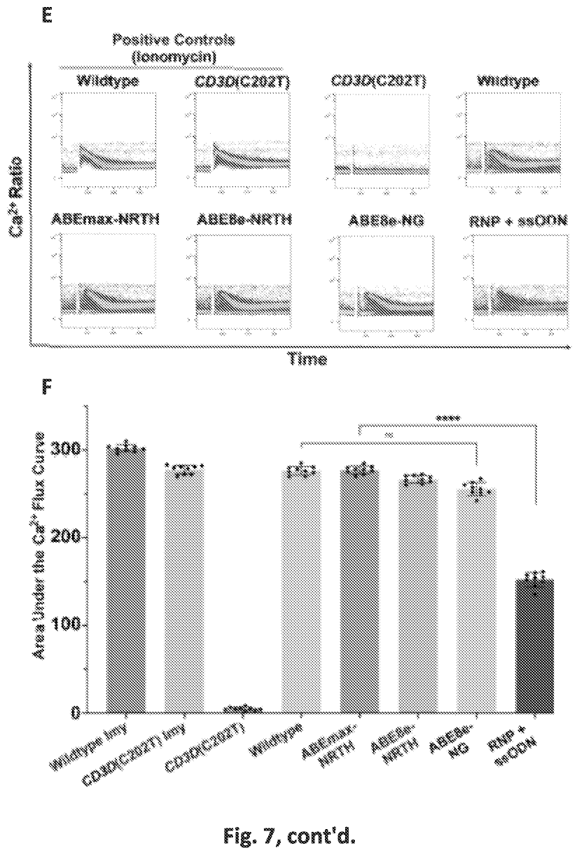

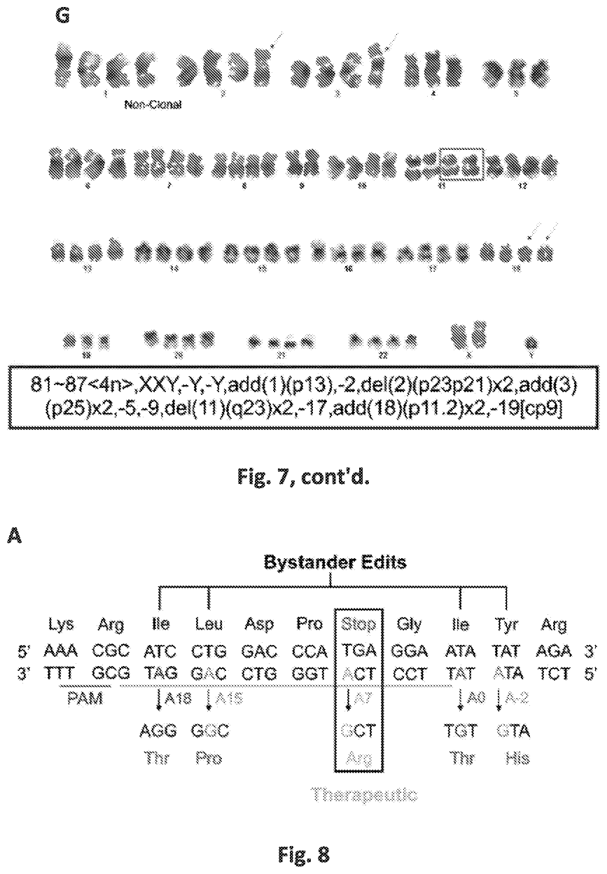

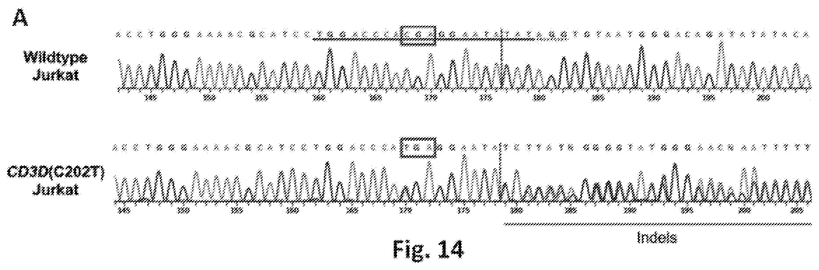

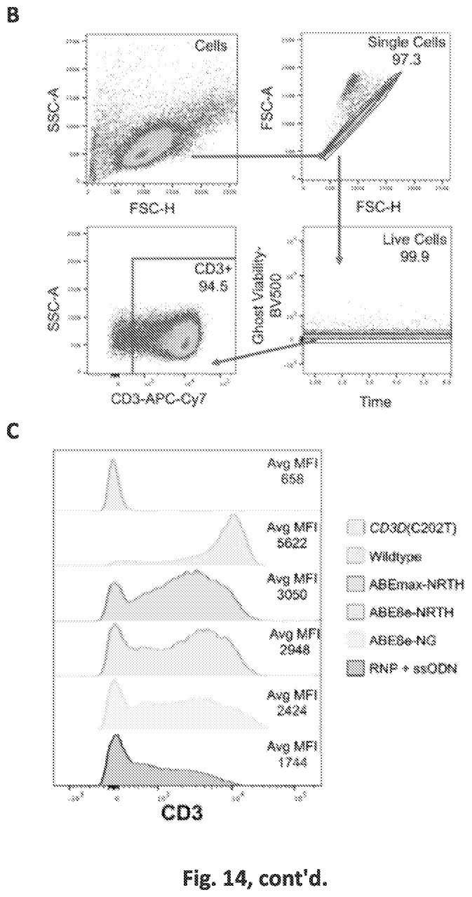

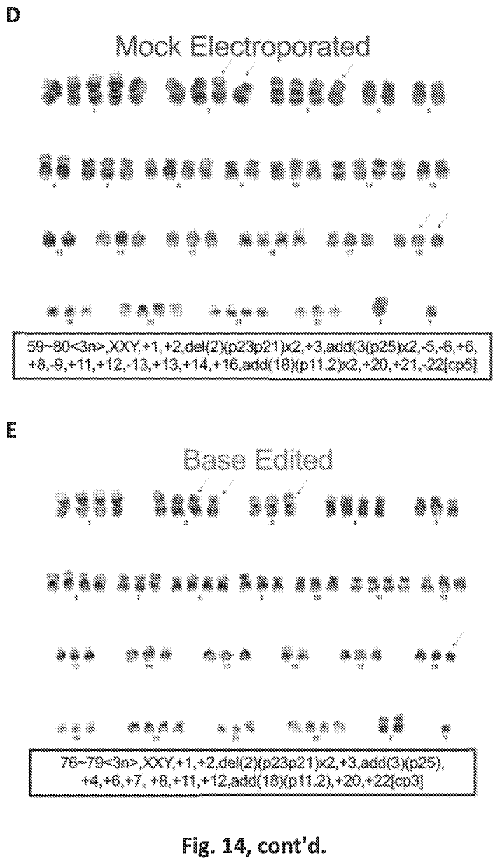

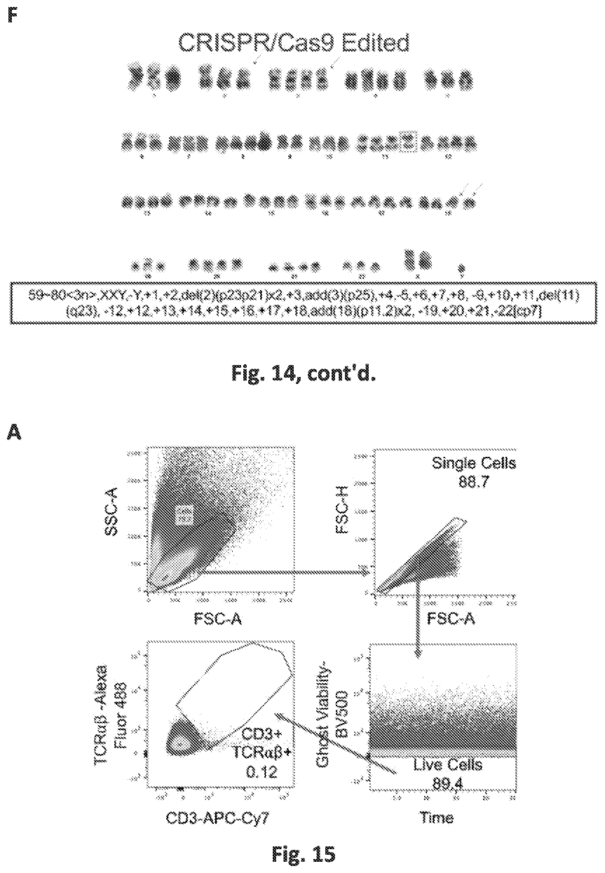

Cas9-mediated HDR and adenine base editing therapies have recently been utilized to eliminate the point mutations causing monogenic diseases such as sickle cell disease and p-thalassemia.^{5,12-14} To determine whether ABE or Cas9 nuclease-mediated HDR gene correction could be suitable strategies for CD3δ SCID, we generated a clonal Jurkat T cell disease model (CD3D (C202T) Jurkat T cells) containing the pathogenic c.202C>T CD3D mutation in one CD3D allele (with deleterious indels in the other three alleles in a pseudo-tetraploid Jurkat T cell line) (see Materials and Methods and FIG. 14, panel A). The disease-causing defect can be corrected by 1) evolved adenine base editors recognizing non-canonical (non-NGG) protospacer-adjacent motifs (PAM) (FIG. 7, panel A) or by 2) Cas9 nuclease-mediated HDR utilizing a single-stranded oligodeoxynucleotide (ssODN) homologous donor and ribonucleoprotein (RNP) complex of rCas9 protein and a single guide RNA (sgRNA). Electroporation of CD3D (C202T) Jurkat T cells with Cas9 nuclease RNP and an ssODN to mediate HDR resulted in 28±4.6% (mean±standard deviation) correction of the pathogenic mutation with 53±5.2% indel byproducts. In contrast, electroporation of the same cells with plasmids encoding CD3D-targeting single-guide RNA (sgRNA) and ABEmax-NRTH, ABE8e-NRTH, ABE8e-NG, ABE8e-VRER, or ABE8e-xCas9 (3.7) produced 93±2.3%, 92±3.1%, 86±2.9%, 33±4.8%, and 18±4.7% correction of the CD3D c.202C>T mutation, respectively, with minimal indels (FIG. 1, panel B). Analysis of edited CD3D (C202T) Jurkat T cells by flow cytometry revealed a positive correlation between CD3D c.202C>T base editing and surface CD3 complex restoration, with rescued CD3 surface expression in up to 85±2.1%, 79.4±1.8%, 77.9%±1.9, and 29.4±2.9% of cells manipulated with ABEmax-NRTH, ABE8e-NRTH, ABE8e-NG, or RNP+ssODN, respectively (FIG. 7, panels C-D; FIG. 14, panels C-D).

During T cell activation, the engagement of a T cell with an antigen-presenting cell results in rapid cytoskeletal rearrangements and an increase of intracellular calcium concentration.¹⁵ Therefore, to assess functional rescue of CD3/TCR signaling, we performed a calcium flux assay with unedited and edited CD3D (C202T) Jurkat T cells, where a flux of intracellular calcium can be used as an indicator of TCR-dependent activation in response to an antigenic stimulus.¹⁵ Consistent with gene editing frequencies and CD3D rescue, adenine base editing with ABEmax-NRTH, ABE8e-NRTH, or ABE8e-NG restored CD3/TCR signaling in response to anti-CD3 and anti-CD28 to wildtype levels, while RNP+ssODN treatment restored calcium flux to only 58% of wildtype (FIG. 7, panels E-F).

Previous studies have reported induction of large chromosomal rearrangements or deletions as on-target consequences of Cas9 nuclease-mediated DSBs. 16 Importantly, chromosomal abnormalities involving the CD3D on-target site, 11q23, have frequently been associated with acute myeloid leukemia and poor prognosis for chronic myeloid leukemia patients. 17,18 Therefore, to evaluate the effects of ABE and CRISPR/Cas9 manipulation on chromosomal integrity, we performed standard karyotype analysis of 20 metaphases each of mock electroporated (without cargo), ABE-treated, and RNP and ssODN-treated (CRISPR/Cas9) CD3D (C202T) Jurkat T cells. Four of 20 metaphase cells treated with Cas9 nuclease and ssODN for HDR demonstrated a large deletion distal to the chromosome 11q23 region [del(11)(q23)], with a subset of cells displaying rearrangements 5 involving 11q23 (FIG. 7, panel G karyotype; Table 2; Table 3, FIG. 14, panels D-F).

TABLE 2
Additional clonal structural abnormalities only
observed in the CRISPR/Cas9-edited Jurkat T cells

| Chromosomal | Mock | Base | CRISPR/Cas9 |
|---|---|---|---|
| Abnormality | Electroporated | edited | edited |
| add(1)(p13) | 0/20 cells | 0/20 cells | 2/20 cells |
| [del(1)(q23)] | 0/20 cells | 0/20 cells | 4/20 cells |
| (On-Target) | | | |

TABLE 3
Observed chromosomal abnormalities.
(CrRNA all TTACATCTATATATTCCTCNGG (SEQ ID NO: 9)).
Bulge type = X, Chr = Chromosome,
Pos = position, Dir = Direction,
Mis = # Mismatches).

| | SEQ | | | | |
|---|---|---|---|---|---|
| | ID | | | | |
| DNA | NO | Chr | Pos | Dir | Mis |
| TTgaATCTATATATTgCTtTGG | 10 | chr1 | 787099 | + | 4 |
| cTtCAgCTATATcTTCCTCTGG | 11 | chr1 | 6650925 | + | 4 |
| TTACATCTATAgATcaCTtTGG | 12 | chr1 | 7306700 | + | 4 |
| TTACcTCcAaATgTTCCTCTGG | 13 | chr1 | 16604236 | + | 4 |
| TTAtATCaATATAaTCaTCTGG | 14 | chr1 | 17048217 | + | 4 |
| TTACcTCcAaATgTTCCTCTGG | 15 | chr1 | 21452441 | − | 4 |
| TTACATgTATATATaCtTtTGG | 16 | chr1 | 23358059 | + | 4 |
| TTtttTCTATcTATTCCTCAGG | 17 | chr1 | 30017926 | + | 4 |
| TTgCATCTATAaATTaCTtTGG | 18 | chr1 | 30156656 | + | 4 |
| TTAaATCTATAgATTgCTtTGG | 19 | chr1 | 35070153 | + | 4 |
| TaACAaCcATcTATTCCTCTGG | 20 | chr1 | 41773619 | + | 4 |
| TTgCATCTATAaATTaCTtTGG | 21 | chr1 | 42562030 | − | 4 |
| TTACATgTATAcATTtCcCGGG | 22 | chr1 | 45933301 | − | 4 |
| TTtaATtTATAcATTCCTCAGG | 23 | chr1 | 48439885 | − | 4 |
| TTACAaCTATtaATTCCTaGGG | 24 | chr1 | 48729212 | + | 4 |
| TgACATaTAatTATTCCTCTGG | 25 | chr1 | 49632424 | − | 4 |
| TTAtATCTATAgATTCtTtTGG | 26 | chr1 | 51662652 | − | 4 |
| TTtCcTCTATtTcTTCCTCAGG | 27 | chr1 | 56046036 | − | 4 |
| TTAaATCTATAaATTgCTCTGG | 28 | chr1 | 56111794 | − | 3 |
| TgtCATCTATATATTCCTaGGG | 29 | chr1 | 57444910 | + | 3 |
| TTACATCTATAaATTaCctTGG | 30 | chr1 | 60217034 | + | 4 |
| TTACATCTATAaATTaCctTGG | 31 | chr1 | 60209187 | − | 4 |
| TTAaATCTATAgATTgCTtTGG | 32 | chr1 | 63291108 | − | 4 |
| TTACATaTATtTATTgCTaTGG | 33 | chr1 | 63273925 | − | 4 |
| TTACATCTATATACTCtcCAGG | 34 | chr1 | 64167705 | + | 3 |
| TTAtcTCTAgATATTCCaCTGG | 35 | chr1 | 64249592 | − | 4 |
| aTACATCTATActTTCtTCAGG | 36 | chr1 | 66646285 | + | 4 |
| TTgaATCTATAaATTaCTCTGG | 37 | chr1 | 69307989 | + | 4 |
| TTAaATCTATAaATTaCTtTGG | 38 | chr1 | 69391014 | + | 4 |
| TTgaATCTATAaATTgCTCTGG | 39 | chr1 | 69560235 | − | 4 |
| cTACATCTATgcATTCtTCTGG | 40 | chr1 | 70138687 | − | 4 |
| TTgCtTCTATATATTCCaaGGG | 41 | chr1 | 71939364 | + | 4 |
| TTgaATCTATATATTaCTtTGG | 42 | chr1 | 72910580 | + | 4 |
| TTAtATCTATAaATTgCTaTGG | 43 | chr1 | 73533379 | + | 4 |
| TTAaATCTATAaATTgCTtTGG | 44 | chr1 | 74046031 | − | 4 |
| TTtCtTaTATAgATTCCTCTGG | 45 | chr1 | 74330375 | − | 4 |
| TTcCAgaTATATATTCaTCTGG | 46 | chr1 | 78759669 | − | 4 |
| TaAaATtTATATATTCCTtGGG | 47 | chr1 | 84044759 | − | 4 |
| gTcaATCTATATATTCITCTGG | 48 | chr1 | 84480398 | − | 4 |
| TTgaATCTATATATTgCTtTGG | 49 | chr1 | 93558866 | + | 4 |
| caACATCTcTATATTCCTgTGG | 50 | chr1 | 95543327 | + | 4 |
| TTACtTCTtTtTcTTCCTCAGG | 51 | chr1 | 96872106 | + | 4 |
| TTgCtTCTtTATtTTCCTCTGG | 52 | chr1 | 96951595 | − | 4 |
| TTACATCTgcATccTCCTCTGG | 53 | chr1 | 97981905 | − | 4 |
| TTcCATCTcTcTtTTCCTCTGG | 54 | chr1 | 98819721 | − | 4 |
| TTAaATCTATAaATTaCTtTGG | 55 | chr1 | 99342122 | − | 4 |
| TTAaATCTATAgATTgCTtTGG | 56 | chr1 | 103920898 | + | 4 |
| TTAtgTaTATATATTCCTtAGG | 57 | chr1 | 104117235 | + | 4 |
| TTAaATCTATAaATTaCTtTGG | 58 | chr1 | 104549647 | − | 4 |
| TTgtATCTATAaATTCCTtTGG | 59 | chr1 | 105019632 | + | 4 |
| TTtCATCTtTATtTTtCTCTGG | 60 | chr1 | 108480888 | + | 4 |
| TTACtTgTATATATTCCTgGGG | 61 | chr1 | 112198768 | + | 3 |
| cTACATCTgTAcATTCCCCAGG | 62 | chr1 | 113697941 | + | 4 |
| TTtCATCTcTATAaTCCTCAGG | 63 | chr1 | 113988785 | − | 3 |
| TTACcTCcAaATgTTCCTCTGG | 64 | chr1 | 120428380 | − | 4 |
| TTACcTCcAaATgTTCCTCTGG | 65 | chr1 | 144448222 | + | 4 |
| TTACcTCcAaATgTTCCTCTGG | 66 | chr1 | 145412197 | + | 4 |
| aTAgATCTAcATATTtCTCAGG | 67 | chr1 | 146810834 | + | 4 |
| TTACcTCcAaATgTTCCTCTGG | 68 | chr1 | 146952019 | − | 4 |
| TTACcTCcAaATgTTCCTCTGG | 69 | chr1 | 148135119 | + | 4 |
| aTACtTtaATATATTCCTCTGG | 70 | chr1 | 157731832 | + | 4 |
| TTAtATtTATATATTttTCTGG | 71 | chr1 | 157879435 | + | 4 |
| TTAaATCTATgaATTCCTgGGG | 72 | chr1 | 160430132 | − | 4 |
| TTAgATCTgTAgATTCCTtTGG | 73 | chr1 | 163526827 | + | 4 |
| aTAtATaTATATATTCCTaTGG | 74 | chr1 | 168847242 | − | 4 |
| aTAtATaTATATATTCaTCAGG | 75 | chr1 | 170299773 | + | 4 |
| TTAgATCTATAaATTaCTtTGG | 76 | chr1 | 170724847 | + | 4 |
| TTgaATCTATAaATTCCTtTGG | 77 | chr1 | 171364111 | − | 4 |
| TgtCATCTATATcTTCtTCTGG | 78 | chr1 | 171863172 | − | 4 |
| TTACAgCTcTATAaTaCTCTGG | 79 | chr1 | 184277373 | + | 4 |
| TTACATITATtTATgCtTCAGG | 80 | chr1 | 186403368 | − | 4 |
| TTAtAaCTAaATATTaCTCTGG | 81 | chr1 | 191957853 | + | 4 |
| TTACATgTATATATTaaTaTGG | 82 | chr1 | 195480086 | − | 4 |
| TTgaATCTATATATTgCTtTGG | 83 | chr1 | 195603731 | + | 4 |
| TTgaATCTATATATTaCTtAGG | 84 | chr1 | 195976989 | + | 4 |
| TTtCATCTtTggATTCCTCAGG | 85 | chr1 | 197417251 | − | 4 |
| TTtCATtTATATATcCaTCAGG | 86 | chr1 | 199347208 | + | 4 |
| TTtCAgCTATgTcTTCCTCGGG | 87 | chr1 | 204354231 | − | 4 |
| TTAaATCTATATATTgCTtTGG | 88 | chr1 | 212280705 | + | 3 |
| aTAaATCTATATATTgCTtTGG | 89 | chr1 | 212280727 | + | 4 |
| TTtCATCTATgTATcCaTCAGG | 90 | chr1 | 213907378 | − | 4 |
| TTACATCTgTATATTCaaCTGG | 91 | chr1 | 219973440 | − | 3 |
| TTgaATCTATAgATTgCTCTGG | 92 | chr1 | 220988851 | + | 4 |
| TTgaATCTATATATTgCTtTGG | 93 | chr1 | 224010030 | + | 4 |
| TTACtTtgATATATTCCaCAGG | 94 | chr1 | 224936044 | − | 4 |
| TTAaATCTATAaATTaCTtTGG | 95 | chr1 | 226998218 | − | 4 |
| TaACATaTATAcATaCCTCAGG | 96 | chr1 | 232837475 | − | 4 |
| TTcCAaCTATATggTCCTCTGG | 97 | chr1 | 233220005 | − | 4 |
| TTACtTCTATATcTTCtTaTGG | 98 | chr1 | 237757450 | + | 4 |
| TTACAcaTATAcATTtCTCAGG | 99 | chr1 | 238819624 | − | 4 |
| TTgaATCTATAaATTaCTCTGG | 100 | chr1 | 238851107 | − | 4 |
| TTAtATCTATtTtTTCCTaTGG | 101 | chr1 | 241214929 | + | 4 |
| cTACAaCTtTtTATTCCTCTGG | 102 | chr1 | 241204271 | − | 4 |
| TTACATCTgTAgATTgCTtTGG | 103 | chr1 | 246028654 | − | 4 |
| TcACATITATATAaTgCTCTGG | 104 | chr1 | 246486511 | + | 4 |
| TTAaATCTATAaATTaCTtTGG | 105 | chr1 | 248133230 | − | 4 |
| TTAaATCTATAaATTaCTCTGG | 106 | chr1 | 248312468 | + | 3 |
| TTACATCTITATATTatTgAGG | 107 | chr22 | 16461864 | − | 4 |
| TTACcTtTATATtTTCaTCAGG | 108 | chr22 | 18230499 | + | 4 |
| TTACcTtTATATtTTCaTCAGG | 109 | chr22 | 18394043 | − | 4 |
| TTACcTtTATATtTTCaTCAGG | 110 | chr22 | 18643656 | − | 4 |
| TTACcTtTATATtTTCaTCAGG | 111 | chr22 | 21351604 | + | 4 |
| TcACATCTATAgcTTgCTCAGG | 112 | chr22 | 21734745 | + | 4 |
| gaACATCTATATATTCaTtTGG | 113 | chr22 | 29253661 | + | 4 |
| TTgaATCTATATATTaCTtTGG | 114 | chr22 | 33175728 | − | 4 |
| TTgCATCTATAaATTgCTtTGG | 115 | chr22 | 34937537 | − | 4 |
| TaAtATaTATATATTCCcCTGG | 116 | chr22 | 40432491 | + | 4 |
| TTgaATCTATATATTgCTtTGG | 117 | chr22 | 45725221 | + | 4 |
| TTACATCTATgacTTCaTCTGG | 118 | chr7 | 7893083 | + | 4 |
| TTAaATCTtTATATTgCTtTGG | 119 | chr7 | 9383481 | + | 4 |
| TTAaATCTATAaATTaCTtTGG | 120 | chr7 | 9826422 | + | 4 |
| TTgaATCTATAaATTaCTCTGG | 121 | chr7 | 9902671 | + | 4 |
| TTACATCTAaATATTCaaaAGG | 122 | chr7 | 9989715 | + | 4 |
| TTAaATCTATAaATTaCTtTGG | 123 | chr7 | 10252418 | − | 4 |
| TaACtTCTAcgTATTCCTCAGG | 124 | chr7 | 10291692 | − | 4 |
| TTAaATCTATAaATTCCTtTGG | 125 | chr7 | 11679769 | − | 3 |
| TTAaATCTATAaATTgCTtTGG | 126 | chr7 | 11883250 | − | 4 |
| TTtCATCTcTATATTCCTaGGG | 127 | chr7 | 12627530 | + | 3 |
| TTAaATCTATAaATTgCTtTGG | 128 | chr7 | 14608986 | − | 4 |
| TTACATtTATATATaCtTaCGG | 129 | chr7 | 14924850 | + | 4 |
| TTgaATCTATATATTaCTtTGG | 130 | chr7 | 14983525 | − | 4 |
| TTgaATCTATAgATTgCTCTGG | 131 | chr7 | 16831902 | − | 4 |
| aTACATCaAaATtTTCCTCTGG | 132 | chr7 | 17572231 | − | 4 |
| TTACATCTATAatcTCCTtGGG | 133 | chr7 | 19315708 | − | 4 |
| aTACATgTATATATTCaTaAGG | 134 | chr7 | 23200820 | + | 4 |
| TTACAaCTAaATgTTCtTCAGG | 135 | chr7 | 30397238 | − | 4 |
| TTAtATgTcTATATTCaTCTGG | 136 | chr7 | 31291777 | − | 4 |
| TTAtATaTATATATTtCTgGGG | 137 | chr7 | 31393092 | + | 4 |
| TgACATCTATATAgTaCTgAGG | 138 | chr7 | 31863074 | + | 4 |
| TTACATCcActTATTtCTCAGG | 139 | chr7 | 34508820 | − | 4 |
| gTACATCTCTATATTtCcCAGG | 140 | chr7 | 34530555 | − | 4 |
| aTAtATaTATATATTtCTCTGG | 141 | chr7 | 36412298 | − | 4 |
| TTgaATCTATAaATTaCTCTGG | 142 | chr7 | 39436189 | + | 4 |
| aTACATCTtTAaATTCCaCTGG | 143 | chr7 | 39718886 | + | 4 |
| TTAaATCTATAaATTgCTtTGG | 144 | chr7 | 51195581 | + | 4 |
| TTgaATCTATAaATTaCTCTGG | 145 | chr7 | 55227352 | + | 4 |
| TTACAaCTtTATtTTCCTtAGG | 146 | chr7 | 55363808 | − | 4 |
| TTtCtTCTAatTATTCCTCTGG | 147 | chr7 | 63903485 | + | 4 |
| TTACcTCTATATtTTCtTCTGG | 148 | chr7 | 76139705 | − | 3 |
| TTACATCaATATAaTCCaaTGG | 149 | chr7 | 78750865 | + | 4 |
| TTAaATCTATATATTgtTCTGG | 150 | chr7 | 82487609 | + | 3 |
| TTAaATCTAcATATTaCTCTGG | 151 | chr7 | 82581237 | + | 3 |
| TTcCATgTATAcATTgCTCTGG | 152 | chr7 | 83600143 | + | 4 |
| TTAtATCTATATgcTCaTCAGG | 153 | chr7 | 83571293 | − | 4 |
| TTACATtTATAaATTaCcCAGG | 154 | chr7 | 84550626 | + | 4 |
| aTAtATCTtTATATTCCCCAGG | 155 | chr7 | 85781651 | − | 4 |
| TTAaATCTgTAgATTCCTtTGG | 156 | chr7 | 86512132 | + | 4 |
| TTAgAaCTAcATtTTCCTCTGG | 157 | chr7 | 88998158 | + | 4 |
| TTAaATCTATAaATTaCTtTGG | 158 | chr7 | 88945656 | + | 4 |
| TTAaATCTATAgATTgCTtTGG | 159 | chr7 | 91737210 | + | 4 |
| TTAtATCcAgATATaCCTCTGG | 160 | chr7 | 92694179 | + | 4 |
| TTgCATCctTAaATTCCTCTGG | 161 | chr7 | 93452536 | − | 4 |
| aTttATCTATAaATTCCTCAGG | 162 | chr7 | 93872722 | − | 4 |
| TTACATtTtTAaATTaCTCTGG | 163 | chr7 | 94578220 | − | 4 |
| TTgCATCTATAaATTaCTtTGG | 164 | chr7 | 103883856 | − | 4 |
| TaACATCTATgaATcCCTCAGG | 165 | chr7 | 107001320 | + | 4 |
| TTAaATtTATATcTgCCTCAGG | 166 | chr7 | 110507410 | − | 4 |
| TTACATCTccATtTTCCTgTGG | 167 | chr7 | 110649283 | − | 4 |
| TTAaATCTATAaATTaCTtAGG | 168 | chr7 | 110748321 | − | 4 |
| TTACATtTcTATcTTCCTtTGG | 169 | chr7 | 115622983 | + | 4 |
| TTgCATCTATAaATTaCTtTGG | 170 | chr7 | 123965868 | + | 4 |
| TTACATCTATAaATTgCTtTGG | 171 | chr7 | 124715527 | − | 3 |
| TgACcTaTATATATTCTCAGG | 172 | chr7 | 127379304 | + | 4 |
| TTAaATCTATAaATTgCTtTGG | 173 | chr7 | 130245447 | − | 4 |
| gTACATCTtTATATTtCTCTGG | 174 | chr7 | 131042112 | − | 3 |
| TTtacaCTATATATTCCTCTGG | 175 | chr7 | 131428559 | + | 4 |
| TTtCAaCTATtTATTCtTCGGG | 176 | chr7 | 136037028 | − | 4 |
| TTgCcTCTtTATATTCCTCAGG | 177 | chr7 | 137132711 | − | 3 |
| TaACATtTATATATTtCTgTGG | 178 | chr7 | 139183790 | − | 4 |
| TTgaATCTATAaATTgCTCTGG | 179 | chr7 | 142174246 | + | 4 |
| TTAaATCTATATAacaCTCAGG | 180 | chr7 | 144835097 | − | 4 |
| TTAaATgTATATATTCaTCAGG | 181 | chr7 | 146978599 | + | 3 |
| TTAgATgTATATATTCtTgGGG | 182 | chr7 | 156845167 | − | 4 |
| TTgaATCTATAaATTaCTCTGG | 183 | chr7 | 159289093 | − | 4 |
| gTACATCTgTcTtTTCCTCAGG | 184 | chr2 | 418732 | − | 4 |
| TTAaATCTgTATATTaCTtTGG | 185 | chr2 | 2009273 | + | 4 |
| TTACATCTATtgcTTCCcCTGG | 186 | chr2 | 3298413 | + | 4 |
| TTAtATaTATATATTgtTCTGG | 187 | chr2 | 6014074 | + | 4 |
| TTACATCTAcAgATTCCagAGG | 188 | chr2 | 6678619 | + | 4 |
| TTAaATCTATAaATTgCTCTGG | 189 | chr2 | 11959911 | − | 3 |
| TTtCtTCTAgATATTaCTCTGG | 190 | chr2 | 13535364 | − | 4 |
| TTAaATCTATAaATTCCTtTGG | 191 | chr2 | 13749719 | − | 3 |
| TTACAaaTATtTATTCCTaGGG | 192 | chr2 | 15039014 | − | 4 |
| TTAaATCTATAaATTaCTtTGG | 193 | chr2 | 15345579 | − | 4 |
| TTACATaTtTAaATTCCCCAGG | 194 | chr2 | 16132118 | + | 4 |
| TTtCcTCTATtTtTTCCTCAGG | 195 | chr2 | 18669143 | − | 4 |
| TTAaATCTATAaATTaCTtTGG | 196 | chr2 | 18955336 | + | 4 |
| TgACATCaAcATATTaCTCAGG | 197 | chr2 | 18870471 | − | 4 |
| TTACtTCTtTATAcTCtTCAGG | 198 | chr2 | 21207911 | − | 4 |
| TTACATCTAgAgATTgtTCTGG | 199 | chr2 | 28202078 | − | 4 |
| aTACATCTcTAaATTgCTCAGG | 200 | chr2 | 29916620 | + | 4 |
| TTtCATCTATATtcTtCTCAGG | 201 | chr2 | 33589119 | + | 4 |
| TaAaATCTATATATTaCTgTGG | 202 | chr2 | 34634463 | − | 4 |
| TTACtTCTATATcTcaCTCAGG | 203 | chr2 | 35998665 | + | 4 |
| cTACATtTATcTATTCaTCTGG | 204 | chr2 | 39088702 | + | 4 |
| TaACATITATtTATaCCTCAGG | 205 | chr2 | 40139257 | + | 4 |
| TTACATaaAcATATTCaTCTGG | 206 | chr2 | 42383663 | − | 4 |
| TTAgAaCTATATAagCCTCTGG | 207 | chr2 | 48822445 | + | 4 |
| TTAaATCTgTAgATTCCTtTGG | 208 | chr2 | 52890529 | + | 4 |
| TTAaAcCTATATATTgCTtTGG | 209 | chr2 | 53041822 | + | 4 |
| TTtCATCTcTAaATTCCCCGGG | 210 | chr2 | 54420284 | + | 4 |
| TTtCATCTATgTATcCCTaGGG | 211 | chr2 | 54248448 | − | 4 |
| TTAaAagTATATgTTCCTCAGG | 212 | chr2 | 55303678 | − | 4 |
| TTAaATCTATAaATTaCTtTGG | 213 | chr2 | 56759957 | − | 4 |
| TTAaATCTATAaATTgCTtTGG | 214 | chr2 | 57977284 | + | 4 |
| ggACAaaTATATATTCCTCTGG | 215 | chr2 | 57875842 | − | 4 |
| TTAtATCTATAaATcaCTCTGG | 216 | chr2 | 59319572 | + | 4 |
| TTgaATCTATAcATTgCTCTGG | 217 | chr2 | 63145773 | + | 4 |
| TTgaATCTATAaATTgCTCTGG | 218 | chr2 | 66657387 | + | 4 |
| TTcCAaCTcTAaATTCCTCAGG | 219 | chr2 | 66725112 | − | 4 |
| TTgCATCTATATATTgCTtTGG | 220 | chr2 | 67118268 | − | 3 |
| TTtCATCTATATgTTCaTtTGG | 221 | chr2 | 67476630 | − | 4 |
| TTgaATCTATATgTTCaTCAGG | 222 | chr2 | 68481032 | + | 4 |
| aTAtATCTAgATATTtCTCAGG | 223 | chr2 | 68282683 | − | 4 |
| aTAtATaTATATAgTCCTCAGG | 224 | chr2 | 73252338 | + | 4 |
| aTACATITATATATTtaTCTGG | 225 | chr2 | 75123876 | + | 4 |
| TTAaATCcATcTAcTCCTCAGG | 226 | chr2 | 76317037 | − | 4 |
| TTgCATCTATAaATTaCTtTGG | 227 | chr2 | 76935417 | + | 4 |
| TTACATCaAagTtTTCCTCTGG | 228 | chr2 | 77599883 | + | 4 |
| TTAaATCTATAaATTgCTtTGG | 229 | chr2 | 77722428 | + | 4 |
| TTACtTaTATAaATTtCTCTGG | 230 | chr2 | 86953103 | − | 4 |
| TTACtTaTATAaATTtCTCTGG | 231 | chr2 | 87816793 | + | 4 |
| TTAaATCTATAaATTgCTtTGG | 232 | chr2 | 87929632 | + | 4 |
| TTACATCTITATATTatTgAGG | 233 | chr2 | 89681250 | + | 4 |
| TTACATCTITATATTatTgAGG | 234 | chr2 | 91915979 | − | 4 |
| TTACtTCTcTATAaTCCaCTGG | 235 | chr2 | 94791088 | − | 4 |
| TTAaAgCTATATATTaCTtTGG | 236 | chr2 | 103216335 | − | 4 |
| TTACATCTgTAaATTgCTtTGG | 237 | chr2 | 103721176 | − | 4 |
| aTACATgTAgATATTCaTCTGG | 238 | chr2 | 104716490 | + | 4 |
| TTACtTaTATAaATTtCTCTGG | 239 | chr2 | 106457494 | + | 4 |
| TTACATCTgTAgATTgCTtTGG | 240 | chr2 | 107791385 | − | 4 |
| TTACtTaTATAaATTtCTCTGG | 241 | chr2 | 107838418 | − | 4 |
| TTACtTaTATAaATTtCTCTGG | 242 | chr2 | 108730935 | − | 4 |
| TTgCATCTATAaATTaCTtTGG | 243 | chr2 | 109724334 | + | 4 |
| TTACtTaTATAaATTtCTCTGG | 244 | chr2 | 109803593 | − | 4 |
| TTAaATaTAcATtTTCCTCAGG | 245 | chr2 | 110141512 | − | 4 |
| TTACtTaTATAaATTtCTCTGG | 246 | chr2 | 110567891 | + | 4 |
| TTACtTaTATAaATTtCTCTGG | 247 | chr2 | 112422475 | + | 4 |
| TTAaATCTATAaATTaCTtTGG | 248 | chr2 | 114690649 | + | 4 |
| aTAtATaTATATATTCCaCAGG | 249 | chr2 | 114782320 | − | 4 |
| TTAaATCTgTATATTCCTaTGG | 250 | chr2 | 119044004 | + | 3 |
| TTgaATCTATAaATTgCTCTGG | 251 | chr2 | 119817731 | + | 4 |
| TTAaATCTATAaATTaCTtTGG | 252 | chr2 | 119666893 | − | 4 |
| TcACATCTtTAgATTCCTaAGG | 253 | chr2 | 120642385 | − | 4 |
| TTgaATCTATATATTgCTtTGG | 254 | chr2 | 125296088 | + | 4 |
| TcACATCTATATATTtCTgTGG | 255 | chr2 | 125468468 | + | 3 |
| TTACATaatTATtTTCCTCTGG | 256 | chr2 | 125076697 | − | 4 |
| TTACATCcAgATAaTCaTCTGG | 257 | chr2 | 125929200 | − | 4 |
| TTAaATCTgTATATTgCTtTGG | 258 | chr2 | 132406539 | + | 4 |
| TTcCATCTccATAcTCCTCTGG | 259 | chr2 | 134505636 | + | 4 |
| TTgaATCTATAaATTCCTtTGG | 260 | chr2 | 135089452 | + | 4 |
| TTAaATtTAgATATTCCTaAGG | 261 | chr2 | 136808706 | − | 4 |
| TTAaATCTATAaATTaCTtTGG | 262 | chr2 | 138790464 | + | 4 |
| TTgCATCTATAaATTaCTtTGG | 263 | chr2 | 140159717 | + | 4 |
| TTAaATCTATAgATTgCTtTGG | 264 | chr2 | 140406766 | − | 4 |
| TTAtATtTATATcTTaCTCAGG | 265 | chr2 | 141101564 | + | 4 |
| TTACtTCTtTATtTTaCTCAGG | 266 | chr2 | 144935024 | − | 4 |
| TTAgATCTATATATTgtcCAGG | 267 | chr2 | 146170732 | − | 4 |
| TTACATgTATATATTgtTtTGG | 268 | chr2 | 148451688 | + | 4 |
| TTAaATCTATAaATTgCTtTGG | 269 | chr2 | 148856926 | − | 4 |
| TTAaATCTATAaATTaCTtTGG | 270 | chr2 | 149252872 | + | 4 |
| TTACATCcATgacTTCCTCAGG | 271 | chr2 | 148942699 | − | 4 |
| TTAaATCTATAaATTaCTtTGG | 272 | chr2 | 149835659 | + | 4 |
| TTAaATCTATAaATTgCTtTGG | 273 | chr2 | 155530108 | + | 4 |
| TTAaATCTATAgATTgCTtTGG | 274 | chr2 | 161743702 | + | 4 |
| TTACAaCTAaATATTtCTgTGG | 275 | chr2 | 163537580 | + | 4 |
| TTACATCTATAaAaTCaaCAGG | 276 | chr2 | 164221623 | + | 4 |
| TTgCATCTcTATgTTCaTCAGG | 277 | chr2 | 164280548 | + | 4 |
| TTACATCaATATATcaCTaAGG | 278 | chr2 | 165254070 | − | 4 |
| TTtCATgTtTATgTTCCTCAGG | 279 | chr2 | 166668583 | − | 4 |
| TTAaAcCTATAgATTgCTCTGG | 280 | chr2 | 171072162 | + | 4 |
| TTAaATCTATAaATTaCTtTGG | 281 | chr2 | 172689423 | − | 4 |
| TTACATITATtTtTTCCTaTGG | 282 | chr2 | 177059208 | + | 4 |
| TTACATaaATAcATTCtTCTGG | 283 | chr2 | 177316125 | + | 4 |
| TTAaATCTgTAgATTgCTCTGG | 284 | chr2 | 179085379 | + | 4 |
| TTgaATCTATAaATTCCTtTGG | 285 | chr2 | 183296947 | + | 4 |
| TTgaATCTATAaATTCCTtTGG | 286 | chr2 | 183421003 | − | 4 |
| TTgaATCTATAaATTCCTtTGG | 287 | chr2 | 183549548 | − | 4 |
| TTgaATCTATAaATTCCTtTGG | 288 | chr2 | 183846401 | − | 4 |
| aTAtcTCTATAgATTCCTCTGG | 289 | chr2 | 184847173 | − | 4 |
| TTAaATCTATAaATTaCTtTGG | 290 | chr2 | 185517552 | + | 4 |
| TTAtATtTATATtTTaCTCAGG | 291 | chr2 | 187710241 | − | 4 |
| TTAaATCcATAaATTCCTCTGG | 292 | chr2 | 188688979 | + | 3 |
| TTAaATaTATATATaCCTgTGG | 293 | chr2 | 193029815 | − | 4 |
| TTgaATCTATAaATTaCTCTGG | 294 | chr2 | 196866046 | − | 4 |
| TTAaATaTATATATgCCTCAGG | 295 | chr2 | 199375468 | + | 3 |
| TTAaATCTATAaATTaCTtTGG | 296 | chr2 | 205205015 | + | 4 |
| TTAaATCTATAggTTgCTCTGG | 297 | chr2 | 206373838 | + | 4 |
| TTACAaCTAaATAgTCCTtAGG | 298 | chr2 | 209445032 | − | 4 |
| TcACATtTATATAaTCCTtTGG | 299 | chr2 | 210569764 | + | 4 |
| TTgCATCTggATATTaCTCTGG | 300 | chr2 | 210994797 | + | 4 |
| aTAtATCTATATtTcCCTCTGG | 301 | chr2 | 213458804 | + | 4 |
| TTgaATCTATAaATTgCTCTGG | 302 | chr2 | 214658212 | + | 4 |
| TTAaATCTATAaATTaCTtTGG | 303 | chr2 | 226842842 | + | 4 |
| TTACATgTtTtTATTCCTtAGG | 304 | chr2 | 227214569 | + | 4 |
| TTACATCaATtTAaTtCTCAGG | 305 | chr2 | 231340005 | − | 4 |
| TTAaATCTATAaATTgCTtTGG | 306 | chr2 | 235475314 | + | 4 |
| cTtCATCTATAccTTCCTCAGG | 307 | chr2 | 239631367 | + | 4 |
| TTAtATCTATAaATTaCTtTGG | 308 | chr12 | 2104143 | + | 4 |
| TTgtATgTATgTATTCCTCAGG | 309 | chr12 | 7236306 | − | 4 |
| TTgaATCTATAaATTgCTCTGG | 310 | chr12 | 8523263 | + | 4 |
| TTAaATCTATAaATTgCTtTGG | 311 | chr12 | 9189071 | − | 4 |
| TTAaATCTATAaATTaCTtTGG | 312 | chr12 | 10352897 | + | 4 |
| TTcCAaCTAcATATTtCTCTGG | 313 | chr12 | 10825909 | − | 4 |
| TcAaATCTATAaATTgCTCTGG | 314 | chr12 | 11453211 | + | 4 |
| aTACATtTATAaATTgCTCTGG | 315 | chr12 | 13128298 | + | 4 |
| TTgCATCTATAcATTgCTtTGG | 316 | chr12 | 16963997 | + | 4 |
| TaAaATtTATATATTCCTgTGG | 317 | chr12 | 18917049 | + | 4 |
| TcACATaaAaATATTCCTCAGG | 318 | chr12 | 20383462 | + | 4 |
| aTAtATaTATATATTCCaCAGG | 319 | chr12 | 22793955 | − | 4 |
| gTAtATaTATATATTCaTCAGG | 320 | chr12 | 22984885 | − | 4 |
| aTACATtaATATATTCCTgTGG | 321 | chr12 | 27403124 | − | 4 |
| TTAtATCTgTAgATTCCTtTGG | 322 | chr12 | 28283276 | + | 4 |
| ccACATCaATATATcCCTCTGG | 323 | chr12 | 29067181 | + | 4 |
| TTAaATCTtTtTtTTCCTCTGG | 324 | chr12 | 29572869 | − | 4 |
| TTAaATCTATAaATTaCTtTGG | 325 | chr12 | 30375829 | + | 4 |
| TTAtATCgATAaATTCtTCAGG | 326 | chr12 | 34042968 | + | 4 |
| TTACAcCTATActTTCCcCTGG | 327 | chr12 | 43831334 | + | 4 |
| TTgCATCTATATcTcCCaCTGG | 328 | chr12 | 43604314 | − | 4 |
| TTAaATCTATAaATTgCTtTGG | 329 | chr12 | 43955712 | − | 4 |
| TTAaATCTATAgATTaCTtTGG | 330 | chr12 | 46017454 | + | 4 |
| TTAtATCTAcAgATTCCTCAGG | 331 | chr12 | 48143897 | − | 3 |
| TTAaATCTATAaATTaCTtTGG | 332 | chr12 | 58836951 | + | 4 |
| aTtCATCTATATATTCaTgGGG | 333 | chr12 | 59082608 | + | 4 |
| gTACtTtTATATgTTCCTCTGG | 334 | chr12 | 60105886 | + | 4 |
| TTgaATCTATATATTaCTtTGG | 335 | chr12 | 60770195 | − | 4 |
| TTtCATCTATACATTCtTtTGG | 336 | chr12 | 66552640 | − | 4 |
| TaAtATgTATATATTgCTCTGG | 337 | chr12 | 67151865 | + | 4 |
| TTAtATCTAgATATTCtTaTGG | 338 | chr12 | 71416055 | − | 4 |
| TTAaATCTATAaATTaCTtTGG | 339 | chr12 | 72852916 | + | 4 |
| aTACATaTATATATTCtTaGGG | 340 | chr12 | 73832820 | + | 4 |
| TTACATgTcTATATTCCaaGGG | 341 | chr12 | 73912975 | + | 4 |
| TTAaATCTATAaATTaCTtTGG | 342 | chr12 | 75618610 | − | 4 |
| TTAaATCTATAaATTaCTtTGG | 343 | chr12 | 78523518 | − | 4 |
| TTgaATCTATAaATTaCTCTGG | 344 | chr12 | 80111813 | + | 4 |
| TTACATgTAgATATagCTCTGG | 345 | chr12 | 85067387 | − | 4 |
| aTAtATaTATATATTCtTCAGG | 346 | chr12 | 86569089 | + | 4 |
| TTAaAcCTATATATTtCTtTGG | 347 | chr12 | 90570906 | + | 4 |
| TTACATggATAcATTCtTCAGG | 348 | chr12 | 90907644 | − | 4 |
| TTAaATCTATAgATTgCTtTGG | 349 | chr12 | 91391600 | + | 4 |
| TTgaATCTATAaATTCCTtTGG | 350 | chr12 | 91549622 | − | 4 |
| TTAaAaCcATATcTTCCTCTGG | 351 | chr12 | 92530040 | − | 4 |
| TTAaATCTATAaATTaCTtTGG | 352 | chr12 | 94003877 | + | 4 |
| TTAaATaaATATATTCCTtTGG | 353 | chr12 | 97898923 | − | 4 |
| aTAtATaTATATATTCCTCTGG | 354 | chr12 | 100439075 | + | 3 |
| TTgaATCTATAaATTtCTCTGG | 355 | chr12 | 102639637 | − | 4 |
| TTAaATCTATAaATTaCTtTGG | 356 | chr12 | 103429543 | − | 4 |
| TTtCATgTATATATTCaTgAGG | 357 | chr12 | 104380754 | + | 4 |
| TTctATCTAaAaATTCCTCTGG | 358 | chr12 | 106745664 | − | 4 |
| aTAaATCTgTATATTCCTtTGG | 359 | chr12 | 109136339 | + | 4 |
| TTAaATCTAgATgTTCCTgAGG | 360 | chr12 | 113564379 | − | 4 |
| aTAtATaTATATATTCCaCAGG | 361 | chr12 | 127561863 | − | 4 |
| TTgaATCTATAaATTaCTCTGG | 362 | chr21 | 17313487 | − | 4 |
| TTAaATCcAaATATgCCTCTGG | 363 | chr21 | 18578705 | − | 4 |
| TTAaATCTATAaATTaCTtTGG | 364 | chr21 | 19428729 | − | 4 |
| TTgaATCTATAaATTCCTtTGG | 365 | chr21 | 19945459 | + | 4 |
| TTAaATCTcTAcATTCCTtTGG | 366 | chr21 | 20500302 | + | 4 |
| TTcCAcCaATgTATTCCTCTGG | 367 | chr21 | 23887224 | − | 4 |
| TTACCTCCATATATCCCcCTGG | 368 | chr21 | 23967758 | + | 4 |
| TTtCATaTATATgTTCaTCAGG | 369 | chr21 | 24917018 | + | 4 |
| TTAtATtTATAaATTCCTCAGG | 370 | chr21 | 26102517 | + | 3 |
| TTACATCTATAaATTaCctTGG | 371 | chr21 | 27662850 | − | 4 |
| aTAtATCTcTATATTtCTCTGG | 372 | chr21 | 28063571 | + | 4 |
| TTAaggCTATATgTTCCTCTGG | 373 | chr21 | 34287141 | − | 4 |
| TTAaATCTATAaATTaCTtTGG | 374 | chr21 | 35184349 | + | 4 |
| TaACATCTACATATTtCTgTGG | 375 | chr21 | 35272716 | + | 4 |
| TTAtccCTATATATTCCaCTGG | 376 | chr21 | 35679573 | − | 4 |
| TTAaATCTATAaATTaCTtCGG | 377 | chr21 | 39069312 | − | 4 |
| TcACATCTATcTcTTgCTCAGG | 378 | chr21 | 43740772 | + | 4 |
| TTAaATCTATAgATTgCTtTGG | 379 | chr4 | 16577644 | − | 4 |
| TTgaATCTATATATTaCTtTGG | 380 | chr4 | 19282509 | − | 4 |
| TTAaATCTATAaATTgCTtTGG | 381 | chr4 | 19626682 | + | 4 |
| TTACAaaTAaATATTCtTCTGG | 382 | chr4 | 21964586 | − | 4 |
| TTgCtTCcATATATaCCTCAGG | 383 | chr4 | 28641347 | − | 4 |
| TagCATtTATATATTCCTCTGG | 384 | chr4 | 29380991 | + | 3 |
| TTtCATaTATtTATTCaTCTGG | 385 | chr4 | 29696057 | − | 4 |
| TTACATCTgTATAgTgCTaTGG | 386 | chr4 | 31513600 | − | 4 |
| TTAaAaaTATATtTTCCTCTGG | 387 | chr4 | 32566858 | + | 4 |
| TTAaATCTATAgATTgCTtTGG | 388 | chr4 | 34276291 | + | 4 |
| aTAtATaTATATATTtCTCTGG | 389 | chr4 | 34454495 | − | 4 |
| TTAtgTtTATATgTTCCTCAGG | 390 | chr4 | 34893218 | − | 4 |
| TTACATCTATATgTaCCcaAGG | 391 | chr4 | 35617003 | + | 4 |
| TTACATagATATATTCaTtTGG | 392 | chr4 | 35835955 | + | 4 |
| TTAtATCTATggATTaCTCTGG | 393 | chr4 | 43494062 | + | 4 |
| TTAaATCTgTATATTgCTtTGG | 394 | chr4 | 55776737 | − | 4 |
| aTAtATaTATATATTCCTaCGG | 395 | chr4 | 60213907 | − | 4 |
| TTACtTtTATAgATTCCTtAGG | 396 | chr4 | 62576827 | + | 4 |
| TTgtATCTgTATgTTCCTCTGG | 397 | chr4 | 63761850 | + | 4 |
| TTtCATtTATATATTCCcaGGG | 398 | chr4 | 64555049 | − | 4 |
| TTAaATCTATAaATTaCTtTGG | 399 | chr4 | 65519951 | − | 4 |
| TTgaATCTATAaATTaCTCTGG | 400 | chr4 | 65857319 | + | 4 |
| TTAaATCTATAaATTaCTtTGG | 401 | chr4 | 67254194 | + | 4 |
| TTgaATCTATAaATTaCTCTGG | 402 | chr4 | 67814245 | − | 4 |
| TTAgATCTATATATTtCTtTGG | 403 | chr4 | 68103724 | + | 3 |
| TTAtATaTATATATTgCTaAGG | 404 | chr4 | 70545495 | + | 4 |
| TTACATCTAcAgATTgCTtTGG | 405 | chr4 | 72162949 | + | 4 |
| TTAaATCTATAgATTgCTtTGG | 406 | chr4 | 72673426 | + | 4 |
| aTtCATCTtTATATTCCTaAGG | 407 | chr4 | 72817412 | − | 4 |
| TTACATCaATtTAaTtCTCAGG | 408 | chr4 | 73032552 | + | 4 |
| TTAaATCTATAaATTaCTtTGG | 409 | chr4 | 74341884 | + | 4 |
| TTAaATCTgTATATTgCTtTGG | 410 | chr4 | 74678889 | + | 4 |
| aTgCATCTACATATTCCCCAGG | 411 | chr4 | 77348208 | + | 4 |
| TTAaATCTgTATATTgCTtTGG | 412 | chr4 | 77646589 | − | 4 |
| TTACATCCATtTtTTCCTtTGG | 413 | chr4 | 82933079 | − | 4 |
| TTAttTCTATATcTTCCTCAGG | 414 | chr4 | 86672807 | − | 3 |
| TaACATtTgTATgTTCCTCAGG | 415 | chr4 | 88386953 | + | 4 |
| TTAaATCTATAgATTgCTtTGG | 416 | chr4 | 88373927 | − | 4 |
| TTAaATCTATAaATTgCTtTGG | 417 | chr4 | 91208779 | + | 4 |
| TTAaATCTATAaATTgCTtTGG | 418 | chr4 | 91807931 | + | 4 |
| TTAaATCTATAaATTgCTCTGG | 419 | chr4 | 91841342 | − | 3 |
| TTACtTCTATATATaCaTgAGG | 420 | chr4 | 92153699 | − | 4 |
| TTAatTtTATATATTCaTCAGG | 421 | chr4 | 93061165 | + | 4 |
| TTgaATCTATAgATcCCTCTGG | 422 | chr4 | 93536129 | − | 4 |
| TTgaATCTATATATTaCTtTGG | 423 | chr4 | 94151163 | + | 4 |
| TTgaATCTATATATTaCTtTGG | 424 | chr4 | 99243786 | + | 4 |
| TTAaATCTgTAgATTCtTCTGG | 425 | chr4 | 101575570 | + | 4 |
| TTACATCTITATATTatTgAGG | 426 | chr4 | 102934074 | − | 4 |
| TggCATCTATAccTTCCTCCGG | 427 | chr4 | 109259807 | + | 4 |
| TTgaATCaATATATTCtTCAGG | 428 | chr4 | 114574800 | + | 4 |
| aTAttTCTATATATTCCTCGGG | 429 | chr4 | 115506828 | + | 3 |
| TTAaATCTATAcATTgCTtTGG | 430 | chr4 | 116416336 | + | 4 |
| TTgaATCTATAaATTCCTtTGG | 431 | chr4 | 119486116 | − | 4 |
| TTgaATCTATAaATTCCTtTGG | 432 | chr4 | 120949636 | − | 4 |
| TaAaATCTATAgATTgCTCTGG | 433 | chr4 | 123427287 | + | 4 |
| TaACATaTATATtTTtCTCTGG | 434 | chr4 | 125781465 | − | 4 |
| TTAgATgTATAaATTgCTCTGG | 435 | chr4 | 129010481 | + | 4 |
| TTAaATCTATAcATTgCTtTGG | 436 | chr4 | 129229177 | − | 4 |
| aTAtATaTATATATTtCTCAGG | 437 | chr4 | 130626547 | + | 4 |
| TTACATCTgTAgATTgCTtTGG | 438 | chr4 | 131058269 | − | 4 |
| TTACATCTATATATTaaTgTGG | 439 | chr4 | 133484939 | − | 3 |
| TTAaATCTATAcATTgCTtTGG | 440 | chr4 | 136596691 | − | 4 |
| TcACATaTATATAaTtCTCTGG | 441 | chr4 | 137872366 | + | 4 |
| TTtCcTCTATATtTTCaTCTGG | 442 | chr4 | 137940397 | − | 4 |
| aTAtATaTATATATTCtTCTGG | 443 | chr4 | 142495515 | + | 4 |
| TTACATCTgTAgATTgCTtTGG | 444 | chr4 | 143041006 | − | 4 |
| TTAaATCTATAaATTaCTtTGG | 445 | chr4 | 144246044 | + | 4 |
| TTAaATCTATAaATTaCTtTGG | 446 | chr4 | 144157439 | − | 4 |
| TTAaATCTtTAaATTaCTCTGG | 447 | chr4 | 149180408 | − | 4 |
| TTAaATCTATAgATTgCTtTGG | 448 | chr4 | 153696246 | + | 4 |
| TTgaATCTATAaATTaCTCTGG | 449 | chr4 | 153855284 | + | 4 |
| TTgCATCTATAaATTaCTtTGG | 450 | chr4 | 158898404 | + | 4 |
| TcACATaaATtTATTCCTCTGG | 451 | chr4 | 164693934 | − | 4 |
| gTACATCTcTAgtTTCCTCAGG | 452 | chr4 | 164825855 | − | 4 |
| TTACCTCTATAaAaTgCTCTGG | 453 | chr4 | 173736570 | − | 4 |
| aTAtATaTATATATTCaTCAGG | 454 | chr4 | 174688354 | + | 4 |
| TTtCAgCTgTtTATTCCTCAGG | 455 | chr4 | 179288109 | + | 4 |
| TTACATCcAcAcATgCCTCTGG | 456 | chr4 | 179794566 | + | 4 |
| TTgCATCTgTAcATTCCTtTGG | 457 | chr4 | 181053295 | − | 4 |
| TTgaATCTATATATTgCTtTGG | 458 | chr4 | 182031618 | + | 4 |
| TTAttTgTATATATTCCTgGGG | 459 | chr4 | 185081469 | + | 4 |
| TTACATgcATATtTTgCTCCGG | 460 | chr4 | 185903078 | − | 4 |
| TTtCATCaAaATATTgCTCTGG | 461 | chr4 | 188670640 | + | 4 |
| TTACATgTtgATATTCCTCAGG | 462 | chr17 | 3398752 | + | 3 |
| aTAtATaTATATATTCCTaAGG | 463 | chr17 | 7699525 | + | 4 |
| TTAtATaTATATtTTtCTCTGG | 464 | chr17 | 11940459 | − | 4 |
| aTACcTtTATATATcCCTCTGG | 465 | chr17 | 20927251 | + | 4 |
| TTACATgTATATATTaaTtTGG | 466 | chr17 | 28565432 | − | 4 |
| TTAaATCTATAgATTgCTtTGG | 467 | chr17 | 32788817 | + | 4 |
| aTAtATaTATATATTCCTaAGG | 468 | chr17 | 41161965 | − | 4 |
| TTACAgaaATATATTCCCCAGG | 469 | chr17 | 42285389 | − | 4 |
| gTAtATaTATATATTtCTCAGG | 470 | chr17 | 44501935 | + | 4 |
| TcACCTCTATcTATTCCaCTGG | 471 | chr17 | 49967211 | + | 4 |
| TTgaATCTATAaATTgCTCTGG | 472 | chr17 | 51897631 | + | 4 |
| TTACCTCTATAaATTCCTtTGG | 473 | chr17 | 55803283 | − | 3 |
| TTAaATCTATAaATTgCTtTGG | 474 | chr17 | 56340870 | + | 4 |
| TTAaATCTATATATatCaCTGG | 475 | chr17 | 60230364 | + | 4 |
| TTAaATCTATAgATTgCTtTGG | 476 | chr17 | 60814094 | + | 4 |
| TTACATgTATtTATTtCTgGGG | 477 | chr17 | 63284528 | + | 4 |
| TTgCATCTATATATTCCTtTGG | 478 | chr17 | 65769343 | − | 2 |
| TTACATCTcTAagTTaCTCAGG | 479 | chr17 | 70076479 | − | 4 |
| TTgaATCTATATATTgCTtTGG | 480 | chr17 | 70285025 | − | 4 |
| TTctATCTATtTATTCaTCAGG | 481 | chr17 | 77223219 | − | 4 |
| TTAaATCTATAgATTgCTtTGG | 482 | chr17 | 82996167 | − | 4 |
| cTcCATCTATATgcTCCTCTGG | 483 | chr16 | 5730332 | − | 4 |
| cTcCAgCTATATtTTCCTCAGG | 484 | chr16 | 10595765 | − | 4 |
| TTACATITATATATgCCTtTGG | 485 | chr16 | 11121049 | − | 3 |
| cTACATCCATAatTTCCTCAGG | 486 | chr16 | 17097736 | − | 4 |
| TTtCATCTtTATAgTaCTCAGG | 487 | chr16 | 20728630 | − | 4 |
| TTtCATtTgTATATTCtTCAGG | 488 | chr16 | 21297456 | − | 4 |
| TTAtgTCTATcTgTTCCTCTGG | 489 | chr16 | 26706744 | − | 4 |
| TgAtATaTATATcTTCCTCCGG | 490 | chr16 | 27616997 | − | 4 |
| TTACATCTITATATTatTgAGG | 491 | chr16 | 32403197 | + | 4 |
| TTAaATCTgTATATTgCTtTGG | 492 | chr16 | 47789956 | + | 4 |
| TTACccCcATATATgCCTCTGG | 493 | chr16 | 51176442 | − | 4 |
| TTAgtTCTATAaATTCCcCTGG | 494 | chr16 | 56288947 | + | 4 |
| TTAgATCTATtTATaCCTtGGG | 495 | chr16 | 56787903 | + | 4 |
| TgcCATCTATATATTttTCTGG | 496 | chr16 | 59684520 | + | 4 |
| TTACAcCTAaATATTaaTCTGG | 497 | chr16 | 59804394 | − | 4 |
| TTAaATCTATAaATTgCTtTGG | 498 | chr16 | 62116373 | + | 4 |
| TTAtATaTATtTtTTCCTCTGG | 499 | chr16 | 62752788 | + | 4 |
| TTtCcTCTATAaATTaCTCGGG | 500 | chr16 | 63129049 | − | 4 |
| TaACcTCTgTtTATTCCTCGGG | 501 | chr16 | 66976892 | − | 4 |
| TTACCTCTATATtTcCCTCAGG | 502 | chr16 | 67035870 | − | 3 |
| aTAgATCTAcATATTtCTCAGG | 503 | chr16 | 70904410 | − | 4 |
| TTtCATtTATATATCCCTaGGG | 504 | chr16 | 72809167 | − | 4 |
| TTAaAgCTgTATATTgCTCTGG | 505 | chr16 | 76807752 | + | 4 |
| TTgCATCTATAaATTaCTtTGG | 506 | chr16 | 77265330 | + | 4 |
| TTAaATCTATAaATTaCTtTGG | 507 | chr16 | 81344185 | − | 4 |
| TgACATCTAcAaAcTCCTCTGG | 508 | chr16 | 83917994 | − | 4 |
| TTAaATCTATAgATTgCTtTGG | 509 | chr16 | 84401896 | + | 4 |
| TaACAgCTATAgtTTCCTCTGG | 510 | chr9 | 2685911 | + | 4 |
| TccCAatTATATATTCCTCCGG | 511 | chr9 | 4277707 | + | 4 |
| TTttATCTATAgcTTCCTCTGG | 512 | chr9 | 5570375 | + | 4 |
| aTACATCTATAatTTCCTgGGG | 513 | chr9 | 6011115 | − | 4 |
| TTAaATCTATAgATTgCTtTGG | 514 | chr9 | 9474507 | + | 4 |
| TTAtATTATATATTtCTgTGG | 515 | chr9 | 9297570 | − | 4 |
| TTACATCTATAgATcaCTtTGG | 516 | chr9 | 15932176 | + | 4 |
| TTcCtTCTATcTATTCCTgTGG | 517 | chr9 | 16152277 | − | 4 |
| TaACATCTATATtgTtCTCTGG | 518 | chr9 | 17743289 | − | 4 |
| TTgaATCTATAaATTaCTCTGG | 519 | chr9 | 19500709 | + | 4 |
| TTttATCcATATATcCCTCAGG | 520 | chr9 | 19945823 | + | 4 |
| TgAaATCTATAgATTaCTCTGG | 521 | chr9 | 21101823 | + | 4 |
| TacCATtTATATtTTCCTCGGG | 522 | chr9 | 21977157 | + | 4 |
| TTAaATCTATAaATTaCTtGGG | 523 | chr9 | 27319564 | + | 4 |
| TTgaATCTATAaATTaCTCTGG | 524 | chr9 | 27796398 | + | 4 |
| cTtaATCTATATATTCCTgTGG | 525 | chr9 | 29925871 | + | 4 |
| TTACATCaATATATTaaTgAGG | 526 | chr9 | 29764430 | − | 4 |
| TTgCATCTATAaATTaCTgTGG | 527 | chr9 | 30475343 | − | 4 |
| TgcCATCTATATATTtCTtTGG | 528 | chr9 | 31710354 | − | 4 |
| TTcCAcCTATATAaTaCTCTGG | 529 | chr9 | 36680819 | − | 4 |
| TTAaATCTATAgATTgCTtTGG | 530 | chr9 | 38447641 | + | 4 |
| TTACtTCTcTATAaTCCaCTGG | 531 | chr9 | 40289267 | + | 4 |
| TTtCATCTATAgATTgCTtTGG | 532 | chr9 | 42263843 | − | 4 |
| TTACtTCTcTATAaTCCaCTGG | 533 | chr9 | 42918615 | + | 4 |
| TTACtTCTcTATAaTCCaCTGG | 534 | chr9 | 64435503 | + | 4 |
| TTACTCTCTATAaTCCaCTGG | 535 | chr9 | 66087016 | − | 4 |
| TaACATCTcTATATTaCTaGGG | 536 | chr9 | 68550144 | + | 4 |
| TTgaATCTATATATTaCTtTGG | 537 | chr9 | 70244890 | − | 4 |
| CTACATCTAaATATaCaTCAGG | 538 | chr9 | 72665789 | − | 4 |
| TcACATCTtTAacTTCCTCAGG | 539 | chr9 | 73581317 | + | 4 |
| TTAtAaaTATATATTCCTaTGG | 540 | chr9 | 73752401 | − | 4 |
| TTAaATCTATAgATTgCTtTGG | 541 | chr9 | 73933166 | − | 4 |
| TTAaATCTATAaATTaCTtAGG | 542 | chr9 | 74224754 | + | 4 |
| TTAaATCTATAaATTaCTtAGG | 543 | chr9 | 74223710 | − | 4 |
| TTAaATCTATAaATTaCTtTGG | 544 | chr9 | 75323265 | + | 4 |
| TTgaATCTATAgATTtCTCTGG | 545 | chr9 | 76078889 | + | 4 |
| TTAaATaTATATATTCaaCTGG | 546 | chr9 | 77067996 | + | 4 |
| TTAgcTCTATAcATTtCTCAGG | 547 | chr9 | 78320871 | + | 4 |
| TTAaATCTATAaATTaCTtTGG | 548 | chr9 | 78460153 | − | 4 |
| TTACATaTATATtTTCCatTGG | 549 | chr9 | 78826001 | + | 4 |
| TTACATtTgTATATTgCTtTGG | 550 | chr9 | 80262434 | − | 4 |
| TTAaATCTATAaATTaCTtTGG | 551 | chr9 | 81348759 | − | 4 |
| TTACCTCTATATAgTatTCTGG | 552 | chr9 | 82855593 | − | 4 |
| TTcCATCTtTATcTTCCaCAGG | 553 | chr9 | 86581737 | + | 4 |
| aTAtATCTATATAcaCCTCAGG | 554 | chr9 | 91264863 | + | 4 |
| TTgCAgCTtTAcATTCCTCTGG | 555 | chr9 | 91930618 | − | 4 |
| ccACATCTgTATcTTCCTCAGG | 556 | chr9 | 100480876 | − | 4 |
| TTAagTCTATATATTgCTtTGG | 557 | chr9 | 102451728 | − | 4 |
| TTAaATCTATAaATTgCTtTGG | 558 | chr9 | 103222321 | − | 4 |
| TTAaATCTATATATTgCTtTGG | 559 | chr9 | 104648991 | − | 3 |
| TTgaATCTATAaATTCCTtTGG | 560 | chr9 | 106570057 | − | 4 |
| TatCATCTATATATgCtTCCGG | 561 | chr9 | 108338282 | + | 4 |
| aTACATaTATATATTCaaCAGG | 562 | chr9 | 111911985 | + | 4 |
| aTACATaTATATATTCaaCAGG | 563 | chr9 | 111912007 | + | 4 |
| TTAaATCTATAaATTaCTtTGG | 564 | chr9 | 115728381 | − | 4 |
| TTACATITAaATcTTtCTCAGG | 565 | chr9 | 115711714 | − | 4 |
| TTAgtTgTATATcTTCCTCAGG | 566 | chr9 | 120425012 | + | 4 |
| TTAaATCTATACATTaCTCTGG | 567 | chr9 | 123729682 | − | 3 |
| cTAaATCTAaATAgTCCTCTGG | 568 | chr9 | 124992919 | + | 4 |
| TTAaATCTgTAcATTgCTCTGG | 569 | chr9 | 134105557 | − | 4 |
| aTcCATCTcTAaATTCCTCTGG | 570 | chrX | 3046517 | − | 4 |
| TTAtATaTATATATaaCTCGGG | 571 | chrX | 16076111 | + | 4 |
| TTACATCTgTAgATTgCTtTGG | 572 | chrX | 25800566 | + | 4 |
| TTgCATCTAaATATTtCTgTGG | 573 | chrX | 26280875 | + | 4 |
| TTgCATCTATATATTCaTCAGG | 574 | chrX | 26289901 | + | 2 |
| TTAaATCTATAaATTgCTtTGG | 575 | chrX | 26542715 | − | 4 |
| TTACATITATATATTtaTtGGG | 576 | chrX | 31842762 | + | 4 |
| TTcCATtTtTATATcCCTCTGG | 577 | chrX | 32132007 | − | 4 |
| gTACcTCTAcATATTCaTCTGG | 578 | chrX | 33202372 | + | 4 |
| TTgaATCTATATATTaCTtTGG | 579 | chrX | 33547842 | + | 4 |
| TTAaATCTgTATATTgCTtTGG | 580 | chrX | 33845026 | + | 4 |
| TTAaATCTgTAaATTCCTtTGG | 581 | chrX | 34598016 | + | 4 |
| TTgCATtTATATtTTCCcCTGG | 582 | chrX | 34312094 | − | 4 |
| TgACATCaATATcTgCCTCTGG | 583 | chrX | 36851718 | + | 4 |
| TTACATgTAaATATcCCTtTGG | 584 | chrX | 37798732 | + | 4 |
| aTACATCTtcATATTCaTCTGG | 585 | chrX | 45063317 | + | 4 |
| TTAaATCTgTATATTtCTtTGG | 586 | chrX | 45588344 | + | 4 |
| TTAaATCTATcTtTTtCTCAGG | 587 | chrX | 46329945 | + | 4 |
| cTACATtTtTATATTCCTCTGG | 588 | chrX | 47733737 | + | 3 |
| TTcCATtgATgTATTCCTCTGG | 589 | chrX | 48710249 | − | 4 |
| aTAtATaTATATATTCCTaAGG | 590 | chrX | 49971205 | − | 4 |
| cTACATCTATAgATTgCTtTGG | 591 | chrX | 52000490 | + | 4 |
| TTACAgCcATATAaTCCcCTGG | 592 | chrX | 53843621 | − | 4 |
| cTACATCTtTATATgtCTCTGG | 593 | chrX | 54748544 | + | 4 |
| TTAaAgCTATATATTggTCAGG | 594 | chrX | 54670926 | − | 4 |
| TTAaATCTATAaATTaCTtTGG | 595 | chrX | 55068604 | + | 4 |
| TTAaATCTATAaATTaCTtTGG | 596 | chrX | 56706991 | + | 4 |
| TTgCATCTATAaATTaCTtTGG | 597 | chrX | 63543497 | − | 4 |
| TTACATCTgTAaATTgCTtTGG | 598 | chrX | 64118478 | + | 4 |
| TTAaATCTATAgATTgCTtTGG | 599 | chrX | 65398194 | − | 4 |
| TTgaATCTATAaATTaCTCTGG | 600 | chrX | 66552418 | + | 4 |
| TTgCATCagTATATTCCTgAGG | 601 | chrX | 70345683 | − | 4 |
| TcACATCTATgTATTtCTtTGG | 602 | chrX | 71530936 | + | 4 |
| TTAaATCTATAaATTaCTaTGG | 603 | chrX | 73651812 | − | 4 |
| TTgaATCTATATATTgCTtTGG | 604 | chrX | 74198054 | + | 4 |
| TTgaATCTATAaATTgCTCTGG | 605 | chrX | 74392613 | − | 4 |
| TTAaATCTATAcATTgCTtTGG | 606 | chrX | 76054197 | + | 4 |
| TTAaATCTATAaATTaCTtTGG | 607 | chrX | 76090808 | + | 4 |
| TTAtATCTATAaATTaCTtTGG | 608 | chrX | 76178866 | + | 4 |
| TTgaATCTATATATTaCTtTGG | 609 | chrX | 79000257 | − | 4 |
| TTAaATCTATAaATTaCTtTGG | 610 | chrX | 79278781 | − | 4 |
| TTAaATCTATAaATTaCTtTGG | 611 | chrX | 79843721 | − | 4 |
| TTgaATCTATAaATTCCTtTGG | 612 | chrX | 80498258 | + | 4 |
| TTgaATCTATAaATTCCTtTGG | 613 | chrX | 81088461 | − | 4 |
| TTAaATCTATAaATTaCTtTGG | 614 | chrX | 81453812 | − | 4 |
| TTAaATCTATAaATTgCTtTGG | 615 | chrX | 82071863 | − | 4 |
| TTAaATCTATAaATTaCTtTGG | 616 | chrX | 82949115 | − | 4 |
| TTAaATCTATAaATTaCTtTGG | 617 | chrX | 83927926 | + | 4 |
| TTAaATCTgTAgATTgCTCTGG | 618 | chrX | 85646896 | − | 4 |
| TTACtTCTcTtTATTtCTCTGG | 619 | chrX | 88815574 | + | 4 |
| TTgaATCTATAgATTCCTtAGG | 620 | chrX | 88594455 | − | 4 |
| TTACATCTgTAaATTgCTtTGG | 621 | chrX | 89318170 | + | 4 |
| aTAtATaTATATATTCCTCTGG | 622 | chrX | 89460593 | − | 3 |
| TTAaAcCTATATATTgCTtTGG | 623 | chrX | 90014274 | + | 4 |
| TTAaATCTATAaATTaCTtTGG | 624 | chrX | 90941100 | − | 4 |
| TTAaATCTATAaATTaCTtTGG | 625 | chrX | 98694712 | + | 4 |
| TTgaATCTATAaATTCCTtTGG | 626 | chrX | 99756486 | + | 4 |
| TgACAgCTATtTATTtCTCAGG | 627 | chrX | 100418691 | − | 4 |
| TTAaATCTATAaATTaCTtTGG | 628 | chrX | 102039772 | − | 4 |
| aTACActTtTATATTCCTCTGG | 629 | chrX | 105255473 | − | 4 |
| TTgCATCTgTATATTaCTtGGG | 630 | chrX | 105642296 | − | 4 |
| TTctATgTgTATATTCCTCTGG | 631 | chrX | 108422547 | − | 4 |
| TTACATCTgatTATTCtTCTGG | 632 | chrX | 109356279 | + | 4 |
| TTAacTCTATATATTCCTgGGG | 633 | chrX | 112234482 | + | 3 |
| TTgaATCTATATATTtCTtAGG | 634 | chrX | 115312324 | + | 4 |
| TTAaATCTATAaATTaCTtTGG | 635 | chrX | 123287839 | − | 4 |
| TTAaATCTATAaATTgCTtTGG | 636 | chrX | 126087444 | − | 4 |
| TTgaATCTATAaATTCCTtTGG | 637 | chrX | 128350758 | − | 4 |
| caAaATCTATATATTCITCTGG | 638 | chrX | 128708253 | + | 4 |
| TTACATtTATActaTCCTCAGG | 639 | chrX | 128851079 | + | 4 |
| TTACAaCTATATAaTatTCTGG | 640 | chrX | 130644660 | + | 4 |
| TaAgATCTATAaATaCCTCCGG | 641 | chrX | 130475229 | − | 4 |
| TTACATaTgTATATaCCTgTGG | 642 | chrX | 136399201 | − | 4 |
| TTAaATCTgTAgATTgCTCTGG | 643 | chrX | 137395378 | + | 4 |
| TTgCATCTATAaATTtCTtTGG | 644 | chrX | 137806744 | + | 4 |
| TTgCATCTATAgATTgCTtTGG | 645 | chrX | 137954527 | + | 4 |
| TTtCATCTAgATATatCTCAGG | 646 | chrX | 138248444 | − | 4 |
| aTAtATCTATgTATcCCTCAGG | 647 | chrX | 139317874 | + | 4 |
| TTAaATCTATAaATTaCTtTGG | 648 | chrX | 139374270 | + | 4 |
| TTgaATCTATAaATTaCTCTGG | 649 | chrX | 140268443 | + | 4 |
| TTgaATCTATAgATTgCTCTGG | 650 | chrX | 140396003 | − | 4 |
| TTACAaCaATATATTgtTCTGG | 651 | chrX | 140738374 | − | 4 |
| TTgCATCTATAaATTaCTtTGG | 652 | chrX | 141250398 | + | 4 |
| TTAtATaTATATATTCaTaTGG | 653 | chrX | 142223359 | + | 4 |
| TTAaATCTATAaATTgCTtTGG | 654 | chrX | 144109508 | − | 4 |
| TTACAaCTAaATATTttTCAGG | 655 | chrX | 145133831 | − | 4 |
| TTACATaTATgTATaCaTCAGG | 656 | chrX | 145814154 | − | 4 |
| TTAtATgaATATATTCCTCTGG | 657 | chrX | 147245613 | − | 3 |
| TTAaATCTATAgATTgCTtTGG | 658 | chrX | 155328573 | − | 4 |
| TcACATCTcTgcATTCCTCTGG | 659 | chr14 | 19895358 | − | 4 |
| TTACAgCTAaATtTTCCTCTGG | 660 | chr14 | 20061666 | + | 3 |
| TTAaATgTcTtTATTCCTCAGG | 661 | chr14 | 20842404 | − | 4 |
| TTAaATCTATAaATTaCTtTGG | 662 | chr14 | 25592085 | + | 4 |
| TTACActTATACATTaCTCTGG | 663 | chr14 | 26463897 | + | 4 |
| TTACATCTgTcTggTCCTCAGG | 664 | chr14 | 29069921 | + | 4 |
| TTgaATCTATAaATTaCTCTGG | 665 | chr14 | 29976906 | − | 4 |
| TTgCATCTATAaATTaCTtTGG | 666 | chr14 | 32289331 | + | 4 |
| TTACtTCTcgATATTCCcCTGG | 667 | chr14 | 33560838 | + | 4 |
| TTAaATtTATATATTtCTITGG | 668 | chr14 | 35593402 | − | 4 |
| TTAaATCTATAaATTaCTtTGG | 669 | chr14 | 37661737 | + | 4 |
| TTgaATCTgTATATTCCTtTGG | 670 | chr14 | 37460927 | − | 4 |
| TTAaATCTATAaATTaCTtTGG | 671 | chr14 | 37664076 | − | 4 |
| TTAaATCTATAaATTaCTtTGG | 672 | chr14 | 40082760 | − | 4 |
| TTACAcCTgTAgATTtCTCTGG | 673 | chr14 | 40273393 | − | 4 |
| TTtaATCTgTATATTCCTtTGG | 674 | chr14 | 41491559 | + | 4 |
| TTgaATCTATAaATTCCTtTGG | 675 | chr14 | 44047077 | − | 4 |
| TTgaATCTATAaATTCCTtTGG | 676 | chr14 | 45491274 | − | 4 |
| caAaATCTATATATTtCTCGGG | 677 | chr14 | 47115434 | − | 4 |
| TTAtgTCTATATAgTCCTaAGG | 678 | chr14 | 51177426 | + | 4 |
| TTAacTCaAcATATTCCTCAGG | 679 | chr14 | 51527306 | + | 4 |
| TTcCtTCTATATATTgCTCTGG | 680 | chr14 | 51631248 | − | 3 |
| TTACAaCTgTATgTTtCTCAGG | 681 | chr14 | 51941500 | − | 4 |
| TTACATCTtTAcATTtCaCTGG | 682 | chr14 | 54921632 | + | 4 |
| TTAaAaaTtTATATTCCTCTGG | 683 | chr14 | 56596947 | + | 4 |
| TTgaATCgATATtTTCCTCAGG | 684 | chr14 | 58347812 | − | 4 |
| TTAaATCTATAaATTaCTtTGG | 685 | chr14 | 58928249 | − | 4 |
| TTtgATaTATAaATTCCTCTGG | 686 | chr14 | 67118379 | + | 4 |
| TTgaATCTATAaATTCCTtTGG | 687 | chr14 | 66877804 | − | 4 |
| TTgaATCTATAaATTaCTCTGG | 688 | chr14 | 67054524 | − | 4 |
| cTACATaTAcATATTCCTaTGG | 689 | chr14 | 73164427 | − | 4 |
| TTgCATCTgTATATcCCTtTGG | 690 | chr14 | 82848824 | − | 4 |
| TTACATCTATATAcTtCcaAGG | 691 | chr14 | 86163502 | − | 4 |
| TTgCATCTATAaATTaCTtTGG | 692 | chr14 | 94607900 | − | 4 |
| TTgaATCTATAaATTtCTCTGG | 693 | chr14 | 95997394 | + | 4 |
| TTAaATCTATAaATTaCTtTGG | 694 | chr14 | 97679275 | − | 4 |
| cTACAcCTATAaATTCCTtTGG | 695 | chr14 | 102500734 | − | 4 |
| aTAtATaTATATATTCCaCAGG | 696 | chr6 | 4395898 | − | 4 |
| TTgCATCTATAaATTaCTtTGG | 69 | chr6 | 8497287 | + | 4 |
| aTACATaTATATATTtCTaAGG | 698 | chr6 | 9645683 | + | 4 |
| aTACATCTATATATTgCcCTGG | 699 | chr6 | 19261263 | − | 3 |
| TTgCATCTATAaATTgCTtTGG | 700 | chr6 | 21319219 | − | 4 |
| TTcaATCTATtTAgTCCTCTGG | 701 | chr6 | 22208591 | − | 4 |
| TTACATCaATtTAaTtCTCAGG | 702 | chr6 | 26302557 | + | 4 |
| TcACATaTAaATATTCaTCTGG | 703 | chr6 | 27739125 | + | 4 |
| TTgaATCTATAaATTCCTtTGG | 704 | chr6 | 29060186 | + | 4 |
| aTtCATCTcTtTATTCCTCCGG | 705 | chr6 | 29085038 | − | 4 |
| TTAaATCTATAaATTgCTtTGG | 706 | chr6 | 32787727 | − | 4 |
| TTgaATCTATATATTgCTtTGG | 707 | chr6 | 39373802 | − | 4 |
| gTACATCTATgTATaCCTaAGG | 708 | chr6 | 42535037 | − | 4 |
| TTcCATCTATtTATctCTCTGG | 709 | chr6 | 44643964 | + | 4 |
| cTgCATCcATATATaCCTCAGG | 710 | chr6 | 47377577 | − | 4 |
| TTgaATCTATAaATTaCTCTGG | 711 | chr6 | 48266330 | + | 4 |
| TTACtaCTATAgATTCCTtTGG | 712 | chr6 | 50686034 | − | 4 |
| TTgaATCTATAaATTgCTCTGG | 713 | chr6 | 51689917 | − | 4 |
| gaACtTCTATtTATTCCTCAGG | 714 | chr6 | 54674658 | − | 4 |
| TTgtATCTAcATATTCCTCTGG | 715 | chr6 | 55870942 | − | 3 |
| TcACATCTATtcATgCCTCTGG | 716 | chr6 | 62678383 | − | 4 |
| TTACATCTgTAaATTgCTtTGG | 717 | chr6 | 64561749 | − | 4 |
| TTAaATCTATAaATTaCTtTGG | 718 | chr6 | 67730163 | + | 4 |
| TTACATCaATATcTTtaTCTGG | 719 | chr6 | 67389479 | − | 4 |
| aTtCATCTATAaATTCCaCTGG | 720 | chr6 | 67687599 | − | 4 |
| TTAaATCTATATATTgtTtTGG | 721 | chr6 | 71814476 | − | 4 |
| aTACATaTATATATTatTCTGG | 722 | chr6 | 72297098 | + | 4 |
| TTACATITACATATTaCTaTGG | 723 | chr6 | 72995744 | − | 4 |
| TTAaATCTATAaATTaCTtTGG | 724 | chr6 | 75162075 | − | 4 |
| TTgaATCTATAaATTaCTCTGG | 725 | chr6 | 75506641 | − | 4 |
| TTcCATCTATcTATTtCaCTGG | 726 | chr6 | 76337545 | + | 4 |
| aTACATCTATgTATcCCaCTGG | 727 | chr6 | 76362451 | + | 4 |
| TTAaATCTATAaATTaCTtTGG | 728 | chr6 | 76978866 | + | 4 |
| TTAaATCTATAaATTaCTtTGG | 729 | chr6 | 78423570 | − | 4 |
| TTACATgTATtaATTCCTgAGG | 730 | chr6 | 79799415 | + | 4 |
| aTACcTCTtTATATTCgTCAGG | 731 | chr6 | 83194558 | − | 4 |
| TTAaATCTATAaATTaCTtTGG | 732 | chr6 | 83946979 | − | 4 |
| gTACcTagATATATTCCTCTGG | 733 | chr6 | 84080932 | − | 4 |
| TTAaATaTATccATTCCTCTGG | 734 | chr6 | 87031826 | + | 4 |
| TTAatTCTAcATATTCCTaAGG | 735 | chr6 | 87617969 | − | 4 |
| TgACATtTtTAgATTCCTCAGG | 736 | chr6 | 91409194 | + | 4 |
| TTACAagTtTATATTtCTCTGG | 737 | chr6 | 91785958 | − | 4 |
| TTAaATCTATAaATTaCTtTGG | 738 | chr6 | 91958428 | + | 4 |
| TTgCATCTATAaATTaCTCTGG | 739 | chr6 | 92884667 | − | 3 |
| TTAaATCTATAaATTaCTtTGG | 740 | chr6 | 93482453 | − | 4 |
| TTgaATCTATATATTaCTtTGG | 741 | chr6 | 93540852 | − | 4 |
| gTAtATaTATATATTCCTtAGG | 742 | chr6 | 95071236 | + | 4 |
| TTAaATCTATAaATTgCTtTGG | 743 | chr6 | 95556022 | + | 4 |
| TTtCccCTATgTATTCCTCTGG | 744 | chr6 | 97476683 | + | 4 |
| TTAaATCTATAgATTgCTtTGG | 745 | chr6 | 97613806 | + | 4 |
| TTAaATCTATAaATTgCTtTGG | 746 | chr6 | 98080678 | + | 4 |
| TatgATCTtTATATTCCTCTGG | 747 | chr6 | 98951978 | + | 4 |
| TTAaATCTATAaATTgCTCTGG | 748 | chr6 | 101324234 | + | 3 |
| gTACATCTgTtTATTCCTtAGG | 749 | chr6 | 104360157 | − | 4 |
| TTACgTaTATATATaCaTCTGG | 750 | chr6 | 107045532 | + | 4 |
| TTAaATCTATAaATTaCTtCGG | 751 | chr6 | 109111092 | + | 4 |
| TgAttTCTATATATTCCTgAGG | 752 | chr6 | 110146798 | − | 4 |
| aTAtATaTATATATTCCTCTGG | 753 | chr6 | 110837759 | + | 3 |
| agACAaCTATATtTTCCTCAGG | 754 | chr6 | 114367252 | − | 4 |
| TTAaATCTgTAgATTaCTCTGG | 755 | chr6 | 114922761 | + | 4 |
| TTgaATCTATAaATTaCTCTGG | 756 | chr6 | 117334419 | − | 4 |
| TTAaATCTATAaATTgCTtTGG | 757 | chr6 | 118413087 | − | 4 |
| TTACcTCTATATATTtCTgAGG | 758 | chr6 | 122567037 | − | 3 |
| TTgCATCTATAaATTaCTtGGG | 759 | chr6 | 122893034 | + | 4 |
| gTgCATCcATATATTCCTtTGG | 760 | chr6 | 126466642 | + | 4 |
| TTggATCTATAaATTaCTCTGG | 761 | chr6 | 129878841 | + | 4 |
| TTAaATCTATATATTaCTITGG | 762 | chr6 | 132639726 | − | 3 |
| TTACAgCTATATATCCCTtTGG | 763 | chr6 | 135236788 | − | 3 |
| TTAaATaTcTAgATTCCTCAGG | 764 | chr6 | 136325896 | + | 4 |
| TgAaATCTATATATTgCTtTGG | 765 | chr6 | 140132930 | − | 4 |
| TTACATCTACATAgTCaTtTGG | 766 | chr6 | 140473000 | + | 4 |
| TTAaATCTATATATTaaTtTGG | 767 | chr6 | 140512828 | + | 4 |
| TTAaATCTgTAaATTaCTCTGG | 768 | chr6 | 142992874 | − | 4 |
| TTACATCTgTAaATTgCTtTGG | 769 | chr6 | 145318127 | + | 4 |
| TTACtTCTgTATATTaCTgTGG | 770 | chr6 | 146170130 | + | 4 |
| TTACATCTcTAaATTgCTtTGG | 771 | chr6 | 146606964 | + | 4 |
| TTAaATCTATAaATTgCTtTGG | 772 | chr6 | 146986985 | − | 4 |
| TTACATacATATATgCCTgTGG | 773 | chr6 | 147301398 | − | 4 |
| TTACgTCTATgTATaCCTaTGG | 774 | chr6 | 149434529 | − | 4 |
| TTACAcCTATAgATTCCctGGG | 775 | chr6 | 149672996 | + | 4 |
| TTgaATCTATATATTaCTtTGG | 776 | chr6 | 153784635 | − | 4 |
| aTACATgTACATATgCCTCTGG | 777 | chr6 | 154757760 | + | 4 |
| aTAtATaTATATATgCCTCGGG | 778 | chr6 | 155958393 | + | 4 |
| TTgCATCTATAaATTaCTtTGG | 779 | chr6 | 165018007 | + | 4 |
| TTAaATCTATAagTTCCTtTGG | 780 | chr6 | 168802155 | − | 4 |
| aTAtATaTATATATTCCTCTGG | 781 | chrY | 3116658 | − | 3 |
| TTAaATCTATAaATTaCTtTGG | 782 | chrY | 4517516 | − | 4 |
| TTACATCTgTAaATTgCTtTGG | 783 | chrY | 6653812 | + | 4 |
| TTACATCTATATATTatTgAGG | 784 | chrY | 11370797 | − | 3 |
| aTcCATCTcTAaATTCCTCTGG | 785 | chrY | 12303831 | + | 4 |
| TTcCATCTATAaATTgCTtTGG | 786 | chrY | 13545181 | − | 4 |
| TTgaATCTATAaATTaCTCTGG | 787 | chrY | 13648023 | + | 4 |
| TTgaATCTATAaATTCCTtTGG | 788 | chrY | 14948292 | + | 4 |
| TaACATCatTAcATTCCTCAGG | 789 | chrY | 16714560 | − | 4 |
| aTAtATaTATATATTCCaCTGG | 790 | chrY | 18493087 | − | 4 |
| aTAtATaTATATATTCCaCTGG | 791 | chrY | 18827292 | + | 4 |
| TTACATCTgcAcATTCtTCTGG | 792 | chrY | 19087050 | + | 4 |
| TTAagTCTATAgAcTCCTCAGG | 793 | chrY | 19880204 | − | 4 |
| TTACATgTATATATTtaTgGGG | 794 | chr11 | 5772107 | + | 4 |
| TTAaATCTATAaATTtCTtTGG | 795 | chr11 | 10146147 | − | 4 |
| ccAtATCaATATATTCCTCTGG | 796 | chr11 | 12230561 | − | 4 |
| TTACtTCTATtTgTTCCTaTGG | 797 | chr11 | 23088988 | − | 4 |
| TTgaATCTATATATTaCTtTGG | 798 | chr11 | 25305639 | − | 4 |
| TTAaATCTATATATTgCTtTGG | 799 | chr11 | 28422261 | − | 3 |
| TTAaATCTgTATATTgCTtTGG | 800 | chr11 | 28411171 | − | 4 |
| catCATCTATATATTCCTtGGG | 801 | chr11 | 30898353 | − | 4 |
| gTAtATaTATATATTCCcCAGG | 802 | chr11 | 32736991 | + | 4 |
| TTACATaTATATATaCtTgGGG | 803 | chr11 | 34699284 | − | 4 |
| TTgaATCTATATATTaCTaTGG | 804 | chr11 | 37031536 | + | 4 |
| TTAtATaTATATATTttTCTGG | 805 | chr11 | 36922884 | − | 4 |
| TTACATCaATAaATTCaTtTGG | 806 | chr11 | 38039415 | − | 4 |
| TTtCATaTgTATATTCCTtTGG | 807 | chr11 | 38980755 | − | 4 |
| TTAaATCTATAaATTaCTtTGG | 808 | chr11 | 40737794 | − | 4 |
| TTtCtTaTATATATTCCTgGGG | 809 | chr11 | 41927606 | + | 4 |
| TTAtATaTATATAaTCCTaTGG | 810 | chr11 | 42062709 | + | 4 |
| TTACATCTATAaATTgCTtTGG | 811 | chr11 | 42303013 | + | 3 |
| TTACATCTgTATATTtCctGGG | 812 | chr11 | 43527123 | + | 4 |
| TTtCATCTcTgTATTtCTCAGG | 813 | chr11 | 55674467 | − | 4 |
| TTAaATCTATAaATTgCTtTGG | 814 | chr11 | 58327603 | + | 4 |
| TTtCtcCTATATgTTCCTCTGG | 815 | chr11 | 59541663 | + | 4 |
| TTACATCTtTATAcTtCTtTGG | 816 | chr11 | 63245293 | + | 4 |
| TTAaATCTATAaATTgCTtTGG | 817 | chr11 | 63292473 | + | 4 |
| TTACATITATATATTatTaAGG | 818 | chr11 | 63567936 | − | 4 |
| TTgaATCTATAaATTgCTCTGG | 819 | chr11 | 75683710 | + | 4 |
| TTtCATtTAaATAgTCCTCTGG | 820 | chr11 | 77419332 | + | 4 |
| TTACATCTATtTATaCtgCAGG | 821 | chr11 | 77368136 | − | 4 |
| cTACATCaATATATTCaTtTGG | 822 | chr11 | 81225291 | + | 4 |
| TTttATgTATATATTCaTCAGG | 823 | chr11 | 85490993 | − | 4 |
| TTACATCTATAacTTaCTtTGG | 824 | chr11 | 87200839 | − | 4 |
| TTAaATCTATAaATTaCTtTGG | 825 | chr11 | 88729451 | − | 4 |
| TTAaATCTAaATATTaCTaTGG | 826 | chr11 | 88646453 | − | 4 |
| TTACAaCTATAgATaCCTtAGG | 827 | chr11 | 92649242 | + | 4 |
| TTgaATCTgTATATTCCTtTGG | 828 | chr11 | 92989067 | − | 4 |
| TTAaATCTATAaATTtCTtTGG | 829 | chr11 | 97439639 | − | 4 |
| aTACATaTATATATTtCTITGG | 830 | chr11 | 97921973 | + | 4 |
| TTgaATCTATATATTaCTtTGG | 831 | chr11 | 98118385 | + | 4 |
| TTgCATCcATAgATTCCTtTGG | 832 | chr11 | 101658819 | + | 4 |
| TTggATCTATATATTgCTgGGG | 833 | chr11 | 101888882 | + | 4 |
| TTgaATCTATAaATTaCTCTGG | 834 | chr11 | 101897921 | + | 4 |
| TTgaATCTATATATTaCTtTGG | 835 | chr11 | 102815415 | + | 4 |
| TggCATCTATATAaTCCTgAGG | 836 | chr11 | 106229383 | − | 4 |
| TcACATCTATAatTTCCcCCGG | 837 | chr11 | 106775023 | + | 4 |
| TTACATgTATAagTTCtTCAGG | 838 | chr11 | 111317408 | − | 4 |
| TcACATtcATAaATTCCTCAGG | 839 | chr11 | 113249876 | + | 4 |
| TTAgATCaATtTATTCCTaGGG | 840 | chr11 | 114592435 | − | 4 |
| aTgCATCTATATATTCCTtGGG | 841 | chr11 | 120267772 | − | 3 |
| aTtCATCTITATATTCCCCAGG | 842 | chr11 | 122422572 | + | 4 |
| TggCATaTtTATATTCCTCAGG | 843 | chr11 | 128260053 | + | 4 |
| TTAtATCTATAaATTaCTtTGG | 844 | chr11 | 130539060 | + | 4 |
| aTAaATtTATATATcCCTCTGG | 845 | chr10 | 2221308 | − | 4 |
| TTAaATgTATATgTTaCTCGGG | 846 | chr10 | 5447094 | + | 4 |
| TTACATgaATATATTgCaCAGG | 847 | chr10 | 7720234 | − | 4 |
| TTACATCTACATgaTtCTCAGG | 848 | chr10 | 8735539 | + | 4 |
| gTACATtTAaATATTCCCCAGG | 849 | chr10 | 14923297 | + | 4 |
| TTACATITATtTAgTCCTgTGG | 850 | chr10 | 17008296 | + | 4 |
| TTAaATCTATAaATTaCTtTGG | 851 | chr10 | 19080192 | − | 4 |
| TTAaATCTATAcATTaCTtTGG | 852 | chr10 | 22509620 | + | 4 |
| TTtCATCcAggTATTCCTCAGG | 853 | chr10 | 26890627 | + | 4 |
| TTAaATCTgTATATTgCTtTGG | 854 | chr10 | 26795818 | − | 4 |
| TTtCATaTcTATATTCaTCGGG | 855 | chr10 | 31231567 | − | 4 |
| TcACATCTgTATATTCtaCTGG | 856 | chr10 | 31288778 | − | 4 |
| aTAtATtTATgTATTCCTCAGG | 857 | chr10 | 33033218 | − | 4 |
| TTAaATCTATAgATTCaTtAGG | 858 | chr10 | 37452913 | + | 4 |
| TTACATCTITATATTatTgAGG | 859 | chr10 | 38673833 | − | 4 |
| TTgaATCTATATATTaCTtTGG | 860 | chr10 | 44144914 | − | 4 |
| TTACAgCTATtcATTCCaCAGG | 861 | chr10 | 51197135 | − | 4 |
| TTACATCTAgAaAaTCCTaAGG | 862 | chr10 | 52476316 | + | 4 |
| TTgaATCTATAaATTaCTCTGG | 863 | chr10 | 53146952 | + | 4 |
| TTcCATCTtTActTTCCTCGGG | 864 | chr10 | 53674673 | + | 4 |
| TTACATCTATATgTTgtTaCGG | 865 | chr10 | 53919637 | − | 4 |
| TcAaATgTATATATTtCTCTGG | 866 | chr10 | 54907151 | + | 4 |
| TTAaATCTATATATTtCTtGGG | 867 | chr10 | 55068657 | + | 3 |
| aTgCATaTATATtTTCCTCAGG | 868 | chr10 | 55850540 | + | 4 |
| TTACATaTATAgATTCaTtTGG | 869 | chr10 | 56953344 | + | 4 |
| TTAaATCTATgTATTgCTtTGG | 870 | chr10 | 57346990 | + | 4 |
| TTgaATCTATATATTaCTtTGG | 871 | chr10 | 57489166 | − | 4 |
| TgAtATCTATATcTTCCTgTGG | 872 | chr10 | 58119820 | − | 4 |
| TTAgATCTATcTATTCaaCAGG | 873 | chr10 | 59714775 | + | 4 |
| TTACATaTAaATATTtCTCAGG | 874 | chr10 | 63916216 | + | 3 |
| TTAacTCTATtTATTCCTaAGG | 875 | chr10 | 64355704 | + | 4 |
| TTAaATCTATAaATTaCTtTGG | 876 | chr10 | 67717884 | + | 4 |
| TTACcTCTggATATTCCaCTGG | 877 | chr10 | 68274834 | + | 4 |
| TTAtATCTgTAaATaCCTCTGG | 878 | chr10 | 70282099 | + | 4 |
| TTACATtaATATATTCaTgTGG | 879 | chr10 | 72145707 | + | 4 |
| TTAaAgaTATATATTCCTgAGG | 880 | chr10 | 73558530 | − | 4 |
| TTACtTgTgTAgATTCCTCGGG | 881 | chr10 | 81477496 | − | 4 |
| TTAgATCaATATAaTCaTCTGG | 882 | chr10 | 82166722 | + | 4 |
| TTAtATCTATAaATTaCTtTGG | 883 | chr10 | 82514782 | + | 4 |
| TTgtATCTATATtTTtCTCAGG | 884 | chr10 | 83127476 | − | 4 |
| TTAaATCTATAaATTaCTtTGG | 885 | chr10 | 83093750 | − | 4 |
| TTAaATCTATAgATTgCTtTGG | 886 | chr10 | 83322641 | − | 4 |
| TTACATCTgTAaATTgCTtTGG | 887 | chr10 | 89149453 | + | 4 |
| TTgaATCTATATATTgCTtTGG | 888 | chr10 | 94907490 | + | 4 |
| TTcCATCTATgTgTTCCTCTGG | 889 | chr13 | 64880272 | + | 3 |
| aTAtATaTATATATTCCTCTGG | 890 | chr13 | 67936978 | + | 3 |
| TTAaATgTATAaATTCCTCTGG | 891 | chr19 | 13468019 | + | 3 |
| aTACATCTtTATATTCCTaTGG | 892 | chr19 | 21295869 | − | 3 |
| TaACATCTATATAaTCCTgAGG | 893 | chr19 | 38582442 | − | 3 |
| TTAaATCTgTATATTgCTCTGG | 894 | chr3 | 30883710 | + | 3 |
| TTACAaaTATATATTCCaCTGG | 895 | chr3 | 32759660 | − | 3 |
| TTgCcTCTtTATATTCCTCAGG | 896 | chr3 | 44599152 | − | 3 |
| TTAaATCTgcATATTCCTCTGG | 897 | chr3 | 62367841 | + | 3 |
| TTAtATCTATATATaaCTCTGG | 898 | chr3 | 66331094 | + | 3 |
| TTACATCTCTATAaTCCTCAGG | 899 | chr3 | 89429002 | + | 2 |
| TTAaATCTATAgATTCCTtTGG | 900 | chr3 | 104680819 | − | 3 |
| TTACATCTATAgATTgCTtTGG | 901 | chr3 | 119137147 | − | 3 |
| aTgCATaTATATATTCCTCAGG | 902 | chr3 | 162862206 | + | 3 |
| TTACATCTgTATtTTCCaaAGG | 903 | chr8 | 4362188 | + | 4 |
| TTAtATgTATATATTaCTaTGG | 904 | chr8 | 4152743 | − | 4 |
| TTgaATCTATATATTaCTtTGG | 905 | chr8 | 4569706 | + | 4 |
| TTAgtTCTATAacTTCCTCTGG | 906 | chr8 | 4688984 | − | 4 |
| TTACAaaTcTtTATTCCTCTGG | 907 | chr8 | 5138809 | − | 4 |
| TTACATaTAaATATTCtTCTGG | 908 | chr8 | 8690470 | + | 3 |
| TcAgATCcAaATATTCCTCAGG | 909 | chr8 | 9360868 | − | 4 |
| aTACATaTATATATTatTCAGG | 910 | chr8 | 14476437 | − | 4 |
| cTACtTCTgTcTATTCCTCTGG | 911 | chr8 | 16156434 | + | 4 |
| TTgCATtTATATATgCCTtTGG | 912 | chr8 | 16023535 | − | 4 |
| TTACATaTATAcAaTCCaCAGG | 913 | chr8 | 22542705 | + | 4 |
| TTtCATaTATATATTCaTgAGG | 914 | chr8 | 25040113 | + | 4 |
| cTAacTCTATATtTTCCTCAGG | 915 | chr8 | 24794917 | − | 4 |
| TTtCATCTgTATtTTCtTCTGG | 916 | chr8 | 26606340 | − | 4 |
| TTAaATCTATAaATTaCTtTGG | 917 | chr8 | 28209618 | − | 4 |
| TaAaATCTATATtTTaCTCTGG | 918 | chr8 | 29492122 | + | 4 |
| TaAgATCTgTATATTtCTCTGG | 919 | chr8 | 32278923 | − | 4 |
| TTAaATCTATAaATTaCTtTGG | 920 | chr8 | 35820558 | − | 4 |
| ggACATCTATATAcTtCTCAGG | 921 | chr8 | 35934306 | − | 4 |
| gTACtTCTATATATTtCaCAGG | 922 | chr8 | 39520408 | − | 4 |
| TgAaATtTATtTATTCCTCTGG | 923 | chr8 | 51435800 | − | 4 |
| TTAaATCTATATATTaCTtTGG | 924 | chr8 | 52848278 | + | 3 |
| TTAaATCTATAaATTaCTtTGG | 925 | chr8 | 56853828 | + | 4 |
| TTAaATCTATAaATTaCTtTGG | 926 | chr8 | 57699670 | − | 4 |
| TTACATaTATAcAcTaCTCAGG | 927 | chr8 | 58078337 | − | 4 |
| TTACATgTgcATtTTCCTCAGG | 928 | chr8 | 60210463 | + | 4 |
| TTcCATtTAaATATTtCTCAGG | 929 | chr8 | 61147681 | + | 4 |
| TTAaATCTATAaATTaCTtTGG | 930 | chr8 | 65342316 | − | 4 |
| TcACAaCTATcTATTCCaCTGG | 931 | chr8 | 68198125 | − | 4 |
| TTgaATCTATATATTaCTtTGG | 932 | chr8 | 70766171 | + | 4 |
| TTgaATCTATAaATTCCTtTGG | 933 | chr8 | 76950426 | − | 4 |
| TTACAgCTtTAaATTCgTCTGG | 934 | chr8 | 78168534 | − | 4 |
| TTACATCTtgAaAgTCCTCTGG | 935 | chr8 | 78356963 | − | 4 |
| TTAaATCTATAaATTaCTtTGG | 936 | chr8 | 79268213 | − | 4 |
| TTAgATCTATAgATTaaTCTGG | 937 | chr8 | 81838290 | − | 4 |
| TTAtATCTATAaATTgCTtCGG | 938 | chr8 | 84112622 | + | 4 |
| TgAttTCTATATATTCCTaGGG | 939 | chr8 | 84586716 | + | 4 |
| TTACATCTAaATAaTCtTaAGG | 940 | chr8 | 84599101 | − | 4 |
| TTAaATCaATATATTtCcCAGG | 941 | chr8 | 86159590 | + | 4 |
| TcACcTaTtTATATTCCTCAGG | 942 | chr8 | 86329561 | − | 4 |
| TTAtATCTATcTcTTCCTaGGG | 943 | chr8 | 86899790 | + | 4 |
| TTAgATCTgTgaATTCCTCTGG | 944 | chr8 | 87151057 | − | 4 |
| TTAaATgTtaATATTCCTCAGG | 945 | chr8 | 89517006 | − | 4 |
| TTAaATCTATAgATTgCTtTGG | 946 | chr8 | 91505872 | + | 4 |
| TTgaATCTATAcATTCCTtTGG | 947 | chr8 | 96192076 | + | 4 |
| cTACATCTAcAgATTCtTCAGG | 948 | chr8 | 102625940 | − | 4 |
| TTgCATCTATAaATTaCTtTGG | 949 | chr8 | 110453591 | − | 4 |
| TTgaATCTATAaATTaCTCTGG | 950 | chr8 | 110777991 | − | 4 |
| TaACgTgTATATtTTCCTCAGG | 951 | chr8 | 111318688 | + | 4 |
| TTAaATCTATAaATTaCTtTGG | 952 | chr8 | 111391733 | − | 4 |
| TTAtATCTgTATATTtCTaTGG | 953 | chr8 | 115584496 | + | 4 |
| TTACcTggAaATATTCCTCTGG | 954 | chr8 | 116354507 | − | 4 |
| TTcCATCTATAaATTaCTtTGG | 955 | chr8 | 120476826 | − | 4 |
| TTAtATaTATgTATgCCTCAGG | 956 | chr8 | 122808193 | + | 4 |
| aTAtATaTATATATTCCTtTGG | 957 | chr8 | 132441606 | + | 4 |
| TTAaATCTATAaATTaCTtTGG | 958 | chr8 | 134678509 | − | 4 |
| TTAtAaCTtTATATTgCTCAGG | 959 | chr15 | 23889664 | − | 4 |
| cTAtATgTAcATATTCCTCTGG | 960 | chr15 | 36932700 | + | 4 |
| TTAaATCTATAaATTaCTtTGG | 961 | chr15 | 39293348 | + | 4 |
| TTACATCTAgATcTTCtTCCGG | 962 | chr15 | 40206366 | − | 3 |
| TTAgtTCTtTATcTTCCTCAGG | 963 | chr15 | 41747323 | + | 4 |
| gTACATITATATAgTCCTgTGG | 964 | chr15 | 45509027 | − | 4 |
| TTACATCTgTAgATTgCTtTGG | 965 | chr15 | 46912399 | − | 4 |
| TTAaATCTgTAgATTCCTtTGG | 966 | chr15 | 49388055 | − | 4 |
| TTACATITATAaATTaCTtTGG | 967 | chr15 | 49514163 | + | 4 |
| TTACATCTgTAaATTgCTtTGG | 968 | chr15 | 49603209 | + | 4 |
| TTACATgTtTATATTCagCAGG | 969 | chr15 | 51273341 | + | 4 |
| TTACATCTgTATcaTtCTCTGG | 970 | chr15 | 53120551 | + | 4 |
| TTACATCTATATgaTatTCAGG | 971 | chr15 | 53069644 | − | 4 |
| TTACATITATATAaTaCTtTGG | 972 | chr15 | 53308854 | − | 4 |
| TTAaATCTATAaATTgCTtTGG | 973 | chr15 | 56334086 | − | 4 |
| TaAaATtaATATATTCCTCTGG | 974 | chr15 | 61700533 | + | 4 |
| TTACATCTtTccATTCCaCTGG | 975 | chr15 | 62156105 | − | 4 |
| TTAaATCctTATAgTCCTCTGG | 976 | chr15 | 68686533 | − | 4 |
| TTACATtTATAcATTaaTCTGG | 977 | chr15 | 70176790 | + | 4 |
| TTAgATCTATAaATTCaTtTGG | 978 | chr15 | 71683639 | + | 4 |
| TTAtAaCTgTATATTCCTtGGG | 979 | chr15 | 72534777 | + | 4 |
| aTACATaTATATATTgCTCAGG | 980 | chr15 | 74660179 | − | 3 |
| TTAaATCTATAaATTgCTtTGG | 981 | chr15 | 76485458 | − | 4 |
| TTACATCTtTAagTTCCTCAGG | 982 | chr15 | 85151456 | − | 3 |
| TTAaATCTATATAcTgCTCAGG | 983 | chr15 | 86380108 | + | 3 |
| TTAaATCTATAaATTgCTtTGG | 984 | chr15 | 86953230 | + | 4 |
| TTcCATCTtTAaATTgCTCAGG | 985 | chr15 | 90199390 | − | 4 |
| gTACAgCTATATtTcCCTCTGG | 986 | chr15 | 91363895 | − | 4 |
| TTgCATCTATAaATTaCTtTGG | 987 | chr15 | 92038676 | − | 4 |
| TTAtATtaATATAgTCCTCAGG | 988 | chr15 | 93896798 | + | 4 |
| TTgCATCTATAaATTaCTtGGG | 989 | chr15 | 97280297 | + | 4 |
| TTAaATCTgTATATTgCTtTGG | 990 | chr15 | 99348842 | + | 4 |
| TTACATITAcAcAaTCCTCTGG | 991 | chr5 | 2114621 | − | 4 |
| TTACcTtTgTATtTTCCTCTGG | 992 | chr5 | 7799951 | − | 4 |
| TTAaATCTATAaATTaCTtTGG | 993 | chr5 | 9276383 | − | 4 |
| TTAaATCTATAgATTaCTtTGG | 994 | chr5 | 9770480 | + | 4 |
| TcACAgCTcTAcATTCCTCTGG | 995 | chr5 | 13382106 | + | 4 |
| aTACATaTATATATTtaTCAGG | 996 | chr5 | 15138012 | − | 4 |
| TTgCATCTATAgATTgtTCAGG | 997 | chr5 | 16028065 | + | 4 |
| cTtCATtTATATATTCCTaAGG | 998 | chr5 | 16503743 | − | 4 |
| TTgaATCTATAaATTCCTtTGG | 999 | chr5 | 22281797 | − | 4 |
| TTgaATCTATATATTaCTtTGG | 1000 | chr5 | 25215557 | − | 4 |
| aTAtATaTATATtTTCCTCTGG | 1001 | chr5 | 26752410 | + | 4 |
| TTgCATCTATAgATTgCTtTGG | 1002 | chr5 | 26697648 | − | 4 |
| TTgaATCTATAaATTaCTCTGG | 1003 | chr5 | 32799737 | − | 4 |
| CTACATacATATATTtCTCTGG | 1004 | chr5 | 33369270 | + | 4 |
| TTtCcTCcATATATTCCaCAGG | 1005 | chr5 | 33244981 | − | 4 |
| TTAaATCTATAaATTaCTtTGG | 1006 | chr5 | 33707376 | − | 4 |
| TTACATCTATATATTagTtTGG | 1007 | chr5 | 33876222 | − | 3 |
| TTcCcTgTgTATATTCCTCTGG | 1008 | chr5 | 34625483 | − | 4 |
| TTtCtTCTgTATtTTCCTCTGG | 1009 | chr5 | 40909740 | − | 4 |
| TTgaATCTATAgATTCCTtTGG | 1010 | chr5 | 41499568 | − | 4 |
| TTgCATCTATAaATTaCTtTGG | 1011 | chr5 | 42265396 | + | 4 |
| TTAaATtTATAaATTCCTtTGG | 1012 | chr5 | 44174589 | − | 4 |
| aTAaATCTATATATTtCTgTGG | 1013 | chr5 | 51366491 | + | 4 |
| TTACATITAcAcATTCCTtGGG | 1014 | chr5 | 52458650 | − | 4 |
| TTAgATCcATATATaCtTCAGG | 1015 | chr5 | 52711946 | + | 4 |
| TTAaATCTATAcATTgCTtTGG | 1016 | chr5 | 52942342 | + | 4 |
| TTAgATCTgcATATTCCTtGGG | 1017 | chr5 | 53071021 | − | 4 |
| TTgaATCTATAgATTgCTCTGG | 1018 | chr5 | 54369846 | + | 4 |
| TTgaATCTATAaATTaCTCTGG | 1019 | chr5 | 54781780 | − | 4 |
| TTACAaCTATAatTTCCaCAGG | 1020 | chr5 | 61558257 | − | 4 |
| TTACtaCTATcTAaTCCTCAGG | 1021 | chr5 | 61743910 | − | 4 |
| TTACtTCTAaATcTTCCTgGGG | 1022 | chr5 | 61799541 | − | 4 |
| TTACATCTgTAgATTgCTtTGG | 1023 | chr5 | 62856195 | + | 4 |
| TTgaATCTATATATTaCTtTGG | 1024 | chr5 | 63365369 | + | 4 |
| TTgCATCTATAaATTaCTtTGG | 1025 | chr5 | 63511855 | − | 4 |
| TTtCATCTATgTtTTCtTCTGG | 1026 | chr5 | 67330458 | + | 4 |
| TaACAcCTAgATATTCaTCTGG | 1027 | chr5 | 72269758 | − | 4 |
| TTAtATtTATtTATTCCaCTGG | 1028 | chr5 | 74515500 | + | 4 |
| TTgaATCTATATATTgCTtTGG | 1029 | chr5 | 78462013 | + | 4 |
| TTACATtgATATATTgCTtAGG | 1030 | chr5 | 80195327 | − | 4 |
| TTAaATCTATAaATTgCTtTGG | 1031 | chr5 | 81665127 | + | 4 |
| TTAaATCTATACATTaCTtAGG | 1032 | chr5 | 81659588 | − | 4 |
| gTAaAcCTATATtTTCCTCTGG | 1033 | chr5 | 82092316 | − | 4 |
| TTAaATCTATAaATTaCTtGGG | 1034 | chr5 | 85093598 | − | 4 |
| TTgCATCTgTATATTgCTtTGG | 1035 | chr5 | 85766367 | + | 4 |
| TTtCATCcATtaATTCCTCTGG | 1036 | chr5 | 85667300 | − | 4 |
| TTtCATgTcTATATTCaTCAGG | 1037 | chr5 | 85913951 | − | 4 |
| TTgCATCTATATcTTgCcCAGG | 1038 | chr5 | 89519164 | − | 4 |
| TTACATgTAgAaATTCtTCTGG | 1039 | chr5 | 91669153 | + | 4 |
| TTcCtTCTtTATATTCtTCAGG | 1040 | chr5 | 92027611 | + | 4 |
| TTgaATCTATAaATTaCTCTGG | 1041 | chr5 | 98665272 | − | 4 |
| TTACATaTATATgTTCaTtGGG | 1042 | chr5 | 99113968 | − | 4 |
| aaAaATCTATATATTCCTtAGG | 1043 | chr5 | 99853353 | + | 4 |
| TTAaATCTATAaATTtCTtTGG | 1044 | chr5 | 99960480 | + | 4 |
| TTtCATCcATAgATcCCTCAGG | 1045 | chr5 | 103909376 | + | 4 |
| TTACATITATATtTTCacCTGG | 1046 | chr5 | 103758266 | − | 4 |
| TTgaATCTATATATTgCTtTGG | 1047 | chr5 | 104804541 | − | 4 |
| aTACATCTATtTAgTaCTCTGG | 1048 | chr5 | 107192716 | + | 4 |
| TgACATCTATATcTTatTCAGG | 1049 | chr5 | 106945520 | − | 4 |
| gTAaATCTATATATTtaTCTGG | 1050 | chr5 | 111059846 | − | 4 |
| TTcCATCTcTAaATTCCTaGGG | 1051 | chr5 | 111530575 | + | 4 |
| TTgCATCTgTATATTgCTtTGG | 1052 | chr5 | 112206867 | − | 4 |
| TTgaATCTATAaATTCCTtTGG | 1053 | chr5 | 114880800 | − | 4 |
| TTgaATCTATAaATTCCTtTGG | 1054 | chr5 | 115075565 | − | 4 |
| TTAaATCTATAaATTaCTtTGG | 1055 | chr5 | 121606724 | − | 4 |
| TTAaATCTATAaATTaCTtTGG | 1056 | chr5 | 121965756 | + | 4 |
| aTAtATCTATATATaCCgCAGG | 1057 | chr5 | 124668237 | + | 4 |
| TTgaATCTATATATTaCTtTGG | 1058 | chr5 | 125843993 | − | 4 |
| TgACATaTATATATatCTCAGG | 1059 | chr5 | 126164518 | + | 4 |
| TgACATaTATATATatCTCAGG | 1060 | chr5 | 126164598 | + | 4 |
| TgACATaTATATATatCTCAGG | 1061 | chr5 | 126164572 | + | 4 |
| aTACATaTATATATTCaTaTGG | 1062 | chr5 | 126701959 | − | 4 |
| aTAaATaTATATATTtCTCTGG | 1063 | chr5 | 127352093 | + | 4 |
| TTACATITATATATgtCaCTGG | 1064 | chr5 | 132744174 | − | 4 |
| TTACATCagcATAcTCCTCTGG | 1065 | chr5 | 143955309 | + | 4 |
| TTAaATCTATAaATTaCTtTGG | 1066 | chr5 | 144590010 | + | 4 |
| aTACATCTAgATtTTCCgCCGG | 1067 | chr5 | 149829582 | − | 4 |
| TgACATCTAcATATTCtcCTGG | 1068 | chr5 | 151615849 | − | 4 |
| TTAaATCTtTtTATTCCTtTGG | 1069 | chr5 | 153135390 | − | 4 |
| TTAaATCTATAgATTgCTtTGG | 1070 | chr5 | 153929018 | − | 4 |
| TTAagTCgAcATATTCCTCTGG | 1071 | chr5 | 159521436 | + | 4 |
| aTAtATCTATATATgCCTaTGG | 1072 | chr5 | 164687761 | − | 4 |
| TTAaATCTATAgATTgCTtTGG | 1073 | chr5 | 166239458 | − | 4 |
| aTAtATgTATATATTCCCCAGG | 1074 | chr5 | 167801147 | + | 4 |
| TTgCtTCTATATATTtCTaAGG | 1075 | chr5 | 169861179 | − | 4 |
| TTAaATCTATAaATTgCTtTGG | 1076 | chr5 | 174908953 | − | 4 |
| TTAatTCTATgTATTtCTCTGG | 1077 | chr5 | 176493059 | − | 4 |
| TTACATCTATtTAgTgCTgTGG | 1078 | chr20 | 2075538 | − | 4 |
| aTACATaTATATATTCCTtAGG | 1079 | chr20 | 2775968 | − | 3 |
| TaACAaCTATATACTCaTCTGG | 1080 | chr20 | 3617015 | − | 4 |
| TTAaATCTATAaATTaCTtTGG | 1081 | chr20 | 5394994 | − | 4 |
| TcACATCTgTccATTCCTCAGG | 1082 | chr20 | 6956182 | + | 4 |
| TaAgATCTATATATTggTCAGG | 1083 | chr20 | 10925668 | − | 4 |
| TTAaATCTATAaATTaCTtTGG | 1084 | chr20 | 19846294 | + | 4 |
| TTACATCTITATATTCtcaGGG | 1085 | chr20 | 39852323 | + | 4 |
| TaAaATCTATATATTCCTgTGG | 1086 | chr20 | 41678035 | − | 3 |
| TTACATgTcTggATTCCTCAGG | 1087 | chr20 | 42766229 | + | 4 |
| TTgaATCTATAaATTaCTCTGG | 1088 | chr20 | 45727828 | − | 4 |
| cTACATgTATAcATgCCTCTGG | 1089 | chr20 | 46533092 | + | 4 |
| TTAgATCTATgTtaTCCTCAGG | 1090 | chr20 | 51899648 | − | 4 |
| TTctcTCTATATATTtCTCTGG | 1091 | chr20 | 53095171 | + | 4 |
| TTgaATCTATAaATTgCTCTGG | 1092 | chr20 | 54034707 | − | 4 |
| TTAaATCTgTATATTgCTtTGG | 1093 | chr20 | 63077209 | + | 4 |
| TacCAaCTtTATATTCCTCAGG | 1094 | chr20 | 63250712 | − | 4 |

Unbalanced rearrangements involving chromosomal region 1p13 [add(1)(p13)] were also observed in CRISPR/Cas9-edited cells, consistent with off-target sites predicted by the in silico Cas-OFFinder tool for the CRISPR/Cas9 sgRNA. Notably, no clonal structural abnormalities in ABE-treated cells were observed beyond those present in all pseudo-tetraploid Jurkat T cells. Thus, these findings suggest that ex vivo ABE manipulation can efficiently correct the pathogenic CD3δ SCID mutation without the deleterious byproducts associated with DSBs.

### Evaluating Local Bystander and Genome-Wide Off-Target Editing in CD3D (C202T) Jurkat T Cells and CD3δ SCID Patient CD34+ HSPCs

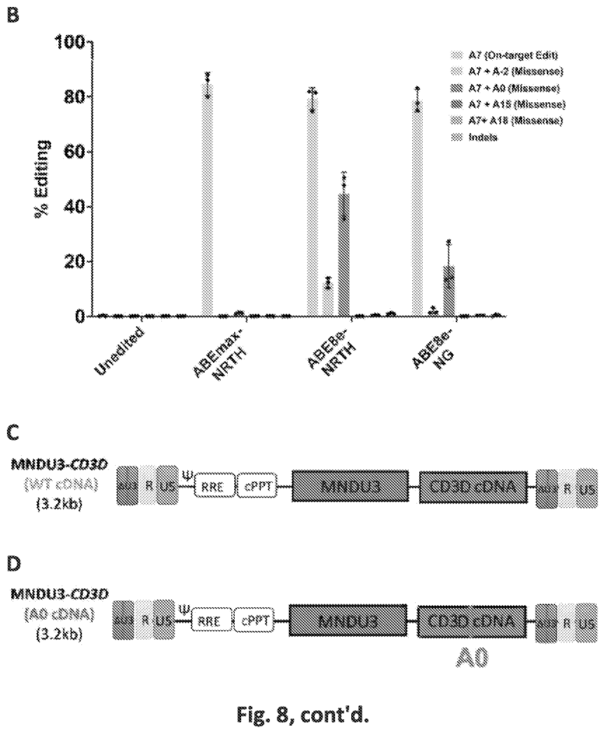

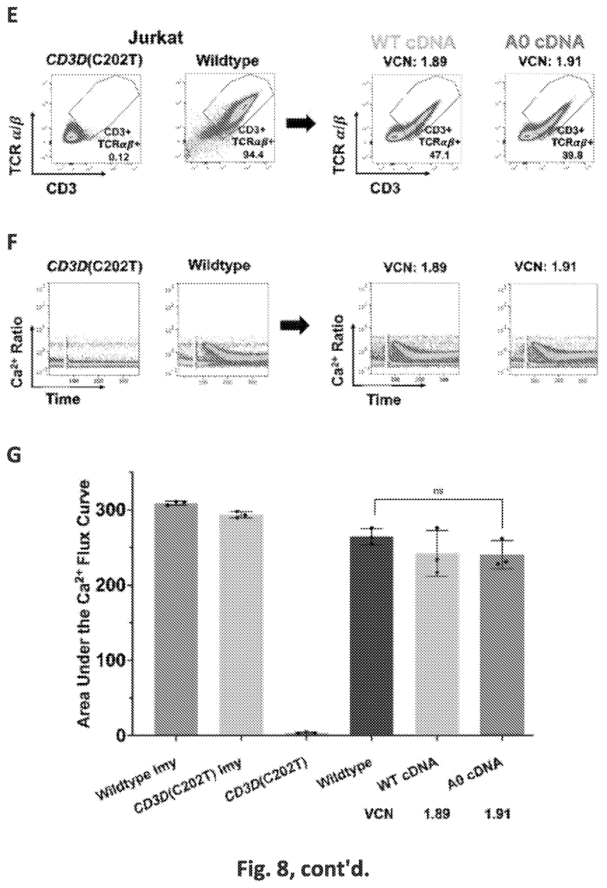

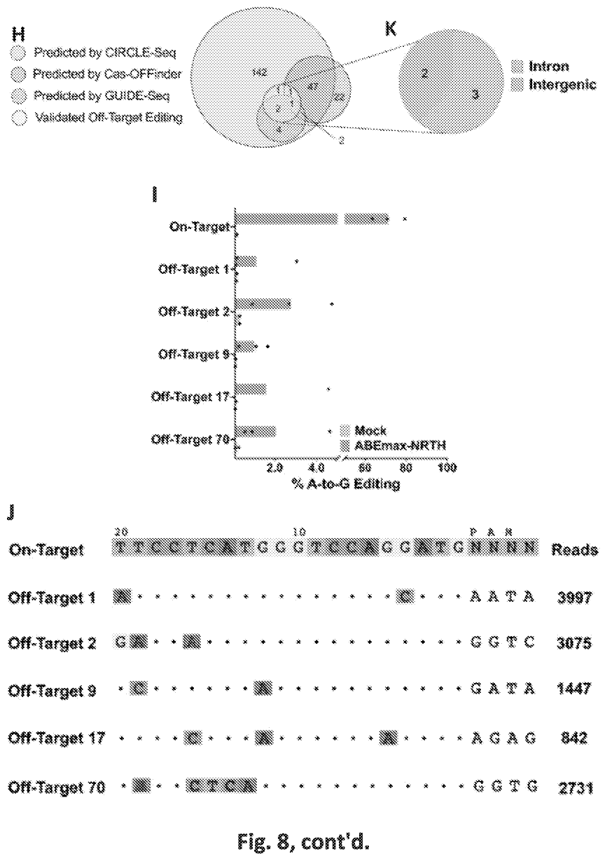

Recognizing local bystander editing, or base editing within or near the protospacer other than the target adenine, as a potential limitation of ABE,¹⁹ we sought to characterize the effects of detectable bystander editing on CD3/TCR signaling. High-throughput sequencing (HTS) analysis of CD3D (C202T) Jurkat T cells treated with plasmids encoding lead candidate base editors, ABEmax-NRTH, ABE8e-NRTH, or ABE8e-NG, revealed less than 1.35% indels, with the only detectable bystander edits occurring at positions A0 and A-2 (FIG. 8, panels A & B). We noted significantly increased levels of bystander editing produced by the highly processive ABE8e variants (up to 50.4% and 13.9% at positions A0 and A-2, respectively) compared to ABEmax treatment (up to 1.4% at position A0), consistent with the increased deaminase activity characteristic of ABE8e-mediated editing 20 (FIG. 8, panel B). These data suggested ABEmax-NRTH as the lead therapeutic candidate for safe and efficient correction of CD3D c.202C>T.

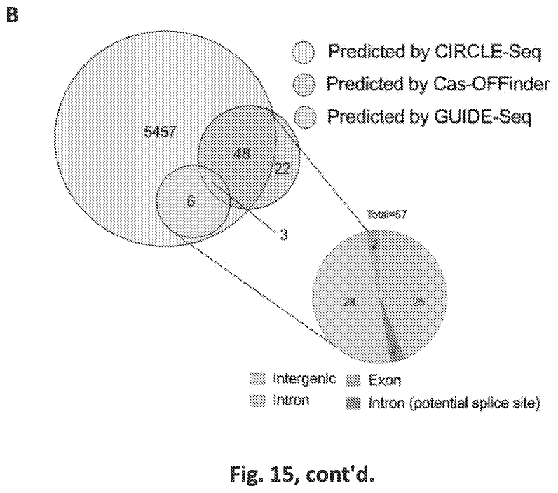

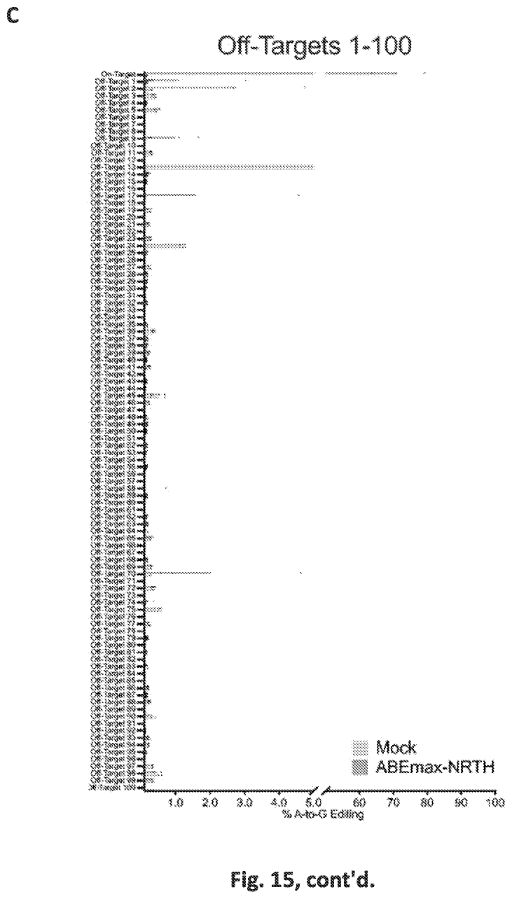

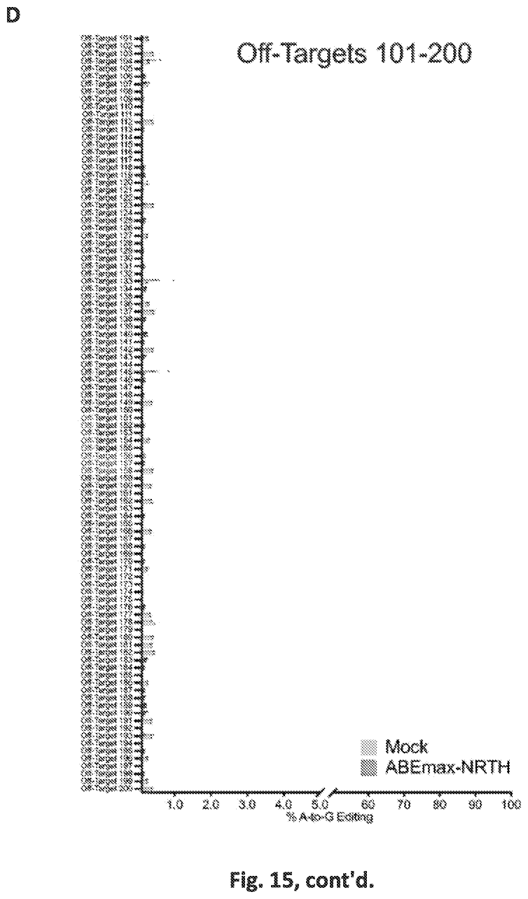

To further investigate the effect of the only detectable bystander edit (A0) induced by ABEmax-NRTH, we transduced CD3D (C202T) Jurkat T cells with one of two lentiviral vectors (LVs) expressing either: 1) a wildtype CD3D cDNA (MNDU3-CD3D WT cDNA) or 2) a CD3D cDNA containing the A0 bystander mutation (MNDU3-CD3D A0 cDNA) (FIG. 8, panels C, D). Encouragingly, CD3D (C202T) Jurkat T cells transduced with MNDU3-CD3D A0 cDNA or MNDU3-CD3D WT cDNA demonstrated wildtype levels of CD3/TCR (FIG. 8, panels E, F; FIG. 15, panel A) signaling in response to anti-CD3 and anti-CD28 stimulation (FIG. 8, panel g). These findings suggest that low levels of bystander editing at position A0 will not interfere with rescue of healthy T cell function.

To identify and characterize genome-wide, Cas-dependent off-target editing resulting from ABEmax-NRTH mRNA and CD3D-directed sgRNA treatment, we utilized experimental and in silico methods including, CIRCLE-seq, 21 GUIDE-seq,²² and Cas-OFFinder.²³ We experimentally performed CIRCLE-seq, a sensitive, in vitro off-target detection method, to identify nuclease-mediated cleavage sites induced by Cas9-NRTH and CD3D-localizing sgRNA in human genomic DNA. Recognizing the relaxed PAM consensus motif of the NRTH nuclease,²⁴ we conducted CIRCLE-seq analysis to permit six mismatched nucleotides or fewer in aligned sequences, without specifying the PAM (NNNN), resulting in 5,514 candidate off-target sites (Table 3). To further validate off-target nominations, we performed GUIDE-seq, an unbiased detection method of off-target events, by electroporating CD3D (C202T) K562 cells with a Cas9-NRTH nuclease complexed to CD3D-targeting sgRNA and a double-stranded DNA oligo for capture at DSBs. GUIDE-seq identified nine candidate sites, all of which overlapped with CIRCLE-seq nominations. The Cas-OFFinder in silico algorithm nominated 73 human genomic sites with <3 mismatches to the target protospacer, 51 of which were also nominated by CIRCLE-seq. Of the 5,514 sites predicted by CIRCLE-seq, the nine sites identified by GUIDE-seq, and the 73 sites nominated Cas-OFFinder, only three sites were shared between all off-target identification methods (FIG. 8, panel H, FIG. 15, panel B).

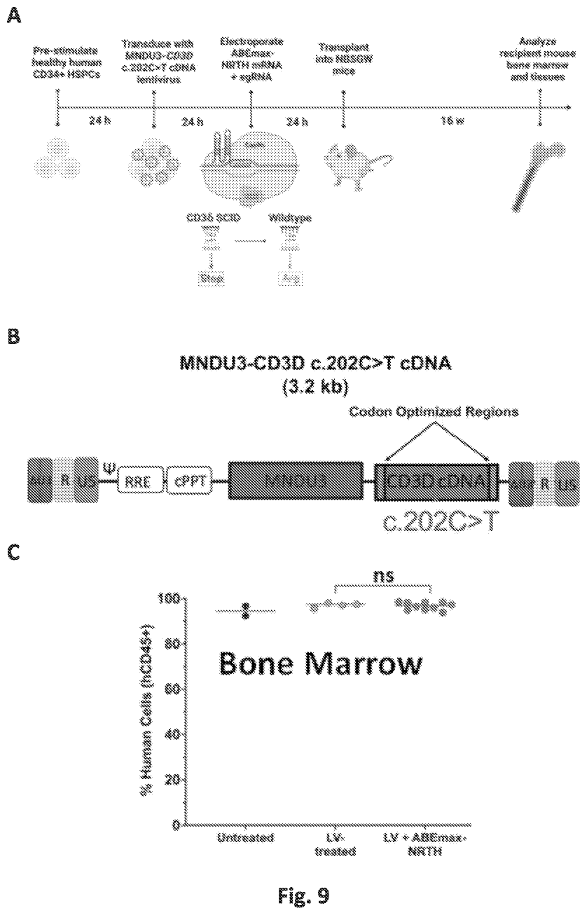

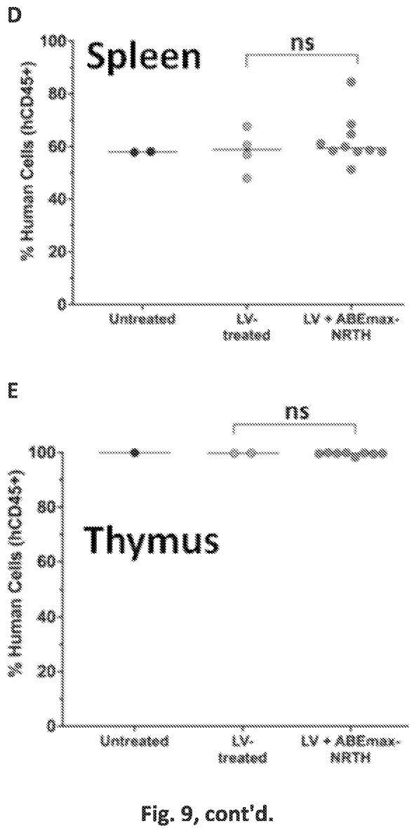

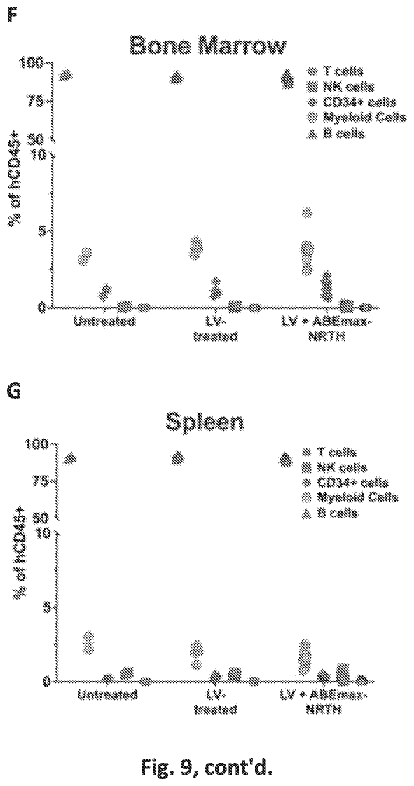

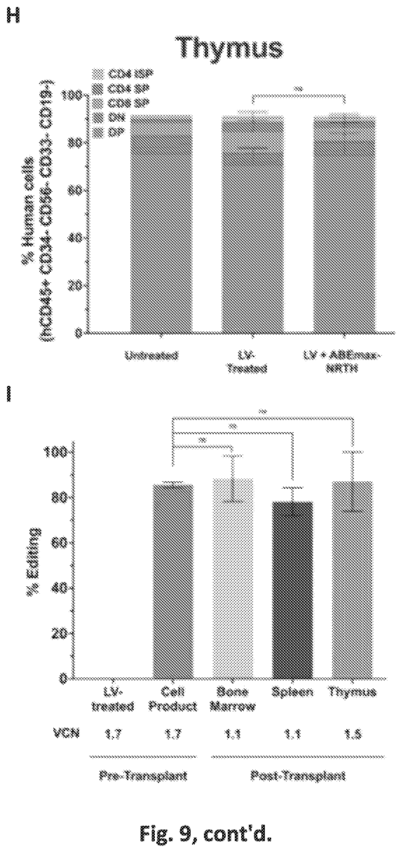

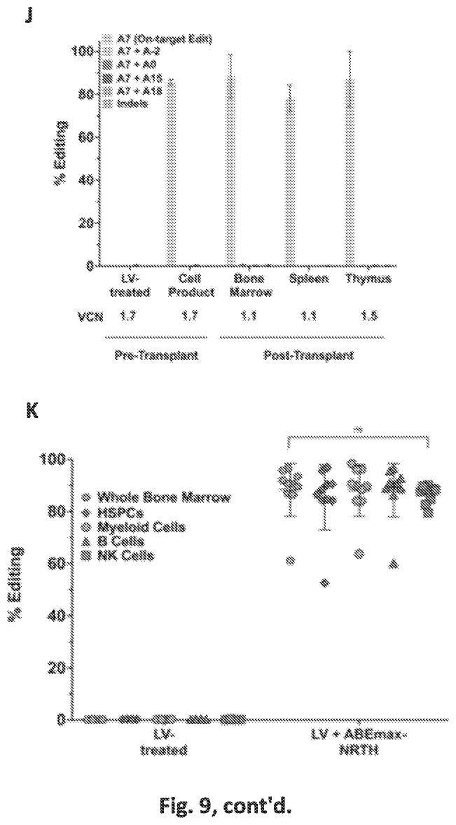

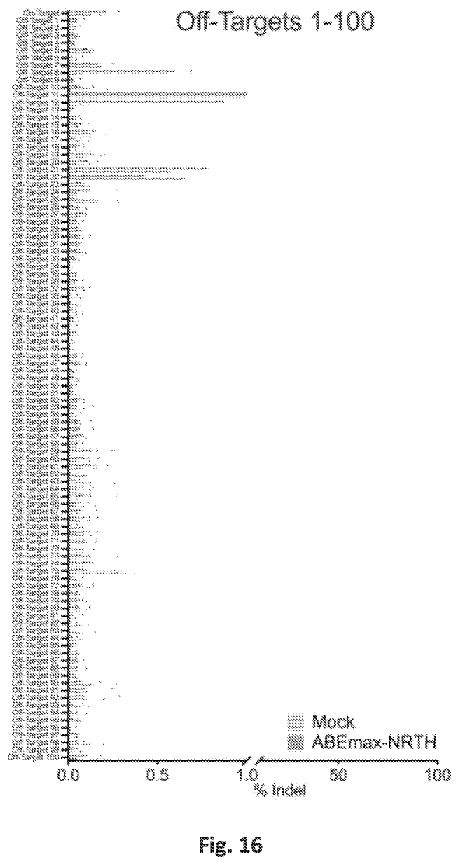

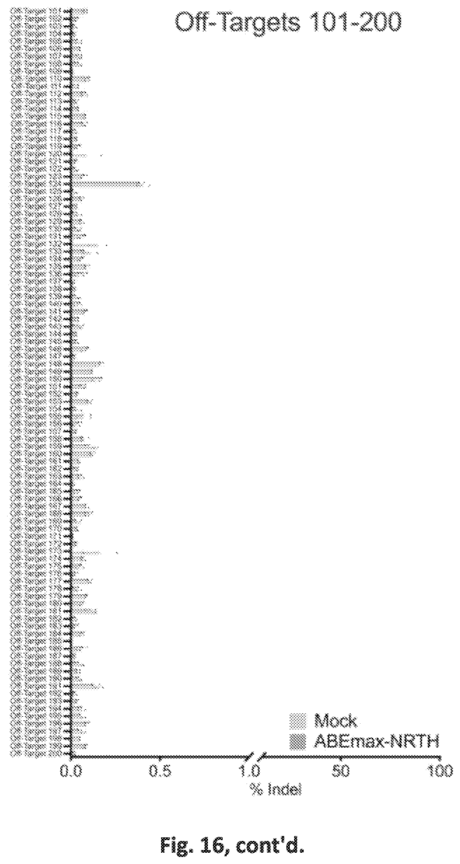

Next, we performed multiplex-targeted high-throughput sequencing in CD38 SCID patient HSPCs treated with ABEmax-NRTH mRNA and sgRNA (described in FIG. 9) at the 57 off-target sites nominated by two or more prediction methods and the remaining top 143 sites nominated by CIRCLE-seq (n=200). Despite high levels of on-target CD3D c.202C>T editing (71.2±7.85%), we observed A⋅T-to-G⋅C point mutations, characteristic of adenine base editing, at 2.5% (5/200) of the sequenced sites (FIG. 8, panels I and J; FIG. 15, panels C and D). All five validated sites were nominated by CIRCLE-seq, with three sites also identified by GUIDE-seq, and two sites predicted by Cas-OFFinder, demonstrating the importance of using experimental methods when investigating off-target sites. Of the five verified sites, three sites were found in introns greater than 100 bp away from any coding region and two sites occurred in intergenic regions (FIG. 8, panel K). Indel frequencies were less than 0.54% at all sequenced sites after subtraction of mock control reads (FIG. 16). Altogether, our assessment of local bystander editing and genome-wide off-target editing did not indicate clinically concerning off-target editing, despite high levels of on-target editing.

### Long-Term Correction of Healthy Human HSPCs in a Humanized Mouse Model

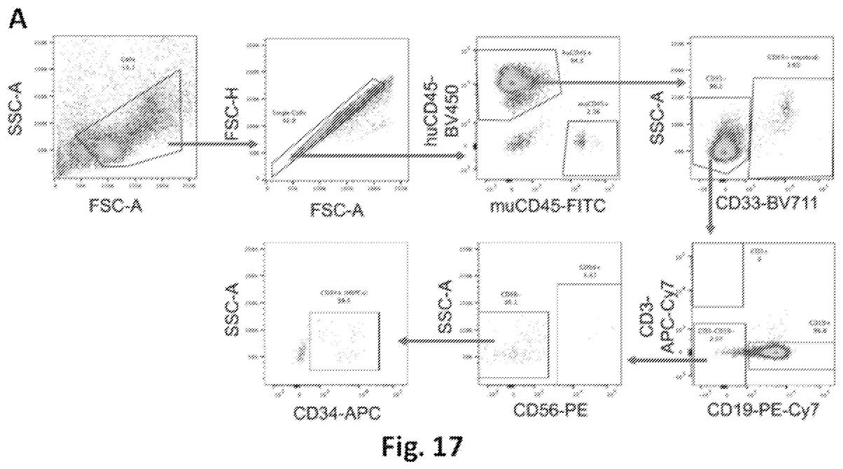

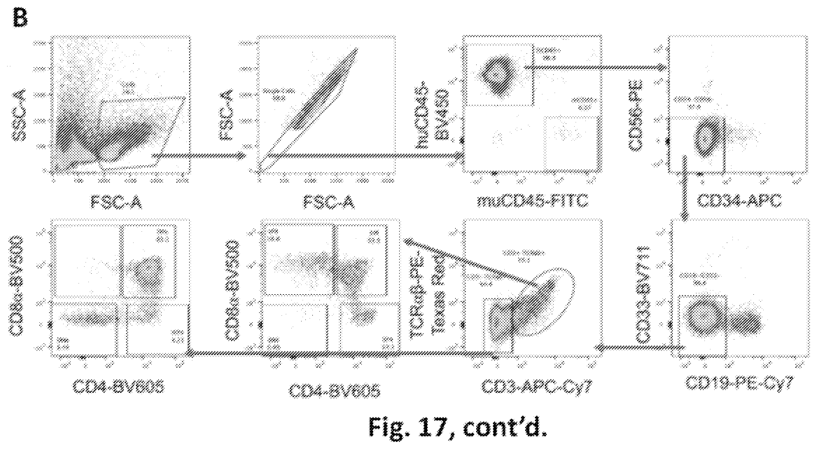

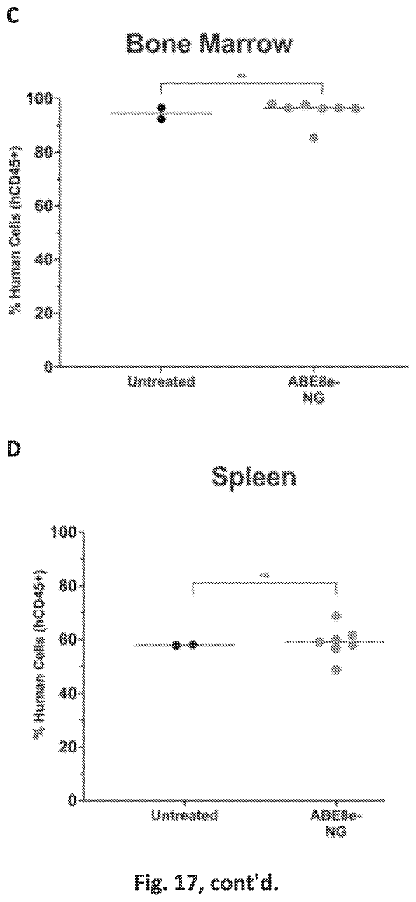

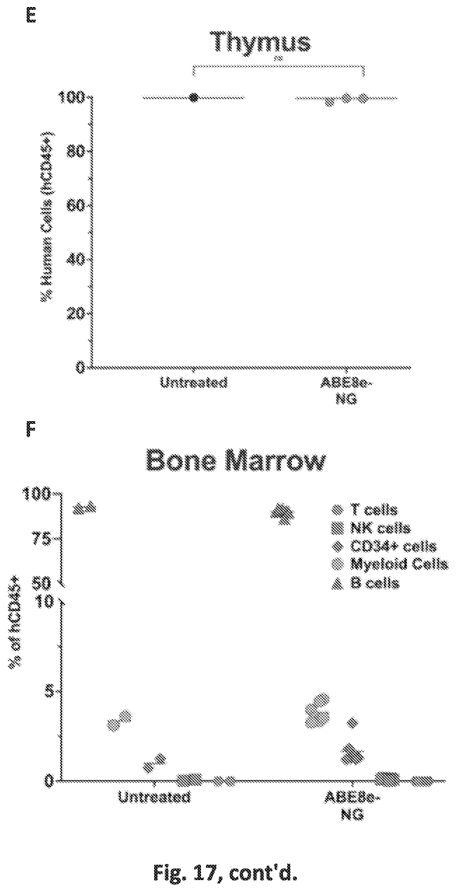

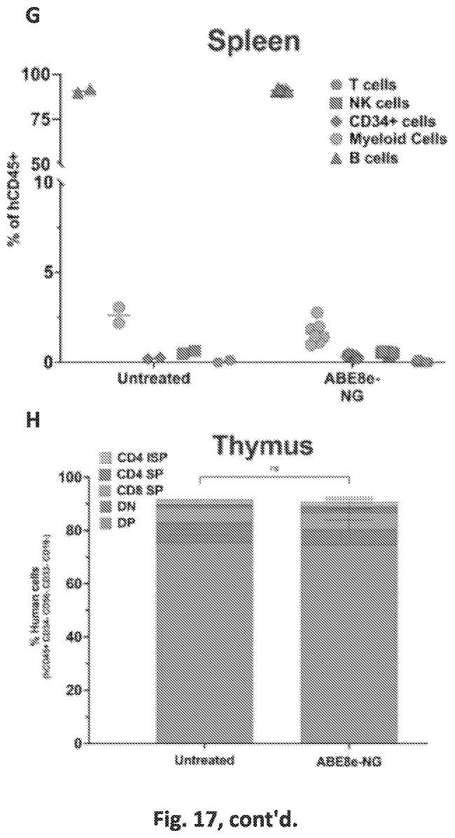

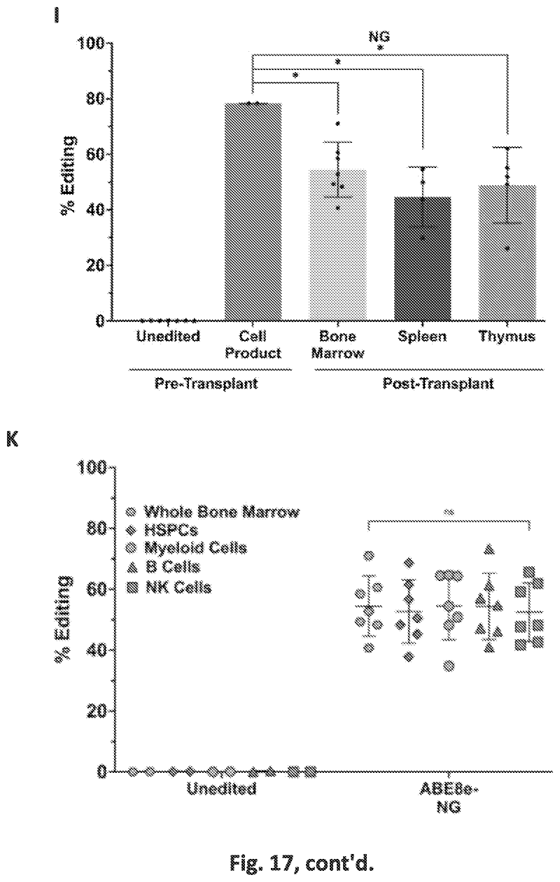

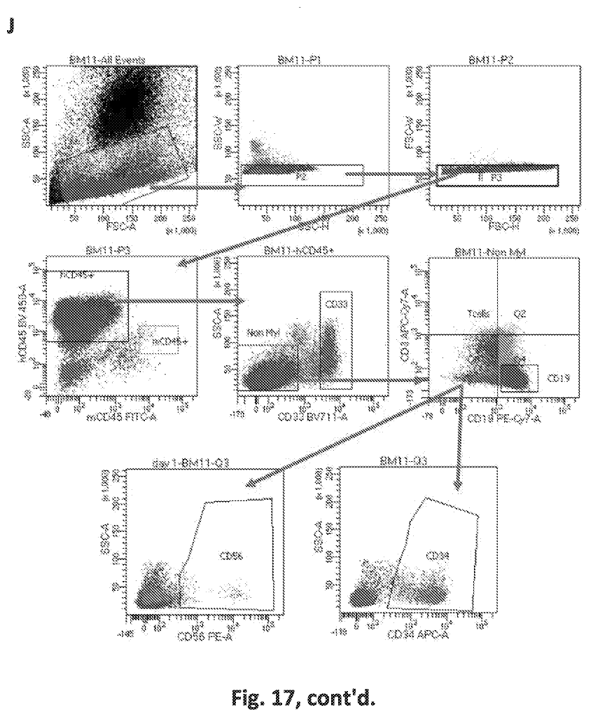

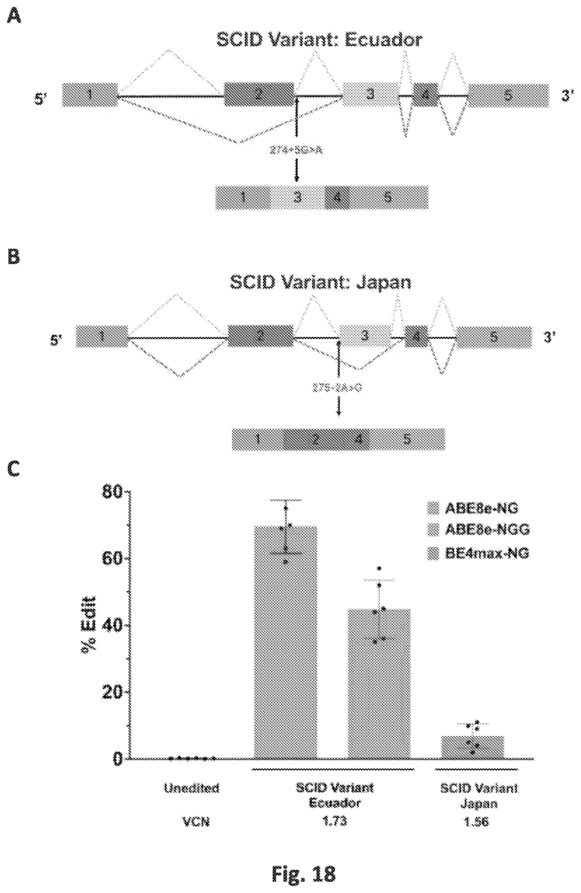

We next explored the ability to base edit the pathogenic CD3D mutation in long-term, repopulating cells in a humanized xenograft model. Healthy human CD34+ HSPCs were transduced with a lentiviral vector expressing a CD3D cDNA disease target containing the CD3D c.202C>T mutation under the control of the MNDU3 promoter (MNDU3-CD3D c.202C>T-cDNA) (FIG. 9, panel A). Codon optimized N- and C-termini (20 bp) of the LV cDNA enabled differentiation of the corrected mutation from endogenous CD3D sequence (FIG. 9, panel B) by PCR amplification (utilizing primers specific to the codon-optimized sequences). Twentyfour hours later, transduced HSPCs were electroporated with mRNA encoding ABEmax-NRTH and sgRNA. The same approach was utilized to revert two other recurrent CD3δ SCID-causing mutations identified in Ecuador and Japan, generating LVs carrying CD3D cDNA with either mutation for correction by base editing in healthy donor (HD) CD34+ cells (FIG. 17). The following day, resulting CD3D c.202C>T LV-transduced and edited HSPCs along with transduced-only control cells were each transplanted into 4-10 NOD, B6.SCID IL2rg−/−KitW41/W41 (NBSGW) immunodeficient mice.²⁵ As a control to ensure LV transduction did not disrupt xenograft studies, HD HSPCs were electroporated with mRNA encoding ABE8e-NG and sgRNA targeting an endogenous adenine base 6 bp downstream of the CD3δ SCID site without a LV. After 24 hours, the resulting edited cells and untreated control cells were additionally transplanted into 2-7 NBSGW mice (FIG. 17).

To assess the effects of base editing on engraftment and lineage maintenance, we extracted bone marrow (BM), spleen, and thymus from the recipient mice for analysis 16 weeks after transplant. Flow cytometry demonstrated 96.2±1.45%, 58.3±0.40%, and 99.8±0.10% of hCD45+ human cells in all mice BM, spleen, and thymus, respectively. Furthermore, we did not observe statistically significant differences in engraftment between untreated, LV− treated, and LV+ BE-treated human cells (p=0.63), indicating that engraftment was not altered by base editing (FIG. 9, panels C, D, E). To determine if CD3D-targeted base editing influenced HSPC differentiation potential and lineage maintenance, we investigated the proportions of human CD19+ B cells, CD33+ myeloid, CD34+ HSPCs, CD56+NK cells, and CD3+ T cells in engrafted mice (FIG. 17, panel A). Relative abundances of hematopoietic lineages were equivalent across control and treatment arms in the BM and spleen, suggesting that base editing did not alter hematopoiesis (FIG. 9, panels F, G). Although mature human T cells develop minimally from healthy stem cells engrafted in the adult NBSGW model due to thymic atrophy,²⁶ analysis of reconstituted donor thymocytes demonstrated no changes in sub-population distribution, indicating that base editing did not disrupt thymocyte differentiation potential (p=0.97) (FIG. 9, panel H; FIG. 17, panel B).

Engraftment of gene-corrected, repopulating HSCs is a critical objective for sustained and effective hematopoiesis and survival following autologous HSCT.²⁷ To investigate whether base editing can effectively correct the pathogenic mutation in long-term HSCs, we quantified CD3D c.202C>T editing efficiencies five days after electroporation (‘pre-transplant’) (85±1.2%) and at the 4-month harvest from the mice (FIG. 9, panels I, J). Notably, 16 weeks after infusion, editing frequencies measured from whole BM, spleen, and thymus of transplant recipients demonstrated durable base editing (84.5±5.52%, 78.2±6.18%, and 87±13.1%, respectively), suggesting high levels of gene correction in repopulating HSCs (p=0.73, p=0.13, and p=0.89) (9, panels I, J).

Additionally, we explored if base editing could influence multipotency of repopulating HSCs. Different lineages of human donor-derived (hCD45+) mononuclear cells (hCD45+ Whole Bone Marrow, CD34+ HSPCs, CD33+ myeloid, CD19+ B cells, and CD56+NK cells) were fluorescence-activated cell sorting (FACS) sorted from recipient mouse bone marrow. HTS of the CD3D disease target revealed no changes in base editing frequencies across all isolated populations (87.0±1.15%; p=0.95); bystander edits were <1%. (FIG. 9, panel K).

Engraftment, differentiation potential, and multipotency were similarly unaffected in cells edited at an endogenous adenine with ABE8e-NG mRNA and wildtype CD3D-targeting sgRNA without LV transduction (FIG. 16, panels C-H). Before transplantation, 78% editing was observed in the HSPC pool, and in repopulating HSCs that engrafted, 54% editing was maintained (FIG. 16, panels J, K). It is possible that this larger drop reflects that it is more challenging to edit the endogenous gene than the lentiviral transgene in repopulating HSCs, or that the SpCas9-NG editing strategy is less efficient. Altogether, these findings suggest that ABEmax-NRTH-treated CD34+ HSPCs can successfully repopulate the hematopoietic system and maintain therapeutic CD3D c.202C>T correction in all hematopoietic progeny.

### Base-Editing of CD3δ SCID HSPCs Rescues T Cell Development

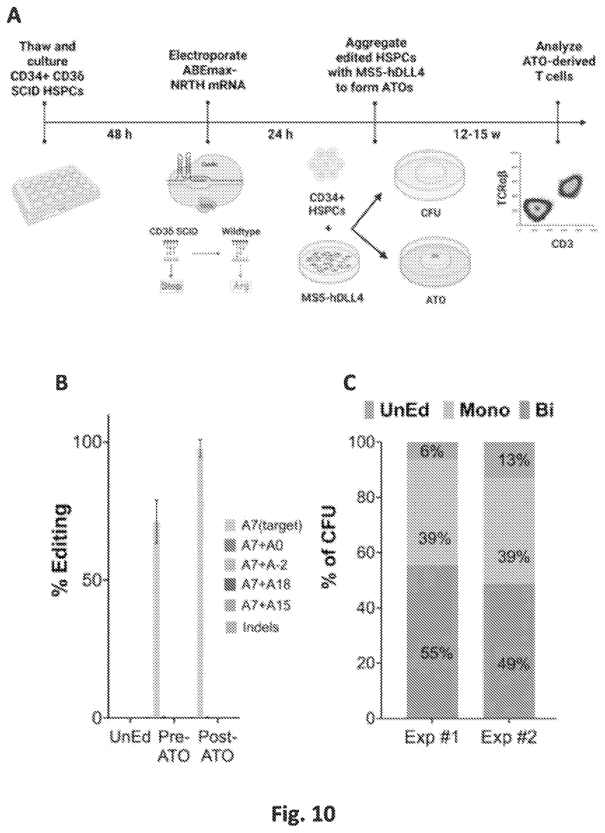

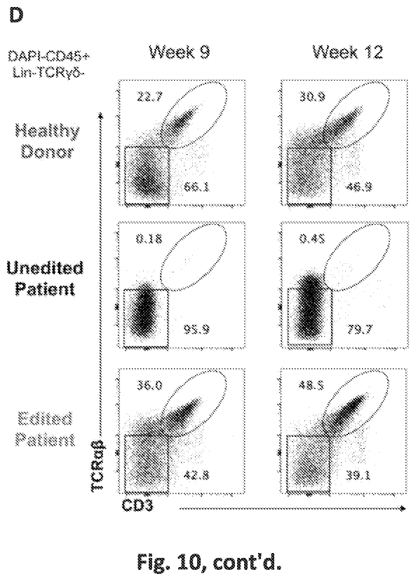

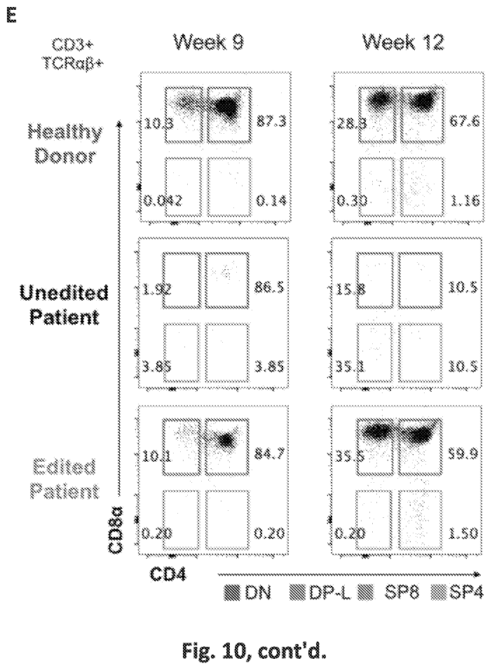

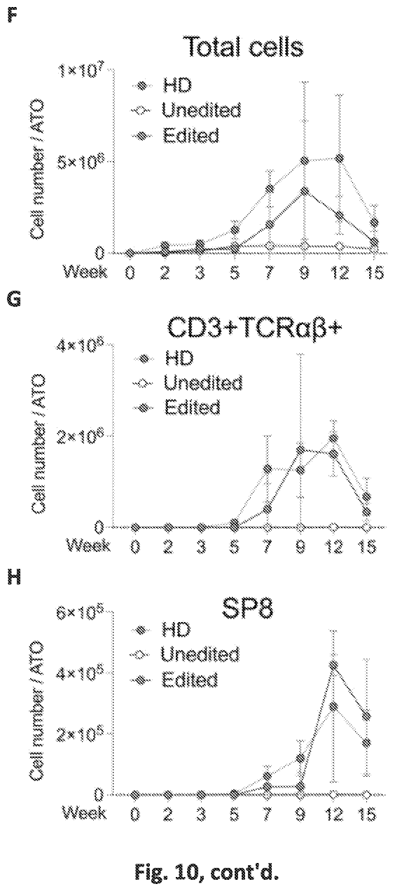

To evaluate whether base editing of CD3δ SCID HSPCs can rescue CD3 and TCR surface expression and normal T cell development, we employed an in vitro T cell differentiation assay (the artificial thymic organoid [ATO] model) that recapitulates normal human thymopoiesis from uncommitted HSPCs^{8,10,28} (FIG. 10, panel A). CD34+ bone marrow cells from an infant with CD3δ SCID were electroporated with ABEmax-NRTH mRNA and the sgRNA and tested for their capacity to generate mature T cells in ATOs with and without base editing and compared to bone marrow CD34+ cells from a healthy donor (HD) control (FIG. 10, panel A).

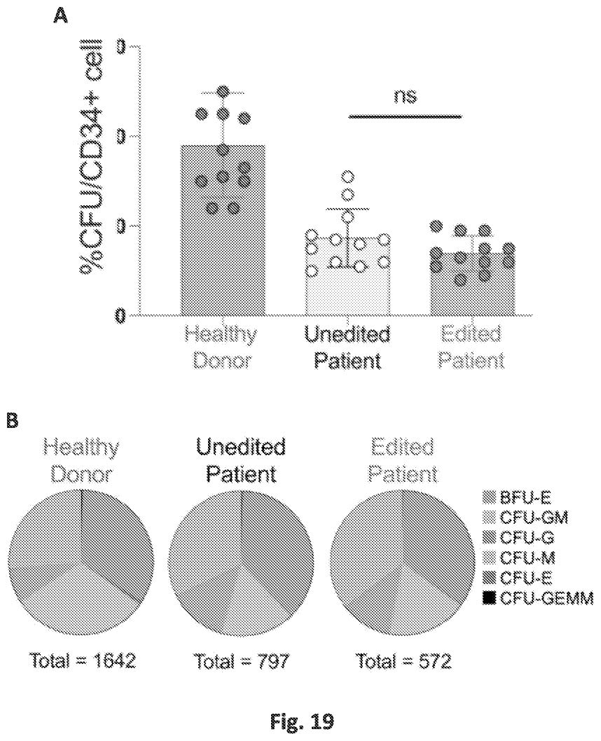

Electroporation of ABEmax-NRTH mRNA and sgRNA achieved 71.2±7.85% correction of the CD3D c.202C>T mutant alleles in HSPC by high throughput sequencing (HTS) prior to plating in ATOs, with minimal bystander editing or indels (FIG. 10, panel B). One day after electroporation, an aliquot of cells from each arm was plated in methylcellulose for a colony forming unit (CFU) assay to assess base editing at the clonal myelo-erythroid progenitor level (FIG. 10, panel C). Sequence analysis of individual CFUs demonstrated that 52±4.24% of cells contained biallelic correction of the CD3D c.202C>T mutation, 39±0.10% of cells showed monoallelic editing, and only 9.5±4.95% of cells remained unedited (n=230) (FIG. 10, panel C). Additionally, no impact of editing was observed on myelo-erythroid differentiation (FIG. 19, panels A, B).

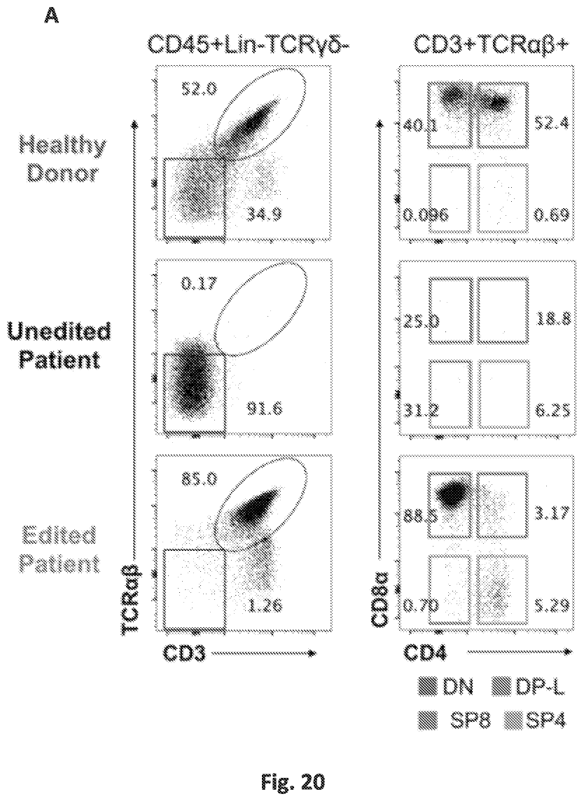

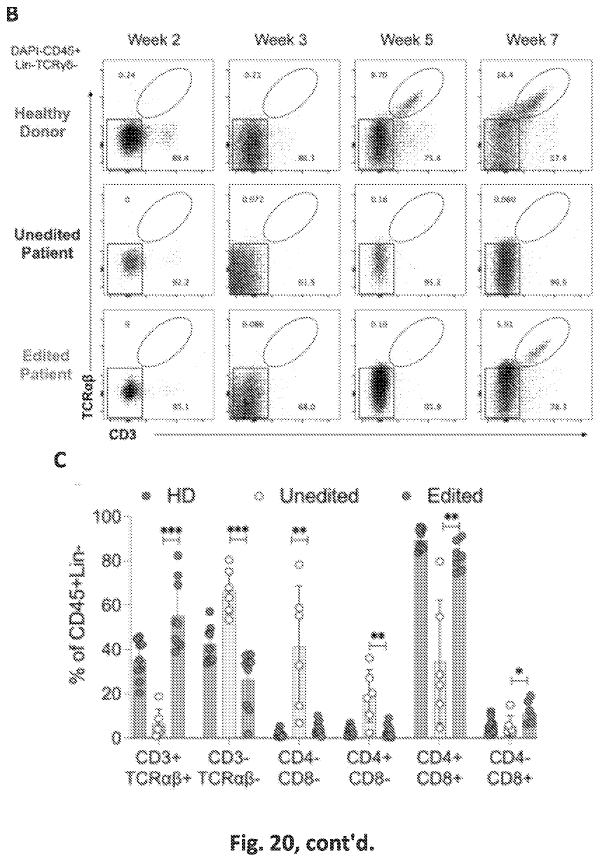

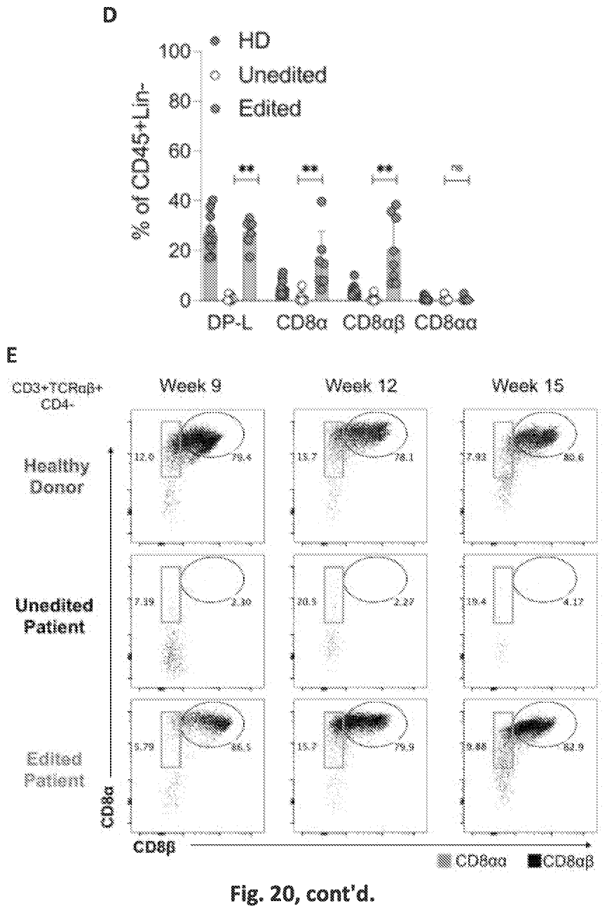

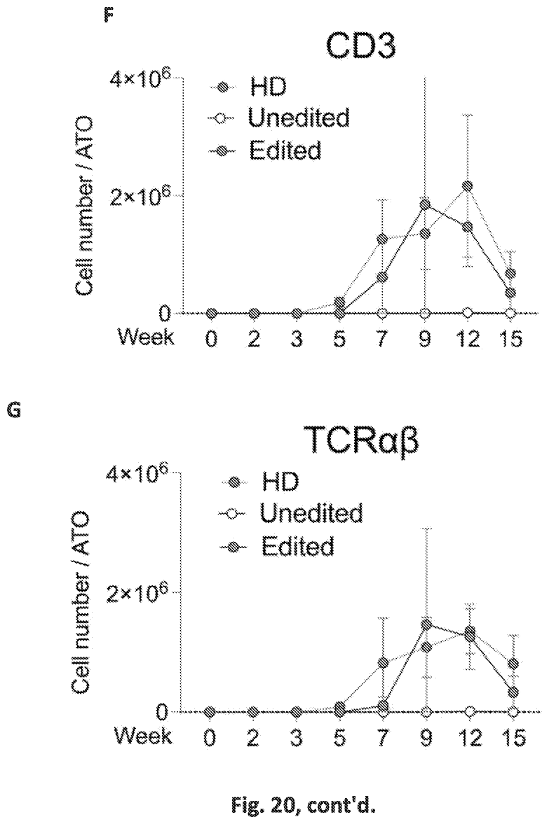

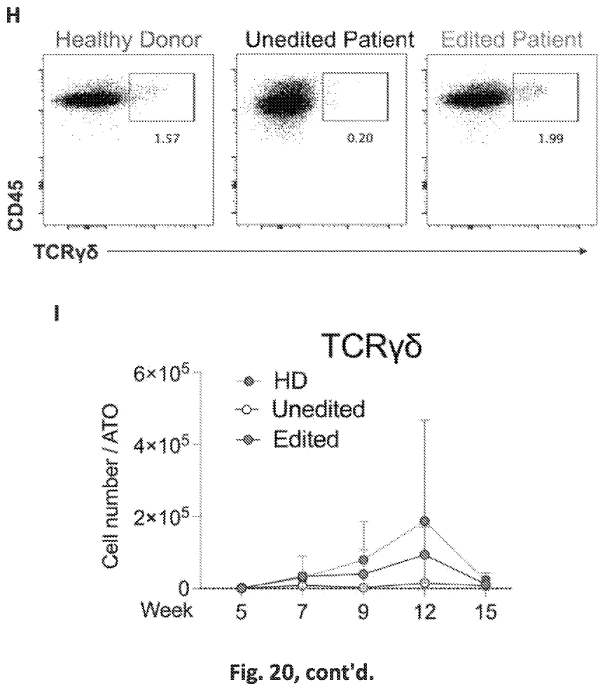

The majority of the cells were grown in ATOs and T cell development was evaluated by flow cytometry at 2, 3, 5, 7, 9, 12, and 15 weeks after electroporation. As expected, HD ATOs generated cells that co-expressed CD3 and TCRαβ at increasing percentages over time (FIG. 10, panel D, FIG. 20, panels A-C), with maturation to late DP (“DP-L” i.e. CD3+TCRαβ+CD4+CD8a+), SP8 T cells (CD3+TCRαβ+CD4−CD8a+CD8b+) and SP4 (CD3+TCRαβ+CD4+CD8−) T cells (FIG. 10, panel E, FIG. 20, panels A-F). In contrast, cells from unedited patient ATOs had undetectable CD3 and TCR surface expression across all time points (FIG. 10, panel D and FIG. 20, panels A-F). Because TCR expression was absent in unedited patient HSPCs, T cell differentiation was severely disrupted with an accumulation of unedited cells in the DN (CD4−CD8−) precursor stage and an inability to progress past the DP (CD4+CD8+) developmental stage into either SP8 T cells or SP4 T cells (FIG. 10, panel F and FIG. 20, panels A, C). Surface CD3 and TCRαβ co-expression was robustly rescued in edited patient ATOs (FIG. 10, panel D, FIG. 20, panels A-C, E, F), appearing first at the DP stage and persisting in SP8 and SP4 T cell populations (FIG. 10, panels E, I; FIG. 20, panels A, D). The total cell output (FIG. 10, panel G) and SP8 output (FIG. 10, panel I) per ATO was similar between edited patient and HD ATO cultures across all time points and dramatically decreased in unedited cells.

Previous reports have described faulty development of TCRγδ+ T cells in patients with CD3δ SCID.^{2,29,30} Unedited patient ATOs recapitulated this clinical finding, demonstrating the absence of TCRγδ+ T cell production across all time points. In contrast, edited patient and HD ATOs supported the development of TCRγδ+ cells to similar extents (FIG. 20, panels G, H).

### Differentiation of Unedited CD3δ SCID HSPCs Cannot Proceed Past DP T Cell Precursor Stage

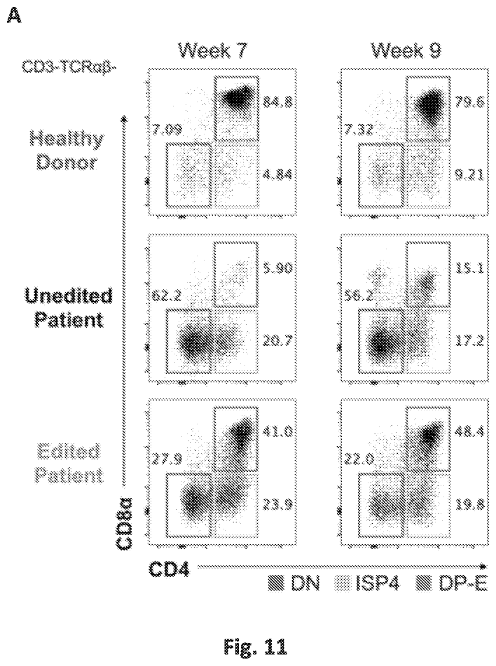

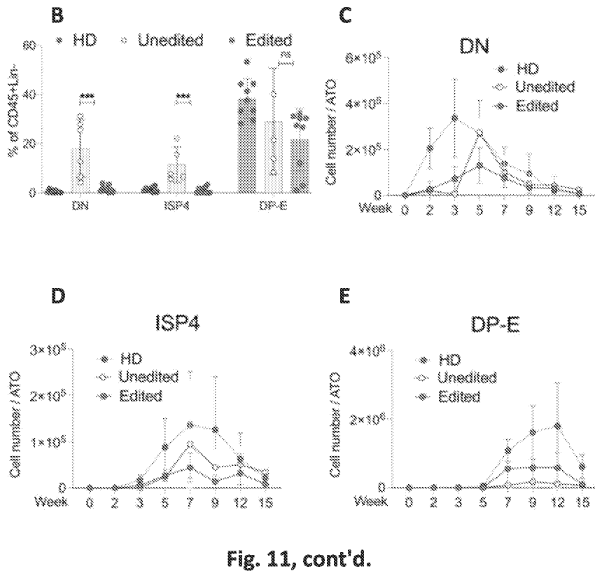

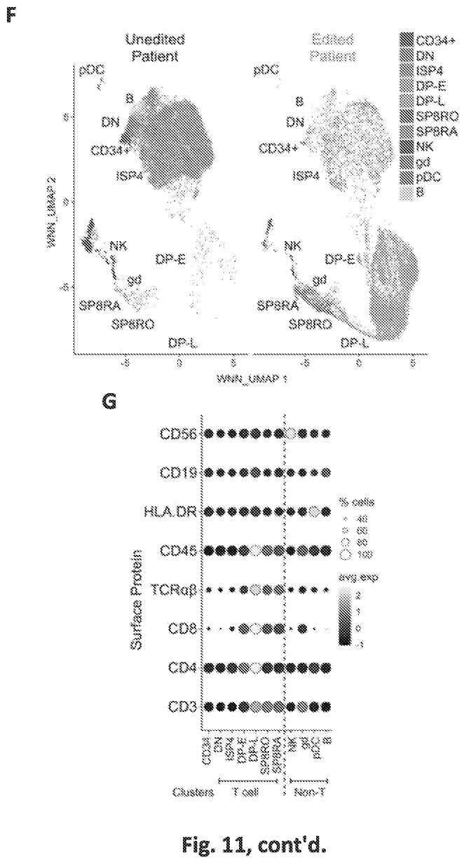

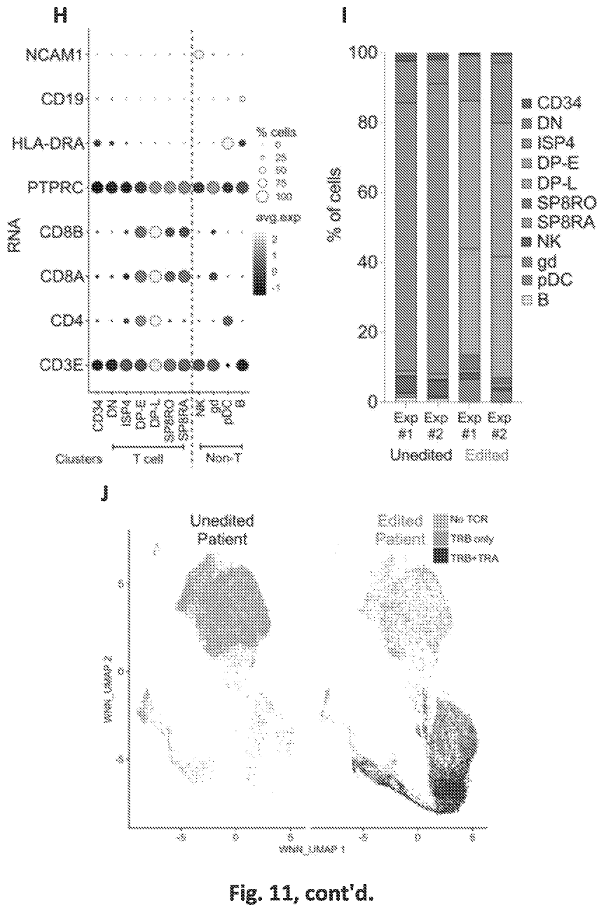

A single prior report of an individual patient with CD3δ SCID characterized the block in thymopoiesis at the DN (CD3−TCRαβ−CD8−CD4−) stage by western blot of a thymic biopsy.² In contrast, the ATO system allowed us to interrogate thymopoiesis kinetics in an unprecedented manner. As previously reported, unedited patient ATOs demonstrated increased DN populations as compared to HD and edited patient ATOs (FIG. 11, panels A-E). However, we identified maturation past the DN stage to the ISP4 and DP stages in unedited patient ATOs. While TCR-CD3-DP cells (DP-E) precursors could be detected in unedited patient ATOs (FIG. 11, panel B), their absolute numbers were low (FIG. 11, panel E).

### Single-Cell RNA Sequencing Identifies Initial TRA Expression in DP-L Precursors

To provide a more detailed analysis of how base editing of CD3δ SCID affected T cell development, cellular indexing of transcriptomes and epitopes by sequencing (CITE-seq)³¹ was utilized to integrate surface protein, transcriptional profile, and TCR clonotype expression at single cell resolution. ˜40,000 cells were isolated from unedited and edited CD3δ SCID ATOs harvested at week 8 (n=2 replicates for each arm, two independent experiments) and sequencing libraries were generated using the 10× Chromium Single Cell Sequencing workflow. Surface antibody staining was performed using Total-seq C cocktail (Biolegend, San Diego, CA, USA) against 130 unique surface antigens. Individual samples were cleaned (Material and Methods) and ˜22,000 cells were aggregated for downstream analysis (Table 4).

TABLE 4
Number of bioinformatically cleaned cells in indicated
cell subsets in unedited and edited patient ATOs harvested
at week 8 from two individual experiments.

| | CD34+ | DN | DP-E | DP-L |
|---|---|---|---|---|
| Unedited Exp#1 | 114 | 820 | 105 | 4 |
| Unedited Exp#2 | 52 | 309 | 69 | 4 |
| Edited Exp#1 | 19 | 20 | 23933 | 1756 |
| Edited Exp#2 | 23 | 95 | 1753 | 1643 |

| | SP8RO | SP8RA | NK | gd |
|---|---|---|---|---|
| Unedited Exp#1 | 0 | 0 | 350 | 83 |
| Unedited Exp#2 | 0 | 0 | 229 | 21 |
| Edited Exp#1 | 203 | 52 | 96 | 324 |
| Edited Exp#2 | 75 | 48 | 48 | 144 |

| | PdC | B | Total |
|---|---|---|---|
| Unedited Exp#1 | 83 | 98 | 7000 |
| Unedited Exp#2 | 21 | 48 | 4561 |
| Edited Exp#1 | 324 | 4 | 5530 |
| Edited Exp#2 | 1 | 9 | 4637 |

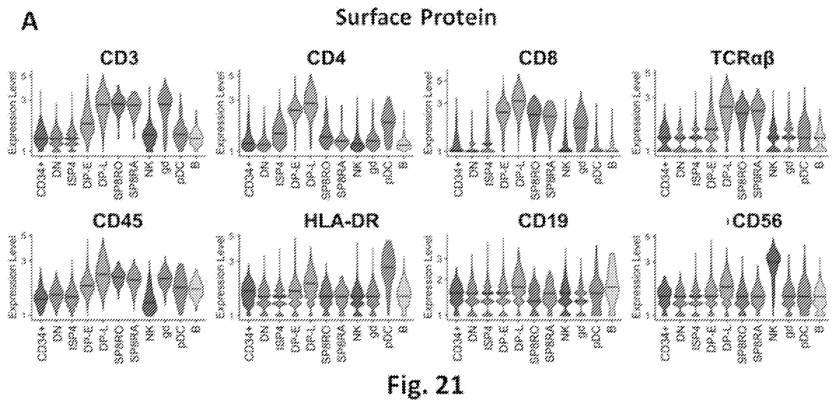

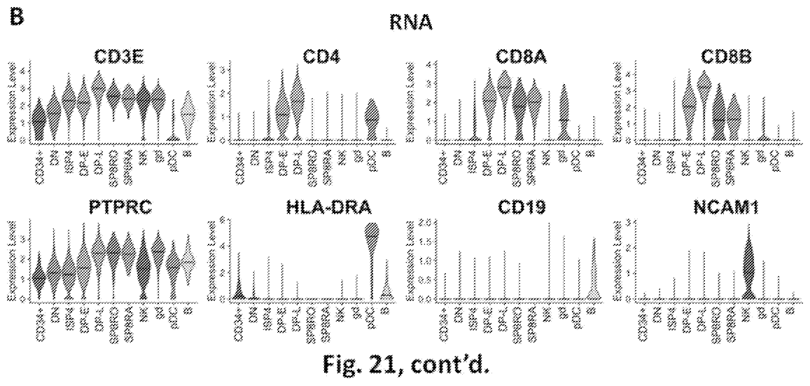

To visualize both surface protein and gene expression changes, we performed Weighted Nearest Neighbor (WNN) multimodal analysis in Seurat (v4.2.0).³² After WNN analysis, we generated a WNN Uniform Manifold Approximation and Projection (WNN_UMAP) visualization (FIG. 11, panel F), and clusters were manually collapsed and assigned to individual cell subsets based on a combination of surface protein (FIG. 11, panel G and FIG. 21, panel B) and RNA gene expression (FIG. 11, panel H and FIG. 21, panel C). The following subsets were identified: CD34+ (CD34+CD4−CD8−TCRαβ−), DN (CD34−CD8−CD4−), ISP4 (CD3−TCRαβ−CD8−CD4+), DP Early (DP-E: CD3−TCRαβ−CD8+CD4+); DP Late (DP-L, CD3+TCRαβ+CD8+CD4+), SP8 (CD3+, TCRαβ+CD8+CD4−; further divided into SP8RO and SP8RA), NK cells (CD56+), Y$ T cells (TCRαβ−TCR.V62), pDC (CD4+RAG1−RAG2−HLADR+), and B cells (defined as PAX5+CD19+).

WNN_UMAP visualization confirmed that unedited patient ATOs contained high proportions of DN and ISP4 subsets (FIG. 11, panels F and I). While FACS analysis identified a higher proportion of DP-E precursors (FIG. 11, panel A, FIG. 20, panel A), than did CITE-Seq analysis, a dead-cell removal kit was applied to ATOs prior to CITE-seq, likely depleting a proportion of rapidly apoptosing DP-E cells. As expected, CITE-seq analysis confirmed that populations defined by the co-expression of CD3 and TCRαβ (DP-L and SP8 subsets) were absent in unedited patient ATOs and restored in edited ATOs.

The TCR comprises two subunits: TRB and TRA, which must undergo rearrangement of germline variable (V), diversity (D), and joining (J) gene segments to generate a mature TCR.³³ TRB rearranges at the DN stage and TRA rearranges at the DP stage.³⁴ Because the development of unedited patient ATOs is blocked at the DP stage, we assessed TRB and TRA usage by single-cell TCR sequencing as described above by CITE-seq. Analysis of each TCR subunit found that single cells expressing both TRA and TRB belonged to cells with CD3/TCR surface expression (i.e. DP-L, SP8RO and SP8RA clusters, whereas single cells expressing only TRB were found in precursor populations that lacked CD3/TCR surface expression: DN, ISP4, and DP-E (FIG. 11, panel J). Unedited patient ATO-derived cells expressed TRB but not TRA and were unable to proceed to the DP-L stage when TRA is normally expressed.

T Cells Derived from Edited CD3δ SCID HSPCs ATOs Show Mature Naïve Phenotype

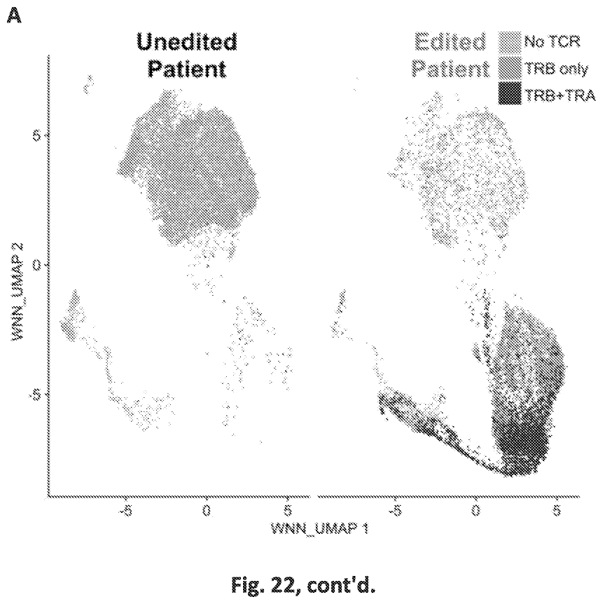

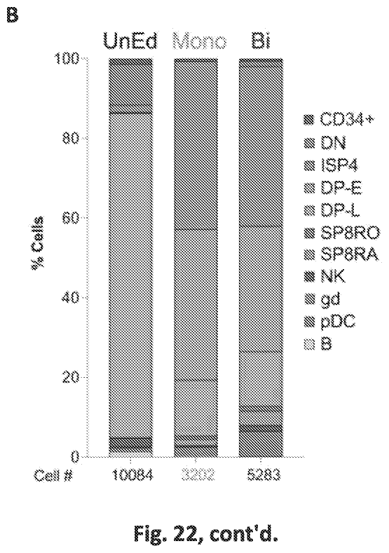

Due to the autosomal recessive nature of CD3δ SCID, correction of a single CD3D allele is expected to rescue disease phenotype. Single-cell monoallelic and biallelic correction frequencies were measured by presence of RNA abundance in both unedited and edited patient ATOs. We observed nonsignificant differences in relative abundances of T cell precursors and in T cell maturation of patient-derived ATO cells containing a monoallelic or biallelic edit (p=0.99) (FIG. 22, panels A, B).

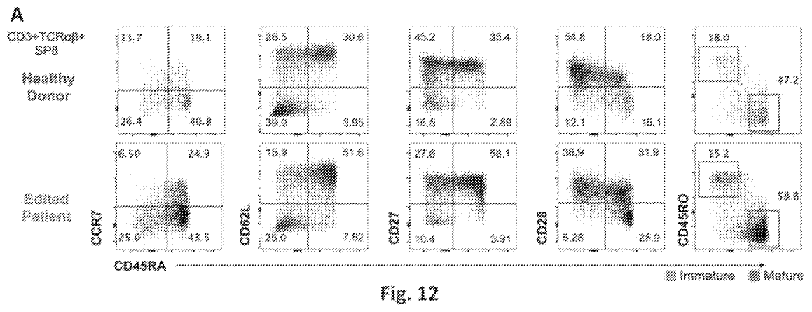

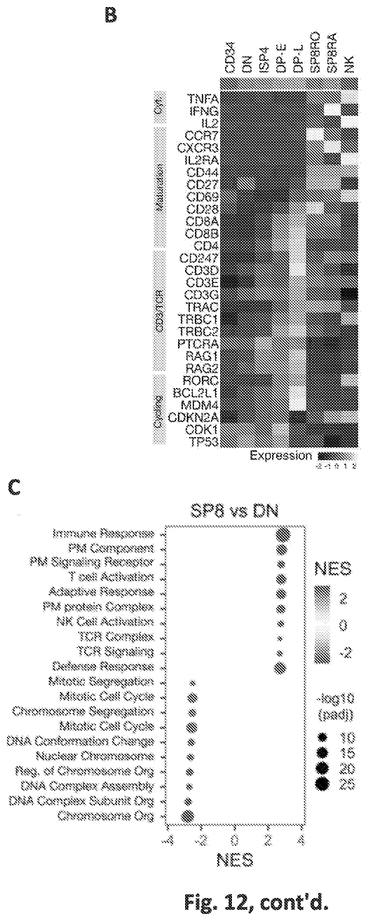

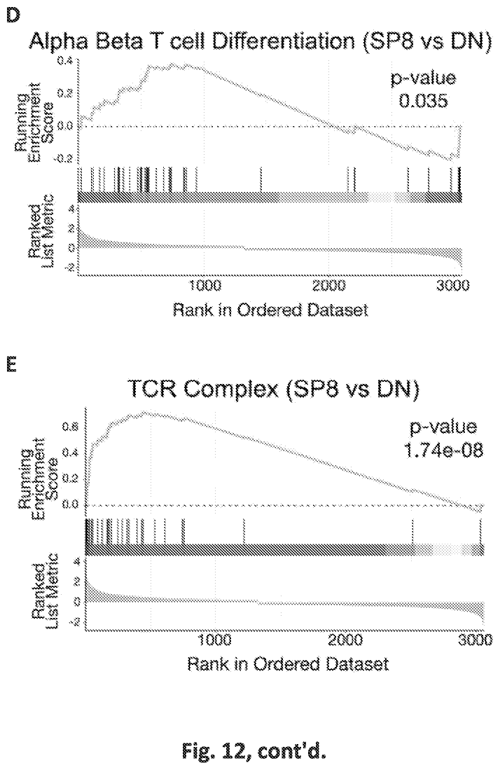

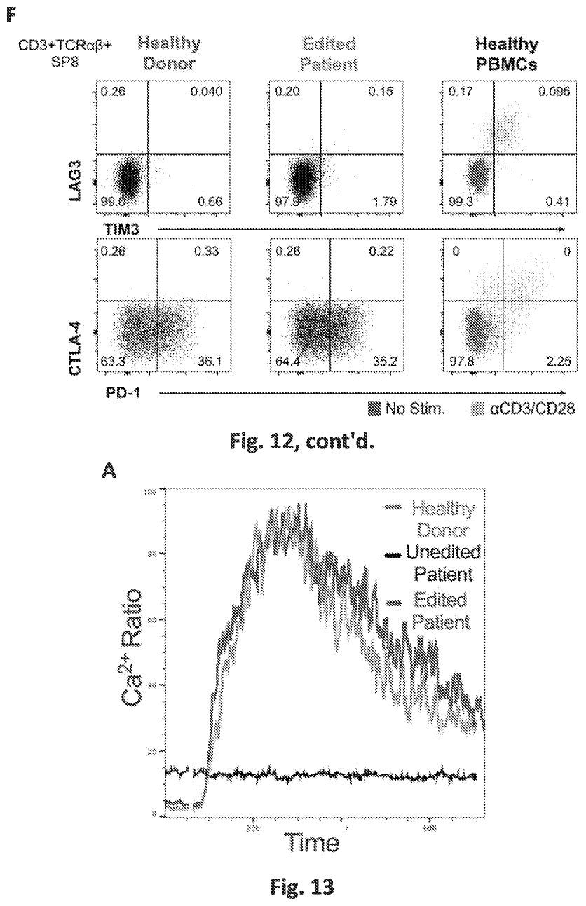

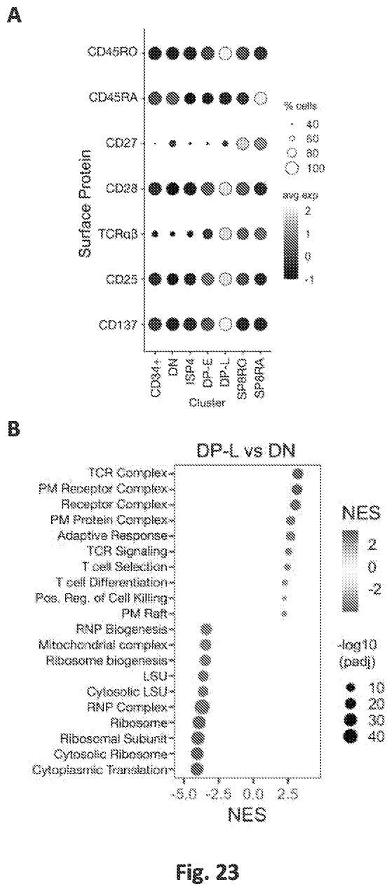

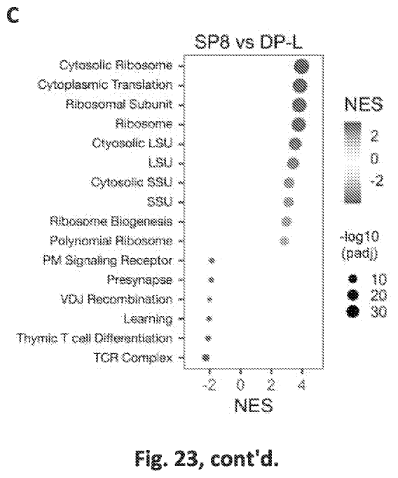

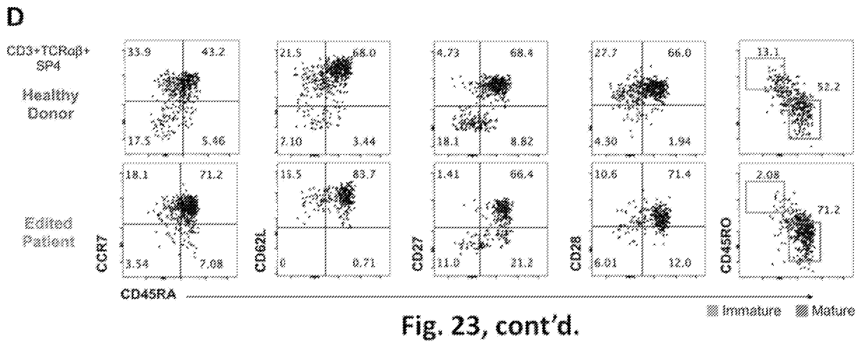

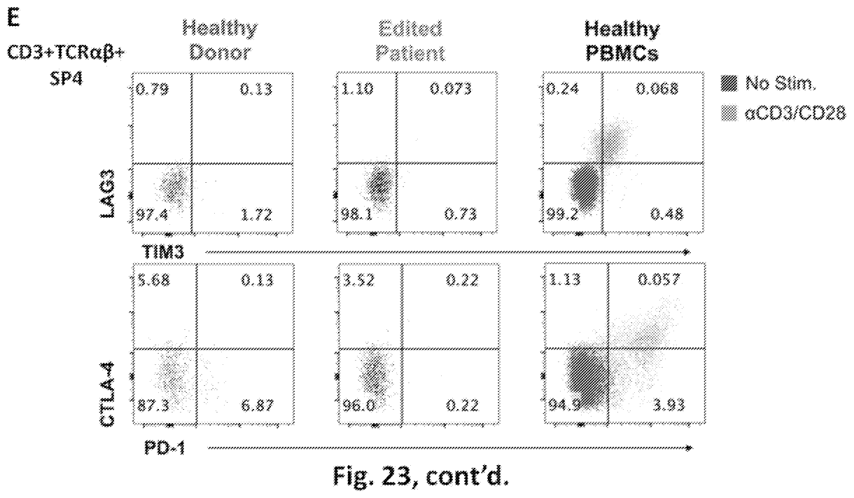

SP8 T cells derived from edited patient ATOs expressed markers consistent with transition from an immature (CD45RO+CD45RA−CD27+CCR7−) to mature (CD45RO-CD45RA+CD27+CCR7+) thymocyte phenotype; both immature and mature subsets co-expressed CD62L and CD28 (FIG. 12, panel A). Expression levels of maturation markers were similar between edited patient and HD ATOs by flow cytometry (FIG. 12, panel A); and CITE-seq analysis of cells derived from edited patient ATOs confirmed expression of maturation markers (CD27, CD28, CD45RA, CD45RO, and TCRαβ), while lacking expression of activation markers CD25 and CD137 in SP8RO/RA cells (FIG. 23, panel A).

Single-cell transcriptomic analysis (FIG. 12, panel B) demonstrated that mature SP8 T cells derived from edited patient ATOs expressed high levels of genes found in mature T cells (CXCR3, IL2RA, CD44), CD3/TCR signaling (CD247, CD3D/E/G, TRA/TRB), and cell cycling/proliferation (RORC, BCL2L1, MDM4, CDKN2A, CDK1, and TP53). Gene Set Enrichment Analysis (GSEA) was utilized to identify relevant biological processes and pathways that differed across developing thymocyte subsets in edited ATO-derived cells, where CD3/TCR expression was rescued. T cell activation, T cell differentiation, and TCR signaling were upregulated in SP8 (both SP8RO and SP8RA) relative to DN cells (FIG. 12, panels C-E). Comparison of DP-L vs. DN cells identified upregulation of T cell differentiation and TCR signaling pathways (FIG. 23, panel B) in DP-L cells. Comparison of SP8 (both SP8RO and SP8RA) T vs. DP-L cells, highlighted upregulation of ribosomal pathways required for protein translation in SP8 T cells (FIG. 23, panel C).

Restoration of T cell development in base edited ATOs resulted in normal production of SP8 T cells in culture. FACS analysis of SP8 T cells from late (15 week) ATO cultures from edited patient cells and healthy donor T cells lacked expression of exhaustion markers LAG3, TIM3, and CTLA-4.35-37 PD-1 expression was detected in both edited patient and HD ATOs at similar levels (FIG. 12, panel F). SP4 T cells derived from edited patient ATOs demonstrated similar expression of maturation markers and lacked expression of exhaustion markers (FIG. 23, panels D, E).

Base Edited CD3δ SCID HSPCs Develop into Functional T Cells with a Diverse TCR Repertoire

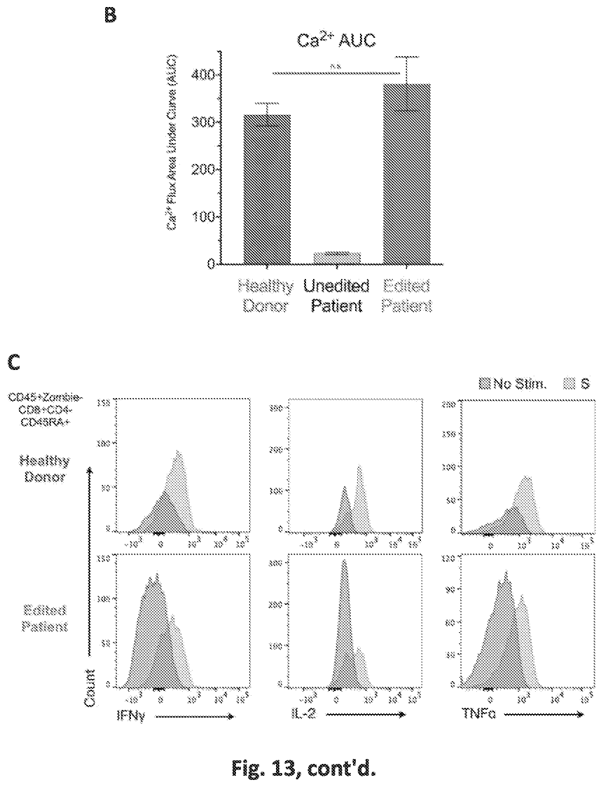

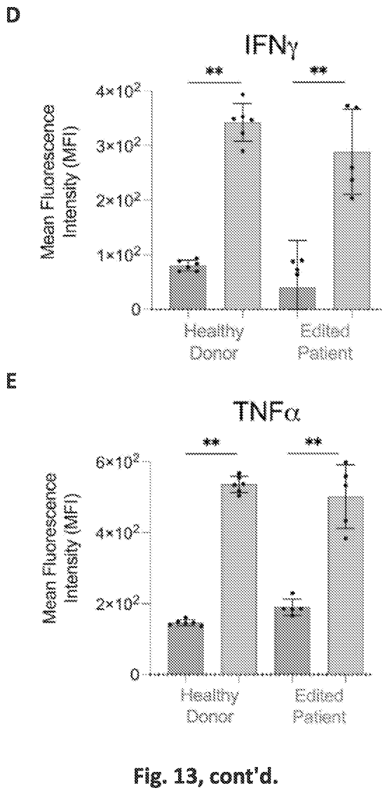

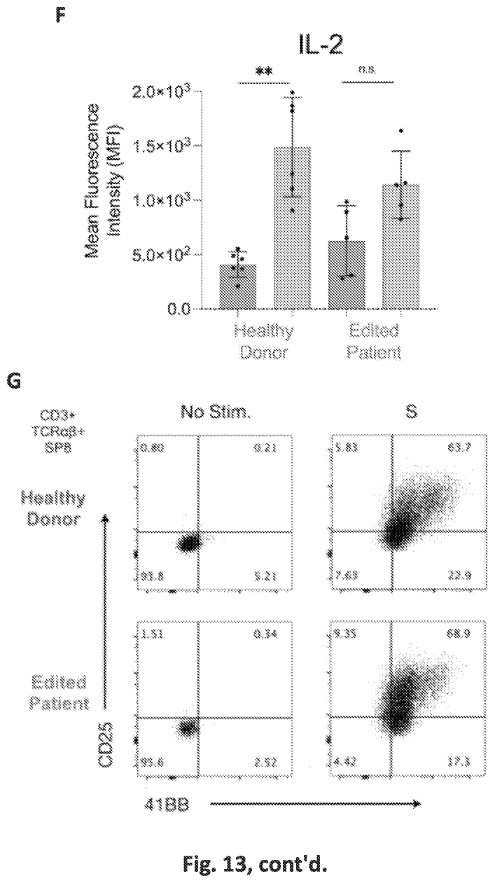

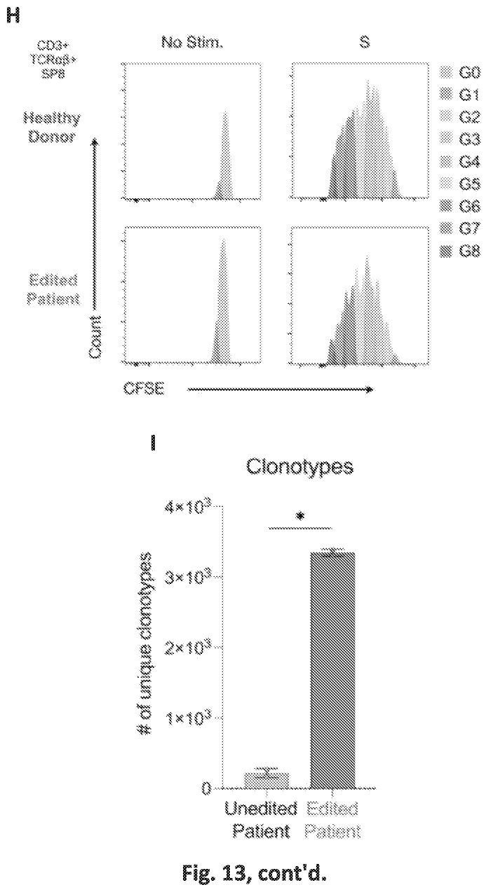

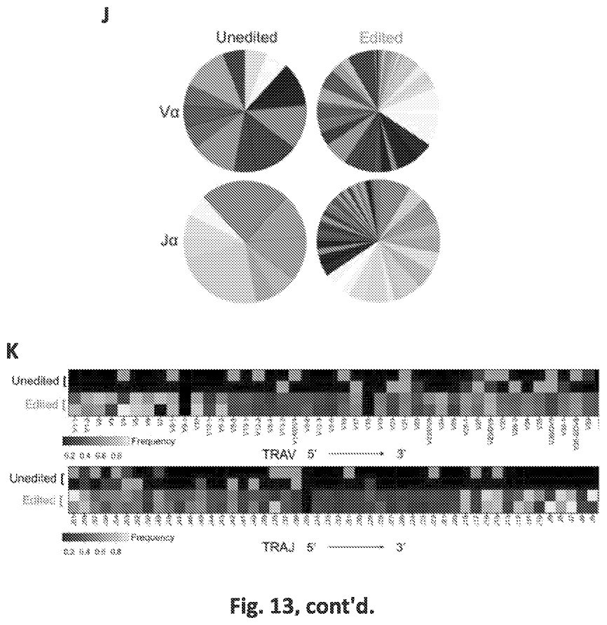

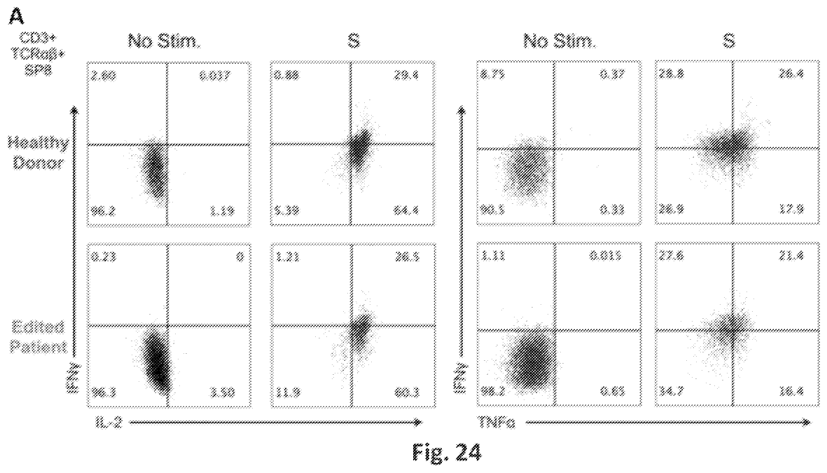

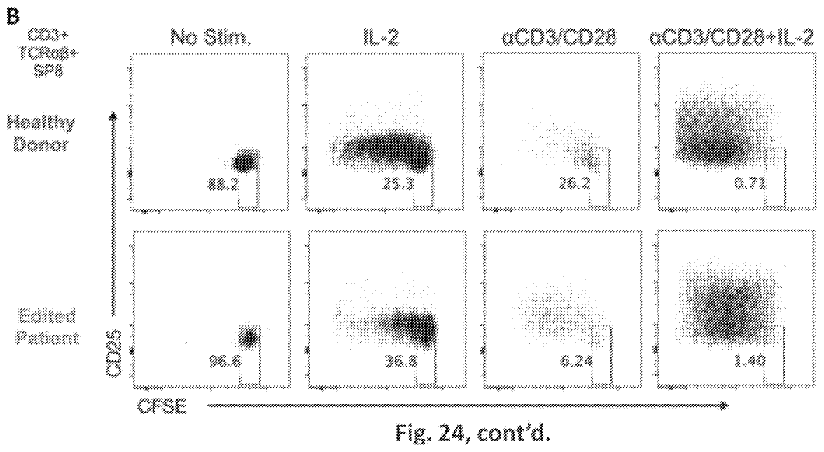

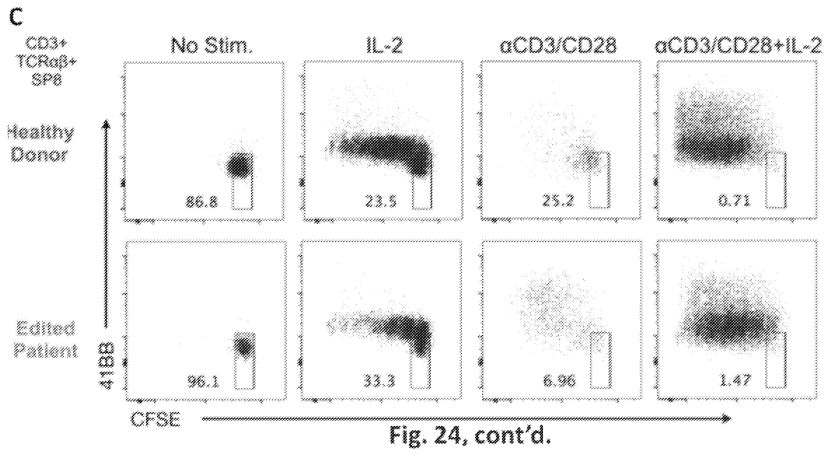

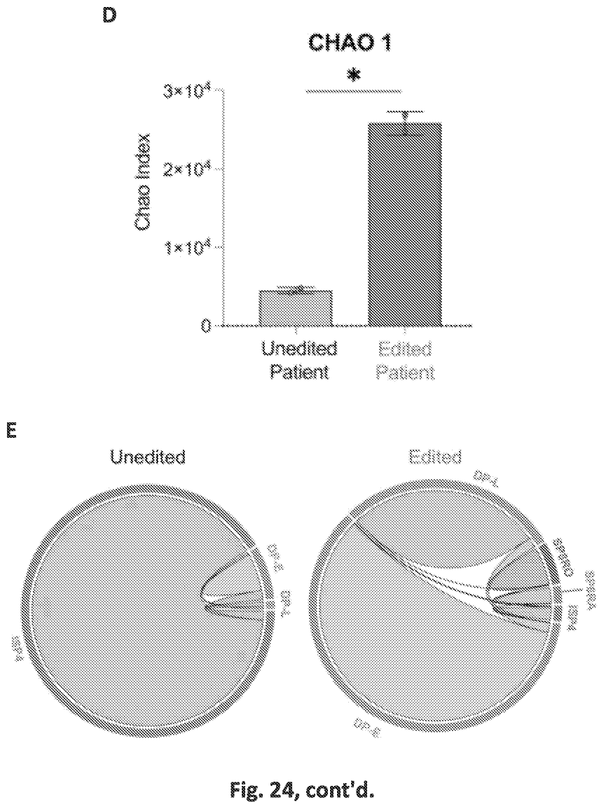

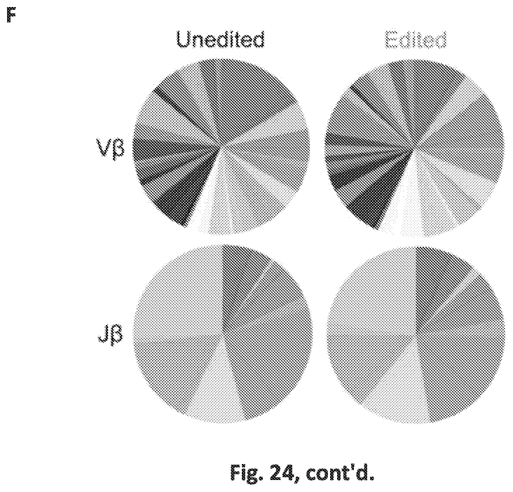

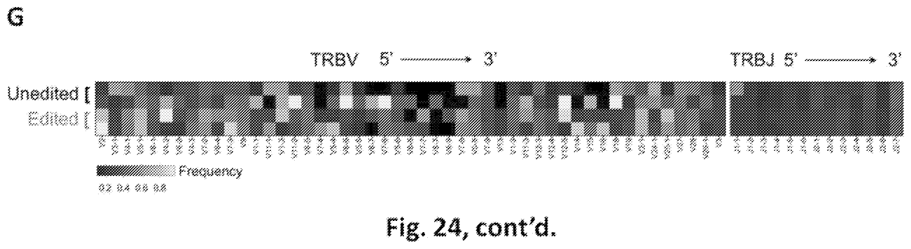

To evaluate the ability of base editing to produce T cells with functional TCRs, week 12-15 ATOs were harvested and calcium flux analysis was performed as a proxy for early CD3/TCR activation (FIG. 13, panel A). Consistent with lack of CD3/TCR, unedited patient ATO cells displayed no calcium flux in response to stimulation with anti-CD3 and anti-CD28 antibodies. Base editing restored calcium flux to similar levels as HD ATO cells (381.0±56.9 and 316±24.1 AUC) (FIG. 13, panel B). Mature SP8 T cells isolated from edited patient ATOs and HD ATOs demonstrated similar polyfunctional production of IFNγ, IL-2, and TNFα in response to stimulation with anti-CD3/CD28 beads+IL-2 for 24 hours (FIG. 13, panels C-F and FIG. 24, panel A). SP8 T cells upregulated CD25 and 4-1BB and proliferated in response to anti-CD3/CD28 beads and IL-2 for 5 days (FIG. 13, panels G and H; FIG. 24, panels B and C).

A diverse TCR repertoire is essential for an effective T cell immune response. Unedited CD3δ SCID ATOs demonstrated significantly fewer TCR clonotypes as compared to edited patient ATOs (217.5±65.8, n=2 vs. 3344±50.1, n=2, p<0.002) (FIG. 13, panel I) as evidenced by decreased Chao 1 index 38 (FIG. 24, panel D). Chord diagrams of T cell populations from unedited and edited patient ATOs illustrate shared TCR clonotypes between developmentally neighboring subsets (FIG. 24, panel E). In unedited patient ATOs, ISP4 precursors expressed the highest diversity of TCR clonotypes, and shared TCR clonotypes with DP-E precursors. In edited patient ATOs, DP-E precursors, yet to undergo positive selection, expressed the highest diversity of TCR clonotypes, and shared TCR clonotypes with DP-L precursors. In contrast, positively selected SP8RO and SP8RA T cells expressed fewer TCR clonotypes.

Further independent analysis of TRA and TRB usage revealed skewed TRA usage towards the 3′ proximal TRAV and 5′ distal TRAJ usage in unedited patient ATOs. These segments represent the regions of Va and Ja that rearrange first during VDJ recombination. Base editing of CD3δ SCID HSPC restored diverse TRAV and TRAJ usage, and corrected TRA skewing in edited patient ATOs (FIG. 13, panels J and K). No significant differences were found in TRBV or TRBJ usage between unedited and edited patient ATOs (FIG. 24, panels F and G).

Taken together, these data demonstrate robust restoration of T cell development from CD3δ SCID HSPCs by ABE-mediated gene therapy. Extensive phenotyping of edited T cells in ATOs revealed rescue of mature T cell function and diverse TCR repertoire, indicating clinical promise in this approach.

### DISCUSSION

The ability to correct pathogenic point mutations that cause life-threatening monogenic diseases is becoming a clinical reality for precision medicine. One promising approach is base editing to efficiently and precisely correct disease-causing alleles.^{5,39,40} Base editing has advantages over approaches using homology-directed repair to correct mutations as it can be achieved without producing DSBs, generating uncontrolled mixtures of indel byproducts, requiring provision of donor DNA templates, or being limited to cells in certain phases of the cell cycle required for HDR. Here, we describe an ABE-mediated approach to revert the mutation underlying most CD3δ SCID cases (CD3D c.202C>T) to wildtype sequence. This approach successfully reverted the premature stop codon in a Jurkat T cell line disease model, in healthy donor (HD) CD34+ HSPCs transduced with a LV carrying a target CD3D cDNA with the c.202C>T mutation, and in CD34+ HSPCs isolated from an affected CD3δ SCID patient's bone marrow. This base editing strategy was precise and efficient in all blood cell types analyzed (up to 85% in CD3D (C202T) Jurkat T cells, 96% in repopulating HSPCs, and 79% in CD3δ SCID patient-derived HSPCs), with minimal bystander edits or indels.

The capacity to precisely position the ABE editing window at the target base may be limited by the availability of an appropriate protospacer adjacent motif (PAM) to direct localization of the base editor by a sgRNA. As demonstrated here, Cas9 variants with expanded targeting scope beyond the canonical NGG PAM of native Sp Cas9 can enable efficient and precise targeting of human pathogenic gene variants. Investigation of five ABE variants including three novel ABEs, ABE8e-xCas9 (3.7)), ABE8e-VRER, and ABE8e-NRTH, and two previously generated editors, ABE8e-NG and ABEmax-NRTH, 20,41 resulted in robust correction of the c.202C>T mutation (18%, 33%, 92%, 86%, and 93%, respectively) whereas a homology-directed repair (HDR) approach using Cas9 nuclease, sgRNA and an ssODN donor only achieved 28% correction to the wildtype sequence, accompanied by an excess of indel byproducts (53%).

Cas-nuclease mediated DSBs are well established to induce chromosomal abnormalities at on- or off-target sites.¹⁶ Indeed, we observed large deletions distal to the on-target CD3D locus (11q23) when CD3D (C202T) Jurkat T cells were treated with RNP+ssODN, but not when treated with ABEmax-NRTH. These deletions are particularly concerning from a clinical standpoint where some chromosomal abnormalities in HSPCs have frequently been associated with AML and poor prognosis for CML patients,⁴² suggesting ABE may be a safer and more efficacious treatment for CD3δ SCID by circumventing the production of DSBs.

We observed infrequent (<1%) adenine editing at position A0 (counting position 1 as the PAM_distal end of the spacer) in cells electroporated with ABEmax-NRTH; whereas ABE8e induced bystander edits at a much higher frequency (18-45%). The rare bystander editing at A0 by the lead candidate ABEmax-NRTH produced an isoleucine to threonine substitution that did not have a clear adverse effect on function of the CD38 protein; expression of this variant corrected the CD3/TCR signaling in CD3D (C202T) Jurkat T cells to be equivalent to cells receiving a wildtype control. Thus, this low-level of bystander editing utilizing ABEmax-NRTH will not likely impair ABE efficacy for CD3D (C202T) correction.

Furthermore, we examined the occurrence of genome-wide off-target base editing in primary CD3δ SCID patient HSPCs treated with ABEmax-NRTH mRNA and sgRNA. Of the 200 sites evaluated, HTS of ABEmax-treated CD3δ SCID patient T cells verified only five sites containing point mutations consistent with adenine base editing, despite high levels of on-target CD3D editing. Of these five validated off-target sites, three sites occurred in intronic regions and the remaining two sites were found in intergenic regions. Without the induction of DSBs necessary for CRISPR/Cas9-mediated editing and the apparent low frequency of off-target edits, base editing is less likely to induce genotoxicity.

Despite its prevalence in rural Mennonite communities of North America (comprising over 20% of SCID-causing genotypes in Alberta, Canada) (N. Wright, personal communication), CD3δ SCID is an ultra-rare disease, thus limiting access to patient-derived HSPCs in numbers sufficient for in vivo xenograft studies of long-term repopulating HSPCs. Therefore, we utilized HD CD34+ HSPCs transduced with a lentiviral vector carrying the CD3D mutation target and then base edited the cells for transplantation into immunodeficient mice as a surrogate model to test engraftment potential of edited repopulating HSCs. Gene correction in long-term HSCs able to repopulate the hematopoietic system is essential to generate a clinical benefit from autologous HSCT. Encouragingly, we did not observe changes in engraftment, multipotency or corrective base editing of human cells treated with ABEmax-NRTH compared to LV-treated controls after 16 weeks in mice.

Although xenografts provide a feasible surrogate assay for long-term HSPC activity, definitive evidence of gene modification in repopulating HSCs can only be determined by longer observations in large animal HSCT models such as canines or nonhuman primates, or in human studies. The precision of base editing does not provide a convenient clonal tag commonly used with randomly integrating LV-based therapies. Nevertheless, the presence of unchanged, high-frequency ABEmax-mediated base editing in unfractionated bone marrow and in four isolated hematopoietic lineages from bone marrow after 16 weeks (CD34+ HSPCs, CD33+ Myeloid, CD19+ B cells, and CD56+NK cells) suggests engraftment of edited long-term HSCs.

Additionally, the method of using LV transduction of disease target mutations into HD CD34+ HSPCs facilitated proof-of-concept studies for correction of two additional pathogenic CD3D mutations reported to cause CD3δ SCID in Japan and Ecuador.^{43,44} These surrogate studies in HD HSPCs demonstrate a base editing pipeline capable of treating the most prevalent CD3δ SCID-causing mutations reported to date.

The ATO platform allows rigorous assessment of the effects of base editing on the CD3δ SCID disease phenotype due to its unprecedented ability to support in vitro development of mature T cells from HSPCs. Comprehensive characterization of ATO-derived mature T cells demonstrated rescue of CD3/TCR surface expression and TCR-dependent function at various stages of TCR activation. Edited ATO-derived T cells exhibited normal levels of calcium flux, cytokine production, and proliferation and revealed a highly diverse TCR repertoire.

Prior characterization of the block in T cell development in CD3δ SCID was hindered by the extreme rarity of the disease and limited patient samples. A thymic biopsy on a single CD3δ SCID patient reported in 2003 showed reduced CD4 and CD8 protein expression by western blot and absent CD4 and CD8 protein expression by immunohistochemistry.² These authors therefore posited a block in T cell development at the DN stage.² Because the ATO system allows for robust in vitro recapitulation of each stage of thymopoiesis, we were able to interrogate this question more deeply and at various stages of development. Our data revealed that unedited CD3δ SCID HSPCs developed past the DN stage to the ISP4 and DP-E stages. While the numbers of DP-E (CD3−TCR−) cells in unedited patient ATOs were lower as compared to edited patient and HD ATOs, a DP-E population is clearly present, in contrast to prior understanding. These data support inefficient development of unedited CD3δ SCID HSPCs to the DP-E stage, and a complete inability to proceed to DP-L stage.

Prior groups have described the disparate role of CD3δ in surface expression of TCRγδ in mice versus humans.⁴⁵ In mice with mutations in CD38, development of TCRαβ+ T cells is blocked, but TCRγδT cells appear normal.⁴⁵ Our data support the conclusion that in human T lymphopoiesis, CD3δ is critical for the development of both TCRαβ+ and TCRγδ+ T cells.⁸

Single cell analysis of TCR usage in ATO-derived cells revealed that unedited patient T cells demonstrated normal TRB rearrangement (completed by the DN stage) but were defective in TRA rearrangement. We describe for the first time that lack of CD3D leads to 3′ proximal TRAV and 5′ distal TRAJ skewing. This spatiotemporal pattern corresponds to skewing toward the genomic position that is rearranged first. RORc deficiency, also an IEI, results in a similarly skewed pattern of TRA usage,⁴⁶ which is believed to result from absent downstream apoptosis regulator BCL2L1, which is highly expressed in DP cells.^{47,48} In the case of CD3δ SCID, our data from patient ATOs suggests that skewing of TRA rearrangement likely results from the requirement for cells to express surface CD3/TCRαβ to survive and proceed through positive selection. Our data from edited patient ATOs further supports this hypothesis because base editing of CD3δ SCID HSPCs restored RORC expression in DPs. The inability for unedited CD3δ SCID HSPCs to efficiently mature to the DP-E stage is likely due to skewed TRA usage resulting in impaired surface expression of diverse TCRs. As such, base editing of CD3δ SCID HSPCs restores CD3/TCRαβ expression and allows for complete TRA rearrangement at the DP stage, leading to restored TCR diversity and positive selection.

Taken together, we demonstrate that highly efficient base editing to correct the CD3δ SCID mutation enabled robust rescue of T cell development and function. These results demonstrate the first potential genome editing approach for autologous HSCT to successfully correct CD3δ SCID. Although this work is limited to a single inborn error of immunity, translation to the clinic will have significant implications for numerous other rare, monogenic diseases, illuminating a potential translational pathway for the one-time treatment of these disorders.

### REFERENCES FOR EXAMPLE 2

1. Garcillan, B., Fuentes, P., Marin, A. v., Megino, R. F., Chacon-Arguedas, D., Mazariegos, M. S., Jimenez-Reinoso, A., Munoz-Ruiz, M., Laborda, R. G., Cardenas, P. P., et al. (2021). CD3G or CD3D Knockdown in Mature, but Not Immature, T Lymphocytes Similarly Cripples the Human TCRαβ Complex. Front Cell Dev Biol 9. 10.3389/fcell.2021.608490.
2. Dadi, H. K., Simon, A. J., and Roifman, C. M. (2003). Effect of CD3 d Deficiency on Maturation of a/b and g/d T-Cell Lineages in Severe Combined Immunodeficiency.
3. Marcus, N., Takada, H., Law, J., Cowan, M. J., Gil, J., Regueiro, J. R., Plaza Lopez De Sabando, D., Lopez-Granados, E., Dalal, J., Friedrich, W., et al. (2011). Hematopoietic stem cell transplantation for CD3δ deficiency. Journal of Allergy and Clinical Immunology 128, 1050-1057. 10.1016/j.jaci.2011.05.031.
4. Pavel-Dinu, M., Wiebking, V., Dejene, B. T., Srifa, W., Mantri, S., Nicolas, C. E., Lee, C., Bao, G., Kildebeck, E. J., Punjya, N., et al. (2019). Gene correction for SCID-X1 in long-term hematopoietic stem cells. Nat Commun 10. 10.1038/s41467-019-09614-y.
5. Newby, G. A., Yen, J. S., Woodard, K. J., Mayuranathan, T., Lazzarotto, C. R., Li, Y., Sheppard-Tillman, H., Porter, S. N., Yao, Y., Mayberry, K., et al. (2021). Base editing of haematopoietic stem cells rescues sickle cell disease in mice. Nature 595, 295-302. 10.1038/s41586-021-03609-w.
6. Romero, Z., Torres, S., Cobo, M., Muoz, P., Unciti, J. D., Martn, F., and Molina, I. J. (2011). A tissue-specific, activation-inducible, lentiviral vector regulated by human CD40L proximal promoter sequences. Gene Ther 18, 364-371. 10.1038/gt.2010.144.
7. Landrum, M. J., Lee, J. M., Benson, M., Brown, G., Chao, C., Chitipiralla, S., Gu, B., Hart, J., Hoffman, D., Hoover, J., et al. (2016). ClinVar: Public archive of interpretations of clinically relevant variants. Nucleic Acids Res 44, D862-D868. 10.1093/nar/gkv1222.
8. Bosticardo, M., Pala, F., Calzoni, E., Delmonte, O. M., Dobbs, K., Gardner, C. L., Sacchetti, N., Kawai, T., Garabedian, E. K., Draper, D., et al. (2020). Artificial thymic organoids represent a reliable tool to study T-cell differentiation in patients with severe T-cell lymphopenia. Blood Adv 4, 2611-2616. 10.1182/bloodadvances.2020001730.
9. Montel-Hagen, A., Sun, V., Casero, D., Tsai, S., Zampieri, A., Jackson, N., Li, S., Lopez, S., Zhu, Y., Chick, B., et al. (2020). In Vitro Recapitulation of Murine Thymopoiesis from Single Hematopoietic Stem Cells. Cell Rep 33. 10.1016/j.celrep.2020.108320.
10. Montel-Hagen, A., Tsai, S., Seet, C. S., and Crooks, G. M. (2022). Generation of Artificial Thymic Organoids from Human and Murine Hematopoietic Stem and Progenitor Cells. Curr Protoc 2. 10.1002/cpz1.403.
11. Montel-Hagen, A., Seet, C. S., Li, S., Chick, B., Zhu, Y., Chang, P., Tsai, S., Sun, V., Lopez, S., Chen, H. C., et al. (2019). Organoid-Induced Differentiation of Conventional T Cells from Human Pluripotent Stem Cells. Cell Stem Cell 24, 376-389.e8. 10.1016/j.stem.2018.12.011.
12. DeWitt, M. A., Magis, W., Bray, N. L., Wang, T., Berman, J. R., Urbinati, F., Heo, S. J., Mitros, T., Munoz, D. P., Boffelli, D., et al. (2016). Selection-free genome editing of the sickle mutation in human adult hematopoietic stem/progenitor cells. Sci Transl Med 8. 10.1126/scitranslmed.aaf9336.
13. Chu, S. H., Packer, M., Rees, H., Lam, D., Yu, Y., Marshall, J., Cheng, L. I., Lam, D., Olins, J., Ran, F. A., et al. (2021). Rationally Designed Base Editors for Precise Editing of the Sickle Cell Disease Mutation. CRISPR Journal 4, 169-177. 10.1089/crispr.2020.0144.
14. Gehrke, J. M., Cervantes, O., Clement, M. K., Wu, Y., Zeng, J., Bauer, D. E., Pinello, L., and Joung, J. K. (2018). An apobec3a-cas9 base editor with minimized bystander and off-target activities. Nat Biotechnol 36, 977. 10.1038/nbt.4199.
15. Joseph, N., Reicher, B., and Barda-Saad, M. (2014). The calcium feedback loop and T cell activation: How cytoskeleton networks control intracellular calcium flux. Biochim Biophys Acta Biomembr 1838, 557-568. 10.1016/j.bbamem.2013.07.009.
16. Leibowitz, M. L., Papathanasiou, S., Doerfler, P. A., Blaine, L. J., Sun, L., Yao, Y., Zhang, C. Z., Weiss, M. J., and Pellman, D. (2021). Chromothripsis as an on-target consequence of CRISPR-Cas9 genome editing. Nat Genet 53, 895-905. 10.1038/s41588-021-00838-7.
17. Wang, W., Tang, G., Cortes, J. E., Liu, H., Ai, D., Yin, C. C., Li, S., Khoury, J. D., Bueso-Ramos, C., Medeiros, L. J., et al. (2015). Chromosomal rearrangement involving 11q23 locus in chronic myelogenous leukemia: A rare phenomenon frequently associated with disease progression and poor prognosis. J Hematol Oncol 8. 10.1186/s13045-015-0128-2.
18. Baer, M. R., Stewart, C. C., Lawrence, D., Arthur, D. C., Mro Zek, M., Strout, M. P., Davey, F. R., and Bloomfield, C. D. (1998). Acute myeloid leukemia with 11q23 translocations: myelomonocytic immunophenotype by multiparameter flow cytometry.
19. Rees, H. A., and Liu, D. R. (2018). Base editing: precision chemistry on the genome and transcriptome of living cells. Nat Rev Genet 19, 770-788. 10.1038/s41576-018-0059-1.
20. Richter, M. F., Zhao, K. T., Eton, E., Lapinaite, A., Newby, G. A., Thuronyi, B. W., Wilson, C., Koblan, L. W., Zeng, J., Bauer, D. E., et al. (2020). Phage-assisted evolution of an adenine base editor with improved Cas domain compatibility and activity. Nat Biotechnol 38, 883-891. 10.1038/s41587-020-0453-z.
21. Tsai, S. Q., Nguyen, N. T., Malagon-Lopez, J., Topkar, V. v., Aryee, M. J., and Joung, J. K. (2017). CIRCLE-seq: A highly sensitive in vitro screen for genome-wide CRISPR-Cas9 nuclease off-targets. Nat Methods 14, 607-614. 10.1038/nmeth.4278.
22. Tsai, S. Q., Zheng, Z., Nguyen, N. T., Liebers, M., Topkar, V. v., Thapar, V., Wyvekens, N., Khayter, C., Iafrate, A. J., Le, L. P., et al. (2015). GUIDE-seq enables genome-wide profiling of off-target cleavage by CRISPR-Cas nucleases. Nat Biotechnol 33, 187-198. 10.1038/nbt.3117.
23. Bae, S., Park, J., and Kim, J. S. (2014). Cas-OFFinder: A fast and versatile algorithm that searches for potential off-target sites of Cas9 RNA-guided endonucleases. Bioinformatics 30, 1473-1475. 10.1093/bioinformatics/btu048.
24. Collias, D., and Beisel, C. L. (2021). CRISPR technologies and the search for the PAM-free nuclease. Nat Commun 12. 10.1038/s41467-020-20633-y.
25. McIntosh, B. E., Brown, M. E., Duffin, B. M., Maufort, J. P., Vereide, D. T., Slukvin, I. I., and Thomson, J. A. (2015). Nonirradiated NOD, B6.SCID I12iy−/−kitW41/W41 (NBSGW) mice support multilineage engraftment of human hematopoietic cells. Stem Cell Reports 4, 171-180. 10.1016/j.stemcr.2014.12.005.
26. Hess, N. J., Lindner, P. N., Vazquez, J., Grindel, S., Hudson, A. W., Stanic, A. K., Ikeda, A., Hematti, P., and Gumperz, J. E. (2020). Different Human Immune Lineage Compositions Are Generated in Non-Conditioned NBSGW Mice Depending on HSPC Source. Front Immunol 11. 10.3389/fimmu.2020.573406.
27. Hutt, D. (2018). Engraftment, Graft Failure, and Rejection. In The European Blood and Marrow Transplantation Textbook for Nurses (Springer International Publishing), pp. 259-270. 10.1007/978-3-319-50026-313.
28. Seet, C. S., He, C., Bethune, M. T., Li, S., Chick, B., Gschweng, E. H., Zhu, Y., Kim, K., Kohn, D. B., Baltimore, D., et al. (2017). Generation of mature T cells from human hematopoietic stem and progenitor cells in artificial thymic organoids. Nat Methods 14, 521-530. 10.1038/nmeth.4237.
29. de Saint Basile, G., Geissmann, F., Flori, E., Uring-Lambert, B., Soudais, C., Cavazzana-Calvo, M., Durandy, A., Jabado, N., Fischer, A., and le Deist, F. (2004). Severe combined immunodeficiency caused by deficiency in either the 8 or the s subunit of CD3. Journal of Clinical Investigation 114, 1512-1517. 10.1172/JCI200422588.
30. Recio, M. J., Moreno-Pelayo, M. A., Kili?, S. S., Guardo, A. C., Sanal, O., Allende, L. M., Perez-Flores, V., Menca, A., Modamio-H0ybj0r, S., Seoane, E., et al. (2007). Differential Biological Role of CD3 Chains Revealed by Human Immunodeficiencies. The Journal of Immunology 178, 2556-2564. 10.4049/jimmunol.178.4.2556.
31. Stoeckius, M., Hafemeister, C., Stephenson, W., Houck-Loomis, B., Chattopadhyay, P. K., Swerdlow, H., Satija, R., and Smibert, P. (2017). Simultaneous epitope and transcriptome measurement in single cells. Nat Methods 14, 865-868. 10.1038/nmeth.4380.
32. Butler, A., Hoffman, P., Smibert, P., Papalexi, E., and Satija, R. (2018). Integrating single-cell transcriptomic data across different conditions, technologies, and species. Nat Biotechnol 36, 411-420. 10.1038/nbt.4096.
33. Davis, M. M., and Bjorkman, P. J. (1988). T-cell antigen receptor genes and T-cell recognition. Nature 334.
34. Taghon, T., and Rothenberg, E. v. (2008). Molecular mechanisms that control mouse and human TCR-a0 and TCR-Y8 T cell development. Semin Immunopathol 30, 383-398. 10.1007/s00281-008-0134-3.
35. Anderson, A. C., Joller, N., and Kuchroo, V. K. (2016). Lag-3, Tim-3, and TIGIT: Co-inhibitory Receptors with Specialized Functions in Immune Regulation. Immunity 44, 989-1004. 10.1016/j.immuni.2016.05.001.
36. Sakuishi, K., Apetoh, L., Sullivan, J. M., Blazar, B. R., Kuchroo, V. K., and Anderson, A. C. (2010). Targeting Tim-3 and PD-1 pathways to reverse T cell exhaustion and restore anti-tumor immunity. Journal of Experimental Medicine 207, 2187-2194.

10.1084/jem.20100643.
37. Mclane, L. M., Abdel-Hakeem, M. S., and Wherry, E. J. (2019). CD8 T Cell Exhaustion During Chronic Viral Infection and Cancer. 10.1146/annurev-immunol-041015.
38. Chao, A. (1984). Board of the Foundation of the Scandinavian Journal of Statistics Nonparametric Estimation of the Number of Classes in a Population Nonparametric Estimation of the Number of Classes in a Population.
39. Siegner, S. M., Ugalde, L., Clemens, A., Garcia-Garcia, L., Bueren, J. A., Rio, P., Karasu, M. E., and Corn, J. E. (2022). Adenine base editing efficiently restores the function of Fanconi anemia hematopoietic stem and progenitor cells. Nat Commun 13, 6900. 10.1038/s41467-022-34479-z.
40. Koblan, L. W., Erdos, M. R., Wilson, C., Cabral, W. A., Levy, J. M., Xiong, Z. M., Tavarez, U. L., Davison, L. M., Gete, Y. G., Mao, X., et al. (2021). In vivo base editing rescues Hutchinson-Gilford progeria syndrome in mice. Nature 589, 608-614. 10.1038/s41586-020-03086-7.
41. Miller, S. M., Wang, T., Randolph, P. B., Arbab, M., Shen, M. W., Huang, T. P., Matuszek, Z., Newby, G. A., Rees, H. A., and Liu, D. R. (2020). Continuous evolution of SpCas9 variants compatible with non-G PAMs. Nat Biotechnol 38, 471-481. 10.1038/s41587-020-0412-8.
42. Bejar, R., Levine, R., and Ebert, B. L. (2011). Unraveling the molecular pathophysiology of myelodysplastic syndromes. Journal of Clinical Oncology 29, 504-515. 10.1200/JCO.2010.31.1175.
43. Gil, J., Busto, E. M., Garcillan, B., Chean, C., Garcia-Rodriguez, M. C., Daz-Alderete, A., Navarro, J., Reine, J., Menca, A., Gurbindo, D., et al. (2011). A leaky mutation in CD3D differentially affects a0 and 78 T cells and leads to a Tap-TY § +B+NK+ human SCID. Journal of Clinical Investigation 121, 3872-3876. 10.1172/JCI44254.
44. Garcillan, B. M., Mazariegos M S, M. S., Fisch, P., Res, P. C., Munoz-Ruiz, M. M., Gil, J., Lopez-Granados, E., Fernandez-Malave, E., and Regueiro, J. R. (2014). Enrichment of the rare CD4+&gamma;&delta; T-cell subset in patients with atypical CD3&delta; deficiency. 10.1016/j.jaci.2013.09.
45. Dave, V. P., Cao, Z., Browne, C., Alarcon, B., Fernandez-Miguel, G., Lafaille, J., de la Hera, A., Tonegawa, S., and Kappes, D. J. (1997). CD38 deficiency arrests development of the ap but not the 78 T cell lineage. EMBO J Vol. 16, 1360-1370.
46. Okada, S., Markle, J. G., Deenick, E. K., Mele, F., Averbuch, D., Lagos, M., Alzahrani, M., Al-Muhsen, S., Halwani, R., Ma, C. S., et al. (2015). Impairment of immunity to Candida and Mycobacterium in humans with bi-allelic RORC mutations. Science (1979) 349, 606-613. 10.1126/science.aaa4282.
47. Sun, Z., Unutmaz, D., Zou, Y. R., Sunshine, M. J., Pierani, A., Brenner-Morton, S., Mebius, R. E., and Littman, D. R. Requirement for ROR in Thymocyte Survival and Lymphoid Organ Development.
48. Ligons, D. L., Hwang, S. J., Waickman, A. T., Park, J. Y., Luckey, M. A., and Park, J. H. (2018). RORt limits the amount of the cytokine receptor c through the prosurvival factor Bcl-xL in developing thymocytes. Sci Signal 11. 10.1126/scisignal.aam8939.
49. Benitez, E. K., Lomova Kaufman, A., Cervantes, L., Clark, D. N., Ayoub, P. G., Senadheera, S., Osborne, K., Sanchez, J. M., Crisostomo, R. V., Wang, X., et al. (2020). Global and Local Manipulation of DNA Repair Mechanisms to Alter Site-Specific Gene Editing Outcomes in Hematopoietic Stem Cells. Front Genome Ed 2. 10.3389/fgeed.2020.601541.
50. Hoban, M. D., Lumaquin, D., Kuo, C. Y., Romero, Z., Long, J., Ho, M., Young, C. S., Mojadidi, M., Fitz-Gibbon, S., Cooper, A. R., et al. (2016). CRISPR/Cas9-mediated correction of the sickle mutation in human CD34+ cells. Molecular Therapy 24, 1561-1569. 10.1038/mt.2016.148.
51. Lomova, A., Clark, D. N., Campo-Fernandez, B., Flores-Bjurstrom, C., Kaufman, M. L., Fitz-Gibbon, S., Wang, X., Miyahira, E. Y., Brown, D., DeWitt, M. A., et al. (2019). Improving Gene Editing Outcomes in Human Hematopoietic Stem and Progenitor Cells by Temporal Control of DNA Repair. Stem Cells 37, 284-294. 10.1002/stem.2935.
52. Camelia Botnar Laboratories, F. C. C. F. Calcium Flux on LSRII Background.
53. Grynkiewicz, G., Poenie, M., and Tsien, R. Y. (1985). A new generation of Ca2+ indicators with greatly improved fluorescence properties. Journal of Biological Chemistry 260, 3440-3450. 10.1016/s0021-9258 (19) 83641-4.
54. Bannwarth, M., Correa, I. R., Sztretye, M., Pouvreau, S., Fella, C., AnninaAebischer, Royer, L., Ros, E., and Johnsson, K. (2009). Indo-1 derivatives for local calcium sensing. ACS Chem Biol 4, 179-190. 10.1021/cb800258g.
55. Morgan, A. J., and Jacob, R. (1994). lonomycin enhances Ca2+ influx by stimulating store-regulated cation entry and not by a direct action at the plasma membrane.
56. Kuo, C. Y., Long, J. D., Campo-Fernandez, B., de Oliveira, S., Cooper, A. R., Romero, Z., Hoban, M. D., Joglekar, A. v., Lill, G. R., Kaufman, M. L., et al. (2018). Site-Specific Gene Editing of Human Hematopoietic Stem Cells for X-Linked Hyper-IgM Syndrome. Cell Rep 23, 2606-2616. 10.1016/j.celrep.2018.04.103.
57. Malinin, N. L., Lee, G. H., Lazzarotto, C. R., Li, Y., Zheng, Z., Nguyen, N. T., Liebers, M., Topkar, V. v., Iafrate, A. J., Le, L. P., et al. (2021). Defining genome-wide CRISPR-Cas genome-editing nuclease activity with GUIDE-seq. Nat Protoc 16, 5592-5615. 10.1038/s41596-021-00626-x.
58. Logan, A. C., Nightingale, S. J., Haas, D. L., Cho, G. J., Pepper, K. A., and Kohn, D. B. (2004). Factors influencing the titer and infectivity of lentiviral vectors. Hum Gene Ther 15, 976-988. 10.1089/hum.2004.15.976.
59. Cooper, A. R., Patel, S., Senadheera, S., Plath, K., Kohn, D. B., and Hollis, R. P. (2011). Highly efficient large-scale lentiviral vector concentration by tandem tangential flow filtration. J Virol Methods 177, 1-9. 10.1016/j.jviromet.2011.06.019.
60. Masiuk, K. E., Laborada, J., Roncarolo, M. G., Hollis, R. P., and Kohn, D. B. (2019). Lentiviral Gene Therapy in HSCs Restores Lineage-Specific Foxp3 Expression and Suppresses Autoimmunity in a Mouse Model of IPEX Syndrome. Cell Stem Cell 24, 309-317.e7. 10.1016/j.stem.2018.12.003.
61. San Jose, E., Borroto, A., Niedergang, F., Alcover, A., and Alarco, B. (2000). Triggering the TCR Complex Causes the Downregulation of Nonengaged Receptors by a Signal Transduction-Dependent Mechanism.
62. Gu, Z., Gu, L., Eils, R., Schlesner, M., and Brors, B. (2014). Circlize implements and enhances circular visualization in R. Bioinformatics 30, 2811-2812. 10.1093/bioinformatics/btu393.
63. Finak, G., McDavid, A., Yajima, M., Deng, J., Gersuk, V., Shalek, A. K., Slichter, C. K., Miller, H. W., McElrath, M. J., Prlic, M., et al. (2015). MAST: A flexible statistical framework for assessing transcriptional changes and characterizing heterogeneity in single-cell RNA sequencing data. Genome Biol 16. 10.1186/s13059-015-0844-5.
64. Takada, H., Nomura, A., Roifman, C. M., and Hara, T. (2005). Severe combined immunodeficiency caused by a splicing abnormality of the CD38 gene. Eur J Pediatr 164, 311-314. 10.1007/s00431-005-1639-6.
65. Blighe, K., Rana, S., Turkes, E., Ostendorf, B., Grioni, A., and Lewis, M. R topics documented.
66. Korotkevich, G., Sukhov, V., Budin, N., Shpak, B., Artyomov, M. N., and Sergushichev, A. Fast gene set enrichment analysis. 10.1101/060012.
67. Dolgalev, I. (2005). MSiMSigDB Gene Sets for Multiple Organisms in a Tidy Data FormatgDB Gene Sets for Multiple Organisms in a Tidy Data Format. Proc Natl Acad Sci USA 102, 15545-15550. 10.1073/pnas.0506580102.
68. Yu, G. (2022). Visualization of Functional Enrichment Result. R package version 1.16.2.

### Materials and Methods

### Jurkat T Cell Culturing and Editing

Wildtype Jurkat and K562 cells were obtained from ATCC (Manassas, VA). Cells were maintained in R10 (RPMI [GIBCO]/10% FBS [GIBCO]/1× Penicillin/Streptomycin/Glutamine [PSG, Gemini Bio Products; Sacramento, CA]) at 37° C. with 5% CO₂.

### Generating CD3D (C202T) Jurkat T Cell Line

Jurkat T cells were modified to contain the pathogenic CD3δ SCID allele by electroporation of SpCas9 recombinant protein (QB3 Macrolab, UC Berkeley; Berkeley, CA) complexed to sgRNA (5′-CGAGGAATATATAGGTGTAA-3′, SEQ ID NO:1095) (Synthego; Redwood City, CA) and ssODN homologous donor (5′-ACCCAAAGGGTTCAGGAAGCA CGTACTTCGATAATGAACTTGCACGGTAGATTCTTTG TCCTTGTATATATC TGTCCCATTACATCTATATATTCCTCATGGGTCCAGGATGCGTTT TCCCAGGTC-3′, SEQ ID NO:1096) (Integrated DNA Technologies {IDT}; Coraville, IA) carrying the pathogenic mutation and FACS single-cell sorted and cultured in R20 (RPMI [GIBCO]/20% FBS [GIBCO]/1× Penicillin/Streptomycin/Glutamine [PSG, Gemini Bio Products]). Primers for amplification of the CD3D locus to confirm knock-in of the pathogenic mutation were CD3DF: 5′-CTTGGTGCAGATCAAAGAGC-3′ (SEQ ID NO:1097); CD3DR: 5′-CTGGTGATGGGCTTGCCAC-3′ (SEQ ID NO:1098). A pseudo-tetraploid clonal cell line containing the CD3δ SCID mutation in ¼ CD3D alleles and deleterious indels in ¾ CD3D alleles (measured by HTS) was established (‘CD3D (C202T) Jurkat T cells’). Absence of CD3 surface expression was confirmed by flow cytometry (CD3-APC-Cy7, SK1, BioLegend; San Diego, CA). Cells were cultured in R10 at 37° C. with 5% CO₂.

### Cloning of Adenine Base Editor Variant Plasmids

pCMV-ABE8e-NG (Plasmid #138491) and pCMV-ABEmax-NRTH (Plasmid #136922) plasmids were obtained from AddGene (Watertown, MA). We generated all base editor variants derived from the same parental pCMV-ABE8e-NG backbone. Key substitutions were introduced to Cas9n genes to allow for alternative PAM recognition (other than canonical NGG). Substitutions were introduced by Q5 site-directed mutagenesis (New England Biolabs {NEB}, Ipswich, MA) and were as follows (relative to NGG-recognizing Cas9n): 1) ABE8e-VRER: D1135V, G1218R, R1335E, and T1337R, 2) ABE8e-xCas9 (3.7) A262T, R324L, S409I, E480K, E543D, M694I, and E1219V. To generate plasmid encoding ABE8e-NRTH, we utilized Gibson Assembly (NEB) cloning to amplify and ligate the ABE8e deaminase gene and Cas9n-NRTH gene.

### CD3D (C202T) Jurkat T Cell Electroporation

CD3D (C202T) Jurkat T cells were electroporated at ˜85% confluency. Cells were counted on ViCell (Beckman Coulter; Brea, CA) and 5×10⁵ cells per condition were centrifuged at 90×g for 15 min at RT, resuspended in 20 μL of SE electroporation buffer (Lonza; Basel, Switzerland), and combined with 1 μg sgRNA and 3 μg of BE expression plasmids. In the case of CRISPR/Cas9-HDR, 200 pmol of sgRNA were combined with 100 pmol of rCas9 nuclease protein for 15 minutes at RT for RNP complex formation. Cells were resuspended in 20 μL of SE electroporation buffer and combined with RNP and 250 pmol of ssODN ultramer donor (5′-TGCAATACCAGCATCACATGGGTAGAGGGAAC GGTGGGAACAC TGCTCTCAGACATT ACAAGACTGGACCTGGGAA AACGCATCCTGGATCCACGAGGAATATATAGATGTAAT GGGACAGATATA-3′, SEQ ID NO:1099). The underlined base represents the target site. Cells were electroporated using the CL-120 setting on the Amaxa 4D Nucleofector X Unit (Lonza). As previously described,⁴⁹ immediately after electroporation, cells were rested in 16-well electroporation strips (Lonza) for 10 min at RT and then recovered with 480 μL of R20 medium. In the case of CRISPR/Cas9-HDR, cells were recovered in 480 μL of R20 medium supplemented with 1.2 pmol of Alt-R HDR Enhancer and washed with phosphate-buffered saline (PBS) 24 hours later according to the manufacturer's instructions (Integrated DNA Technologies {IDT}; Coraville, IA). Editing outcomes were measured by HTS, 5 days after electroporation from gDNA extracted using PureLink Genomic DNA Mini Kit (Invitrogen/ThermoFisher Scientific; Waltham MA).

### Karyotype

24 hours post-electroporation, CD3D (C202T) Jurkat T cells treated with RNP+ssODN (CRISPR/Cas9-edited) or plasmids encoding ABEmax-NRTH and CD3D-localizing sgRNA were exposed to mitotic arresting agents to collect metaphases and harvested for G-banded karyotype analysis adhering to standard cytogenetics procedures (UCLA Cytogenetics Laboratory, Los Angeles CA). Twenty cells were analyzed per experimental condition. Composite karyotype nomenclature (not all indicated abnormalities were identified in all abnormal cells analyzed) was used to describe the abnormal clones according to the International System for Human Cytogenomic Nomenclature (ISCN).

### Illumina MiSeq Library Preparation for the CD3D Locus in CD3D (C202T) Jurkat T Cells and CD34+CD3δ SCID HSPCS

DNA libraries for HTS were prepared as previously described.^{50,51} Five days after editing, an outer PCR was performed on genomic DNA to amplify 608 bp of the CD3D locus using CD3DF: 5′-CTTGGTGCAGATCAAAGAGC-3′ (SEQ ID NO:1100); CD3DR: 5′-CTGGTGATGGGCTTGCCAC-3′ (SEQ ID NO:1101). A second PCR was performed to add a unique index to the PCR product of each sample; CD3D_LibF: 5′-ACACGACG CTCTTCCGA TCTNNNN GAGGACAGAGTGTTTGTGAA-3′ (SEQ ID NO:1102); CD3D_LibR 5′-GTGACTGGAGTTCAGACGTGTGCTCTTCCGATCTC TCTAGCCAGAAAGTTCTCAC-3′ (SEQ ID NO:1103). Underlined sequences represent Illumina adapter sequences. Following Illumina barcoding, PCR products were pooled at equal concentrations. The pooled library was purified twice using AMPure XP beads (Beckman Coulter; Brea, CA) and then quantified using ddPCR (QX 200; Bio-Rad Laboratories Inc.; Hercules, CA). HTS was performed at the UCLA Technology Center for Genomics & Bioinformatics (TCGB) using an Illumina MiSeq instrument 2×150 paired-end reads (Illumina; San Diego, CA). The sequences for all HSPC editing experiments were deposited to NCBI Sequence Read Archive.

### Calcium Flux Assay

As previously described,⁵² cells were suspended at 106/mL in cell loading medium (CLM; RPMI, 2% BSA, 25 mM HEPES (pH 7.4)). Cells were stained at a 1.5-5 uM concentration with cell permeable Indo-1 acetoxymethyl ester (AM) (ThermoFisher Scientific; Waltham, MA). Cells were incubated for 50 min at 37° C. in the dark and then washed 2× with CLM. Cells were gently resuspended by pipetting in CLM at 1×10⁶/mL and samples were protected from light until flow cytometric analysis. Individual samples were warmed at 37° C. in the dark for 10 min prior to analysis. A baseline Ca²⁺ ratio was recorded for 60 seconds after which purified NA/LE mouse anti-human CD3 (HIT3a) and purified NA/LE mouse anti-human CD28 (CD28.2) antibodies were added to stimulate cells (10 pg and 30 μg of each antibody for stimulating Jurkat T cells and ATO-derived thymocytes, respectively) (BD Biosciences; Franklin Lakes NJ). Intracellular esterases cleave Indo-1 AM, producing non-cell permeable Indo-1, a high affinity calcium indicator. Once excited by UV light, the emission spectrum of Indo-1 changes from blue (510 nm) to violet (420 nm) when bound to calcium, allowing for ratiometric measurements of calcium flux. 53 The stimulus was added 60 seconds after a baseline ratio was recorded.⁵⁴ Ionomycin (Imy), a calcium ionophore which rapidly increases intracellular calcium concentration by releasing calcium from its intracellular stores and facilitating transport of calcium across the plasma membrane, was used as a positive control.⁵⁵

ABE mRNA

ABE8e-NG and ABEmax-NRTH template plasmids were cloned via USER cloning to encode a dT7 promoter¹³ followed by a 5′ UTR, Kozak sequence, ORF, and 3′UTR. BE portions of the template plasmids were PCR amplified using Q5 Hot Start Mastermix (NEB) and PCR products were purified using QiaQuick PCR Purification Kit (Qiagen Inc., Valencia CA). ABE8e-NG and ABEmax-NRTH mRNA were in vitro transcribed according to manufacturer's guidelines from the purified PCR product using T7 HiScribe Kit (NEB) with full substitution of N1-methylpseudouridine for uridine and co-transcriptional 5′ capping using CleanCap AG analogue (TriLink Biotechnologies; San Diego, CA). Lastly, mRNA was purified according to manufacturer's instructions using LiCl Precipitation Solution (Thermo Fisher). Resulting mRNA was run on the Agilent Bioanalyzer to confirm mRNA integrity and identity.

### Colony-Forming Unit Assay

CFU assays were performed as previously described⁵⁶ using Methocult H4435 Enriched Methylcellulose (StemCell Technologies; Vancouver, Canada. Cat. #04445) according to the manufacturer's instructions with minor modifications. Briefly, 100, 300, and 900 CD34+ PBSCs were plated in duplicates into 35 mm gridded cell culture dishes. After 14 days of culture at 5% CO₂, 37° C. and humidified atmosphere, mature colonies were counted and identified based on their specific morphology. CFUs were then plucked for genomic DNA isolation (NucleoSpin Tissue XS, Clontech Laboratories Inc.; Mountain View, CA).

### CIRCLE-Seq Off-Target Editing Analysis

CIRCLE-Seq off-target editing analysis was performed as previously described. 5 Genomic DNA from HEK293T cells was isolated using Gentra Puregene Kit (Qiagen; Hilden, Germany) according to the manufacturer's instructions. Purified genomic DNA was sheared with a Covaris S2 instrument to an average length of 300 bp. The fragmented DNA was end repaired, A-tailed, and ligated to a uracil-containing stem-loop adaptor, using the KAPA HTP Library Preparation Kit, PCR Free (KAPA Biosystems; Wilmington MA). Adaptor-ligated DNA was treated with Lambda Exonuclease (NEB) and Escherichia coli Exonuclease I (NEB) and then with USER enzyme (NEB) and T4 poly-nucleotide kinase (NEB). Intramolecular circularization of the DNA was performed with T4 DNA ligase (NEB) and residual linear DNA was degraded by Plasmid-Safe ATP-dependent DNase (Lucigen; Middleton WI). In vitro cleavage reactions were performed with 250 ng Plasmid-Safe-treated circularized DNA, 90 nM Cas9-NRTH protein, Cas9 nuclease buffer (NEB) and 90 nM synthetic chemically modified sgRNA (Synthego; Redwood City, CA), in a 100-pl volume. Cleaved products were A-tailed, ligated with a hairpin adaptor (NEB), treated with USER enzyme (NEB) and amplified by PCR with barcoded universal primers (NEBNext Multiplex Oligos for Illumina (NEB)), using Kapa HiFi Polymerase (KAPA Biosystems). Libraries were sequenced with 150-bp paired-end reads with an Illumina MiSeq instrument. CIRCLE-seq data analyses were performed using open-source CIRCLE-seq analysis software and default recommended parameters (//github.com/tsailabSJ/circleseq).

### Generating CD3D (C202T) K562 Cell Line

K562 cells were modified to contain the pathogenic CD3δ SCID allele by electroporation of RNP and ssODN homologous donor (5′-ACCCAAAGGGTTCAGGA AGCACGTACTTCGATAATGAACTTGCACGGTAGATTCTTTG TCCTTGTATATATC TGTCCCATTACATCTATATATTCCTCATGGGTCCAGGATGCGTTT TCCCAGGTC-3′, SEQ ID NO:1104) carrying the pathogenic mutation were FACS single-cell sorted and cultured in R10. Primers for amplification of the CD3D locus to confirm knockin of the pathogenic mutation were CD3DF: 5′-CTTGGTGCAGATCAAAGAGC-3′ (SEQ ID NO: 1105); CD3DR: 5′-CTGGTGATGGGCTTGCCAC-3′ (SEQ ID NO:1106). A clonal cell line containing the CD3δ SCID mutation in all CD3D alleles (measured by HTS) was established (′CD3D (C202T) K562 cells). Cells were cultured in R10 at 37C with 5% O2.

### GUIDE-Seq Off-Target Editing Analysis

CD3D (C202T) K562 cells were electroporated with plasmids encoding CD3D-targeting sgRNA and ABEmax-NRTH and a DS oligo for capture at DSBs. Two weeks after electroporation, cells were harvested and genomic DNA was extracted to prepare a library for Illumina HTS as previously described.⁵⁷ In summary, genomic DNA was sonicated to an average size of 500 bp using a Bioruptor Pico Sonication Device (Diagenode; Liege, Belgium) and was 1× purified using AMPure XP beads (Beckman Coulter, Brea, CA). Purified product was then end-repaired and A-tailed (Fisher Scientific, Carlsbad, CA). Y-adapters were ligated using T4 DNA ligase (Fisher Scientific) according to manufacturer's instructions. The ligated product was purified using 0.9× volumes of AMPure XP beads and the adapter ligated product was split into two PCR reactions for sense and antisense reactions. Site specific PCR1 was performed using Platinum Taq polymerase (Fisher Scientific,) and the product was purified using 1.2× volumes of AMPure XP beads. The purified product was utilized as a template for a second PCR (PCR2) to add P7 Illumina indexes for sequencing. PCR2 product was quantified by densitometry and pooled at equal concentrations. The pooled library was purified using 0.7× volumes of AMPure XP beads and then quantified using ddPCR (QX 200). HTS was performed at UCLA Technology Center for Genomics & Bioinformatics (TCGB) using an Illumina MiSeq instrument 2×150 paired-end reads. The sequences for all HSPC editing experiments were deposited to NCBI Sequence Read Archive.

### CasOFFinder Off-Target Editing Analysis

Computational prediction of potential off-target sites with minimal mismatches relative to the intended target site (three or fewer mismatches overall, or two or fewer mismatches allowing G: U wobble base pairings with the guide RNA) was performed using CasOFFinder.²³

Multiplex-Targeted Sequencing by rhAMPseg

On- and off-target sites identified by CIRCLE-seq, GUIDE-seq, and CasOFFinder were amplified from genomic DNA from ABEmax-NRTH edited CD34+CD3δ SCID cells or unedited control CD3δ SCID cells using rhAMPSeq multiplexed library preparation (IDT), with amplification coordinates. Sequencing libraries were generated according to the manufacturer's instructions and sequenced with 150-bp paired-end reads using an Illumina NextSeq instrument.

### Quantification of Base Editing Efficiency at Off-Target Sites

The A⋅T-to-G⋅C editing frequency for each position in the protospacer was quantified as previously described5 using CRISPResso Pooled (v2.0.41) (//github.com/pinellolab/CRISPResso2) with quantification_window_size10, quantification_window_centre-10, base_editor_output, conversion_nuc_from A, conversion_nuc_to G. The genomic features of off-target sites were initially annotated using HOMER (v4.10) (//homer.ucsd.edu/homer/). Confirmed off-target sites were inspected manually and annotated using the NCBI Genome Data Browser. The editing frequency for each site was calculated as the ratio between the number of reads containing the edited base in a window from position 4 to 10 of each protospacer and the total number of reads. To calculate the statistical significance of off-target editing for the ABEmax-NRTH mRNA treatment compared to control samples, we applied a ×2 test for each of three samples (one donor, with three replicates). The 2×2 contingency table was constructed using the number of edited reads and the number of unedited reads in treated and untreated groups and the false discovery rate (FDR) was calculated using the Benjamini-Hochberg method as previously described.⁵ The code used to conduct off-target quantification and statistical analysis was customized from Newby et al. 2020 (//github.com/tsailabSJ/MKSR_off_targets).

### Lentiviral Vector Packaging, Titering, and Transduction

LVs are pCCL HIV-derived LVs of self-inactivating (SIN) LTR configurations. Construction of pCCL-MND-GFP has been described58 and wild-type CD3D cDNA, CD3D cDNA containing the A0 bystander edit, and CD3D cDNA containing the c.202C>T mutation were cloned into the multi-cloning site of the vector. The CCL-MND-CD3D LV was packaged in a VSV-G pseudotype using HEK293T cells and titered as previously described.⁵⁹

### Determination of Vector Copy Number (VCN) per Cell

Genomic DNA was extracted using PureLink Genomic DNA Mini Kit (Invitrogen/ThermoFisher Scientific). Average VCN was measured using ddPCR with primers and probes specific to the HIV-1 Psi region and normalized using primers to the autosomal human gene SDC4 ddPCR as previously described.⁶⁰

### Isolation and Culture of Healthy CD34+ Human HSPCs

Leukopaks from healthy donors were purchased from HemaCare (HemaCare BioResearch Products; Van Nuys, CA). Mobilized peripheral blood (mPB) was collected from normal, healthy donors on days 5 and 6 after 5 days of stimulation with granulocyte-colony stimulating factor (G-CSF) as described.⁵¹ Platelet depletion was performed from the centrifuged bags at each wash step using a plasma expressor extractor (Fenwal). CD34+ cell enrichment was performed using the CliniMACS Plus (Miltenyi; Bergish Gladbach, Germany). CD34+ cells were cryopreserved in CryoStor CS5 (StemCell Technologies; Vancouver, Canada) using a CryoMed controlled-rate freezer (Thermo Fisher Scientific).

ABEmax-NRTH mRNA Electroporation in Human HSPCs

Cells were pre-stimulated for two days in X-VIVO 15 medium (50 ng/ml each of hSCF, hFLT3-L, and hTPO) with 2×10⁵ cells per condition that were washed 2× and pelleted at 300× g for 8 min at RT. Cells were resuspended in electroporation buffer (P3 buffer) (Lonza) (CD3δ SCID cells) or, in the case of HD HSPCs for in vivo studies, EP Buffer (Maxcyte, Gaithersburg, MD), and combined with 1 μg of sgRNA and 4.5 pg of BE mRNA. Cells were electroporated using programs DS-130 (Lonza) or HSC-3 (ATX MaxCyte). Electroporated cells were recovered in the same medium at 37° C., 5% CO₂. 24 hours post-electroporation, samples of the cells were diluted 1:2 with trypan blue and counted manually using a hemocytometer to determine viability (number of live cells/number of total cells×100). Cells were re-plated into 1 mL (or 5 mL, for 1×10⁶ cells) of myeloid expansion medium (Iscove's Modified Dulbecco's Medium (IMDM, Thermo Fisher Scientific)+20% FBS [HI FBS, Gibco/ThermoFisher)+5 ng/mL Interleukin 3 (IL3), 10 ng/mL Interleukin 6 (IL6), 25 ng/mL SCF (Peprotech; Rocky Hill, NJ)], and cultured for 5 days prior to harvesting for genomic DNA (gDNA). gDNA was extracted using PureLink Genomic DNA Mini Kit (Invitrogen/ThermoFisher Scientific).

### Ethical Approval for Studies Involving Mice

The NOD, B6.SCID IL2rg−/−KitW41/W41 (NBSGW) murine xenografts were performed under an approved protocol (2008-167) by the UCLA Animal Research Committee (Jackson Laboratory; Bar Harbor, ME).

### In Vivo Studies

Animals were handled in laminar flow hoods and housed in a pathogen-free colony in a biocontainment vivarium. Adult females (5-7 weeks old) were injected with 5×10⁵-1×10⁶ cells/mouse via retro-orbital injection of untreated, LV-treated, or LV and BE human CD34+ cells, and allowed to engraft over 12-16 weeks. After 12-16 weeks, mice were sacrificed by CO2 inhalation followed by cervical dislocation. Bone marrow, thymus, and spleen were harvested for subsequent analysis of chimerism and cell lineage composition. Lineage distribution was measured using cell-type specific antibodies on the Fortessa flow cytometer (BD Biosciences) and sorted using an Aria H cell sorter (BD Biosciences). The antibodies used were: anti-human CD45 (BD Biosciences, Cat. No. 560367), anti-mouse CD45 (Biolegend, Cat. No. 103107), anti-human CD34 (Biolegend, Cat. No. 343607), anti-human CD19 (Biolegend, Cat. No. 302215), anti-human CD56 (BD Biosciences, Cat. No. 555516), anti-human CD3 (Biolegend, Cat. No. 344817), anti-human CD33 (Biolegend, Cat. No. 303423), anti-human CD4 (Biolegend, Cat. No. 300501), and anti-human CD8 (Biolegend, Cat. No. 980902).

### Patient Bone Marrow Collection

Bone-marrow cells were collected following local Research Ethics Board (REB) approval and informed parental consent (study ID #REB21-0375). Procedure was performed under general anesthetic at the same time as central line placement. Using sterile technique, 10 mL of bone marrow was aspirated from the right posterior superior iliac spine with a 16 gauge×2.688 inch bone marrow aspirate needle (Argon medical Devices, Inc). Specimen was anticoagulated with preservative free heparin (100 units/mL). The use of bone marrow samples from CD3δ SCID patients was approved under UCLA IRB #2010-001399.

CD34+ HSPC Isolation from Patient Bone Marrow

CD34+ cells were isolated using microbeads conjugated to monoclonal mouse anti-human CD34 antibodies (Milteny Biotech CD34 MicroBead Kit. Cat #130-046-702) according to manufacturer's instructions. Briefly, mononuclear cells (MNC) obtained from patient bone marrow were isolated using Ficoll-Paque (Sigma) gradient centrifugation according to established methods. A total of 10⁸ cells were collected, washed with sterile phosphate-buffered saline (PBS) to remove platelets and re-suspended in MACS buffer (PBS, pH 7.2, 0.5% bovine serum albumin [BSA], and 2 mM EDTA). To the cell pellet (10⁸ cells), 100 l of FcR blocking reagent and 100 l of CD34 microbeads were added to the cell pellet, mixed well and incubated at 40C for 30 minutes. Cells were then washed with 10 ml of MACS buffer by centrifugation at 300 g for 10 minutes and re-suspended in in 500 l of the same buffer and loaded onto a prepared MACS column placed in a magnetic field. Flow through cell fraction (CD34 negative population) was collected. The column was then washed and removed from the magnet, placed on a collection tube and the bound cells were eluted using a plunger. The collected CD34+ cell fraction was then washed, viability checked and re-suspended in 1 ml of MACS buffer containing 10% DMSO and stored frozen in liquid nitrogen until processing. For transportation, cells in freezer vials were shipped by overnight courier in containers with excess dry ice.

### Bone Marrow Artificial Thymic Organoid (ATO) Cultures

Bone Marrow ATOs were generated as previously described. 28 MS5-hDLL4 cells were harvested by trypsinization and resuspended in serum free ATO culture medium (“RB27”) composed of RPMI 1640 (Corning, Manassas, VA), 4% B27 supplement (ThermoFisher Scientific, Grand Island, NY), 30 pM L-ascorbic acid 2-phosphate sesquimagnesium salt hydrate (Sigma-Aldrich, St. Louis, MO) reconstituted in PBS, 1% penicillin/streptomycin (Gemini Bio-Products, West Sacramento, CA), 2% Glutamax (ThermoFisher Scientific, Grand Island, NY), 5 ng/ml rhFLT3L and 2.5 ng/ml rhIL-7 (Peprotech, Rocky Hill, NJ). RB27 was made fresh weekly. 1.5×10⁵ MS5-hDLL1 cells were combined with 1.5×10³ CD34+ cells per ATO in 1.5 ml Eppendorf tubes (up to 12 ATOs per tube) and centrifuged at 300 g for 5 min at 4° C. in a swinging bucket centrifuge. Supernatants were carefully removed, and the cell pellet was resuspended in 6 pl RB27 per ATO and mixed by brief vortexing. ATOs were plated on a 0.4 pm Millicell transwell insert (EMD Millipore, Billerica, MA; Cat. PICM0RG50) placed in a 6-well plate containing 1 ml RB27 per well. Medium was changed completely every 3-4 days by aspiration from around the cell insert followed by replacement with 1 ml with fresh RB27/cytokines. ATO cells were harvested by adding FACS buffer (PBS/0.5% bovine serum album/2 mM EDTA) to each well and briefly disaggregating the ATO by pipetting with a 1 ml “P1000” pipet, followed by passage through a 50 pm nylon strainer.

### T Cell Cytokine Assays

ATOs were harvested at week 12 (as above) and resuspended in 48-well plates in 1 ml AIM V (ThermoFisher Scientific, Grand Island, NY) with 5% human AB serum (Gemini Bio-Products, West Sacramento, CA) at a concentration of 1×10⁶ cells/ml anti-CD3/CD28 beads (ThermoFisher Scientific, Grand Island, NY) in AIM V/5% human AB serum with 20 IU/ml rhIL-2 (Peprotech, Rocky Hill, NJ), were added to cells for 24 hours. Because anti-CD3/CD28 bead stimulation is known to down-regulate surface CD3 and TCRαβ expression,⁶¹ mature SP8s T cells are defined as CD45+CD8+CD4−CD45RA+. Cells were stained for CD3, TCRαβ, CD45, CD4, CD8, CD45RA, and Zombie Aqua fixable viability dye (Biolegend, San Diego, CA) prior to fixation and permeabilization with an intracellular staining buffer kit (eBioscience, San Diego, CA) and intracellular staining with antibodies against IFNγ, TNFα, and IL-2 (Biolegend, San Diego, CA).

### T Cell Proliferation Assays

For CFSE proliferation assays, at least 100,000 ATO-derived CD8SP T cells were isolated by negative selection MACS using CD8+ T cell Isolation Kit, human (Miltenyi, Cat. 130-09-495) and labeled with 5 pM CFSE (Biolegend, San Diego, CA). Labeled cells were incubated with anti-CD3/CD28 beads (ThermoFisher Scientific, Grand Island, NY) in AIM V/5% human AB serum with 20 IU/ml rhIL-2 (Peprotech, Rocky Hill, NJ), co-stained for CD25 and 4-1BB (Biolegend, San Diego, CA) and analyzed by flow cytometry on day 5.

### Flow Cytometry and Antibodies

All flow cytometry stains were performed in PBS/0.5% BSA/2 mM EDTA for 30 min on ice. FcX (Biolegend, San Diego, CA) was added to all samples during antibody staining. DAPI or Zombie Aqua fixable viability dye (Biolegend, San Diego, CA) was added to all samples prior to analysis. Analysis was performed on an LSRII Fortessa, and FACS on an ARIA or ARIA-H instrument (BD Biosciences, San Jose, CA) at the UCLA Broad Stem Cell Research Center Flow Cytometry Core. For all analyses DAPI+ or Zombie Aqua+ cells were gated out, and single cells were gated based on FSC-H vs. FSC-W. Antibody clones used for surface and intracellular staining were obtained from Biolegend (San Diego, CA): CD3 (UCHT1), CD4 (RPA-T4), CD5 (UCHT2), CD7 (CD7-6B7), CD8a (SK1), CD14 (M5E2), CD25 (BC96), CD27 (O323), CD28 (CD28.2), CD34 (581), CD45 (HI30), CD45RA (HI100), CD45RO (UCHL1), CD56 (HCD56), CD62L (DREG-56) CCR7 (G043H7), CTLA-4 (BNI3), IFNg (4S.B3), IL-2 (MQ1-17H12), LAG3 (11-C3C65), PD-1 (EH12.2H7), TCRαβ (IP26), TCRγδ (B1), TIM-3 (F38-2E2), TNFα (Mab11); and Miltenyi (Auburn, CA): CD8b (REA-715).

scRNA-Seq and CITE-Seq Library Preparation and Sequencing

ATOs were harvested at week 8 (as above) and subjected to MACs Dead Cell Removal Kit (Miltenyi, Cat. 130-090-101), and ˜5×10⁵ cells were stained with TotalSeq-C Human Universal Cocktail, V1.0 (Biolegend, Cat. 399905) per the manufacturer's protocol. Labeled cells were submitted to the UCLA Technology Center for Bioinformatics and Genomics for unique molecular identifier (UMI) tagging and generation of gene expression (GEX), human TCR repertoire (VDJ), and Feature Barcoding libraries using the 10× Chromium Next GEM Single Cell 5′ Kit v2 (10× Genomics, Pleasanton, CA). Fully constructed libraries for all samples were run in one S4 flowcell on the Illumina Novaseq platform.

scRNA-Seq and CITE-Seq Data Filtration and Integration

Sequenced reads from each sample were aligned to the human reference genome GRCh38 and processed using the Cell Ranger v7.0.0 (10× Genomics) “multi” pipeline that generated count matrices from the GEX libraries, and assembled full TCR contigs from the VDJ libraries along with cell-surface protein expression from the Feature Barcoding libraries. On average, we achieved >70K mean reads per cell with >9000 mean UMIs per cell, and a median of >3,300 genes per cell. GEX (RNA) and Feature Barcoding (protein) count matrices from each sample were combined and loaded with Seurat v4.2.0 (Satija Lab), and barcoded cells were filtered for cells with outlier UMI counts <3000 (low quality cells) and >45000 (indicative of doublets), high mitochondrial gene expression (due to cellular stress or loss of cytoplasmic RNA), and low number of sequenced genes (<1200).

After initial data filtration for low-quality and outlier cells, the combined Seurat object was split by each modality, RNA and Protein, and then batch corrected for technical and biological variations using the Reciprocal Principal Component Analysis (RPCA) integration method in Seurat. Seurat utilized an unsupervised framework to learn cell-specific modality weights that allows integrated cell clustering based on both modalities. For integration of the combined RNA modality, molecular count data for each sample were individually normalized and variance stabilized using SCTransform, which bypasses the need for pseudocount addition and log-transformation, and then cell cycle phase scores were calculated for each individual sample based on the expression of canonical cell cycle genes within a specific barcoded cell. Following cell cycle scoring, raw counts were normalized and variance stabilized again using SCTransform with the additional step of regressing calculated cell cycle scores in order to mitigate the effects of cell cycle heterogeneity. In order to perform RPCA integration, highly variable genes (nfeatures=3000) were then identified from each sample and then used to find integration anchors between datasets (k.anchor=10). For integration of the protein modality, samples were individually normalized using centered log ratio transformation (CLR) prior to identification of highly variable features (nfeatures=3000). Samples were then scaled and PCAs were calculated for log-normalized integration of datasets.

Weighted Nearest Neighbor Multimodal Analysis of scRNA-Seq and CITE-Seq Data scRNA-Seq Clustering and Visualization

Integrated Seurat objects of all samples from both modalities (RNA and surface protein) were combined and PCA were calculated for both modalities with the first 50 PCs taken for gene expression (RNA) and first 20 PCs for feature barcoding (surface protein) datasets. Visualization and clustering of both modalities was performed using Weighted Nearest Neighbor (WNN) multimodal analysis in Seurat v4.2.0, which utilizes an unsupervised framework to learn cell-specific modality weights that allow integrated cell clustering on both modalities (RNA and surface protein) at multiple resolutions (0.6, 0.8, and 1.0). Using the 1.0 resolution, clusters were labeled and collapsed into T cell developmental subsets (CD34, DN, ISP4, DP Early, DP Late, SP8+TY5, NK, pDC) based on expression of surface protein as well as RNA expression of key T cell developmental markers. Notably, two populations were removed from the dataset based on irregular gene expression: one population expressed both hCD45 and hDLL4, which could have been epithelial or stromal cells carried over from bone marrow aspirate collection of CD34+ cells used for generation of ATOs; and the other population stained for most antibodies, indicating the presence of a myeloid-lineage cell population.

Following initial labeling, specific subpopulations were subset out of the combined datasets and individually examined for key T cell developmental markers from surface protein and RNA expression profiles at high clustering resolutions in order to confirm cell identities, and correct for any grouping errors as a result of high order clustering of all cells from the combined datasets: the “CD34+” cluster was redefined, as only a specific subset expressed CD34 RNA within the cluster, with the remaining cells categorized as “DN”; a population of “B” cells were identified within the “DN” population, which expressed both CD19 transcriptionally and on the cell surface; and all DP populations (DP Early, DP Late) were redefined at higher resolution based on WNN_UMAP mapping coordinates (DP Early) and surface expression of TCRαβ and CD3 (DP Late).

To identify “SP8” T cells from the “SP8+Tγδ” population from high order clustering, fully reconstructed TCR contigs from VDJ sequencing libraries were added as metadata for their corresponding cell identities into the Seurat object using scRepertoire v1.7.2 (//www.ncbi.nlm.nih.gov/pmc/articles/PMC7400693/). Based on cell surface expression of TCRαβ and metadata from full TCR contigs, “SP8” T cells were separated from “γδ” T cells, as sequencing of Tγδ TCRs was not performed. Further analysis of the “SP8” T cell population identified the “SP8RA” (CD45RA+CD45RO−) and “SP8RO” (CD45RA−CD45RA+) subsets.

Visualization and Identification of Gene-Edited Cells from scRNA-Seq

Cellular barcodes from cleaned datasets were extracted from the integrated Seurat object and exported as individual lists for the identification of cells that were gene-corrected from scRNA-seq datasets. Cellular barcode lists were used by cb_sniffer (//github.com/sridnona/cb_sniffer) to call mutant and edited RNA transcripts for CD3D (Chr 11:118340447-118340447, G [“Reference”]→A [“Mutant”]) from BAM outputs from the Cell Ranger v7.0.0 (10× Genomics) “multi” pipeline alignment to the GRCh38 reference genome. Cells were assigned as “Biallelic” (Reference>0, Mutant=0), “Monoallelic” (Reference>0, Mutant>0), and “Uncorrected” (Reference=0, Mutant>0) based on the presence of reference and mutant CD3D RNA from BAM alignments. Cells that did not have read for CD3D RNA were labeled as “Dropout” due to dropouts that can occur stochastically from scRNA-sequencing. Cellular labels were added back into the Seurat object as metadata, and visualization was performed on the WNN_UMAP.

Visualization and Identification of TCR Rearrangements within scRNA-Seq Datasets

The integrated Seurat object including fully reconstructed TCRs in the metadata from VDJ sequencing was analyzed in order to visualize and identify cells that expressed no TRAV or TRBV, only TRBV, and both TRAV+TRBV. From the GEX sequencing data (RNA) in the integrated Seurat object, cells expressing no TRAV or TRBV, only TRBV, and both TRBV+TRAV were identified and labeled in a separate column of the metadata. As RNA sequencing of total genes could lead to dropouts, fully reconstructed TCRs from VDJ sequencing within the metadata of the Seurat object were also analyzed to determine cells that had no TRAV or TRBV, only TRBV, and both TRBV+TRAV in an additional column of the metadata. After identifying the intersections between both columns of the metadata (GEX and VDJ), visualization of TCR rearrangements within the datasets was performed on the WNN_UMAP. Circle plots were generated using the circlize v0.4.15 package⁶² using VDJ sequencing data embedded in the Seurat object with scRepetoire, as described above.

Differentially Expressed Gene Analysis of scRNA-Seq Datasets

Differentially expressed genes (DEGs) were calculated using the “MAST” algorithm 63 which is tailored to scRNA-seq data DEG analysis using a model that parameterizes both stochastic dropout and characteristic bimodal expression distributions, for the FindMarkers function of Seurat (min.pct=0.1, log fc.threshold=0.25), and DEGs were visualized using EnhancedVolcano v1.14.065 (//github.com/kevinblighe/EnhancedVolcano) DEGs from FindMarkers were used to generate ranked gene lists ordered by log-fold change for Gene Set Enrichment Analysis (GSEA) using the fgsea v1.22.066 package and gene signatures were pulled from the Molecular Signatures Database (MSigDB) using msigdbr v7.15.167 (<//CRAN.R-project.org/package=msigdbr>). Visualization of GSEA results was performed using the enrichplot v1.16.2 package68 (//yulab-smu.top/biomedical-knowledge-mining-book/).

### Quantification and Statistical Analysis

In all figures, n represents independent biological replicates and data are represented as mean±standard deviation (SD). Statistical analysis was performed using GraphPad Prism software and p-values were calculated from the two-tailed unpaired t test or multiple t test, unless otherwise noted in figure legend. p-values of *p<0.05; **p<0.01; and ***p<0.001, ****p<0.0001 were considered statistically significant, unless otherwise noted in figure legend.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### SEQUENCE LISTING

SEQ ID NO: 3:
NRTH-ABE8e: A10T, I322V, S409I, E427G, R654L, R753G, R1114G,
D1135N, D1180G, G1218S, E1219V, Q1221H, P1249S, E1253K,
P1321S, D1332G, and R1335L″ sequence including
SV40 NLS and bGH Poly(A) signal
(5′-atgaaacggacagccgacggaagcgagttcgagtcaccaaagaagaagcggaaagt
ctctgaggtggagttttcccacgagtactggatgagacatgccctgaccctggccaagag
ggcacgggatgagagggaggtgcctgtgggagccgtgctggtgctgaacaatagagtgat
cggcgagggctggaacagagccatcggcctgcacgacccaacagcccatgccgaaattat
ggccctgagacagggcggcctggtcatgcagaactacagactgattgacgccaccctgta
cgtgacattcgagccttgcgtgatgtgcgccggcgccatgatccactctaggatcggccg
cgtggtgtttggcgtgaggaactcaaaaagaggcgccgcaggctccctgatgaacgtgct
gaactaccccggcatgaatcaccgcgtcgaaattaccgagggaatcctggcagatgaatg
tgccgccctgctgtgcgatttctatcggatgcctagacaggtgttcaatgctcagaagaa
ggcccagagctccatcaactccggaggatctagcggaggctcctctggctctgagacacc
tggcacaagcgagagcgcaacacctgaaagcagcgggggcagcagcggggggtcagacaa
gaagtacagcatcggcctgaccatcggcaccaactctgtgggctgggccgtgatcaccga
cgagtacaaggtgcccagcaagaaattcaaggtgctgggcaacaccgaccggcacagcat
caagaagaacctgatcggagccctgctgttcgacagcggcgaaacagccgaggccacccg
gctgaagagaaccgccagaagaagatacaccagacggaagaaccggatctgctatctgca
agagatcttcagcaacgagatggccaaggtggacgacagcttcttccacagactggaaga
gtccttcctggtggaagaggataagaagcacgagcggcaccccatcttcggcaacatcgt
ggacgaggtggcctaccacgagaagtaccccaccatctaccacctgagaaagaaactggt
ggacagcaccgacaaggccgacctgcggctgatctatctggccctggcccacatgatcaa
gttccggggccacttcctgatcgagggcgacctgaaccccgacaacagcgacgtggacaa
gctgttcatccagctggtgcagacctacaaccagctgttcgaggaaaaccccatcaacgc
cagcggcgtggacgccaaggccatcctgtctgccagactgagcaagagcagacggctgga
aaatctgatcgcccagctgcccggcgagaagaagaatggcctgttcggaaacctgattgc
cctgagcctgggcctgacccccaacttcaagagcaacttcgacctggccgaggatgccaa
actgcagctgagcaaggacacctacgacgacgacctggacaacctgctggcccagatcgg
cgaccagtacgccgacctgtttctggccgccaagaacctgtccgacgccatcctgctgag
cgacatcctgagagtgaacaccgagatcaccaaggcccccctgagcgcctctatggtgaa
gagatacgacgagcaccaccaggacctgaccctgctgaaagctctcgtgcggcagcagct
gcctgagaagtacaaagagattttcttcgaccagagcaagaacggctacgccggctacat
tgacggcggagccagccaggaagagttctacaagttcatcaagcccatcctggaaaagat
ggacggcaccgaggaactgctcgtgaagctgaacagagaggacctgctgcggaagcagcg
gaccttcgacaacggcattatcccccaccagatccacctgggagagctgcacgccattct
gcggcggcagggcgatttttacccattcctgaaggacaaccgggaaaagatcgagaagat
cctgaccttccgcatcccctactacgtgggccctctggccaggggaaacagcagattcgc
ctggatgaccagaaagagcgaggaaaccatcaccccctggaacttcgaggaagtggtgga
caagggcgcttccgcccagagcttcatcgagcggatgaccaacttcgataagaacctgcc
caacgagaaggtgctgcccaagcacagcctgctgtacgagtacttcaccgtgtataacga
gctgaccaaagtgaaatacgtgaccgagggaatgagaaagcccgccttcctgagcggcga
gcagaaaaaggccatcgtggacctgctgttcaagaccaaccggaaagtgaccgtgaagca
gctgaaagaggactacttcaagaaaatcgagtgcttcgactccgtggaaatctccggcgt
ggaagatcggttcaacgcctccctgggcacataccacgatctgctgaaaattatcaagga
caaggacttcctggacaatgaggaaaacgaggacattctggaagatatcgtgctgaccct
gacactgtttgaggacagagagatgatcgaggaacggctgaaaacctatgcccacctgtt
cgacgacaaagtgatgaagcagctgaagcggctgagatacaccggctggggcaggctgag
ccggaagctgatcaacggcatccgggacaagcagtccggcaagacaatcctggatttcct
gaagtccgacggcttcgccaacagaaacttcatgcagctgatccacgacgacagcctgac
ctttaaagaggacatccagaaagcccaggtgtccggccagggcgatagcctgcacgagca
cattgccaatctggccggcagccccgccattaagaagggcatcctgcagacagtgaaggt
ggtggacgagctcgtgaaagtgatgggcggccacaagcccgagaacatcgtgatcgaaat
ggccagagagaaccagaccacccagaagggacagaagaacagccgcgagagaatgaagcg
gatcgaagagggcatcaaagagctgggcagccagatcctgaaagaacaccccgtggaaaa
cacccagctgcagaacgagaagctgtacctgtactacctgcagaatgggcgggatatgta
cgtggaccaggaactggacatcaaccggctgtccgactacgatgtggaccatatcgtgcc
tcagagctttctgaaggacgactccatcgacaacaaggtgctgaccagaagcgacaagaa
ccggggcaagagcgacaacgtgccctccgaagaggtcgtgaagaagatgaagaactactg
gcggcagctgctgaacgccaagctgattacccagagaaagttcgacaatctgaccaaggc
cgaaagaggcggcctgagcgaactggataaggccggcttcatcaagagacagctggtgga
aacccggcagatcacaaagcacgtggcacagatcctggactcccggatgaacactaagta
cgacgagaatgacaagctgatccgggaagtgaaagtgatcaccctgaagtccaagctggt
gtccgatttccggaaggatttccagttttacaaagtgcgcgagatcaacaactaccacca
cgcccacgacgcctacctgaacgccgtcgtgggaaccgccctgatcaaaaagtaccctaa
gctggaaagcgagttcgtgtacggcgactacaaggtgtacgacgtgcggaagatgatcgc
caagagcgagcaggaaatcggcaaggctaccgccaagtacttcttctacagcaacatcat
gaactttttcaagaccgagattaccctggccaacggcgagatccggaagcggcctctgat
cgagacaaacggcgaaaccggggagatcgtgtgggataagggccgggattttgccaccgt
gcggaaagtgctgagcatgccccaagtgaatatcgtgaaaaagaccgaggtgcagacagg
cggcttcagcaaagagtctatcctgcccaagggcaacagcgataagctgatcgccagaaa
gaaggactgggaccctaagaagtacggcggcttcaacagccccaccgtggcctattctgt
gctggtggtggccaaagtggaaaagggcaagtccaagaaactgaagagtgtgaaagagct
gctggggatcaccatcatggaaagaagcagcttcgagaagaatcccatcggctttctgga
agccaagggctacaaagaagtgaaaaaggacctgatcatcaagctgcctaagtactccct
gttcgagctggaaaacggccggaagagaatgctggcctctgccagcgtgctgcataaggg
aaacgaactggccctgccctccaaatatgtgaacttcctgtacctggccagccactatga
gaagctgaagggctccagcgaggataataaacagaaacagctgtttgtggaacagcacaa
gcactacctggacgagatcatcgagcagatcagcgagttctccaagagagtgatcctggc
cgacgctaatctggacaaagtgctgtccgcctacaacaagcaccgggataagcccatcag
agagcaggccgagaatatcatccacctgtttaccctgaccaatctgggagccagcgccgc
cttcaagtactttgacaccaccatcggccggaagctgtacaccagcaccaaagaggtgct
ggacgccaccctgatccaccagagcatcaccggcctgtacgagacacggatcgacctgtc
tcagctgggaggtgactctggcggctcaaaaagaaccgccgacggcagcgaattcgagcc
caagaagaagaggaaagtctaaccggtcatcatcaccatcaccattgagtttaaacccgc
tgatcagcctcgactgtgccttctagttgccagccatctgttgtttgcccctcccccgtg
ccttccttgaccctggaaggtgccactcccactgtcctttcctaataaaatgaggaaatt
gcatcgcattgtctgagtaggtgtcattctattctggggggtggggggggcaggacagca
agggggaggattgggaagacaatagcaggcatgctggggatgcggtgggctctatgg-3′);
SEQ ID NO: 4: NRTH-ABE8e: A10T, I322V, S409I, E427G, R654L,
R753G, R1114G, D1135N, D1180G, G1218S, E1219V, Q1221H,
P1249S, E1253K, P1321S, D1332G, and
R1335L″
MKRTADGSEFESPKKKRKVSEVEFSHEYWMRHALTLAKRARDEREVPVGAVLVLNNRVIG
EGWNRAIGLHDPTAHAEIMALRQGGLVMQNYRLIDATLYVTFEPCVMCAGAMIHSRIGRV
VFGVRNSKRGAAGSLMNVLNYPGMNHRVEITEGILADECAALLCDFYRMPRQVFNAQKKA
QSSINSGGSSGGSSGSETPGTSESATPESSGGSSGGSDKKYSIGLTIGTNSVGWAVITDE
YKVPSKKFKVLGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPTIYHLRKKLVD
STDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMVKR
YDEHHQDLTLLKALVRQQLPEKYKEIFFDQSKNGYAGYIDGGASQEEFYKFIKPILEKMD
GTEELLVKLNREDLLRKQRTFDNGIIPHQIHLGELHAILRRQGDFYPFLKDNREKIEKIL
TFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQL
KEDYFKKIECFDSVEISGVEDRFNASLGTYHDLLKIIKDKDFLDNEENEDILEDIVLTLT
LFEDREMIEERLKTYAHLFDDKVMKQLKRLRYTGWGRLSRKLINGIRDKQSGKTILDFLK
SDGFANRNFMQLIHDDSLTFKEDIQKAQVSGQGDSLHEHIANLAGSPAIKKGILQTVKVV
DELVKVMGGHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRSDKNR
GKSDNVPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNIVKKTEVQTGG
FSKESILPKGNSDKLIARKKDWDPKKYGGFNSPTVAYSVLVVAKVEKGKSKKLKSVKELL
GITIMERSSFEKNPIGFLEAKGYKEVKKDLIIKLPKYSLFELENGRKRMLASASVLHKGN
ELALPSKYVNFLYLASHYEKLKGSSEDNKQKQLFVEQHKHYLDEIIEQISEFSKRVILAD
ANLDKVLSAYNKHRDKPIREQAENIIHLFTLTNLGASAAFKYFDTTIGRKLYTSTKEVLD
ATLIHQSITGLYETRIDLSQLGGDSGGSKRTADGSEFEPKKKRKV*PVIITITIEFKPAD
QPRLCLLVASHLLFAPPPCLP*PWKVPLPLSFPNKMRKLHRIV*VGVILFWGVGWGRTAR
GRIGKTIAGMLGMRWALW-3′)
(SEQ ID NO: 5) VRER-ABE8e: D1135V, G1218R, R1335E, and T1337R:
5′-atgaaacggacagccgacggaagcgagttcgagtcaccaaagaagaagcggaaagtc
tctgaggtggagttttcccacgagtactggatgagacatgccctgaccctggccaagagg
gcacgggatgagagggaggtgcctgtgggagccgtgctggtgctgaacaatagagtgatc
ggcgagggctggaacagagccatcggcctgcacgacccaacagcccatgccgaaattatg
gccctgagacagggcggcctggtcatgcagaactacagactgattgacgccaccctgtac
gtgacattcgagccttgcgtgatgtgcgccggcgccatgatccactctaggatcggccgc
gtggtgtttggcgtgaggaactcaaaaagaggcgccgcaggctccctgatgaacgtgctg
aactaccccggcatgaatcaccgcgtcgaaattaccgagggaatcctggcagatgaatgt
gccgccctgctgtgcgatttctatcggatgcctagacaggtgttcaatgctcagaagaag
gcccagagctccatcaactccggaggatctagcggaggctcctctggctctgagacacct
ggcacaagcgagagcgcaacacctgaaagcagcgggggcagcagcggggggtcagacaag
aagtacagcatcggcctggccatcggcaccaactctgtgggctgggccgtgatcaccgac
gagtacaaggtgcccagcaagaaattcaaggtgctgggcaacaccgaccggcacagcatc
aagaagaacctgatcggagccctgctgttcgacagcggcgaaacagccgaggccacccgg
ctgaagagaaccgccagaagaagatacaccagacggaagaaccggatctgctatctgcaa
gagatcttcagcaacgagatggccaaggtggacgacagcttcttccacagactggaagag
tccttcctggtggaagaggataagaagcacgagcggcaccccatcttcggcaacatcgtg
gacgaggtggcctaccacgagaagtaccccaccatctaccacctgagaaagaaactggtg
gacagcaccgacaaggccgacctgcggctgatctatctggccctggcccacatgatcaag
ttccggggccacttcctgatcgagggcgacctgaaccccgacaacagcgacgtggacaag
ctgttcatccagctggtgcagacctacaaccagctgttcgaggaaaaccccatcaacgcc
agcggcgtggacgccaaggccatcctgtctgccagactgagcaagagcagacggctggaa
aatctgatcgcccagctgcccggcgagaagaagaatggcctgttcggaaacctgattgcc
ctgagcctgggcctgacccccaacttcaagagcaacttcgacctggccgaggatgccaaa
ctgcagctgagcaaggacacctacgacgacgacctggacaacctgctggcccagatcggc
gaccagtacgccgacctgtttctggccgccaagaacctgtccgacgccatcctgctgagc
gacatcctgagagtgaacaccgagatcaccaaggcccccctgagcgcctctatgatcaag
agatacgacgagcaccaccaggacctgaccctgctgaaagctctcgtgcggcagcagctg
cctgagaagtacaaagagattttcttcgaccagagcaagaacggctacgccggctacatt
gacggcggagccagccaggaagagttctacaagttcatcaagcccatcctggaaaagatg
gacggcaccgaggaactgctcgtgaagctgaacagagaggacctgctgcggaagcagcgg
accttcgacaacggcagcatcccccaccagatccacctgggagagctgcacgccattctg
cggcggcaggaagatttttacccattcctgaaggacaaccgggaaaagatcgagaagatc
ctgaccttccgcatcccctactacgtgggccctctggccaggggaaacagcagattcgcc
tggatgaccagaaagagcgaggaaaccatcaccccctggaacttcgaggaagtggtggac
aagggcgcttccgcccagagcttcatcgagcggatgaccaacttcgataagaacctgccc
aacgagaaggtgctgcccaagcacagcctgctgtacgagtacttcaccgtgtataacgag
ctgaccaaagtgaaatacgtgaccgagggaatgagaaagcccgccttcctgagcggcgag
cagaaaaaggccatcgtggacctgctgttcaagaccaaccggaaagtgaccgtgaagcag
ctgaaagaggactacttcaagaaaatcgagtgcttcgactccgtggaaatctccggcgtg
gaagatcggttcaacgcctccctgggcacataccacgatctgctgaaaattatcaaggac
aaggacttcctggacaatgaggaaaacgaggacattctggaagatatcgtgctgaccctg
acactgtttgaggacagagagatgatcgaggaacggctgaaaacctatgcccacctgttc
gacgacaaagtgatgaagcagctgaagcggcggagatacaccggctggggcaggctgagc
cggaagctgatcaacggcatccgggacaagcagtccggcaagacaatcctggatttcctg
aagtccgacggcttcgccaacagaaacttcatgcagctgatccacgacgacagcctgacc
tttaaagaggacatccagaaagcccaggtgtccggccagggcgatagcctgcacgagcac
attgccaatctggccggcagccccgccattaagaagggcatcctgcagacagtgaaggtg
gtggacgagctcgtgaaagtgatgggccggcacaagcccgagaacatcgtgatcgaaatg
gccagagagaaccagaccacccagaagggacagaagaacagccgcgagagaatgaagcgg
atcgaagagggcatcaaagagctgggcagccagatcctgaaagaacaccccgtggaaaac
acccagctgcagaacgagaagctgtacctgtactacctgcagaatggggggatatgtacg
tggaccaggaactggacatcaaccggctgtccgactacgatgtggaccatatcgtgcctc
agagctttctgaaggacgactccatcgacaacaaggtgctgaccagaagcgacaagaacc
ggggcaagagcgacaacgtgccctccgaagaggtcgtgaagaagatgaagaactactggc
ggcagctgctgaacgccaagctgattacccagagaaagttcgacaatctgaccaaggccg
agagaggcggcctgagcgaactggataaggccggcttcatcaagagacagctggtggaaa
cccggcagatcacaaagcacgtggcacagatcctggactcccggatgaacactaagtacg
acgagaatgacaagctgatccgggaagtgaaagtgatcaccctgaagtccaagctggtgt
ccgatttccggaaggatttccagttttacaaagtgcgcgagatcaacaactaccaccacg
cccacgacgcctacctgaacgccgtcgtgggaaccgccctgatcaaaaagtaccctaagc
tggaaagcgagttcgtgtacggcgactacaaggtgtacgacgtgcggaagatgatcgcca
agagcgagcaggaaatcggcaaggctaccgccaagtacttcttctacagcaacatcatga
actttttcaagaccgagattaccctggccaacggcgagatccggaagcggcctctgatcg
agacaaacggcgaaaccggggagatcgtgtgggataagggccgggattttgccaccgtgc
ggaaagtgctgagcatgccccaagtgaatatcgtgaaaaagaccgaggtgcagacaggcg
gcttcagcaaagagtctatcctgcccaagaggaacagcgataagctgatcgccagaaaga
aggactgggaccctaagaagtacggcggcttcgtgagccctacagttgcctattctgtcc
tagtagtggcaaaagttgagaagggaaaatccaagaaactgaagtcagtcaaagaattat
tggggataacgattatggagcgctcgtcttttgaaaagaaccccatcgacttccttgagg
cgaaaggttacaaggaagtaaaaaaggatctcataattaaactaccaaagtatagtctgt
ttgagttagaaaatggccgaaaacggatgttggctagcgccagagagcttcaaaagggga
acgaactcgcactaccgtctaaatacgtgaatttcctgtatttagcgtcccattacgaga
agttgaaaggttcacctgaagataacgaacagaagcaactttttgttgagcagcacaaac
attatctcgacgaaatcatagagcaaatttcggaattcagtaagagagtcatcctagctg
atgccaatctggacaaagtattaagcgcatacaacaagcacagggataaacccatacgtg
agcaggcggaaaatattatccatttgtttactcttaccaacctcggcgctccagccgcat
tcaagtattttgacacaacgatagatcgcaaagagtacagatctaccaaggaggtgctag
acgcgacactgattcaccaatccatcacgggattatatgaaactcggatagatttgtcac
agcttgggggtgactctggcggctcaaaaagaaccgccgacggcagcgaattcgagccca
agaagaagaggaaagtctaaccggtcatcatcaccatcaccattgagtttaaacccgctg
atcagcctcgactgtgccttctagttgccagccatctgttgtttgcccctcccccgtgcc
ttccttgaccctggaaggtgccactcccactgtcctttcctaataaaatgaggaaattgc
atcgcattgtctgagtaggtgtcattctattctggggggggggggggcaggacagcaagg
gggaggattgggaagacaatagcaggcatgctggggatgcggtgggctctatgg-3′
(SEQ ID NO: 6)
VRER-ABE8e: D1135V, G1218R, R1335E, and T1337R Protein Seq:
MKRTADGSEFESPKKKRKVSEVEFSHEYWMRHALTLAKRARDEREVPVGAVLVLNNRVIG
EGWNRAIGLHDPTAHAEIMALRQGGLVMQNYRLIDATLYVTFEPCVMCAGAMIHSRIGRV
VFGVRNSKRGAAGSLMNVLNYPGMNHRVEITEGILADECAALLCDFYRMPRQVFNAQKKA
QSSINSGGSSGGSSGSETPGTSESATPESSGGSSGGSDKKYSIGLAIGTNSVGWAVITDE
YKVPSKKFKVLGNTDRHSIKKNLIGALLFDSGETAEATRLKRTARRRYTRRKNRICYLQE
IFSNEMAKVDDSFFHRLEESFLVEEDKKHERHPIFGNIVDEVAYHEKYPTIYHLRKKLVD
STDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKL
QLSKDTYDDDLDNLLAQIGDQYADLFLAAKNLSDAILLSDILRVNTEITKAPLSASMIKR
YDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQL
KEDYFKKIECFDSVEISGVEDRFNASLGTYHDLLKIIKDKDFLDNEENEDILEDIVLTLT
LFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRSDKNR
GKSDNVPSEEVVKKMKNYWRQLLNAKLITQRKFDNLTKAERGGLSELDKAGFIKRQLVET
RQITKHVAQILDSRMNTKYDENDKLIREVKVITLKSKLVSDFRKDFQFYKVREINNYHHA
HDAYLNAVVGTALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNIVKKTEVQTGG
FSKESILPKRNSDKLIARKKDWDPKKYGGFVSPTVAYSVLVVAKVEKGKSKKLKSVKELL
GITIMERSSFEKNPIDFLEAKGYKEVKKDLIIKLPKYSLFELENGRKRMLASARELQKGN
ELALPSKYVNFLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRKEYRSTKEVLD
ATLIHQSITGLYETRIDLSQLGGDSGGSKRTADGSEFEPKKKRKV*PVIITITIEFKPAD
QPRLCLLVASHLLFAPPPCLP*PWKVPLPLSFPNKMRKLHRIV*VGVILFWGVGWGRTAR
GRIGKTIAGMLGMRWALW
(SEQ ID NO: 7)
A262T, R324L, S4091, E480K, E543D, M6941, and E1219V″ DNA
atgaaacggacagccgacggaagcgagttcgagtcaccaaagaagaagcggaaagtctct
gaggtggagttttcccacgagtactggatgagacatgccctgaccctggccaagagggca
cgggatgagagggaggtgcctgtgggagccgtgctggtgctgaacaatagagtgatcggc
gagggctggaacagagccatcggcctgcacgacccaacagcccatgccgaaattatggcc
ctgagacagggcggcctggtcatgcagaactacagactgattgacgccaccctgtacgtg
acattcgagccttgcgtgatgtgcgccggcgccatgatccactctaggatcggccgcgtg
gtgtttggcgtgaggaactcaaaaagaggcgccgcaggctccctgatgaacgtgctgaac
taccccggcatgaatcaccgcgtcgaaattaccgagggaatcctggcagatgaatgtgcc
gccctgctgtgcgatttctatcggatgcctagacaggtgttcaatgctcagaagaaggcc
cagagctccatcaactccggaggatctagcggaggctcctctggctctgagacacctggc
acaagcgagagcgcaacacctgaaagcagcgggggcagcagcggggggtcagacaagaag
tacagcatcgggacaagaagtactccattgggctcgctatcggcacaaacagcgtcggct
gggccgtcattacggacgagtacaaggtgccgagcaaaaaattcaaagttctgggcaata
ccgatcgccacagcataaagaagaacctcattggcgccctcctgttcgactccggggaga
cggccgaagccacgcggctcaaaagaacagcacggcgcagatatacccgcagaaagaatc
ggatctgctacctgcaggagatctttagtaatgagatggctaaggtggatgactctttct
tccataggctggaggagtcctttttggtggaggaggataaaaagcacgagcgccacccaa
tctttggcaatatcgtggacgaggtggcgtaccatgaaaagtacccaaccatatatcatc
tgaggaagaagcttgtagacagtactgataaggctgacttgcggttgatctatctcgcgc
tggcgcatatgatcaaatttcggggacacttcctcatcgagggggacctgaacccagaca
acagcgatgtcgacaaactctttatccaactggttcagacttacaatcagcttttcgaag
agaacccgatcaacgcatccggagttgacgccaaagcaatcctgagcgctaggctgtcca
aatcccggcggctcgaaaacctcatcgcacagctccctggggagaagaagaacggcctgt
ttggtaatcttatcgccctgtccctcgggctgacccccaactttaaatctaacttcgacc
tggccgaagataccaagcttcaactgagcaaagacacctacgatgatgatctcgacaatc
tgctggcccagatcggcgaccagtacgcagacctttttttggcggcaaagaacctgtcag
acgccattctgctgagtgatattctgcgagtgaacacggagatcaccaaagctccgctga
gcgctagtatgatcaagctctatgatgagcaccaccaagacttgactttgctgaaggccc
ttgtcagacagcaactgcctgagaagtacaaggaaattttcttcgatcagtctaaaaatg
gctacgccggatacattgacggcggagcaagccaggaggaattttacaaatttattaagc
ccatcttggaaaaaatggacggcaccgaggagctgctggtaaagcttaacagagaagatc
tgttgcgcaaacagcgcactttcgacaatggaatcatcccccaccagattcacctgggcg
aactgcacgctatcctcaggcggcaagaggatttctacccctttttgaaagataacaggg
aaaagattgagaaaatcctcacatttcggataccctactatgtaggccccctcgcccggg
gaaattccagattcgcgtggatgactcgcaaatcagaagagaccatcactccctggaact
tcgagaaagtcgtggataagggggcctctgcccagtccttcatcgaaaggatgactaact
ttgataaaaatctgcctaacgaaaaggtgcttcctaaacactctctgctgtacgagtact
tcacagtttataacgagctcaccaaggtcaaatacgtcacagaagggatgagaaagccag
cattcctgtctggagatcagaagaaagctattgtggacctcctcttcaagacgaaccgga
aagttaccgtgaaacagctcaaagaagactatttcaaaaagattgaatgtttcgactctg
ttgaaatcagcggagtggaggatcgcttcaacgcatccctgggaacgtatcacgatctcc
tgaaaatcattaaagacaaggacttcctggacaatgaggagaacgaggacattcttgagg
acattgtcctcacccttacgttgtttgaagatagggagatgattgaagaacgcttgaaaa
cttacgctcatctcttcgacgacaaagtcatgaagcagctcaagaggcgccgatatacag
gatgggggcggctgtcaagaaaactgatcaatgggatccgagacaagcagagtggaaaga
caatcctggattttcttaagtccgatggatttgccaaccggaacttcattcagttgatcc
atgatgactctctcacctttaaggaggacatccagaaagcacaagtttctggccaggggg
acagtcttcacgagcacatcgctaatcttgcaggtagcccagctatcaaaaagggaatac
tgcagaccgttaaggtcgtggatgaactcgtcaaagtaatgggaaggcataagcccgaga
atatcgttatcgagatggcccgagagaaccaaaccacccagaagggacagaagaacagta
gggaaaggatgaagaggattgaagagggtataaaagaactggggtcccaaatccttaagg
aacacccagttgaaaacacccagcttcagaatgagaagctctacctgtactacctgcaga
acggcagggacatgtacgtggatcaggaactggacatcaatcggctctccgactacgacg
tggatcatatcgtgccccagtcttttctcaaagatgattctattgataataaagtgttga
caagatccgataaaaacagagggaagagtgataacgtcccctcagaagaagttgtcaaga
aaatgaaaaattattggcggcagctgctgaacgccaaactgatcacacaacggaagttcg
ataatctgactaaggctgaacgaggtggcctgtctgagttggataaagccggtttcatca
aaaggcagcttgttgagacacgccagatcaccaagcacgtggcccaaattctcgattcac
gcatgaacaccaagtacgatgaaaatgacaaactgattcgagaggtgaaagttattactc
tgaagtctaagctggtctcagatttcagaaaggactttcagttttataaggtgagagaga
tcaacaattaccaccatgcgcatgatgcctacctgaatgcagtggtaggcactgcactta
tcaaaaaatatcccaagcttgaatctgaatttgtttacggagactataaagtgtacgatg
ttaggaaaatgatcgcaaagtctgagcaggaaataggcaaggccaccgctaagtacttct
tttacagcaatattatgaattttttcaagaccgagattacactggccaatggagagattc
ggaagcgaccacttatcgaaacaaacggagaaacaggagaaatcgtgtgggacaagggta
gggatttcgcgacagtccggaaggtcctgtccatgccgcaggtgaacatcgttaaaaaga
ccgaagtacagaccggaggcttctccaaggaaagtatcctcccgaaaaggaacagcgaca
agctgatcgcacgcaaaaaagattgggaccccaagaaatacggcggattcgattctccta
cagtcgcttacagtgtactggttgtggctaaagtggagaaagggaagtctaaaaaactca
aaagcgtcaaggaactgctgggcatcacaatcatggagcgatcaagcttcgaaaaaaacc
ccatcgactttctcgaggcgaaaggatataaagaggtcaaaaaagacctcatcattaagc
ttcccaagtactctctctttgagcttgaaaacggccggaaacgaatgctcgctagtgcgg
gcgtgctgcagaaaggtaacgagctggcactgccctctaaatacgttaatttcttgtatc
tggccagccactatgaaaagctcaaagggtctcccgaagataatgagcagaagcagctgt
tcgtggaacaacacaaacactaccttgatgagatcatcgagcaaataagcgaattctcca
aaagagtgatcctcgccgacgctaacctcgataaggtgctttctgcttacaataagcaca
gggataagcccatcagggagcaggcagaaaacattatccacttgtttactctgaccaact
tgggcgcgcctgcagccttcaagtacttcgacactaccatagacagaaagcggtacacct
ctacaaaggaggtcctggacgccacactgattcatcagtcaattacggggctctatgaaa
caagaatcgacctctctcagctcggtggagacccggcctgtacgagacacggatcgacct
gtctcagctgggaggtgactctggcggctcaaaaagaaccgccgacggcagcgaattcga
gcccaagaagaagaggaaagtctaaccggtcatcatcaccatcaccattgagtttaaacc
cgctgatcagcctcgactgtgccttctagttgccagccatctgttgtttgcccctccccc
gtgccttccttgaccctggaaggtgccactcccactgtcctttcctaataaaatgaggaa
attgcatcgcattgtctgagtaggtgtcattctattctggggggtggggggggcaggaca
gcaagggggaggattgggaagacaatagcaggcatgctggggatgcggtgggctctatg
g-3′
(SEQ ID NO: 8)
A262T, R324L, S4091, E480K, E543D, M694I, and E1219V″ Protein
MKRTADGSEFESPKKKRKVSEVEFSHEYWMRHALTLAKRARDEREVPVGAVLVLNNRVIG
EGWNRAIGLHDPTAHAEIMALRQGGLVMQNYRLIDATLYVTFEPCVMCAGAMIHSRIGRV
VFGVRNSKRGAAGSLMNVLNYPGMNHRVEITEGILADECAALLCDFYRMPRQVFNAQKKA
QSSINSGGSSGGSSGSETPGTSESATPESSGGSSGGSDKKYSIGTRSTPLGSLSAQTASA
GPSLRTSTRCRAKNSKFWAIPIATA*RRTSLAPSCSTPGRRPKPRGSKEQHGADIPAERI
GSATCRRSLVMRWLRWMTLSSIGWRSPFWWRRIKSTSATQSLAISWTRWRTMKSTQPYII
*GRSL*TVLIRLTCG*SISRWRI*SNFGDTSSSRGT*TQTTAMSTNSLSNWFRLTISFSK
RTRSTHPELTPKQS*ALGCPNPGGSKTSSHSSLGRRRTACLVILSPCPSG*PPTLNLTST
WPKIPSFN*AKTPTMMISTICWPRSATSTQTFFWRQRTCQTPFC*VIFCE*TRRSPKLR*
ALV*SSSMMSTTKT*LC*RPLSDSNCLRSTRKESSISLKMATPDTLTAEQARRNFTNLLS
PSWKKWTAPRSCW*SLTEKICCANSALSTMESSPTRFTWANCTLSSGGKRISTPF*KITG
KRLRKSSHFGYPTM*APSPGEIPDSRG*LANQKRPSLPGTSRKSWIRGPLPSPSSKG*LT
LIKICLTKRCFLNTLCCTSTSQFITSSPRSNTSQKG*ESQHSCLEIRRKLLWTSSSRRTG
KLP*NSSKKTISKRLNVSTLLKSAEWRIASTHPWERITIS*KSLKTRTSWTMRRTRTFLR
TLSSPLRCLKIGR*LKNA*KLTLISSTTKS*SSSRGADIQDGGGCQEN*SMGSETSRVER
QSWIFLSPMDLPTGTSFS*SMMTLSPLRRTSRKHKFLARGTVFTSTSLILQVAQLSKREY
CRPLRSWMNSSK*WEGISPRISLSRWPERTKPPRRDRRTVGKG*RGLKRV*KNWGPKSLR
NTQLKTPSFRMRSSTCTTCRTAGTCTWIRNWTSIGSPTTTWIISCPSLFSKMILLIIKC*
QDPIKTEGRVITSPQKKLSRK*KIIGGSC*TPN*SHNGSSII*LRLNEVACLSWIKPVSS
KGSLLRHARSPSTWPKFSIHA*TPSTMKMTN*FER*KLLL*SLSWSQISERTFSFIR*ER
STITTMRMMPT*MQW*ALHLSKNIPSLNLNLFTETIKCTMLGK*SQSLSRK*ARPPLSTS
FTAIL*IFSRPRLHWPMERFGSDHLSKQTEKQEKSCGTRVGISRQSGRSCPCRR*TSLKR
PKYRPEASPRKVSSRKGTATS*SHAKKIGTPRNTADSILLQSLTVYWLWLKWRKGSLKNS
KASRNCWASQSWSDQASKKTPSTFSRRKDIKRSKKTSSLSFPSTLSLSLKTAGNECSLVR
ACCRKVTSWHCPLNTLISCIWPATMKSSKGLPKIMSRSSCSWNNTNTTLMRSSSK*ANSP
KE*SSPTLTSIRCFLLTISTGISPSGSRQKTLSTCLL*PTWARLQPSSTSTLP*TESGTP
LQRRSWTPH*FISQLRGSMKQESTSLSSVEPACTRHGSTCLSWEVTLAAQKEPPTAANSS
PRRRGKSNRSSSPSPLSLNPLISLDCAF*LPAICCLPLPRAFLDPGRCHSHCPFLIK*GN
CIALSE*VSFYSGGWGGAGQQGGGLGRQ*QACWGCGGLY
(SEQ ID NO: 1107) Peclc-MNDU3-CD3D c.202C > T
gggtctctctggttagaccagatctgagcctgggagctctctggctaactagggaaccca
ctgcttaagcctcaataaagcttgccttgagtgcttcaagtagtgtgtgcccgtctgttg
tgtgactctggtaactagagatccctcagacccttttagtcagtgtggaaaatctctagc
agtggcgcccgaacagggacctgaaagcgaaagggaaaccagaggagctctctcgacgca
ggactcggcttgctgaagcgcgcacggcaagaggcgaggggcggcgactggtgagtacgc
caaaaattttgactagcggaggctagaaggagagagatgggtgcgagagcgtcagtatta
agcgggggagaattagatcgcgatgggaaaaaattcggttaaggccagggggaaagaaaa
aatataaattaaaacatatagtatgggcaagcagggagctagaacgattcgcagttaatc
ctggcctgttagaaacatcagaaggctgtagacaaatactgggacagctacaaccatccc
ttcagacaggatcagaagaacttagatcattatataatacagtagcaaccctctattgtg
tgcatcaaaggatagagataaaagacaccaaggaagctttagacaagatagaggaagagc
aaaacaaaagtaagaccaccgcacagcaagcggccgctgatcttcagacctggaggagga
gatatgagggacaattggagaagtgaattatataaatataaagtagtaaaaattgaacca
ttaggAGTAGCACCCACCAAGGCAAAGAGAAGAGTGGTGCAGAGAGAAAAAAGAGCAGTG
GGAATAGGAGCTTTGTTCCTTGGGTTCTTGGGAGCAGCAGGAAGCACTATGGGCGCAGCC
TCAATGACGCTGACGGTACAGGCCAGACAATTATTGTCTGGTATAGTGCAGCAGCAGAAC
AATTTGCTGAGGGCTATTGAGGCGCAACAGCATCTGTTGCAACTCACAGTCTGGGGCATC
AAGCAGCTCCAGGCAAGAATCCTGGCTGTGGAAAGATACCTAAAGGATCAACAGCTCCTG
GGGATTTGGGGTTGCTCTGGAAAACTCATTTGCACCACTGCTGTGCCTTGGAATGCTAGT
TGGAGTAATAAATCTCTGGAACAGATTGGAATCACACGACCTGGATGGAGTGGGACAGAG
AAATTAACAATTACACAAGCTTAATACACTCCTTAATTGAAGAATCGCAAAACCAGCAAG
AAAAGAATGAACAAGAATTATTGGAATTAGATAAATGGGCAAGTTTGTGGAATTGGTTTA
ACATAACAAATTGGCTGTGGTATATAAAATTATTCATAATGATAGTAGGAGGCTTGGTAG
GTTTAAGAATAGTTTTTGCTGTACTTTCTATAGTGAATAGAGTTAGGCAGGGATATTCAC
CATTATCGTTTCAGACCCACCTCCCAACCCCGAGGGGACCCGACAGGCCCGAAGGAATAG
AAGAAGAAGGTGGAGAGAGAGACAGAGACAGATCCATTCGATTAGTGAACGGATCTCGAC
GGTATCGATCTCGACACAAATGGCAGTATTCATCCACAATTTTAAAAGAAAAGGGGGGAT
TGGGGGGTACAGTGCAGGGGAAAGAATAGTAGACATAATAGCAACAGACATACAAACTAA
AGAATTACAAAAACAAATTACAAAAATTCAAAATTTTCGGGTTTATTACAGGGACAGCAG
AGATCCAGTTTGGGTCGAGGATATCGGATCTAGATCGATTAGTCCAATTTGTTAAAGACA
GGATATCAGTGGTCCAGGCTCTAGTTTTGACTCAACAATATCACCAGCTGAAGCCTATAG
AGTACGAGCCATAGATAAAATAAAAGATTTTATTTAGTCTCCAGAAAAAGGGGGGAATGA
AAGACCCCACCTGTAGGTTTGGCAAGCTAGGATCAAGGTCAGGAACAGAGAAACAGGAGA
ATATGGGCCAAACAGGATATCTGTGGTAAGCAGTTCCTGCCCCGCTCAGGGCCAAGAACA
GTTGGAACAGGAGAATATGGGCCAAACAGGATATCTGTGGTAAGCAGTTCCTGCCCCGCT
CAGGGCCAAGAACAGATGGTCCCCAGATGCGGTCCCGCCCTCAGCAGTTTCTAGAGAACC
ATCAGATGTTTCCAGGGTGCCCCAAGGACCTGAAATGACCCTGTGCCTTATTTGAACTAA
CCAATCAGTTCGCTTCTCGCTTCTGTTCGCGCGCTTCTGCTCCCCGAGCTCAATAAAAGA
GCCCACAACCCCTCACTCGGCGCGATCGATGAATTCGAGCTCGGTACCCGGGGATCCCGG
GTGATCAGTCGAGCTCAAGCTTCGAATTCTGCAGTCGACGGTACCGCGGGCCCGGGATCC
ACCGGTCGCCACCATGGAGCACAGCACCTTCCTGTCTGGCCTGGTACTGGCTACCCTTCT
CTCGCAAGTGAGCCCCTTCAAGATACCTATAGAGGAACTTGAGGACAGAGTGTTTGTGAA
TTGCAATACCAGCATCACATGGGTAGAGGGAACGGTGGGAACACTGCTCTCAGACATTAC
AAGACTGGACCTGGGAAAACGCATCCTGGACCCATGAGGAATATATAGGTGTAATGGGAC
AGATATATACAAGGACAAAGAATCTACCGTGCAAGTTCATTATCGAATGTGCCAGAGCTG
TGTGGAGCTGGATCCAGCCACCGTGGCTGGCATCATTGTCACTGATGTCATTGCCACTCT
GCTCCTTGCTTTGGGAGTCTTCTGCTTTGCTGGACATGAGACTGGAAGGCTGTCTGGGGC
TGCCGACACACAAGCTCTGTTGAGGAATGACCAGGTCTATCAGCCCCTCCGAGATCGAGA
TGATGCTCAGTACAGCCACCTGGGCGGCAACTGGGCTCGGAACAAGTGATAAAGCGGCCA
ACTCGACGGGCCCGCGGAATTCGAGCTCGGTACCTTTAAGACCAATGACTTACAAGGCAG
CTGTAGATCTTAGCCACTTTTTAAAAGAAAAGGGGGGACtggaagggctaattcactccc
aacgaagacaagatctgctttttgcttgtactgggtctctctggttagaccagatctgag
cctgggagctctctggctaactagggaacccactgcttaagcctcaataaagcttgcctt
gagtgcttcaagtagtgtgtgcccgtctgttgtgtgactctggtaactagagatccctca
gacccttttagtcagtgtggaaaatctctagca

1. A system for homology-directed repair (HDR)-mediated correction of the C202T mutation that produces CD3δ SCID disease, said system comprising:
a first single-guide RNA (sgRNA) that directs Cas9 cutting upstream of the C2020T mutation;
a second single-guide RNA (sgRNA) that directs Cas9 cutting downstream of the C2020T mutation; and
a single-strand oligodeoxynucleotide (ssODN) homologous donor comprising a nucleotide sequence that corrects the C202T mutation.

2. The system of claim 1, wherein said first single-guide RNA comprises a nucleotide sequence that directs Cas9 cutting two base pairs (bp) upstream C202T mutation or wherein said second single-guide RNA comprises a nucleotide sequence that directs Cas9 cutting five bp downstream of the C202T mutation or a combination thereof.

3. (canceled)

4. The system according to claim 1, wherein said ssODN is complementary to the nontarget strand with asymmetric homology arms, and optionally wherein said asymmetric homology arms extend 33 bp downstream and 60 bp upstream of the respective sgRNA-guided Cas9 cut site; or said ssODN further comprises a silent PAM mutation to prevent continual nuclease activity; or a combination thereof.

5. (canceled)

6. (canceled)

7. The system according to claim 1, wherein said system comprises a CRISPR protein or a nucleic acid encoding the CRISPR protein, or a CRISPR/cas9 protein or a nucleic acid encoding the CRISPR/cas9 protein.

8. (canceled)

9. (canceled)

10. (canceled)

11. (canceled)

12. (canceled)

13. The system according to claim 1, wherein said system is provided as kit comprising one or more containers containing:
said first single-guide RNA (sgRNA);
said second single-guide RNA (sgRNA); and
said single-strand oligodeoxynucleotide (ssODN); wherein said kit optionally comprises a container containing a CRISPR protein or a nucleic acid encoding a CRISPR protein or a CRISPR/Cas9 protein or a nucleic acid encoding the CRISPR/Cas9 protein.

14. (canceled)

15. (canceled)

16. A method of correcting a C202T mutation in a mammalian cell using homology-directed repair, said method comprising:
introducing a CRISPR protein, or a nucleic acid comprising the CRISPR protein, or a CRISPR/Cas9 protein, or a nucleic acid comprising the CRISPR/Cas9 protein, and the system according to claim 1 into said cell; and
culturing said cell to permit homology-directed repair (HDR-mediated correction) of the C202T mutation in said cell to provide a corrected cell; wherein said cell is from a human subject identified as having CD3δ severe combined immunodeficiency (SCID).

17. (canceled)

18. (canceled)

19. (canceled)

20. (canceled)

21. The method according to claim 16, wherein the cell is a stem/progenitor cell, wherein optionally said stem cell is derived from bone marrow, and/or from umbilical cord blood, and/or from peripheral blood, or said progenitor cell is a human hematopoietic progenitor cell.

22. (canceled)

23. (canceled)

24. (canceled)

25. (canceled)

26. The method according to claim 16, wherein said method further comprises introducing said corrected cell into a subject identified as having CD3δ severe combined immunodeficiency (SCID).

27. (canceled)

28. A method of treating a human subject for CD3δ severe combined immunodeficiency (SCID), said method comprising:
providing stem/progenitor cells from said subject;
correcting a C202T mutation in said cells ex vivo using the method according to claim 16 to produce corrected cells; and
introducing said corrected cells into said subject.

29. (canceled)

30. (canceled)

31. (canceled)

32. (canceled)

33. (canceled)

34. An adenosine base editor, wherein said base editor is a variant of the wildtype NGG-recognizing Cas9 (D10A) nickase (Cas9n) comprising a combination of amino acid substitutions selected from the group consisting of:
(1) NRTH-ABE8e: A10T, I322V, S409I, E427G, R654L, R753G, R1114G, D1135N, D1180G, G1218S, E1219V, Q1221H, P1249S, E1253K, P1321S, D1332G, and R1335L;
(2) VRER-ABE8e: D1135V, G1218R, R1335E, and T1337R; and
(3) A262T, R324L, S409I, E480K, E543D, M694I, and E1219V.

35. (canceled)

36. The base editor of claim 34, wherein
when said editor comprises the combination of amino acid substitutions: A10T, I322V, S409I, E427G, R654L, R753G, R1114G, D1135N, D1180G, G1218S, E1219V, Q1221H, P1249S, E1253K, P1321S, D1332G, and R1335L, said base editor comprises the amino acid sequence of SEQ ID NO:4 or is encoded by the nucleic acid sequence of SEQ ID NO:3; or
when said editor comprises the combination of amino acid substitutions: D1135V, G1218R, R1335E, and T1337R, said base editor comprises the amino acid sequence of SEQ ID NO:6 or is encoded by the nucleic acid sequence of SEQ ID NO:5; or
when said editor comprises the combination of amino acid substitutions: A262T, R324L, S409I, E480K, E543D, M694I, and E1219V, said base editor comprises the amino acid sequence of SEQ ID NO:8 or is encoded by the nucleic acid sequence of SEQ ID NO:7.

37. (canceled)

38. (canceled)

39. (canceled)

40. (canceled)

41. (canceled)

42. (canceled)

43. (canceled)

44. A nucleic acid encoding a base editor according to claim 34.

45. A system for base-editor-directed repair (BE-mediated correction) of a C202T mutation that produces CD3δ SCID disease, said system comprising:
a base editor according to claim 34, or a nucleic acid encoding a base editor according to claim 34; and
a single-guide RNA (sgRNA) that directs said base editor to the location of the nucleic acids encoding the C202T mutation.

46. The system of claim 45, wherein said sgRNA comprises the sequence of the G1 (Guide 2T) sgRNA (SEQ ID NO:1) or wherein said sgRNA comprises the sequence of the Guide 5T) sgRNA (SEQ ID NO:2).

47. (canceled)

48. A method of correcting a C202T mutation in a mammalian cell using Adenine Base Editing (ABE)-correction, said method comprising:
introducing a base editor according to claim 34, or a nucleic acid encoding a base editor according to claim 34, and a single-guide RNA (sgRNA) that directs said base editor to the location of the nucleic acids encoding the C202T mutation into said cell; and
culturing said cell to permit base editor (BE) mediated correction of the C202T mutation in said cell to provide a corrected cell, wherein said cell is from a human subject identified as having CD3δ severe combined immunodeficiency (SCID).

49. (canceled)

50. (canceled)

51. (canceled)

52. (canceled)

53. The method according to claim 48, wherein the cell is a stem/progenitor cell, wherein optionally said stem cell is derived from bone marrow and/or from umbilical cord blood and/or from peripheral blood, or said progenitor cell is a human hematopoietic progenitor cell.

54. (canceled)

55. (canceled)

56. (canceled)

57. (canceled)

58. The method according to claim 48, wherein said method further comprises introducing said corrected cell into a subject identified as having CD3δ severe combined immunodeficiency (SCID).

59. (canceled)

60. A method of treating a subject for CD3δ severe combined immunodeficiency (SCID), said method comprising:
providing stem/progenitor cells from said subject;
correcting a C202T mutation in said cells ex vivo using the method according to claim 48 to produce corrected cells; and
introducing said corrected cells into said subject.

61. (canceled)

62. (canceled)

63. (canceled)

64. (canceled)

65. (canceled)

66. A lentivirus for evaluating gene editing correction of the CD3δ SCID-causing C202T mutation, said lentivirus construct comprising the elements illustrated in FIG. 3.

67. The lentivirus of claim 66, wherein said lentivirus comprises the sequence of SEQ ID NO:1107.
